(19) United States
(12) Patent Application Publication
(10) Pub. No.: US 2021/0015764 A1
(43) Pub. Date: Jan. 21, 2021

(54) METHODS OF REDUCING INFLAMMATION OF THE DIGESTIVE SYSTEM WITH INHIBITORS OF HIF-2- ALPHA

(71) Applicant: Peloton Therapeutics Inc., Dallas, TX (US)

(72) Inventors: John A. Josey, Dallas, TX (US); Rajeev Shrimali, Plano, TX (US); Eli M. Wallace, Richardson, TX (US); Tai Wong, Dallas, TX (US)

(73) Assignee: Peloton Therapeutics Inc., Dallas, TX (US)

(21) Appl. No.: 16/980,648

(22) PCT Filed: Mar. 27, 2019
(86) PCT No.: PCT/US2019/024276

§ 371 Date: Sep. 14, 2020

Related U.S. Application Data
(60) Provisional application No. 62/649,338, filed on Mar. 28, 2018.

(51) Int. Cl. A61K 31/085; A61K 31/277; A61K 31/435

(52) U.S. Cl. CPC A61K 31/085; A61K 45/06; A61K 31/435; A61K 31/277

(57) ABSTRACT

The present disclosure provides methods of reducing inflammation of the digestive system in a subject in need thereof, including subjects suffering from inflammatory bowel disease. Compositions for use in these methods are also provided.

### CROSS-REFERENCE

This application claims the benefit of U.S. Provisional Application No. 62/649,338, filed Mar. 28, 2018, incorporated herein by reference in its entirety.

### BACKGROUND OF THE INVENTION

Inflammatory bowel disease is a chronic inflammatory disorder of the gastrointestinal tract affecting more than 10.5 million people worldwide, with approximately 3.1 million cases in the United States. Inflammatory bowel disease comprises Crohn's disease and ulcerative colitis. Crohn's disease affects the entire gastrointestinal tract, often with patches of damaged areas affecting multiple layers, while ulcerative colitis continuously affects the inner most layer of the colon and rectum. Abdominal pain, diarrhea, rectal bleeding and weight loss are common side effects of inflammatory bowel disease. Further complications include fistulas, toxic megacolon, anemia, intestinal fibrosis and even colon associated cancer. The intestinal epithelial barrier is disrupted in subjects suffering from inflammatory bowel disease, leading to loss of tissue integrity and exposure of intestinal microbiome to the underlying immune system, leading to excessive activation of the immune response and inflammation.

Currently, there is no curative treatment for Crohn's disease or ulcerative colitis. The treatment goal is typically to induce and maintain disease remission. The type of treatment depends on the severity of the disease. Anti-inflammatory drugs are generally used to treat Crohn's disease and ulcerative colitis, though some common treatments are associated with undesired side-effects. Additionally, the disease can become dependent upon the treatment or develop resistance to the treatment. In severe cases that fail to respond to therapeutic agents, surgical intervention may be required.

An adequate supply of oxygen to tissues is essential in maintaining mammalian cell function and physiology. A deficiency in oxygen supply to tissues is a characteristic of a number of pathophysiologic conditions in which there is insufficient blood flow to provide adequate oxygenation. The hypoxic (low oxygen) environment of tissues activates a signaling cascade that drives the induction or repression of the transcription of a multitude of genes implicated in events such as angiogenesis (neo-vascularization), glucose metabolism, and cell survival/death. A key to this hypoxic transcriptional response lies in the transcription factors, the hypoxia-inducible factors (HIF). HIFs are disregulated in a vast array of cancers through hypoxia-dependent and independent mechanisms and expression is associated with poor patient prognosis.

HIFs consist of an oxygen-sensitive HIFα subunit and a constitutively expressed HIFβ subunit. When HIFs are activated, the HIFα and HIF subunits assemble a functional heterodimer (the a subunit heterodimerizes with the Rsubunit). Both HIFα and HIFβ have two identical structural characteristics, a basic helix-loop-helix (bHLH) and PAS domains (PAS is an acronym referring to the first proteins, PER, ARNT, SIM, in which this motif was identified). There are three human HIFα subunits (HIF-1α, HIF-2α, and HIF-3α) that are oxygen sensitive. Among the three subunits, HIF-1α is the most ubiquitously expressed and induced by low oxygen concentrations in many cell and tissue types. HIF-2a is highly similar to HIF-1α in both structure and function, but exhibits more restricted cell and tissue-specific expression, and might also be differentially regulated by nuclear translocation. HIF-3a also exhibits conservation with HIF-1α and HIF-2a in the HLH and PAS domains. HIF-1β (also referred to as ARNT—Aryl Hydrocarbon Receptor Nuclear Translocator), the dimerization partner of the HIFα subunits, is constitutively expressed in all cell types and is not regulated by oxygen concentration.

### SUMMARY OF THE INVENTION

There remains a need for alternative therapeutic agents for reducing inflammation in the digestive system, especially in subjects suffering from moderate or severe Crohn's disease or ulcerative colitis. The present disclosure addresses this need by providing HIF-2a inhibitors and methods of using the HIF-2a inhibitors as described herein.

In certain aspects, the present disclosure provides a method of reducing inflammation of the digestive system in a subject in need thereof, comprising administering to the subject an effective amount of a HIF-2a inhibitor. In some embodiments, the subject suffers from inflammatory bowel disease. In some embodiments, the subject suffers from Crohn's disease or colitis, such as ulcerative colitis. In some embodiments, said administering induces remission of the inflammation. In some embodiments, the HIF-2a inhibitor reduces intestinal inflammation. In some embodiments, the HIF-2a inhibitor inhibits recruitment of inflammatory cells. In some embodiments, the HIF-2a inhibitor inhibits one or more biological effects selected from the group consisting of heterodimerization of HIF-2a to HIF-1β, HIF-2a target gene expression, VEGF gene expression, and VEGF protein secretion. In some embodiments, the HIF-2α inhibitor inhibits heterodimerization of HIF-2α to HIF-1β but not heterodimerization of HIF-1α to HIF-1β. In some embodiments, the HIF-2α inhibitor binds the PAS-B domain cavity of HIF-2α.

In practicing any of the subject methods, the HIF-2α inhibitor may be a compound of Formula I′:

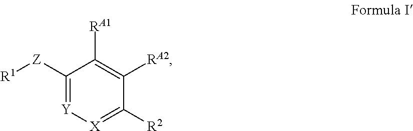

or a pharmaceutically acceptable salt or prodrug thereof, wherein:

X is selected from CR³ and N;

Y is selected from CR⁴ and N;

Z is selected from —O—, —S—, —S(O)—, —S(O)₂—, —C(O)—, —C(HR)—, —N(R⁶)—, C₁-C₃ alkylene, C₁-C₃ heteroalkylene, C₁-C₃ alkenylene or absent;

R¹ is selected from C₁₋₆ alkyl, C₃₋₁₂ carbocycle and 3- to 12-membered heterocycle, each of which is optionally substituted with one or more R²⁰;

R², R³, R⁴ and R⁵ are each independently selected from hydrogen and R²⁰;

R⁶ is selected from R²¹;

R^{A1} and R^{A2} are each independently selected from hydrogen and R²⁰, or R^{A1} and R^{A2} are taken together with the carbon atoms to which they are attached to form C₃₋₁₂ carbocycle or 3- to 12-membered heterocycle, each of which is optionally substituted with one or more R²⁰;

R²⁰ is independently selected at each occurrence from:
halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, and ═N(R²¹);
C₁₋₁₀ alkyl, C₂₋₁₀ alkenyl, and C₂₋₁₀ alkynyl, each of which is independently optionally substituted at each occurrence with one or more substituents selected from R²⁴; and
C₃₋₁₂ carbocycle and 3- to 12-membered heterocycle, each of which is independently optionally substituted at each occurrence with one or more substituents selected from R²⁵;

R²¹ is independently selected at each occurrence from hydrogen; and C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;

R²² and R²³ are taken together with the nitrogen atom to which they are attached to form a heterocycle, optionally substituted with one or more R²⁰;

R²⁴ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₃₋₁₂ carbocycle, and 3- to 12-membered heterocycle, wherein each C₃₋₁₂ carbocycle and 3- to 12-membered heterocycle is independently optionally substituted with one or more substituents selected from R²⁵; and

R²⁵ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₁₋₆ alkyl, C₁₋₆ haloalkyl, C₂₋₆ alkenyl, and C₂₋₆ alkynyl.

In practicing any of the subject methods, the HIF-2α inhibitor may be a compound of Formula I:

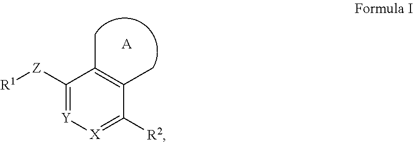

or a pharmaceutically acceptable salt or prodrug thereof, wherein:

X is selected from CR³ and N;

Y is selected from CR⁴ and N;

Z is selected from —O—, —S—, —S(O)—, —S(O)₂—, —C(O)—, —C(HR)—, —N(R⁶)—, C₁-C₃ alkylene, C₁-C₃ heteroalkylene, C₁-C₃ alkenylene or absent;

A is selected from C₃₋₁₂ carbocycle and 3- to 12-membered heterocycle, each of which is optionally substituted with one or more R²⁰;

R¹ is selected from C₁₋₆ alkyl, C₃₋₁₂ carbocycle and 3- to 12-membered heterocycle, each of which is optionally substituted with one or more R²⁰;

R², R³, R⁴ and R⁵ are each independently selected from hydrogen and R²⁰;

R⁶ is selected from R²¹;

R²⁰ is independently selected at each occurrence from:
halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, and ═N(R²¹);
C₁₋₁₀ alkyl, C₂₋₁₀ alkenyl, and C₂₋₁₀ alkynyl, each of which is independently optionally substituted at each occurrence with one or more substituents selected from R²⁴; and
C₃₋₁₂ carbocycle and 3- to 12-membered heterocycle, each of which is independently optionally substituted at each occurrence with one or more substituents selected from R²⁵;

R²¹ is independently selected at each occurrence from hydrogen; and C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;

R²² and R²³ are taken together with the nitrogen atom to which they are attached to form a heterocycle, optionally substituted with one or more R²⁰;

R²⁴ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₃₋₁₂ carbocycle, and 3- to 12-membered heterocycle, wherein each C₃₋₁₂ carbocycle and 3- to 12-membered heterocycle is independently optionally substituted with one or more substituents selected from R²⁵; and

R²⁵ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —R²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₁₋₆ alkyl, C₁₋₆ haloalkyl, C₂₋₆ alkenyl, and C₂₋₆ alkynyl.

In some embodiments, for a compound of Formula I, A is selected from C₅ carbocycle and 5-membered heterocycle. In some embodiments, A is substituted with at least one substituent selected from halogen, —OH, —OR²¹, —N(R²¹)₂, —NR²²R²³, C₁₋₁₀ alkyl, C₂₋₁₀ alkenyl, and C₂₋₁₀ alkynyl. In some embodiments, A is substituted with at least one substituent selected from —F and —OH.

In practicing any of the subject methods, the HIF-2α inhibitor may be a compound of Formula I-A:

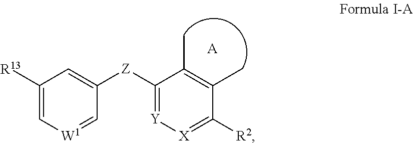

or a pharmaceutically acceptable salt or prodrug thereof, wherein:

X is selected from CR³ and N;

Y is selected from CR⁴ and N;

Z is selected from —O—, —S—, —S(O)—, —S(O)₂—, —C(O)—, —C(HR⁵)—, —N(R⁶)—, C₁-C₃ alkylene, C₁-C₃ heteroalkylene, C₁-C₃ alkenylene or absent;

W 1 is N or CR¹⁴;

A is selected from C₃₋₁₂ carbocycle and 3- to 12-membered heterocycle, each of which is optionally substituted with one or more R²⁰;

R², R³, R⁴, R⁵, R¹³ and R¹⁴ are each independently selected from hydrogen and R²⁰;

R⁶ is selected from R²¹;

R²⁰ is independently selected at each occurrence from:
halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, and ═N(R²¹);
C₁₋₁₀ alkyl, C₂₋₁₀ alkenyl, and C₂₋₁₀ alkynyl, each of which is independently optionally substituted at each occurrence with one or more substituents selected from R²⁴; and
C₃₋₁₂ carbocycle and 3- to 12-membered heterocycle, each of which is independently optionally substituted at each occurrence with one or more substituents selected from R²⁵;

R²¹ is independently selected at each occurrence from hydrogen; and C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;

R²² and R²³ are taken together with the nitrogen atom to which they are attached to form a heterocycle, optionally substituted with one or more R²⁰;

R²⁴ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₃₋₁₂ carbocycle, and 3- to 12-membered heterocycle, wherein each C₃₋₁₂ carbocycle and 3- to 12-membered heterocycle is independently optionally substituted with one or more substituents selected from R²⁵; and

R²⁵ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —R²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₁₋₆ alkyl, C₁₋₆ haloalkyl, C₂₋₆ alkenyl, and C₂₋₆ alkynyl.

In practicing any of the subject methods, the HIF-2α inhibitor may be a compound of Formula I-B:

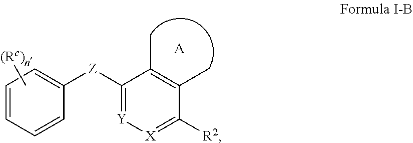

or a pharmaceutically acceptable salt or prodrug thereof, wherein:

X is selected from CR³ and N;

Y is selected from CR⁴ and N;

Z is selected from —O—, —S—, —S(O)—, —S(O)₂—, —C(O)—, —C(HR)—, —N(R⁶)—, C₁-C₃ alkylene, C₁-C₃ heteroalkylene, C₁-C₃ alkenylene or absent;

A is selected from C₃₋₁₂ carbocycle and 3- to 12-membered heterocycle, each of which is optionally substituted with one or more R²⁰;

R², R³, R⁴, R⁵ and R^{c} are each independently selected at each occurrence from hydrogen and R²⁰;

n is 0, 1, 2, 3 or 4;

R⁶ is selected from R²¹;

R²⁰ is independently selected at each occurrence from:
halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, and ═N(R²¹);
C₁₋₁₀ alkyl, C₂₋₁₀ alkenyl, and C₂₋₁₀ alkynyl, each of which is independently optionally substituted at each occurrence with one or more substituents selected from R²⁴; and
C₃₋₁₂ carbocycle and 3- to 12-membered heterocycle, each of which is independently optionally substituted at each occurrence with one or more substituents selected from R²⁵;

R²¹ is independently selected at each occurrence from hydrogen; and C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;

R²² and R²³ are taken together with the nitrogen atom to which they are attached to form a heterocycle, optionally substituted with one or more R²⁰;

R²⁴ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₃₋₁₂ carbocycle, and 3- to 12-membered heterocycle, wherein each C₃₋₁₂ carbocycle and 3- to 12-membered heterocycle is independently optionally substituted with one or more substituents selected from R²⁵; and

R²⁵ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₁₋₆ alkyl, C₁₋₆ haloalkyl, C₂₋₆ alkenyl, and C₂₋₆ alkynyl.

In practicing any of the subject methods, the HIF-2α inhibitor may be a compound of Formula I-C:

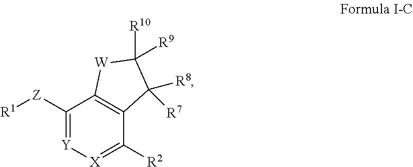

wherein:

W is selected from O, S, CR¹¹R¹² and NR⁶; and

R⁷, R⁸, R⁹, R¹⁰, R¹¹ and R¹² are each independently selected from hydrogen and R²⁰, or R⁷ and R⁸ in combination form oxo or oxime.

In some embodiments, for a compound of Formula I-C, R⁷ is selected from hydrogen, halogen, —OR²¹, —N(R²¹)₂ and —NR²²R²³; R is selected from hydrogen, C₁₋₁₀ alkyl, C₂₋₁₀ alkenyl, and C₂₋₁₀ alkynyl; and R⁹, R¹⁰, R¹¹ and R¹² are each independently selected from hydrogen, halogen, —OR²¹, C₁₋₁₀ alkyl and 2- to 10-membered heteroalkyl. In some embodiments, R⁸ is hydrogen. In some embodiments, at least one of R⁹, R¹⁰, R¹¹ and R¹² is fluoro. In some embodiments, W is selected from O and CR¹¹R¹². In some embodiments, W is CR¹¹R¹².

In practicing any of the subject methods, the HIF-2α inhibitor may be a compound of Formula I-D, I-E, I-F or I-G:

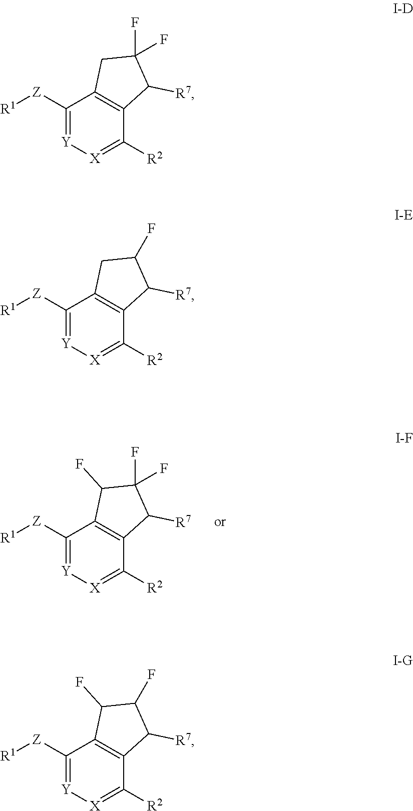

or a pharmaceutically acceptable salt or prodrug thereof.

In practicing any of the subject methods, the HIF-2α inhibitor may be a compound of Formula I-H, I-I, I-J or I-K:

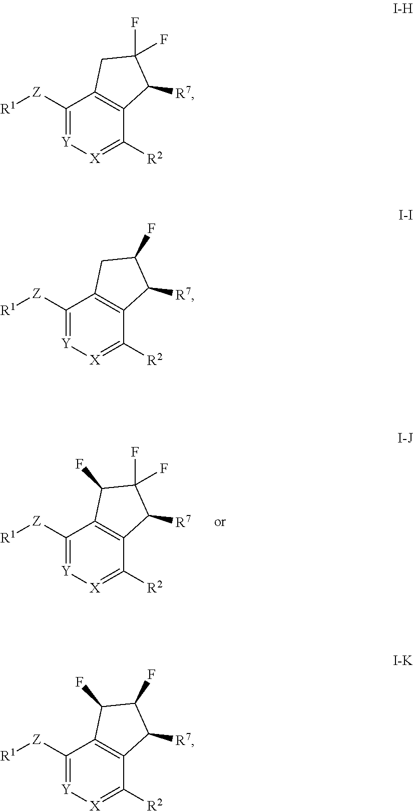

or a pharmaceutically acceptable salt or prodrug thereof.

In some embodiments, for a compound of Formula I-C, I-D, I-E, I-F, I-G, I-H, I-I, I-J or I-K, R⁷ is selected from —OR²¹ and —N(R²¹)₂, such as —OH and —NH₂. In some embodiments, R⁷ is —OH.

In some embodiments, for a compound of Formula I′, I, I-A, I-B, I-C, I-D, I-E, I-F, I-G, I-H, I-I, I-J, I-K, II, II-A or II-B, R¹ is selected from C₆₋₁₀ aryl, 5- to 8-membered heteroaryl, C₃₋₈ cycloalkyl and 3- to 8-membered heterocycloalkyl, such as R¹ is selected from phenyl and pyridyl. In some embodiments, R¹ is substituted with at least one substituent selected from R²⁰. In some embodiments, R is substituted with at least one substituent selected from halogen, —CN, C₁₋₄ alkyl and C₁₋₄ alkoxy.

In some embodiments, for a compound of Formula I′, I, I-A, I-B, I-C, I-D, I-E, I-F, I-G, I-H, I-I, I-J, I-K, II, II-A or II-B, R² is selected from —CN, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, C₁₋₁₀ fluoroalkyl, C₃₋₁₂ carbocycle and 3- to 12-membered heterocycle. In some embodiments, R² is selected from —CN, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹ and C₁₋₁₀ fluoroalkyl. In some embodiments, R² is selected from —S(═O)₂CH₃, —S(═O)₂CHF₂, —S(═O)(═N—CN)CH₃ and CF₃. In some embodiments, R² is selected from C₆₋₁₀ aryl and 5- to 8-membered heteroaryl, such as 5-membered heteroaryl.

In some embodiments, for a compound of Formula I′, I, I-A, I-B, I-C, I-D, I-E, I-F, I-G, I-H, I-I, I-J, I-K, II, II-A or II-B, Z is —O—.

In some embodiments, for a compound of Formula I-C, I-D, I-E, I-F, I-G, I-H, I-I, I-J or I-K, R² is selected from —S(═O)₂R²¹, —S(═O)(═NR²¹)R²¹ and C₁₋₃ fluoroalkyl; Z is —O—; R⁷ is —OH; and R⁸ is hydrogen. In some embodiments, R¹ is selected from C₆₋₁₀ aryl, 5- to 8-membered heteroaryl, C₃₋₈ cycloalkyl and 3- to 8-membered heterocycloalkyl.

In some embodiments, for a compound of Formula I′, I, I-A, I-B, I-C, I-D, I-E, I-F, I-G, I-H, I-I, I-J or I-K, X is N and Y is CR⁴. In some embodiments, X is CR³ and Y is N. In some embodiments, X is N and Y is N. In some embodiments, X is CR³ and Y is CR⁴.

In practicing any of the subject methods, the HIF-2α inhibitor may be selected from Table 1 or Table 2. In some embodiments, the HIF-2α inhibitor is

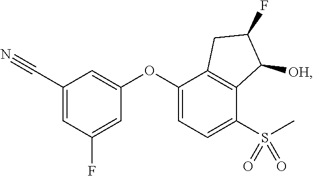 [SMILES: CS(=O)(=O)c1ccc(Oc2cc(F)cc(C#N)c2)c2c1[C@H](O)[C@H](F)C2]

or a pharmaceutically acceptable salt thereof. In some embodiments, the HIF-2α inhibitor is selected from the group consisting of

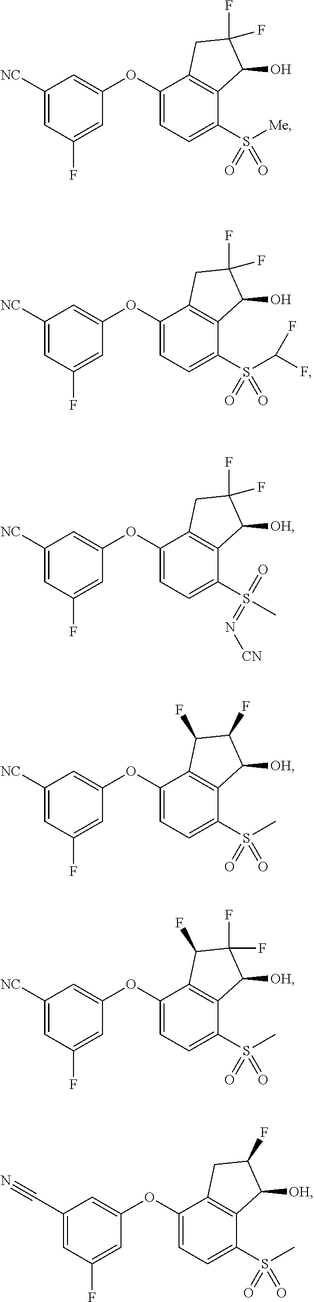 [SMILES: CS(=O)(=O)c1ccc(Oc2cc(F)cc(C#N)c2)c2c1[C@H](O)C(F)(F)C2 ; N#Cc1cc(F)cc(Oc2ccc(S(=O)(=O)C(F)F)c3c2CC(F)(F)[C@H]3O)c1 ; CS(=O)(=NC#N)c1ccc(Oc2cc(F)cc(C#N)c2)c2c1[C@H](O)C(F)(F)C2 ; CS(=O)(=O)c1ccc(Oc2cc(F)cc(C#N)c2)c2c1[C@H](O)[C@H](F)[C@@H]2F ; CS(=O)(=O)c1ccc(Oc2cc(F)cc(C#N)c2)c2c1[C@H](O)C(F)(F)[C@@H]2F ; CS(=O)(=O)c1ccc(Oc2cc(F)cc(C#N)c2)c2c1[C@H](O)[C@H](F)C2]

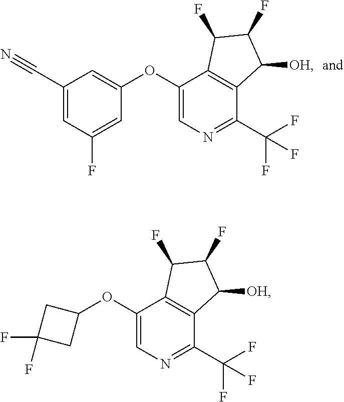 [SMILES: N#Cc1cc(F)cc(Oc2cnc(C(F)(F)F)c3c2[C@@H](F)[C@@H](F)[C@H]3O)c1 ; O[C@H]1c2c(C(F)(F)F)ncc(OC3CC(F)(F)C3)c2[C@@H](F)[C@H]1F]

or a pharmaceutically acceptable salt thereof.

In practicing any of the subject methods, the HIF-2α inhibitor may be a compound of Formula II:

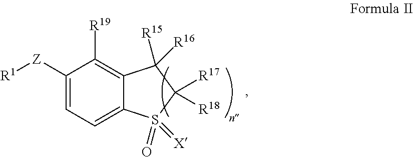

or a pharmaceutically acceptable salt or prodrug thereof, wherein:

Z is selected from —O—, —S—, —S(O)—, —S(O)₂—, —C(O)—, —C(HR)—, —N(R⁶)—, C₁-C₃ alkylene, C₁-C₃ heteroalkylene, C₁-C₃ alkenylene or absent;

R¹ is selected from C₁₋₆ alkyl, C₃₋₁₂ carbocycle and 3- to 12-membered heterocycle, each of which is optionally substituted with one or more R²⁰;

R⁵ is selected from hydrogen and R²⁰;

R⁶ is selected from R²¹;

R¹⁵ is selected from hydrogen, —OH, and —N(R²¹)₂;

R¹⁶ is selected from hydrogen, deuterium and C₁₋₆ alkyl, wherein said C₁₋₆ alkyl is optionally substituted with one or more R²⁰; or R¹⁵ and R¹⁶ in combination form oxo or methylene;

R¹⁷ and R¹⁸ are independently selected from hydrogen and halogen; and C₁₋₆ alkyl, 2- to 6-membered heteroalkyl and C₃₋₁₀ cycloalkyl, each of which is optionally substituted with one or more R²⁰; or R¹ and R¹⁸ and the carbon to which they are attached form C₃-C₈ cycloalkyl or C₅-C₈ heterocycloalkyl, each of which is optionally substituted with one or more R²⁰;

R¹⁹ is selected from hydrogen, halogen, —CN, —NO₂, —C(O)R²¹, —C(O)OR²¹, —OC(O)R²¹, —OC(O)N(R²¹)₂, —OC(O)NR²²R²³, —NR²¹C(O)R²¹ and —S(═O)₂R²¹; and C₁₋₆ alkyl, 2- to 6-membered heteroalkyl, C₁₋₁₀ alkenyl, and C₁₋₁₀ alkynyl, each of which is optionally substituted with one or more R²⁰;

X′ is O or NR^{18′}, wherein R¹⁸ is selected from the group consisting of hydrogen, C₁₋₆ alkyl and —CN;

n″ is 1 or 2;

R²⁰ is independently selected at each occurrence from:
halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, and ═N(R²¹);
C₁₋₁₀ alkyl, C₂₋₁₀ alkenyl, and C₂₋₁₀ alkynyl, each of which is independently optionally substituted at each occurrence with one or more substituents selected from R²⁴; and
C₃₋₁₂ carbocycle and 3- to 12-membered heterocycle, each of which is independently optionally substituted at each occurrence with one or more substituents selected from R²⁵;

R²¹ is independently selected at each occurrence from hydrogen; and C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;

R²² and R²³ are taken together with the nitrogen atom to which they are attached to form a heterocycle, optionally substituted with one or more R²⁰;

R²⁴ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₃₋₁₂ carbocycle, and 3- to 12-membered heterocycle, wherein each C₃₋₁₂ carbocycle and 3- to 12-membered heterocycle is independently optionally substituted with one or more substituents selected from R²⁵; and

R²⁵ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₁₋₆ alkyl, C₁₋₆ haloalkyl, C₂₋₆ alkenyl, and C₂₋₆ alkynyl.

In practicing any of the subject methods, the HIF-2α inhibitor may be a compound of Formula II-A:

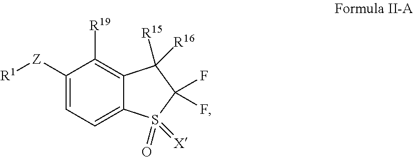

or a pharmaceutically acceptable salt or prodrug thereof.

In practicing any of the subject methods, the HIF-2α inhibitor may be a compound of Formula II-B:

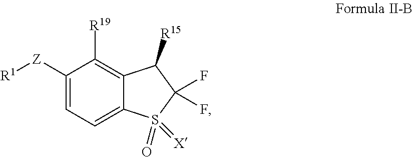

or a pharmaceutically acceptable salt or prodrug thereof.

In some embodiments, the enantiomeric excess of the HIF-2α inhibitor is at least about 85%. In some embodiments, the HIF-2α inhibitor is provided in a pharmaceutical composition. In some embodiments, the pharmaceutical composition is provided in a unit dose. In some embodiments, the pharmaceutical composition is formulated for oral or topical administration. In some embodiments, the pharmaceutical composition is provided as a suppository, enema or oral formulation.

In some embodiments, a method described herein further comprises administering a second therapeutic agent, such as a second therapeutic agent selected from 5-aminosalicylates, corticosteroids, thiopurines, anti-TNF-α agents and anti-integrin agents.

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

FIG. 1 depicts the disease activity index score of mice treated with vehicle, Compound 231 or filgotinib in a DSS-induced model of colitis.

FIG. 2 shows the colon lengths of mice treated with vehicle, Compound 231 or filgotinib in a DSS-induced model of colitis.

### DETAILED DESCRIPTION OF THE INVENTION

While preferred embodiments of the present invention have been shown and described herein, it will be obvious to those skilled in the art that such embodiments are provided by way of example only. Numerous variations, changes and substitutions will now occur to those skilled in the art without departing from the invention. It should be understood that various alternatives to the embodiments of the invention described herein may be employed in practicing the invention. It is intended that the appended claims define the scope of the invention and that methods and structures within the scope of these claims and their equivalents be covered thereby.

Unless defined otherwise, all technical and scientific terms used herein have the same meaning as is commonly understood by one of skill in the art to which this invention belongs.

As used in the specification and claims, the singular form “a”, “an” and “the” include plural references unless the context clearly dictates otherwise.

The term “C_{x-y}” or “Cₓ-C_{y}” when used in conjunction with a chemical moiety, such as alkyl, alkenyl, or alkynyl is meant to include groups that contain from x to y carbons in the chain. For example, the term “C_{x-y} alkyl” refers to substituted or unsubstituted saturated hydrocarbon groups, including straight-chain alkyl and branched-chain alkyl groups that contain from x to y carbons in the chain.

“Alkyl” refers to substituted or unsubstituted saturated hydrocarbon groups, including straight-chain alkyl and branched-chain alkyl groups. An alkyl group may contain from one to twelve carbon atoms (e.g., C₁₋₁₂ alkyl), such as one to eight carbon atoms (C₁₋₈ alkyl) or one to six carbon atoms (C₁₋₆ alkyl). Exemplary alkyl groups include methyl, ethyl, n-propyl, isopropyl, n-butyl, isobutyl, sec-butyl, tert-butyl, pentyl, isopentyl, neopentyl, hexyl, septyl, octyl, nonyl, and decyl. An alkyl group is attached to the rest of the molecule by a single bond. Unless stated otherwise specifically in the specification, an alkyl group is optionally substituted by one or more substituents such as those substituents described herein.

“Haloalkyl” refers to an alkyl group that is substituted by one or more halogens. Exemplary haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl.

“Alkenyl” refers to substituted or unsubstituted hydrocarbon groups, including straight-chain or branched-chain alkenyl groups containing at least one double bond. An alkenyl group may contain from two to twelve carbon atoms (e.g., C₂₋₁₂ alkenyl). Exemplary alkenyl groups include ethenyl (i.e., vinyl), prop-1-enyl, but-1-enyl, pent-1-enyl, penta-1,4-dienyl, and the like. Unless stated otherwise specifically in the specification, an alkenyl group is optionally substituted by one or more substituents such as those substituents described herein.

“Alkynyl” refers to substituted or unsubstituted hydrocarbon groups, including straight-chain or branched-chain alkynyl groups containing at least one triple bond. An alkynyl group may contain from two to twelve carbon atoms (e.g., C₂₋₁₂ alkynyl). Exemplary alkynyl groups include ethynyl, propynyl, butynyl, pentynyl, hexynyl, and the like. Unless stated otherwise specifically in the specification, an alkynyl group is optionally substituted by one or more substituents such as those substituents described herein.

“Alkylene” or “alkylene chain” refers to substituted or unsubstituted divalent saturated hydrocarbon groups, including straight-chain alkylene and branched-chain alkylene groups that contain from one to twelve carbon atoms. Exemplary alkylene groups include methylene, ethylene, propylene, and n-butylene. Similarly, “alkenylene” and “alkynylene” refer to alkylene groups, as defined above, which comprise one or more carbon-carbon double or triple bonds, respectively. The points of attachment of the alkylene, alkenylene or alkynylene chain to the rest of the molecule can be through one carbon or any two carbons within the chain. Unless stated otherwise specifically in the specification, an alkylene, alkenylene, or alkynylene group is optionally substituted by one or more substituents such as those substituents described herein.

“Heteroalkyl”, “heteroalkenyl” and “heteroalkynyl” refer to substituted or unsubstituted alkyl, alkenyl and alkynyl groups which respectively have one or more skeletal chain atoms selected from an atom other than carbon, e.g., O, N, P, Si, S or combinations thereof, and wherein the nitrogen, phosphorus, and sulfur atoms may optionally be oxidized and the nitrogen heteroatom may optionally be quaternized. If given, a numerical range refers to the chain length in total. For example, a 3- to 8-membered heteroalkyl has a chain length of 3 to 8 atoms. Connection to the rest of the molecule may be through either a heteroatom or a carbon in the heteroalkyl, heteroalkenyl or heteroalkynyl chain. Unless stated otherwise specifically in the specification, a heteroalkyl, heteroalkenyl, or heteroalkynyl group is optionally substituted by one or more substituents such as those substituents described herein.

“Heteroalkylene”, “heteroalkenylene” and “heteroalkynylene” refer to substituted or unsubstituted alkylene, alkenylene and alkynylene groups which respectively have one or more skeletal chain atoms selected from an atom other than carbon, e.g., O, N, P, Si, S or combinations thereof, and wherein the nitrogen, phosphorus, and sulfur atoms may optionally be oxidized and the nitrogen heteroatom may optionally be quaternized. The points of attachment of the heteroalkylene, heteroalkenylene or heteroalkynylene chain to the rest of the molecule can be through either one heteroatom or one carbon, or any two heteroatoms, any two carbons, or any one heteroatom and any one carbon in the heteroalkyl, heteroalkenyl or heteroalkynyl chain. Unless stated otherwise specifically in the specification, a heteroalkylene, heteroalkenylene, or heteroalkynylene group is optionally substituted by one or more substituents such as those substituents described herein.

“Carbocycle” refers to a saturated, unsaturated or aromatic ring in which each atom of the ring is a carbon atom. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Unless stated otherwise specifically in the specification, a carbocycle is optionally substituted by one or more substituents such as those substituents described herein.

“Heterocycle” refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. Each ring of a bicyclic heterocycle may be selected from saturated, unsaturated, and aromatic rings. The heterocycle may be attached to the rest of the molecule through any atom of the heterocycle, valence permitting, such as a carbon or nitrogen atom of the heterocycle. In some embodiments, the heterocycle is a heteroaryl. In some embodiments, the heterocycle is a heterocycloalkyl. In an exemplary embodiment, a heterocycle, e.g., pyridyl, may be fused to a saturated or unsaturated ring, e.g., cyclohexane, cyclopentane, or cyclohexene. Exemplary heterocycles include pyrrolidinyl, pyrrolyl, imidazolyl, pyrazolyl, triazolyl, piperidinyl, pyridinyl, pyrimidinyl, pyridazinyl, pyrazinyl, thiophenyl, oxazolyl, thiazolyl, morpholinyl, indazolyl, indolyl, and quinolinyl. Unless stated otherwise specifically in the specification, a heterocycle is optionally substituted by one or more substituents such as those substituents described herein.

“Heteroaryl” refers to a 3- to 12-membered aromatic ring that comprises at least one heteroatom wherein each heteroatom may be independently selected from N, O, and S. As used herein, the heteroaryl ring may be selected from monocyclic or bicyclic and fused or bridged ring systems wherein at least one of the rings in the ring system is aromatic, i.e., it contains a cyclic, delocalized (4n+2) π-electron system in accordance with the Hückel theory. The heteroatom(s) in the heteroaryl may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, a heteroaryl is optionally substituted by one or more substituents such as those substituents described herein.

The term “substituted” refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, a carbocycle, a heterocycle, a cycloalkyl, a heterocycloalkyl, an aromatic and heteroaromatic moiety. In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R—S(O)ₜOR^{a} (where t is 1 or 2), and —R—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, hydroxy, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a}), —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a}), —R^{b}—N(R^{a})C(O)OR^{a}, —R^{b}—N(R^{a})C(O)R^{a}, —R^{b}—N(R^{a})S(O)ₜR^{a} (where t is 1 or 2), —R—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R—S(O)R^{a} (where t is 1 or 2), —R—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain. In some embodiments, a substituent is selected from R²⁰ as defined herein below.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

Where substituent groups are specified by their conventional chemical formulae, written from left to right, they equally encompass the chemically identical substituents that would result from writing the structure from right to left, e.g., —CH₂O— is equivalent to —OCH₂—.

“Optional” or “optionally” means that the subsequently described event of circumstances may or may not occur, and that the description includes instances where the event or circumstance occurs and instances in which it does not. For example, “optionally substituted aryl” means that the aryl group may or may not be substituted and that the description includes both substituted aryl groups and aryl groups having no substitution.

Compounds of the present disclosure also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Isotopically-enriched compounds may be prepared by conventional techniques well known to those skilled in the art.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, chemical entities described herein are intended to include all Z-, E- and tautomeric forms as well.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When stereochemistry is not specified, certain small molecules described herein include, but are not limited to, when possible, their isomers, such as enantiomers and diastereomers, mixtures of enantiomers, including racemates, mixtures of diastereomers, and other mixtures thereof, to the extent they can be made by one of ordinary skill in the art by routine experimentation. In those situations, the single enantiomers or diastereomers, i.e., optically active forms, can be obtained by asymmetric synthesis or by resolution of the racemates or mixtures of diastereomers. Resolution of the racemates or mixtures of diastereomers, if possible, can be accomplished, for example, by conventional methods such as crystallization in the presence of a resolving agent, or chromatography, using, for example, a chiral high-pressure liquid chromatography (HPLC) column. Furthermore, a mixture of two enantiomers enriched in one of the two can be purified to provide further optically enriched form of the major enantiomer by recrystallization and/or trituration. In addition, such certain small molecules include Z- and E-forms (or cis- and trans-forms) of certain small molecules with carbon-carbon double bonds or carbon-nitrogen double bonds. Where certain small molecules described herein exist in various tautomeric forms, the term “certain small molecule” is intended to include all tautomeric forms of the certain small molecule.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

“Pharmaceutically acceptable carrier, diluent or excipient” includes without limitation any adjuvant, carrier, excipient, glidant, sweetening agent, diluent, preservative, dye, colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The term “effective amount” or “therapeutically effective amount” refers to that amount of a compound described herein that is sufficient to affect the intended application, including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder, or medical condition including but not limited to a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

The terms “antagonist” and “inhibitor” are used interchangeably, and they refer to a compound having the ability to inhibit a biological function (e.g., activity, expression, binding, protein-protein interaction) of a target protein or enzyme (e.g., HIF-2α). Accordingly, the terms “antagonist” and “inhibitor” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition. A preferred biological activity inhibited by an antagonist is associated with inflammation.

The term “agonist” as used herein refers to a compound having the ability to initiate or enhance a biological function of a target protein, whether by inhibiting the activity or expression of the target protein. Accordingly, the term “agonist” is defined in the context of the biological role of the target polypeptide. While preferred agonists herein specifically interact with (e.g., bind to) the target, compounds that initiate or enhance a biological activity of the target polypeptide by interacting with other members of the signal transduction pathway of which the target polypeptide is a member are also specifically included within this definition.

“Signal transduction” is a process during which stimulatory or inhibitory signals are transmitted into and within a cell to elicit an intracellular response. A modulator of a signal transduction pathway refers to a compound which modulates the activity of one or more cellular proteins mapped to the same specific signal transduction pathway. A modulator may augment (agonist) or suppress (antagonist) the activity of a signaling molecule.

The term “heterodimerization” as used herein refers to the complex formed by the non-covalent binding of HIF-2α to HIF-1β (ARNT). Heterodimerization of HIF-2α to HIF-1β (ARNT) is required for HIF-2α DNA binding and transcriptional activity and is mediated by the HLH and PAS-B domains. Transcriptional activity following heterodimerization of HIF-2α to HIF-1β (ARNT) can affect five groups of target genes including angiogenic factors, glucose transporters and glycolytic enzymes, stem-cell factors, survival factors, and invasion factors.

The term “HIF-2α” refers to a monomeric protein that contains three conserved structured domains: basic helix-loop-helix (bHLH), and two Per-ARNT-Sim (PAS) domains designated PAS-A and PAS-B, in addition to C-terminal regulatory regions. “HIF-2a” is also alternatively known by several other names in the scientific literature, most commonly endothelial PAS domain-containing protein 1 (EPAS-1) which is encoded by the EPAS1 gene. Alternative names include basic-helix-loop-helix-PAS protein (MOP2). As a member of the bHLH/PAS family of transcription factors, “HIF-2α” forms an active heterodimeric transcription factor complex by binding to the ARNT (also known as HIF-1β) protein through non-covalent interactions.

The term “HIF-2α PAS-B domain cavity” refers to an internal cavity within the PAS-B domain of HIF-2α. The crystal structure of the PAS-B domain can contain a large (approximately 290 Å) cavity in its core. However, the amino acid side chains in the solution structure are dynamic. For example, those side chains can tend to intrude more deeply in the core, and can shrink the cavity to 1 or 2 smaller cavities or can even expand the cavity. The cavity is lined by amino acid residues comprising PHE-244, SER-246, HIS-248, MET-252, PHE-254, ALA-277, PHE-280, TYR-281, MET-289, SER-292, HIS-293, LEU-296, VAL-302, VAL-303, SER-304, TYR-307, MET-309, LEU-319, THR-321, GLN-322, GLY-323, ILE-337, CYS-339, and ASN-341 of HIF-2α PAS-B domain. The numbering system is from the known structures reported in the RCSB Protein Data Bank with PDB code 3H7W. Other numbering systems in the PDB could define the same amino acids, expressed above, that line the cavity.

The term “cell proliferation” refers to a phenomenon by which the cell number has changed as a result of division. This term also encompasses cell growth by which the cell morphology has changed (e.g., increased in size) consistent with a proliferative signal.

The term “selective inhibition” or “selectively inhibit” refers to the ability of a biologically active agent to preferentially reduce the target signaling activity as compared to off-target signaling activity, via direct or indirect interaction with the target.

“Subject” refers to an animal, such as a mammal, for example a human. The methods described herein can be useful in both human therapeutics and veterinary applications. In some embodiments, the subject is a mammal, and in some embodiments, the subject is human. “Mammal” includes humans and both domestic animals such as laboratory animals and household pets (e.g., cats, dogs, swine, cattle, sheep, goats, horses, rabbits), and non-domestic animals such as wildlife and the like.

“Prodrug” is meant to indicate a compound that may be converted under physiological conditions or by solvolysis to a biologically active compound described herein (e.g., a compound of Formula I, I-A, I-B, I-C, I-D, I-E, I-F, I-G, I-H, I-I, I-J, I-K, II, II-A or II-B). Thus, the term “prodrug” refers to a precursor of a biologically active compound that is pharmaceutically acceptable. In some aspects, a prodrug is inactive when administered to a subject but is converted in vivo to an active compound, for example, by hydrolysis. In some aspects, a prodrug has reduced activity compared to that of the parent compound. The prodrug compound often offers advantages of oral bioavailability, solubility, tissue compatibility or delayed release in a mammalian organism (see, e.g., Bundgard, H., Design of Prodrugs (1985), pp. 7-9, 21-24 (Elsevier, Amsterdam); Higuchi, T., et al., “Pro-drugs as Novel Delivery Systems,” (1987) A.C.S. Symposium Series, Vol. 14; and Bioreversible Carriers in Drug Design, ed. Edward B. Roche, American Pharmaceutical Association and Pergamon Press) each of which is incorporated in full by reference herein. The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of a hydroxy functional group, or acetamide, formamide and benzamide derivatives of an amine functional group in the active compound and the like.

The term “in vivo” refers to an event that takes place in a subject's body.

The term “in vitro” refers to an event that takes places outside of a subject's body. For example, an in vitro assay encompasses any assay run outside of a subject. In vitro assays encompass cell-based assays in which cells alive or dead are employed. In vitro assays also encompass a cell-free assay in which no intact cells are employed.

The disclosure is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the disclosure includes compounds produced by a process comprising administering a compound of this disclosure to a mammal for a period of time sufficient to yield a metabolic product thereof. Such products are typically identified by administering a radiolabelled compound of the disclosure in a detectable dose to an animal, such as rat, mouse, guinea pig, monkey, or to human, allowing sufficient time for metabolism to occur, and isolating its conversion products from the urine, blood or other biological samples.

The chemical naming protocol and structure diagrams used herein are a modified form of the I.U.P.A.C. nomenclature system, using ChemDraw Professional 15.1 or OpenEye Scientific Software's mol2nam application. For complex chemical names employed herein, a substituent group is typically named before the group to which it attaches. For example, cyclopropylethyl comprises an ethyl backbone with a cyclopropyl substituent. Except as described below, all bonds are identified in the chemical structure diagrams herein, except for all bonds on some carbon atoms, which are assumed to be bonded to sufficient hydrogen atoms to complete the valency.

In one aspect, the present disclosure provides a method of treating an inflammatory disease of the digestive system in a subject in need thereof. In some embodiments, the method comprises administering to the subject an effective amount of a HIF-2α inhibitor. In one aspect, the present disclosure provides a method of reducing inflammation of the digestive system in a subject in need thereof, comprising administering to the subject an effective amount of a HIF-2α inhibitor. In some embodiments, the HIF-2α inhibitor is administered in an amount effective to delay progression of, reduce the incidence of, or reduce the degree of one or more characteristics associated with the inflammation or the inflammatory disease. In some embodiments, the HIF-2α inhibitor is administered, either in a single dose or over multiple doses, in an amount effective to induce remission of the inflammation or the inflammatory disease.

The digestive system consists of the gastrointestinal tract plus accessory organs of digestion, including the tongue, salivary glands, esophagus, stomach, pancreas, liver, gallbladder, small intestine, large intestine, colon, anus and rectum. Some embodiments of the present disclosure refer specifically to the lower gastrointestinal tract, including the small intestine and large intestine. The term “small intestine” encompasses the duodenum, jejunum and ileum, and the term “large intestine” includes the cecum, appendix, colon, ascending colon, right colic flexure, transverse colon, left colic flexure, descending colon, sigmoid colon, rectum, anal canal and anus.

The present disclosure provides both methods of reducing inflammation of the digestive system and methods of treating an inflammatory disease of the digestive system. As used herein, the term “inflammation” refers to the general term for local accumulation of fluids, plasma proteins, and/or white blood cells initiated by an autoimmune response, physical injury, infection, vascular disease, chemical exposure, radiation or a local immune response. Generally, inflammation is characterized by one or more signs, including, for example, redness, pain, heat, swelling and/or loss of function. Inflammation may be associated with chronic (long term) inflammatory diseases or disorders or acute (short term) inflammatory diseases or disorders.

In practicing any of the subject methods, a HIF-2α inhibitor may reduce inflammation of the digestive system, such as inflammation of one or more of the tongue, salivary glands, esophagus, stomach, pancreas, liver, gallbladder, small intestine, large intestine, colon, anus and rectum. In some embodiments, a HIF-2α inhibitor reduces inflammation of the lower gastrointestinal tract, such as inflammation of the small intestine, large intestine or colon. In some exemplary embodiments, a HIF-2α inhibitor reduces inflammation of the colon. The inflammation may be characterized as enteritis, gastritis, gastroenteritis, colitis, enterocolitis, duodenitis, jejunitis, ileitis, proctitis, or appendicitis. The inflammation may be acute or chronic. In some preferred embodiments, the inflammation is classified as colitis.

Under physiological conditions, the gastrointestinal tract can be characterized by a steep oxygen gradient. Chronic inflammatory bowel disease is typically characterized by an active consumption of O₂ by the recruited inflammatory cells including macrophages, dendritic cells and neutrophils. The resulting imbalance between oxygen consumption and supply renders the inflamed intestinal mucosa severely hypoxic. This “inflammatory hypoxia” typically leads to elevated levels of hypoxia inducible factors 1α and 2α (HIF-1α and HIF-2α) in the intestinal epithelium of subjects suffering from inflammation of the digestive system, including subjects suffering from inflammatory bowel disease and in a murine model of colitis. In inflammatory bowel disease, HIF-1α and HIF-2α have been shown to play opposing roles in disease progression. Elevation of HIF-1α expression has been shown to be protective during inflammatory bowel disease, including for epithelial cell survival, induction of several barrier protective and tight junction proteins, increase in antimicrobial β-defensin and prevention of excessive immune response by upregulation of CD39/CD73 and T regulatory cells.HIF-2α chronic expression can lead to robust spontaneous intestinal inflammation and injury in inflammatory bowel disease including epithelial cell apoptosis, dysregulation of barrier protective and tight junction protein, increased pro-inflammatory cytokines and excessive immune response. HIF-2α has been shown to regulate recruitment and function of myeloid cells, including neutrophils and macrophages, facilitating the progression of inflammation and inflammation mediated colon cancer. Recently, HIF-1α was reported to be a driver of inflammation in a colitis model when knocked down in myeloid cell lineage cells.

Not wishing to be bound by any particular theory, the present inventors hypothesized that HIF-2α expression in colon epithelial and myeloid cells is a major driver of initiation, progression and maintenance of chronic inflammation. Blockade of HIF-2α is expected to reduce or inhibit recruitment of inflammatory cell types and also their pro-inflammatory products, leading to regression or prevention of inflammation of the digestive system, especially in subjects suffering from Crohn's disease and ulcerative colitis.

A subject exhibiting inflammation of the digestive system may suffer from inflammatory bowel disease, Crohn's disease, colitis, celiac disease, eosinophilic enteropathy or appendicitis. As used herein the term “inflammatory bowel disease” refers to a pathology characterized by an inflammatory condition of the colon and/or the small intestine. Crohn's disease and colitis are two types of inflammatory bowel disease.

In some embodiments, the inflammatory bowel disease comprises colitis, such as ulcerative colitis. “Colitis” is an inflammation of the colon. The colitis may be acute or chronic. As used herein, colitis includes ulcerative colitis, microscopic colitis, lymphocytic colitis, collagenous colitis, diversion colitis, chemical colitis, ischemic colitis, infections colitis, pancolitis, left-sided colitis, extensive colitis, segmental colitis, microscopic colitis, radiation-induced colitis, medication-induced colitis and proctitis. “Ulcerative colitis” is a chronic inflammatory disease affecting the colon. It is characterized by mucosal inflammation of the colon. Symptoms can range from mild to severe and may include blood in the stool, diarrhea, bloody diarrhea, rectal urgency, tenesmus, incontinence, fatigue, increased frequency of bowel movements, mucosal discharge, nocturnal defecations, abdominal discomfort, fever, weight loss, paradoxical constipation, anemia, and abdominal tenderness. Ulcerative colitis is an intermittent disease, with most patients having a relapsing and remitting disease course with periodic flares. The terms “flare” or “relapse” refer to an increase in symptoms of ulcerative colitis, for example increased stool frequency, increased rectal bleeding and/or appearance of abnormal mucosa evidenced by endoscopy. Although the symptoms of ulcerative colitis may diminish without intervention, the disease usually requires treatment to go into remission.

The term “active” ulcerative colitis as used herein refers to ulcerative colitis that is biologically active. Patients with active disease may be symptomatic and exhibit one or more sign or symptom of ulcerative colitis, for example, rectal bleeding, increased stool frequency, mucosal inflammation or abnormal laboratory tests (e.g., elevated ESR or CRP values or decreased hemoglobin). “Refractory” ulcerative colitis with respect to a particular therapy refers ulcerative colitis that is active or that relapses or flares in spite of being treated with that therapy.

As used herein, the term “Crohn's disease” refers to a type of inflammatory bowel disease characterized by inflammation of the lining of the gastrointestinal tract. Symptoms may include diarrhea, abdominal pain, fever, fatigue, bloody stool and weight loss.

When ulcerative colitis is suspected in a patient, the initial diagnosis generally is based on a combination of symptoms, endoscopic findings and histology. Diagnoses typically include stool samples, urinalysis, and tests for anemia, iron deficiency, leukocytosis and/or thrombocytosis. Markers of inflammation, such as erythrocyte sedimentation rate (ESR) and C-reactive protein, may be elevated, depending on the severity of the disease. However, endoscopy with biopsies is generally considered to be the only definitive method for establishing an ulcerative colitis diagnosis. Endoscopic findings that support a diagnosis of ulcerative colitis may include erythema, loss of normal vascular pattern, erosions, bleeding, granularity, friability, ulcerations, and pseudopolyps. Biopsies may also be taken at the time of endoscopy to differentiate ulcerative colitis from Crohn's disease. The biopsy samples are examined for distortion of crypt architecture, inflammation of the crypts, crypt shortening, increased lymphocytes and plasma cells in the lamina propria, crypt abscesses, mucin depletion, and hemorrhage or inflammation in the lamina propia.

The ulcerative colitis may affect part of the colon, or substantially the entire colon. The ulcerative colitis may be proctitis, where the ulcerative colitis is limited to the anus and lining of the rectum. The ulcerative colitis may be left-sided colitis, where the colitis is limited to the proportion of the colon distal to the splenic flexure, more particularly ulcerative colitis that extends from the rectum and as far proximally as the splenic flexure. The ulcerative colitis may be extensive colitis, wherein substantially the entire colon is affected. Accordingly, in some embodiments, the present disclosure provides a method of reducing inflammation in a subject suffering from ulcerative colitis, including proctitis, left-sided colitis, and extensive colitis.

Ulcerative colitis is generally further characterized by the severity of the disease, such as remission, mild, moderate or severe ulcerative colitis. The methods of the present disclosure may be applied to the treatment of mild, moderate or severe ulcerative colitis, or ulcerative colitis that is in remission. For example, an effective amount of a HIF-2α inhibitor may be administered to a subject suffering from mild ulcerative colitis. The HIF-2α inhibitor may be administered to a subject suffering from moderate ulcerative colitis. The HIF-2α inhibitor may be administered to a subject suffering from severe ulcerative colitis. The HIF-2α inhibitor may be administered to a subject suffering from mild or moderate ulcerative colitis. The HIF-2α inhibitor may be administered to a subject suffering from moderate or severe ulcerative colitis. The HIF-2α inhibitor may be administered to a subject suffering from ulcerative colitis that is in remission.

Numerous indices exist for assessing the severity of ulcerative colitis, including the Mayo score, Lichtiger score and Simple Clinical Colitis Activity Index. These indices typically factor in an endoscopy subscore, such as the subscore of the Mayo score or the Ulcerative Colitis Endoscopic Index of severity. Typical histological classifications include the Robarts Histopathology index and the Nancy index. A composite criteria may be used to assess the disease severity, incorporating one or more of these indices, the effect of the disease on the subject's quality of life, measurable markers of the disease activity and extent and the overall disease course, such as extraintestinal manifestations, intestinal damage and frequency of flares.

The Mayo scoring system is a 0 to 12 point composite index that is composed of inputs from the subject and the treatment provider, such as a physician. Each sub-score of the Mayo system ranges from 0 to 3, depending upon the severity. The sum of the individual sub-scores provides the total Mayo score. The Mayo scoring system is summarized in the table below. This scoring system may be used to determine the Mayo score mentioned in any of the embodiments described herein.

Mayo Score

| | Score |
|---|---|
| Stool Frequency | |
| Normal for patient | 0 |
| 1-2 more than normal | 1 |
| 3-4 more than normal | 2 |
| 5 or more stools than normal | 3 |
| Rectal Bleeding | |
| No blood | 0 |
| Streaks of blood in stool less than half of the time | 1 |
| Obvious blood in stool most of the time | 2 |
| Blood alone passed | 3 |
| Endoscopy Findings | |
| Normal or inactive disease | 0 |
| Mild (erythema, decreased vascular pattern, | 1 |
| mild friability) | |
| Moderate (marked erythema, absent vascular | 2 |
| pattern, friability, erosions) | |
| Severe (spontaneous bleeding, ulceration) | 3 |
| Physician's Global Assessment | |
| (includes patient symptoms including abdominal | |
| discomfort and sense of well-being as well as | |
| performance status and physical exam) | |
| Normal | 0 |
| Mild disease | 1 |
| Moderate disease | 2 |
| Severe disease | 3 |

In some embodiments, a HIF-2α inhibitor is administered to a subject having a total Mayo score of 2 or more, such as 3 or more, 4 or more, 5 or more, 6 or more, 7 or more, 8 or more, 9 or more, or 10 or more. In some embodiments, a HIF-2α inhibitor may be administered to a subject having a total Mayo score of 2 to 12, such as 4 to 12 or 6 to 12. In some embodiments, a HIF-2α inhibitor is administered to a subject having a combined daily stool frequency and rectal bleeding Mayo score of 2 or more, such as 3 or more or 4 or more. In some embodiments, a HIF-2α inhibitor is administered to a subject having a total Mayo score of 0.

The Lichtiger scoring system is a 0 to 21 point composite index that is composed of inputs from the subject and the treatment provider, such as a physician. The sum of the individual sub-scores provides the total Lichtiger score. In some embodiments, a score of less than 10 on two consecutive days is considered a clinical response. The Lichtiger scoring system is summarized in the table below. This scoring system may be used to determine the Lichtiger score mentioned in any of the embodiments described herein.

Lichtiger Score

| | Score |
|---|---|
| Diarrhea (number of daily stools) | |
| 0-2 | 0 |
| 3-4 | 1 |
| 5-6 | 2 |
| 7-9 | 3 |
| 10 or more | 4 |
| Nocturnal Diarrhea | |
| No | 0 |
| Yes | 1 |
| Visible Blood in Stool | |
| (% of movements) | |
| 0 | 0 |
| <50 | 1 |
| ≥50 | 2 |
| 100 | 3 |
| Fecal Incontinence | |
| No | 0 |
| Yes | 1 |
| Abdominal Pain or Cramping | |
| None | 0 |
| Mild | 1 |
| Moderate | 2 |
| Severe | 3 |
| General Well-Being | |
| Perfect | 0 |
| Very Good | 1 |
| Good | 2 |
| Average | 3 |
| Poor | 4 |
| Terrible | 5 |
| Abdominal Tenderness | |
| None | 0 |
| Mild and Localized | 1 |
| Mild to Moderate and Diffuse | 2 |
| Severe or Rebound | 3 |
| Need for Antidiarrheal Drugs | |
| No | 0 |
| Yes | 1 |

In some embodiments, a HIF-2α inhibitor is administered to a subject having a total Lichtiger score of 4 or more, such as 6 or more, 8 or more, 10 or more, 12 or more, 14 or more, 16 or more, or 18 or more. In some embodiments, a HIF-2α inhibitor may be administered to a subject having a total Lichtiger score of 4 to 21, such as 6 to 21, 8 to 21, 10 to 21, 12 to 21 or 14 to 21. In some embodiments, a HIF-2α inhibitor is administered to a subject having a total Lichtiger score of 0 to 10.

In practicing any of the subject methods, the HIF-2α inhibitor administered to the subject achieves remission of the inflammatory disease, such as Crohn's disease or ulcerative colitis. In some embodiments, the HIF-2α inhibitor administered to the subject maintains remission of the inflammatory disease, such as Crohn's disease or ulcerative colitis. In some embodiments, the HIF-2α inhibitor administered to the subject reduces or prevents flare-ups of the inflammatory disease, such as flare-ups of Crohn's disease or ulcerative colitis. In some embodiments, a HIF-2α inhibitor is administered to a subject experiencing a flare of ulcerative colitis.

In practicing any of the subject methods, the HIF-2α inhibitor administered to the subject may induce remission of inflammation of the digestive system. In some embodiments, the HIF-2α inhibitor administered to the subject reduces intestinal inflammation. In some embodiments, the HIF-2α inhibitor administered to the subject inhibits recruitment of inflammatory cells. In some embodiments, the HIF-2α inhibitor administered to the subject induces remission of an inflammatory disease of the digestive system. In some embodiments, the HIF-2α inhibitor administered to the subject induces remission of ulcerative colitis. In some embodiments, the amount of HIF-2α inhibitor administered to a subject (either in a single dose or over multiple doses) is effective in one or more of inducing remission of intestinal inflammation, reducing intestinal inflammation, inducing remission of ulcerative colitis, inhibiting recruitment of inflammatory cells, and reducing severity or incidence of symptoms associated with inflammation of the digestive system, such as inflammation associated with ulcerative colitis. The degree of one or more of these therapeutic effects may be about or more than about 10%, 20%, 30%, 40%, 50%, 60%, 70%, 80%, 90%, 95%, or more. In some embodiments, therapeutic efficacy is measured by an increased time in disease progression, such as between the appearance of one or more first symptoms, and the appearance of one or more second symptoms, or delay between two or more occurrences of the same symptoms. Delay may be about or more than about days, weeks, months, or years (e.g. 1, 2, 3, 4, 5, 6, 7, or more days; 1, 2, 3, 4, 5, 6, 7, 8, or more weeks; 1, 2, 3, 4, 5, 6, or more months; or 1, 2, 3, 4, 5, or more years). In the case of prevention, the subject may be an individual at risk of a disease flare, such as a subject in remission.

The degree of therapeutic efficacy may be with respect to a starting condition of the subject (e.g. the baseline Mayo score, baseline Lichtiger score, or severity or incidence of one or more symptoms), or with respect to a reference population (e.g. an untreated population, or a population treated with a different agent). Efficacy in reducing inflammation or treating an inflammatory disease of the digestive system can be ascertained using any suitable method, such as those methods currently used in the clinic to monitor disease severity (e.g., the Mayo score or Lichtiger score).

In practicing any of the subject methods, the HIF-2α inhibitor administered to the subject may induce a remission of ulcerative colitis. The severity of ulcerative colitis may be any of remission, mild, moderate or severe. Remission typically refers to a stool frequency of less than 3 per day, with no visible blood in the stool. Remission may result in complete resolution of symptoms and mucosal healing, which may be determined by endoscopic examination. It is possible to further qualify a remission by reference to a suitable symptom scoring method, for example the Mayo scoring system. For example remission of ulcerative colitis may also be defined to be a total Mayo score of 2 points or less and with no individual sub-score exceeding 1. Induction of a remission may require treatment of the subject for one or more weeks, such as for at least 4 weeks, at least 6 weeks, at least 8 weeks or at least 12 weeks, optionally from 1 week to 12 weeks. For example the subject may be treated for 1, 2, 3, 4, 5, 6, 7, 8, 9, 10, 11, 12 or more weeks to induce remission of the ulcerative colitis.

Desirably, a clinical response is observed following administration of the HIF-2a inhibitor. In some embodiments, the clinical response is evidenced by a decrease in either the Mayo or Lichtiger score of 10% or more, such as 30% or more. In some embodiments, the clinical response is evidenced by a decrease in stool frequency. In some embodiments, the clinical response is evidenced by a decrease in rectal bleeding. In some embodiments, the clinical response is evidenced by a decrease in the endoscopy sub-score of the Mayo index. In some embodiments, the clinical response is evidenced by a decrease in the Mayo score of at least 2, such as at least 3, at least 4, at least 5, at least 6, at least 7, at least 8, at least 9, or at least 10. In some embodiments, the clinical response is evidenced by a decrease in the Lichtiger score of at least 2, such as at least 3, at least 4, at least 5, at least 6, at least 7, at least 8, at least 9, at least 10, at least 11, at least 12, at least 13, at least 14, at least 15, at least 16, at least 17, or at least 18. In some embodiments, the clinical response is evidenced by a Lichtiger score of less than 10 on two consecutive days. In some embodiments, the clinical response is evidenced by a decrease from baseline in the total Mayo score of 3, or a 30% reduction from the baseline Mayo score, and an accompanying decrease in the Mayo sub-score for rectal bleeding of at least 1 point compared to the baseline score or an absolute sub-score for rectal bleeding of 0 or 1.

In some embodiments, mucosal healing is observed in the subject following administration of the HIF-2α inhibitor. The mucosal healing may be evidenced by a reduction in the Mayo endoscopy sub-score of at least 1 point, or an absolute Mayo sub-score for endoscopy of 0 or 1. In some embodiments, rectal bleeding is reduced in the subject following administration of the HIF-2α inhibitor. The reduction in rectal bleeding may be evidenced by a reduction in the Mayo rectal bleeding sub-score of at least 1 point, or an absolute Mayo sub-score for rectal bleeding of 0 or 1. In some embodiments, stool frequency is reduced in the subject following administration of the HIF-2α inhibitor. The reduction in stool frequency may be evidenced by a reduction in the Mayo stool frequency sub-score of at least 1 point, or an absolute Mayo sub-score for stool frequency of 0 or 1.

In some embodiments, a HIF-2α inhibitor is administered to a subject suffering from steroid refractory ulcerative colitis. The ulcerative colitis may be mild, moderate or severe, particularly moderate or severe steroid refractory ulcerative colitis. Steroid refractory ulcerative colitis refers to colitis that remains active following steroid treatment. For example, a subject may suffer from ulcerative colitis following treatment with prednisolone (e.g., 0.75/mg/kg/day over a 4 week period). In some embodiments, a HIF-2α inhibitor is administered to a subject that fails to respond to a 7 or more day course of steroid treatment. In some embodiments, a HIF-2α inhibitor is administered to a subject that does not go in to remission following a 7 or more day course of steroid treatment.

In some embodiments, a HIF-2α inhibitor is administered to a subject suffering from steroid dependent ulcerative colitis. The ulcerative colitis may be mild, moderate or severe, particularly moderate or severe steroid dependent ulcerative colitis. Steroid dependent ulcerative colitis refers to ulcerative colitis that is being treated with a steroid, wherein the ulcerative colitis relapses (or flares) when the steroid dose is reduced or stopped. Thus, a subject suffering from steroid dependent ulcerative colitis cannot be weaned off steroids without a relapse of the ulcerative colitis. In some embodiments, steroid dependent ulcerative colitis includes ulcerative colitis wherein either: (i) it is not possible to reduce the steroid dose below the equivalent of prednisolone 10 mg/day within three months from initiating steroid treatment without a relapse or flare of the ulcerative colitis; or (ii) ulcerative colitis that relapses or flares within 3 months of stopping steroid treatment.

In some embodiments, a HIF-2α inhibitor is administered to a subject suffering from immunomodulator refractory ulcerative colitis, such as thiopurine refractory ulcerative colitis. The ulcerative colitis may be mild, moderate or severe, particularly moderate or severe immunomodulator dependent ulcerative colitis. Immunomodulator dependent ulcerative colitis refers to ulcerative colitis that is active or that relapses or flares in spite of the immunomodulator treatment. In particular, thiopurine refractory ulcerative colitis refers to ulcerative colitis that is active, relapses or flares in spite of being treated with a thiopurine for at least 3 months, for example azathioprine 2-2.5 mg/kg/day or mercaptopurine 1-1.5 mg/kg/day.

When an inflammatory disease of the digestive system, such as Crohn's disease or ulcerative colitis, is in remission, a maintenance therapy may be required to keep the disease in remission. For example, a maintenance therapy may prevent or reduce the risk of a flare or relapse of the disease. A maintenance therapy may also be used to reduce the frequency and/or severity of a flare or relapse of the disease. In some embodiments, a HIF-2a inhibitor is administered to a subject having an inflammatory disease of the digestive system, such as Crohn's disease or ulcerative colitis, in remission.

Currently, there are no curative drug treatments for Crohn's disease and ulcerative colitis. Therefore, maintenance of remission treatment of Crohn's disease or ulcerative colitis may be required for long periods of time, of many weeks, months, years or possible for the life-time of the subject in order to maintain the disease in remission. Generally, long term steroid usage is undesirable due to the side effects associated with chronic steroid treatment. Use of a HIF-2α inhibitor in accordance with the subject methods may enable the steroid dose required to maintain remission to be reduced or eliminated.

It is generally desirable to reduce or eliminate the use of steroids in the treatment of inflammatory diseases of the digestive system to reduce the undesirable side effects associated with steroid use. The use of a HIF-2α inhibitor for the treatment of an inflammatory disease may enable the dose of steroid administered to a patient to be reduced or eliminated.

In some embodiments, a HIF-2α inhibitor is administered to a subject that suffers from an inflammatory disease of the digestive system, but does not suffer from cancer. In some embodiments, the present disclosure provides a method of reducing inflammation of the digestive system in a subject in need thereof, comprising administering to the subject an effective amount of a HIF-2α inhibitor, wherein the inflammation is not associated with cancer.

In some embodiments, the HIF-2α inhibitor is administered as part of a therapeutic regimen that comprises administering one or more second agents (e.g. 1, 2, 3, 4, 5, or more second agents), either simultaneously or sequentially with the HIF-2α inhibitor. When administered sequentially, the HIF-2α inhibitor may be administered before or after the one or more second agents. When administered simultaneously, the HIF-2α inhibitor and the one or more second agents may be administered by the same route (e.g. injections to the same location; tablets taken orally at the same time), by a different route (e.g. a tablet taken orally while receiving an intravenous infusion), or as part of the same formulation (e.g. a solution comprising the HIF-2α inhibitor and the one or more second agents).

A variety of second agents and therapies suitable for combination therapy for reducing inflammation of the digestive system are available, and may be combined with one or more HIF-2α inhibitors. Examples of second agents include, but are not limited to, aminosalicylates, including 5-aminosalicylic acid (5-ASA) medications such as sulfasalazine, mesalamine, olsalazine, or balsalazide. 5-ASA medications may be administered in combination with a HIF-2α inhibitor by any suitable route, including oral, topical and rectal administration. In some embodiments, the 5-ASA is administered as a suppository, an enema, or an oral formulation. Preferably, the 5-ASA is administered as a suppository to subjects suffering from proctitis. In some embodiments, the subject suffers from left-sided colitis, and the 5-ASA is administered as an enema. In some embodiments, a HIF-2α inhibitor is administered in combination with a 5-ASA. In some embodiments, a HIF-2α inhibitor is administered in combination with a 5-ASA and a corticosteroid.

The second agent may include a steroid, such as a corticosteroid, including prednisone, methylprednisolone, hydrocortisone, budesonide, or beclomethasone dipropionate. The steroid may be administered in combination with a HIF-2α inhibitor by any suitable route, including oral, topical and rectal administration. In some embodiments, the steroid is administered as a suppository, an enema, a foam, or an oral formulation. In some embodiments, a HIF-2α inhibitor is administered in combination with a steroid, such as a corticosteroid. In some embodiments, a HIF-2α inhibitor is administered in combination with a 5-ASA and a corticosteroid.

The second agent may include a biologic agent, such as an anti-TNF-α or anti-integrin agent, including adalimumab, certolizumab pegol, golimumab, infliximab, infliximab-dyyb, natalizumab, or vedolizumab. The biologic may be administered in combination with a HIF-2α inhibitor by any suitable route, including intravenous infusion, intravenous injection and subcutaneous injection. In some embodiments, a HIF-2α inhibitor is administered in combination with a biologic, such as an anti-TNF-α or anti-integrin agent. In some embodiments, a HIF-2α inhibitor is administered in combination with a 5-ASA and a biologic. In some embodiments, a HIF-2α inhibitor is administered in combination with a corticosteroid and a biologic. In some embodiments, a HIF-2α inhibitor is administered in combination with a 5-ASA, a corticosteroid and a biologic.

The second agent may include an immunomodulator, such as a calcineurin inhibitor, azathioprine, 6-mercaptopurine, cyclosporine A, tacrolimus, or methotrexate. The immunomodulator may be administered in combination with a HIF-2α inhibitor by any suitable route, including oral, injection and topical administration. In some embodiments, a HIF-2α inhibitor is administered in combination with an immunomodulator. In some embodiments, a HIF-2α inhibitor is administered in combination with a 5-ASA and an immunomodulator. In some embodiments, a HIF-2α inhibitor is administered in combination with a corticosteroid and an immunomodulator. In some embodiments, a HIF-2α inhibitor is administered in combination with a 5-ASA, a corticosteroid and an immunomodulator. In some embodiments, a HIF-2α inhibitor is administered in combination with a biologic and an immunomodulator. In some embodiments, a HIF-2α inhibitor is administered in combination with a 5-ASA, a corticosteroid, a biologic and an immunomodulator.

The second agent may include an antibiotic, such as metronidazole or ciprofloxacin. The antibiotic may be administered in combination with a HIF-2α inhibitor by any suitable route, including oral and intravenous administration. In some embodiments, a HIF-2α inhibitor is administered in combination with an antibiotic. In some embodiments, a HIF-2α inhibitor is administered in combination with a 5-ASA and an antibiotic. In some embodiments, a HIF-2α inhibitor is administered in combination with a corticosteroid and an antibiotic. In some embodiments, a HIF-2α inhibitor is administered in combination with a 5-ASA, a corticosteroid and an antibiotic. In some embodiments, a HIF-2α inhibitor is administered in combination with a biologic and an antibiotic. In some embodiments, a HIF-2a inhibitor is administered in combination with a 5-ASA, a corticosteroid, a biologic and an antibiotic. In some embodiments, a HIF-2α inhibitor is administered in combination with an immunomodulator and an antibiotic. In some embodiments, a HIF-2α inhibitor is administered in combination with a 5-ASA, a corticosteroid, a biologic, an immunomodulator and an antibiotic.

In some embodiments, the HIF-2α inhibitor is administered in combination with surgery, such as proctocolectomy, ileal pouch anal-anastomosis, colectomy and ileostomy. In some embodiments, the HIF-2α inhibitor is administered in combination with one or more treatments selected from an aminosalicylate, a steroid, an anti-TNF-α agent, an anti-integrin agent, an immunomodulator, an antibiotic, and surgery. In some embodiments, the HIF-2α inhibitor is administered in combination with one or more treatments selected from an aminosalicylate, a steroid, an anti-TNF-α agent, an anti-integrin agent, an immunomodulator, and an antibiotic.

In some embodiments, the amount of HIF-2α inhibitor (either in a single dose or over multiple doses) is effective in reducing the disease activity index relative to untreated populations. For example, the HIF-2α inhibitor may prevent weight loss, improve stool consistency, and/or reduce blood in stool samples. The reduction in the disease activity index can be about or more than about 10%, 20%, 30%, 40%, 50%, 60%, 70%, 80%, 90%, 95%, or more. In some embodiments, the amount of HIF-2α inhibitor reverses shortening of colon length. In some embodiments, the amount of HIF-2α inhibitor (either in a single dose or over multiple doses) is effective in improving the disease activity index and/or improving the colon length in a mouse model of DSS induced colitis relative to vehicle treated populations. The improvement in the disease activity index can be about or more than about 10%, 20%, 30%, 40%, 50%, 60%, 70%, 80%, 90%, 95%, or more. One example of a colitis mouse model is a DSS induced colitis model. Dextran sulfate sodium (DSS) treatment leads to chemically induced damage of the epithelial monolayer lining the large intestine, allowing dissemination of pro-inflammatory intestinal content to the immune system of underlying tissue, leading to recruitment and activation of inflammatory immune cells.

A subject being treated with a HIF-2α inhibitor may be monitored to determine the effectiveness of treatment, and the treatment regimen may be adjusted based on the subject's physiological response to treatment. For example, if inhibition of a biological effect of HIF-2α inhibition is above or below a threshold, the dosing amount or frequency may decreased or increased, respectively. Alternatively, the treatment regimen may be adjusted to include, remove, or adjust an amount of a second agent. In some embodiments, treatment with the HIF-2α is discontinued if inhibition of the biological effect is above or below a threshold, such as in a lack of response. The biological effect may be a change in any of a variety of indicators associated with inflammation of the digestive system and the treatment thereof, or the severity or incidence of one or more symptoms of inflammation of the digestive system, examples of which are provided herein. The methods can further comprise continuing the therapy if the therapy is determined to be efficacious. The methods can comprise maintaining, tapering, reducing, or stopping the administered amount of a compound or compounds in the therapy if the therapy is determined to be efficacious. The methods can comprise increasing the administered amount of a compound or compounds in the therapy if it is determined not to be efficacious. Alternatively, the methods can comprise stopping therapy if it is determined not to be efficacious.

Any of a variety of HIF-2α inhibitors may be advantageously employed in the methods of the present disclosure. In general, a HIF-2α inhibitor is a compound that inhibits one or more biological effects of HIF-2α. Examples of biological effects of HIF-2α include, but are not limited to, heterodimerization of HIF-2α to HIF-1β, HIF-2α target gene expression, VEGF gene expression, and VEGF protein secretion. In some embodiments, the HIF-2α inhibitor is selective for HIF-2α, such that the inhibitor inhibits heterodimerization of HIF-2α to HIF-1β but not heterodimerization of HIF-1α to HIF-1β. Such biological effects may be inhibited by about or more than about 10%, 20%, 30%, 40%, 50%, 60%, 70%, 80%, 90%, 95%, or more.

Hypoxia-inducible factors (HIFs), like HIF-2α, are transcription factors that respond to changes in available oxygen in the cellular environment (e.g. a decrease in oxygen, or hypoxia). The HIF signaling cascade mediates the effects of hypoxia, the state of low oxygen concentration, on the cell. Hypoxia often keeps cells from differentiating. However, hypoxia promotes the formation of blood vessels, and is important for the formation of a vascular system in embryos, and cancer tumors. The hypoxia in wounds also promotes the migration of keratinocytes and the restoration of the epithelium. A HIF-2α inhibitor of the present disclosure may be administered in an amount effective in reducing any one or more of such effects of HIF-2α activity.

HIF-2α activity can be inhibited by inhibiting heterodimerization of HIF-2α to HIF-1β (ARNT), such as with inhibitor compounds disclosed herein. A variety of methods for measuring HIF-2α dimerization are available. For example, inhibition of heterodimerization of HIF-2α to HIF-1β (ARNT) may be determined in an Amplified Luminescent Proximity Homogeneous Assay (AlphaScreen). AlphaScreen, an in vitro assay, employs “PAS-B*” variants (R247E HIF-2α and E362R ARNT; Scheuermann et al, PNAS 2009) to assess functional disruption of PAS-PAS interactions in a high throughput screening (HTS) format. Inhibition of heterodimerization may also be determined by a reduction in HIF-2α target gene mRNA expression, and/or co-immunoprecipitation. In some embodiments, a HIF-2α inhibitor inhibits heterodimerization of HIF-2α to HIF-1β (ARNT) with an IC₅₀ value not exceeding 30 μM, for example, ranging from 10 to 30 μM, and further, for example, ranging from 1 to 30 μM, as determined by AlphaScreen. In some embodiments, the HIF-2α inhibitor has an IC₅₀ value not exceeding 1 μM as determined by AlphaScreen. A further description of methods for determining inhibition of heterodimerization are described in WO2014078479A2. In some embodiments, the HIF-2α inhibitor binds the PAS-B domain cavity of HIF-2α. Binding may be covalent or non-covalent, including but not limited to Van der Waals, hydrogen bond, and electrostatic interaction. In some embodiments, the binding is determined by co-crystallography.

Inhibition of heterodimerization of HIF-2α to HIF-1β (ARNT) may also be determined by a reduction in HIF-2α target gene mRNA expression. mRNA quantitation can be performed using real-time PCR technology. (Wong, et al, “Real-time PCR for mRNA quantitation”, 2005. BioTechniques 39, 1: 1-1.). Yet another method for determining inhibition of heterodimerization of HIF-2α to HIF-1β (ARNT) is by co-immunoprecipitation.

As described herein, HIF-2α is a transcription factor that plays important roles in regulating expression of target genes. Non-limiting examples of HIF-2α target gene include HMOX1, SFTPA1, CXCR4, PAI1, BDNF, hTERT, ATP7A, and VEGF. For instance, HIF-2α is an activator of VEGFA. Further non-limiting examples of HIF-2α target genes include HMOX1, EPO, CXCR4, PAI1, CCND1, CLUT1, IL6, and VEGF. A HIF-2α inhibitor of the present disclosure may be administered in an amount effective in reducing expression of any one or more of genes induced by HIF-2α activity. A variety of methods are available for the detection of gene expression level, and include the detection of gene transcription products (polynucleotides) and translation products (polypeptides). For example, gene expression can be detected and quantified at the DNA, RNA or mRNA level. Various methods that have been used to quantify mRNA include in situ hybridization techniques, fluorescent in situ hybridization techniques, reporter genes, RNase protection assays, Northern blotting, reverse transcription (RT)-PCR, SAGE, DNA microarray, tiling array, and RNA-seq. Examples of methods for the detection of polynucleotides include, but are not limited to selective colorimetric detection of polynucleotides based on the distance-dependent optical properties of gold nanoparticles, and solution phase detection of polynucleotides using interacting fluorescent labels and competitive hybridization. Examples for the detection of proteins include, but are not limited to microscopy and protein immunostaining, protein immunoprecipitation, immunoelectrophoresis, western blot, BCA assay, spectrophotometry, mass spectrophotometry and enzyme assay.

In some embodiments, inhibition of HIF-2α is characterized by a decrease in VEGF gene expression. The decrease may be measure by any of a variety of methods, such as those described herein. As a further example, the mRNA expression level of VEGF can be measured by quantitative PCR (QT-PCR), microarray, RNA-seq and nanostring. As another example, an ELISA assay can be used to measure the level VEGF protein secretion.

Measuring inhibition of biological effects of HIF-2α can comprise performing an assay on a biological sample, such as a sample from a subject. Any of a variety of samples may be selected, depending on the assay. Examples of samples include, but are not limited to whole blood (or portions thereof, including plasma), urine, saliva, and tissue biopsy.

In certain aspects, the HIF-2α inhibitor is a compound of Formula I′:

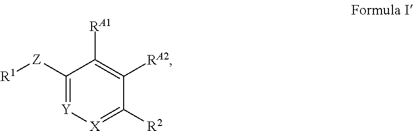

or a pharmaceutically acceptable salt or prodrug thereof, wherein:

X is selected from CR³ and N;

Y is selected from CR⁴ and N;

Z is selected from —O—, —S—, —S(O)—, —S(O)₂—, —C(O)—, —C(HR)—, —N(R⁶)—, C₁-C₃ alkylene, C₁-C₃ heteroalkylene, C₁-C₃ alkenylene or absent; R¹ is selected from C₁₋₆ alkyl, C₃₋₁₂ carbocycle and 3- to 12-membered heterocycle, each of which is optionally substituted with one or more R²⁰;

R², R³, R⁴ and R⁵ are each independently selected from hydrogen and R²⁰; R⁶ is selected from R²¹;

R^{A1} and R^{A2} are each independently selected from hydrogen and R²⁰, or R^{A1} and R^{A2} are taken together with the carbon atoms to which they are attached to form C₃₋₁₂ carbocycle or 3- to 12-membered heterocycle, each of which is optionally substituted with one or more R²⁰;

R²⁰ is independently selected at each occurrence from:
halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, and ═N(R²¹);
C₁₋₁₀ alkyl, C₂₋₁₀ alkenyl, and C₂₋₁₀ alkynyl, each of which is independently optionally substituted at each occurrence with one or more substituents selected from R²⁴; and
C₃₋₁₂ carbocycle and 3- to 12-membered heterocycle, each of which is independently optionally substituted at each occurrence with one or more substituents selected from R²⁵;

R²¹ is independently selected at each occurrence from hydrogen; and C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;

R²² and R²³ are taken together with the nitrogen atom to which they are attached to form a heterocycle, optionally substituted with one or more R²⁰;

R²⁴ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₃₋₁₂ carbocycle, and 3- to 12-membered heterocycle, wherein each C₃₋₁₂ carbocycle and 3- to 12-membered heterocycle is independently optionally substituted with one or more substituents selected from R²⁵; and

R²⁵ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₁₋₆ alkyl, C₁₋₆ haloalkyl, C₂₋₆ alkenyl, and C₂₋₆ alkynyl.

In some embodiments, for a compound of Formula I′, R^{A1} is selected from halogen, —CN, and C₁₋₁₀ alkyl. In some embodiments, R^{A1} is selected from halogen and C₁₋₆ alkyl. In some embodiments, R^{A1} is selected from —F, —Cl, —Br, and —I. In some embodiments, R^{A1} is fluoroalkyl, such as —CH₂F, —CHF₂ or —CF₃. In some embodiments, R^{A1} is hydrogen. In some embodiments, R^{A1} is 2- to 10-membered heteroalkyl, C₂₋₁₀ alkenyl, or C₂₋₁₀ alkynyl.

In some embodiments, for a compound of Formula I′, R^{A2} is selected from hydrogen, halogen, —CN, C₁₋₁₀ alkyl, C₂₋₁₀ alkenyl, 2- to 10-membered heteroalkyl, and —C(O)R²¹. In some embodiments, R^{A2} is —(CH₂)_{A3}OH, wherein A3 is 1, 2 or 3, such as —CH₂OH.

In some embodiments, for a compound of Formula I′, R^{A1} and R^{A2} are taken together with the carbon atoms to which they are attached to form C₃₋₁₂ carbocycle or 3- to 12-membered heterocycle, each of which is optionally substituted with one or more R²⁰. In some embodiments, R^{A1} and R^{A2} are taken together with the carbon atoms to which they are attached to form C₅₋₆ carbocycle or 5- to 6-membered heterocycle. In some embodiments, R^{A1} and R^{A2} are taken together with the carbon atoms to which they are attached to form C₅ carbocycle or 5-membered heterocycle. In some embodiments, R^{A1} and R^{A2} are taken together with the carbon atoms to which they are attached to form a 5- or 6-membered heterocycle comprising a lactone or lactol. Representative compounds include, but are not limited to, the following:

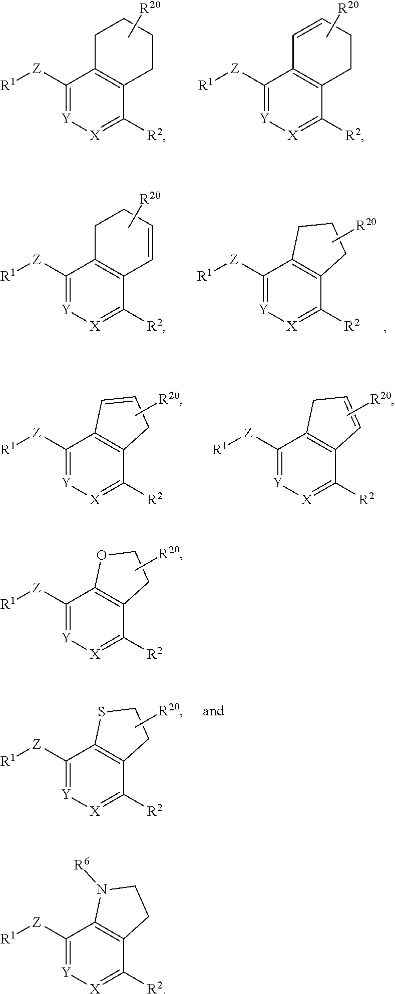

In some embodiments, for a compound of Formula I′, the ring formed by R^{A1} and R^{A2} is substituted with at least one substituent selected from R²⁰. In some embodiments, the ring is substituted with at least two substituents selected from R²⁰. In some embodiments, the ring is substituted with 1, 2, 3, 4, 5 or 6 substituents selected from R²⁰. In some embodiments, the ring is substituted with at least one substituent selected from halogen, —OH, —OR²¹, —N(R²¹)₂, —NR²²R²³, C₁₋₁₀ alkyl, C₂₋₁₀ alkenyl, and C₂₋₁₀ alkynyl. In some embodiments, the ring is substituted with at least one substituent selected from —F and —OH. In some embodiments, the ring is substituted with at least one —F. In some embodiments, the ring is substituted with —OH and at least one —F. In some embodiments, the ring is substituted with —F, —Cl, —OH, C₁₋₆ alkyl or C₁₋₆ heteroalkyl. In some embodiments, the ring is substituted with halogen, C₁₋₄ alkyl, C₁₋₄ alkoxy, or cyano. In some embodiments, the ring is substituted with oxo.

In certain aspects, the HIF-2α inhibitor is a compound of Formula I:

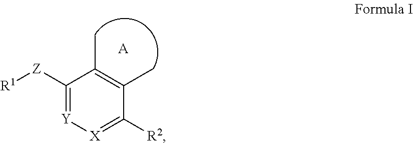

or a pharmaceutically acceptable salt or prodrug thereof, wherein:

X is selected from CR³ and N;

Y is selected from CR⁴ and N;

Z is selected from —O—, —S—, —S(O)—, —S(O)₂—, —C(O)—, —C(HR)—, —N(R⁶)—, C₁-C₃ alkylene, C₁-C₃ heteroalkylene, C₁-C₃ alkenylene or absent;

A is selected from C₃₋₁₂ carbocycle and 3- to 12-membered heterocycle, each of which is optionally substituted with one or more R²⁰;

R¹ is selected from C₁₋₆ alkyl, C₃₋₁₂ carbocycle and 3- to 12-membered heterocycle, each of which is optionally substituted with one or more R²⁰;

R², R³, R⁴ and R⁵ are each independently selected from hydrogen and R²⁰;

R⁶ is selected from R²¹;

R²⁰ is independently selected at each occurrence from:
halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, and ═N(R²¹);
C₁₋₁₀ alkyl, C₂₋₁₀ alkenyl, and C₂₋₁₀ alkynyl, each of which is independently optionally substituted at each occurrence with one or more substituents selected from R²⁴; and
C₃₋₁₂ carbocycle and 3- to 12-membered heterocycle, each of which is independently optionally substituted at each occurrence with one or more substituents selected from R²⁵;

R²¹ is independently selected at each occurrence from hydrogen; and C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;

R²² and R²³ are taken together with the nitrogen atom to which they are attached to form a heterocycle, optionally substituted with one or more R²⁰;

R²⁴ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₃₋₁₂ carbocycle, and 3- to 12-membered heterocycle, wherein each C₃₋₁₂ carbocycle and 3- to 12-membered heterocycle is independently optionally substituted with one or more substituents selected from R²⁵; and

R²⁵ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₁₋₆ alkyl, C₁₋₆ haloalkyl, C₂₋₆ alkenyl, and C₂₋₆ alkynyl.

In some embodiments, for a compound of Formula I, A is selected from C₅₋₆ carbocycle and 5- to 6-membered heterocycle. In some embodiments, A is selected from C₅ carbocycle and 5-membered heterocycle. In some embodiments, A is a 5- or 6-membered heterocycle comprising a lactone or lactol. Representative compounds include, but are not limited to, the following:

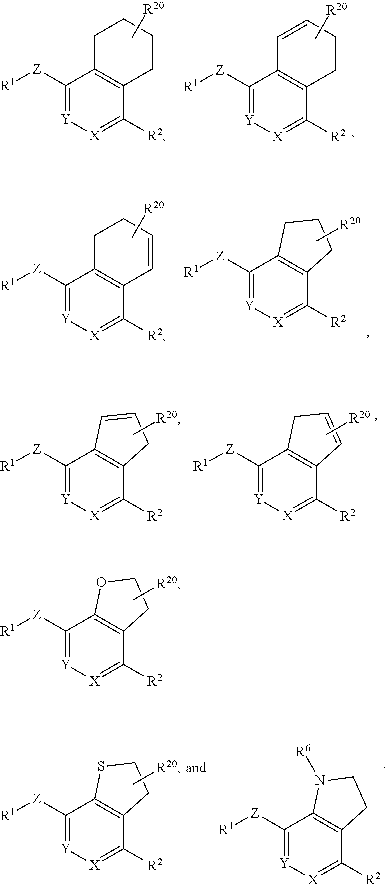

In some embodiments, for a compound of Formula I, A is substituted with at least one substituent selected from R²⁰. In some embodiments, A is substituted with at least two substituents selected from R²⁰. In some embodiments, A is substituted with 1, 2, 3, 4, 5 or 6 substituents selected from R²⁰. In some embodiments, A is substituted with at least one substituent selected from halogen, —OH, —OR²¹, —N(R²¹)₂, —NR²²R²³, C₁₋₁₀ alkyl, C₂₋₁₀ alkenyl, and C₂₋₁₀ alkynyl. In some embodiments, A is substituted with at least one substituent selected from —F and —OH. In some embodiments, A is substituted with at least one —F. In some embodiments, A is substituted with —OH and at least one —F. In some embodiments, A is substituted with —F, —Cl, —OH, C₁₋₆ alkyl or C₁₋₆ heteroalkyl. In some embodiments, A is substituted with halogen, C₁₋₄ alkyl, C₁₋₄ alkoxy, or cyano. In some embodiments, A is substituted with oxo.

In certain aspects, the HIF-2α inhibitor is a compound of Formula I-A:

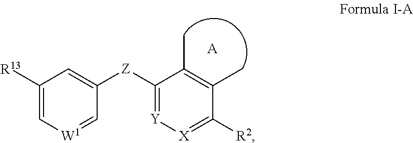

or a pharmaceutically acceptable salt or prodrug thereof, wherein:

X is selected from CR³ and N;

Y is selected from CR⁴ and N;

Z is selected from —O—, —S—, —S(O)—, —S(O)₂—, —C(O)—, —C(HR⁵)—, —N(R⁶)—, C₁-C₃ alkylene, C₁-C₃ heteroalkylene, C₁-C₃ alkenylene or absent;

W¹ is N or CR¹⁴;

A is selected from C₃₋₁₂ carbocycle and 3- to 12-membered heterocycle, each of which is optionally substituted with one or more R²⁰;

R², R³, R⁴, R⁵, R¹³ and R¹⁴ are each independently selected from hydrogen and R²⁰;

R⁶ is selected from R²¹;

R²⁰ is independently selected at each occurrence from:
halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, and ═N(R²¹);
C₁₋₁₀ alkyl, C₂₋₁₀ alkenyl, and C₂₋₁₀ alkynyl, each of which is independently optionally substituted at each occurrence with one or more substituents selected from R²⁴; and
C₃₋₁₂ carbocycle and 3- to 12-membered heterocycle, each of which is independently optionally substituted at each occurrence with one or more substituents selected from R²⁵;

R²¹ is independently selected at each occurrence from hydrogen; and C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;

R²² and R²³ are taken together with the nitrogen atom to which they are attached to form a heterocycle, optionally substituted with one or more R²⁰;

R²⁴ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₃₋₁₂ carbocycle, and 3- to 12-membered heterocycle, wherein each C₃₋₁₂ carbocycle and 3- to 12-membered heterocycle is independently optionally substituted with one or more substituents selected from R²⁵; and

R²⁵ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —R²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₁₋₆ alkyl, C₁₋₆ haloalkyl, C₂₋₆ alkenyl, and C₂₋₆ alkynyl.

In some embodiments, for a compound of Formula I-A, W¹ is CR¹⁴. In some embodiments, W¹ is N. In some embodiments, R¹⁴ is selected from hydrogen, halogen, —CN, C₁₋₆ alkyl and C₁₋₆ alkoxy. In some embodiments, R¹⁴ is selected from halogen, —CN, C₁₋₆ alkyl and C₁₋₆ alkoxy. In some embodiments, R¹⁴ is selected from halogen and —CN. In some embodiments, R¹⁴ is —F. In some embodiments, R¹³ is selected from halogen, —CN, C₁₋₆ alkyl and C₁₋₆ alkoxy. In some embodiments, R¹³ is selected from halogen and —CN. In some embodiments, R¹³ is —CN. In some embodiments, R¹³ is —CN and R¹⁴ is —F.

In some embodiments, for a compound of Formula I-A, A is selected from C. 6 carbocycle and 5- to 6-membered heterocycle. In some embodiments, A is selected from C₅ carbocycle and 5-membered heterocycle. In some embodiments, A is a 5- or 6-membered heterocycle comprising a lactone or lactol. Representative compounds include, but are not limited to, the following:

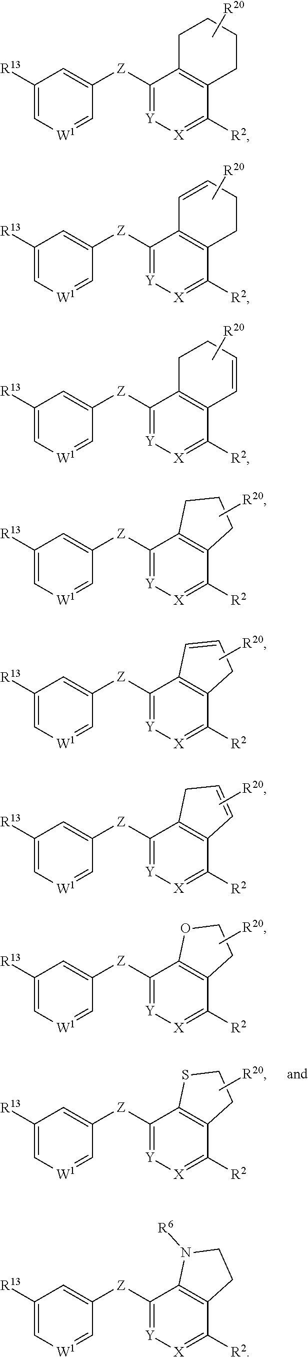

In some embodiments, for a compound of Formula I-A, A is substituted with at least one substituent selected from R²⁰. In some embodiments, A is substituted with at least two substituents selected from R²⁰. In some embodiments, A is substituted with 1, 2, 3, 4, 5 or 6 substituents selected from R²⁰. In some embodiments, A is substituted with at least one substituent selected from halogen, —OH, —OR²¹, —N(R²¹)₂, —NR²²R²³, C₁₋₁₀ alkyl, C₂₋₁₀ alkenyl, and C₂₋₁₀ alkynyl. In some embodiments, A is substituted with at least one substituent selected from —F and —OH. In some embodiments, A is substituted with at least one —F. In some embodiments, A is substituted with —OH and at least one —F. In some embodiments, A is substituted with —F, —Cl, —OH, C₁₋₆ alkyl or C₁₋₆ heteroalkyl. In some embodiments, A is substituted with halogen, C₁₋₄ alkyl, C₁₋₄ alkoxy, or cyano. In some embodiments, A is substituted with oxo.

In certain aspects, the HIF-2α inhibitor is a compound of Formula I-B:

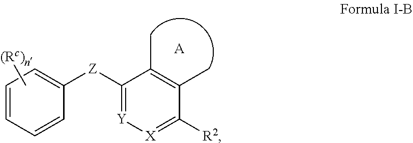

or a pharmaceutically acceptable salt or prodrug thereof, wherein:

X is selected from CR³ and N;

Y is selected from CR⁴ and N;

Z is selected from —O—, —S—, —S(O)—, —S(O)₂—, —C(O)—, —C(HR⁵)—, —N(R⁶)—, C₁-C₃ alkylene, C₁-C₃ heteroalkylene, C₁-C₃ alkenylene or absent;

A is selected from C₃₋₁₂ carbocycle and 3- to 12-membered heterocycle, each of which is optionally substituted with one or more R²⁰;

R², R³, R⁴, R⁵ and R^{c} are each independently selected at each occurrence from hydrogen and R²⁰;

n is 0, 1, 2, 3 or 4;

R⁶ is selected from R²¹;

R²⁰ is independently selected at each occurrence from:
halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, and ═N(R²¹);
C₁₋₁₀ alkyl, C₂₋₁₀ alkenyl, and C₂₋₁₀ alkynyl, each of which is independently optionally substituted at each occurrence with one or more substituents selected from R²⁴; and
C₃₋₁₂ carbocycle and 3- to 12-membered heterocycle, each of which is independently optionally substituted at each occurrence with one or more substituents selected from R²⁵;

R²¹ is independently selected at each occurrence from hydrogen; and C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;

R²² and R²³ are taken together with the nitrogen atom to which they are attached to form a heterocycle, optionally substituted with one or more R²⁰;

R²⁴ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₃₋₁₂ carbocycle, and 3- to 12-membered heterocycle, wherein each C₃₋₁₂ carbocycle and 3- to 12-membered heterocycle is independently optionally substituted with one or more substituents selected from R²⁵; and

R²⁵ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₁₋₆ alkyl, C₁₋₆ haloalkyl, C₂₋₆ alkenyl, and C₂₋₆ alkynyl.

In some embodiments, for a compound of Formula I-B, R^{c} is independently selected at each occurrence from hydrogen, halogen, —CN, C₁₋₆ alkyl and C₁₋₆ alkoxy. In some embodiments, R is selected from halogen, —CN, C₁₋₆ alkyl and C₁₋₆ alkoxy. In some embodiments, R^{c} is selected from halogen and —CN. In some embodiments, R^{c} is —F and —CN. In some embodiments, n′ is 1, 2 or 3. In some embodiments, n′ is 2. In some embodiments, n′ is 2 and R^{c} is selected from halogen and —CN.

In some embodiments, for a compound of Formula I-B, A is selected from C₅₋₆ carbocycle and 5- to 6-membered heterocycle. In some embodiments, A is selected from C₅ carbocycle and 5-membered heterocycle. In some embodiments, A is a 5- or 6-membered heterocycle comprising a lactone or lactol. Representative compounds include, but are not limited to, the following:

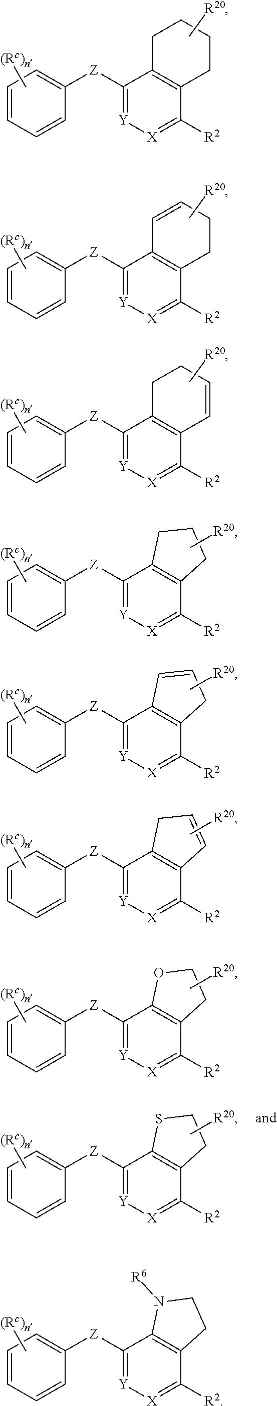

In some embodiments, for a compound of Formula I-B, A is substituted with at least one substituent selected from R²⁰. In some embodiments, A is substituted with at least two substituents selected from R²⁰. In some embodiments, A is substituted with 1, 2, 3, 4, 5 or 6 substituents selected from R²⁰. In some embodiments, A is substituted with at least one substituent selected from halogen, —OH, —OR²¹, —N(R²¹)₂, —NR²²R²³, C₁₋₁₀ alkyl, C₂₋₁₀ alkenyl, and C₂₋₁₀ alkynyl. In some embodiments, A is substituted with at least one substituent selected from —F and —OH. In some embodiments, A is substituted with at least one —F. In some embodiments, A is substituted with —OH and at least one —F. In some embodiments, A is substituted with —F, —Cl, —OH, C₁₋₆ alkyl or C₁₋₆ heteroalkyl. In some embodiments, A is substituted with halogen, C₁₋₄ alkyl, C₁₋₄ alkoxy, or cyano. In some embodiments, A is substituted with oxo.

In certain aspects, the HIF-2α inhibitor is a compound of Formula I-C:

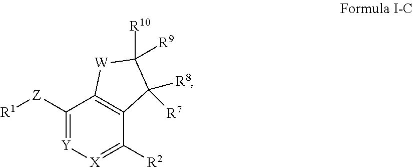

or a pharmaceutically acceptable salt or prodrug thereof, wherein:

X is selected from CR³ and N;

Y is selected from CR⁴ and N;

Z is selected from —O—, —S—, —S(O)—, —S(O)₂—, —C(O)—, —C(HR⁵)—, —N(R⁶)—, C₁-C₃ alkylene, C₁-C₃ heteroalkylene, C₁-C₃ alkenylene or absent;

W is selected from O, S, CR¹¹R¹² and NR⁶;

R¹ is selected from C₁₋₆ alkyl, C₃₋₁₂ carbocycle and 3- to 12-membered heterocycle, each of which is optionally substituted with one or more R²⁰;

R², R³, R⁴, R⁵, R⁷, R⁸, R⁹, R¹⁰, R¹¹ and R¹² are each independently selected from hydrogen and R²⁰, or R⁷ and R⁸ in combination form oxo or oxime;

R⁶ is selected from R²¹;

R²⁰ is independently selected at each occurrence from:
halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, and ═N(R²¹);
C₁₋₁₀ alkyl, C₂₋₁₀ alkenyl, and C₂₋₁₀ alkynyl, each of which is independently optionally substituted at each occurrence with one or more substituents selected from R²⁴; and
C₃₋₁₂ carbocycle and 3- to 12-membered heterocycle, each of which is independently optionally substituted at each occurrence with one or more substituents selected from R²⁵;

R²¹ is independently selected at each occurrence from hydrogen; and C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;

R²² and R²³ are taken together with the nitrogen atom to which they are attached to form a heterocycle, optionally substituted with one or more R²⁰;

R²⁴ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₃₋₁₂ carbocycle, and 3- to 12-membered heterocycle, wherein each C₃₋₁₂ carbocycle and 3- to 12-membered heterocycle is independently optionally substituted with one or more substituents selected from R²⁵; and

R²⁵ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₁₋₆ alkyl, C₁₋₆ haloalkyl, C₂₋₆ alkenyl, and C₂₋₆ alkynyl.

In some embodiments, for a compound of Formula I-C, R⁷ is selected from hydrogen, —OH, C₁₋₆ alkoxy, —N(R²¹)₂ and —NR²²R²³. In some embodiments, R⁷ is selected from —OH and —N(R²¹)₂. In some embodiments, R⁷ is —OH.

In some embodiments, for a compound of Formula I-C, R⁸ is selected from hydrogen, halogen, —OH, —CN, C₁₋₆ alkyl and C₁₋₆ alkoxy. In some embodiments, R⁸ is hydrogen. In some embodiments, R⁸ is selected from C₁₋₆ alkyl and C₁₋₆ alkenyl.

In some embodiments, for a compound of Formula I-C, R⁹ and R¹⁰ are each independently selected from hydrogen, halogen, —OH, C₁₋₆ alkyl and 1- to 6-membered heteroalkyl. In some embodiments, R⁹ and R¹⁰ in combination form oxo, oxime or methylene. In some embodiments, R⁹, R¹⁰ and the carbon atom to which they are attached form C₃₋₈ cycloalkyl or 3- to 8-membered heterocycloalkyl. In some embodiments, at least one of R⁹ and R¹⁰ is halogen, such as —F.

In some embodiments, for a compound of Formula I-C, R¹¹ and R¹² are each independently selected from hydrogen, halogen, —OH, C₁₋₆ alkyl and 1- to 6-membered heteroalkyl. In some embodiments, R¹¹ and R¹² in combination form oxo, oxime or methylene. In some embodiments, R¹¹, R¹² and the carbon atom to which they are attached form C₃₋₈ cycloalkyl or 3- to 8-membered heterocycloalkyl. In some embodiments, at least one of R¹¹ and R¹² is halogen, such as —F.

In some embodiments, for a compound of Formula I-C, R⁹, R¹¹ and the carbon atoms to which they are attached form C₃₋₈ cycloalkyl or 3- to 8-membered heterocycloalkyl.

In some embodiments, for a compound of Formula I-C, at least one of R⁹, R¹⁰, R¹¹ and R¹² is halogen. In some embodiments, at least two of R⁹, R¹⁰, R¹¹ and R¹² are halogen. In some embodiments, at least three of R⁹, R¹⁰, R¹¹ and R¹² are halogen. In some embodiments, R⁹ is halogen. In some embodiments, R⁹ and R¹⁰ are each halogen. In some embodiments, R⁹ and R¹¹ are each halogen. In some embodiments, R⁹, R¹⁰ and R¹¹ are each halogen. In some embodiments, at least one of R⁹, R¹⁰, R¹¹ and R¹² is —F. In some embodiments, at least two of R⁹, R¹⁰, R¹¹ and R¹² are —F. In some embodiments, at least three of R⁹, R¹⁰, R¹¹ and R¹² are —F. In some embodiments, R⁹ is —F. In some embodiments, R⁹ and R¹⁰ are each —F. In some embodiments, R⁹ and R¹¹ are each —F. In some embodiments, R⁹, R¹⁰ and R¹¹ are each —F.

In some embodiments for a compound of Formula I-C, at least one of R⁹, R¹⁰, R¹¹ and R¹² is —F, R⁷ is —OH and R⁸ is hydrogen. In some embodiments, at least two of R⁹, R¹⁰, R¹¹ and R¹² are —F, R⁷ is —OH and R⁸ is hydrogen. In some embodiments, at least three of R⁹, R¹⁰, R¹¹ and R¹² are —F, R⁷ is —OH and R⁸ is hydrogen. In some embodiments, R⁹ is —F, R⁷ is —OH and R⁸ is hydrogen. In some embodiments, R⁹ and R¹⁰ are each —F, R⁷ is —OH and R⁸ is hydrogen. In some embodiments, R⁹ and R¹¹ are each —F, R⁷ is —OH and R⁸ is hydrogen. In some embodiments, R⁹, R¹⁰ and R¹¹ are each —F, R⁷ is —OH and R⁸ is hydrogen. In some embodiments, R⁹ and R¹⁰ are each —F, R¹¹ and R¹² are each hydrogen, R⁷ is —OH and R⁸ is hydrogen. In some embodiments, R⁹, R¹⁰ and R¹¹ are each —F, R¹² is hydrogen, R⁷ is —OH and R⁸ is hydrogen. In some embodiments, R⁹ and R¹¹ are each —F, R¹⁰ and R¹² are each hydrogen, R⁷ is —OH and R⁸ is hydrogen.

In some embodiments, for a compound of Formula I-C, R⁷ is selected from hydrogen, halogen, —OR²¹, —N(R²¹)₂ and —NR²²R²³; R⁸ is selected from hydrogen, C₁₋₁₀ alkyl, C₂₋₁₀ alkenyl, and C₂₋₁₀ alkynyl; and R⁹, R¹⁰, R¹¹ and R¹² are each independently selected from hydrogen, halogen, —OR²¹, C₁₋₁₀ alkyl and 2- to 10-membered heteroalkyl. In some embodiments, R⁸ is hydrogen. In some embodiments, R⁹ is fluoro. In some embodiments, R² is selected from —S(═O)₂R²¹, —S(═O)(═NR²¹)R²¹ and C₁₋₃ fluoroalkyl; Z is —O—; R¹¹ is —OH; and R¹² is hydrogen, optionally wherein R¹ is selected from C₆₋₁₀ aryl, 5- to 8-membered heteroaryl, C₃₋₈ cycloalkyl and 3- to 8-membered heterocycloalkyl.

In some embodiments, for a compound of Formula I-C, W is selected from O and CR¹¹R¹². In some embodiments, W is CR¹¹R²¹, such as —CH₂—, —CHF—, or —CF₂—. In some embodiments, W is O.

In some embodiments, for a compound of Formula I-C:

X is CH;

Y is CH;

Z is —O—;

W is selected from O and CR¹¹R¹²;

R¹ is phenyl, pyridyl or C₃₋₆ cycloalkyl, each of which is optionally substituted with one or more R²⁰;

R² is selected from —CN, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, C₁₀ fluoroalkyl, C₃₋₁₂ carbocycle and 3- to 12-membered heterocycle, wherein said C₃₋₁₂ carbocycle and 3- to 12-membered heterocycle are optionally substituted with one or more R²⁰;

R⁷ is selected from hydrogen, halogen, —OR²¹, —N(R²¹)₂ and —NR²²R²³;

R⁸ is selected from hydrogen, C₁₋₁₀ alkyl, C₂₋₁₀ alkenyl, and C₂₋₁₀ alkynyl;

R⁹, R¹⁰, R¹¹ and R¹² are each independently selected from hydrogen, halogen, —OR²¹, C₁₋₁₀ alkyl and 2- to 10-membered heteroalkyl R²⁰ is independently selected at each occurrence from:
halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, and ═N(R²¹);
C₁₋₁₀ alkyl, C₂₋₁₀ alkenyl, and C₂₋₁₀ alkynyl, each of which is independently optionally substituted at each occurrence with one or more substituents selected from R²⁴; and
C₃₋₁₂ carbocycle and 3- to 12-membered heterocycle, each of which is independently optionally substituted at each occurrence with one or more substituents selected from R²⁵;

R²¹ is independently selected at each occurrence from hydrogen; and C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;

R²² and R²³ are taken together with the nitrogen atom to which they are attached to form a heterocycle, optionally substituted with one or more R²⁰;

R²⁴ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₃₋₁₂ carbocycle, and 3- to 12-membered heterocycle, wherein each C₃₋₁₂ carbocycle and 3- to 12-membered heterocycle is independently optionally substituted with one or more substituents selected from R²⁵; and R²⁵ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₁₋₆ alkyl, C₁₋₆ haloalkyl, C₂₋₆ alkenyl, and C₂₋₆ alkynyl.

In certain aspects, the HIF-2α inhibitor is a compound of Formula I-D, I-E, I-F or I-G:

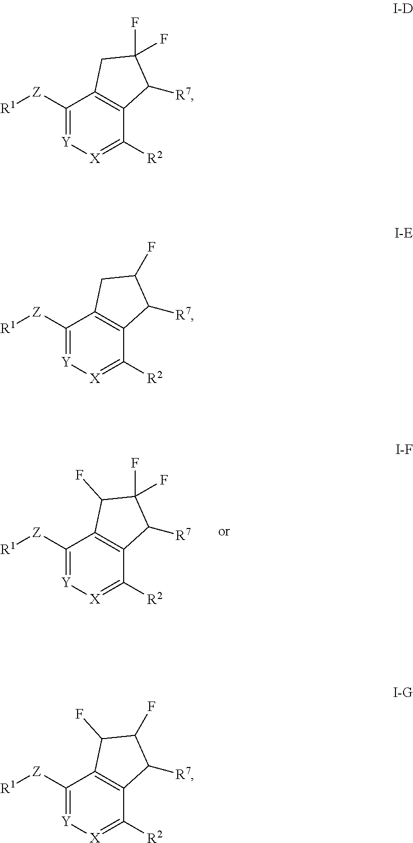

or a pharmaceutically acceptable salt or prodrug thereof, wherein:

X is selected from CR³ and N;

Y is selected from CR⁴ and N;

Z is selected from —O—, —S—, —S(O)—, —S(O)₂—, —C(O)—, —C(HR⁵)—, —N(R⁶)—, C₁-C₃ alkylene, C₁-C₃ heteroalkylene, C₁-C₃ alkenylene or absent;

R¹ is selected from C₁₋₆ alkyl, C₃₋₁₂ carbocycle and 3- to 12-membered heterocycle, each of which is optionally substituted with one or more R²⁰;

R², R³, R⁴, R⁵ and R⁷ are each independently selected from hydrogen and R²⁰; R⁶ is selected from R²¹;

R²⁰ is independently selected at each occurrence from:
halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, and ═N(R²¹);
C₁₋₁₀ alkyl, C₂₋₁₀ alkenyl, and C₂₋₁₀ alkynyl, each of which is independently optionally substituted at each occurrence with one or more substituents selected from R²⁴; and
C₃₋₁₂ carbocycle and 3- to 12-membered heterocycle, each of which is independently optionally substituted at each occurrence with one or more substituents selected from R²⁵;

R²¹ is independently selected at each occurrence from hydrogen; and C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;

R²² and R²³ are taken together with the nitrogen atom to which they are attached to form a heterocycle, optionally substituted with one or more R²⁰;

R²⁴ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₃₋₁₂ carbocycle, and 3- to 12-membered heterocycle, wherein each C₃₋₁₂ carbocycle and 3- to 12-membered heterocycle is independently optionally substituted with one or more substituents selected from R²⁵; and

R²⁵ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₁₋₆ alkyl, C₁₋₆ haloalkyl, C₂₋₆ alkenyl, and C₂₋₆ alkynyl.

In some embodiments, for a compound of Formula I-D, I-E, I-F or I-G, R⁷ is selected from hydrogen, —OH, C₁₋₆ alkoxy, —N(R²¹)₂ and —NR²²R²³. In some embodiments, R⁷ is selected from —OH and —N(R²¹)₂. In some embodiments, R⁷ is —OH.

In certain aspects, the HIF-2α inhibitor is a compound of Formula I-H, I-I, I-J or I-K:

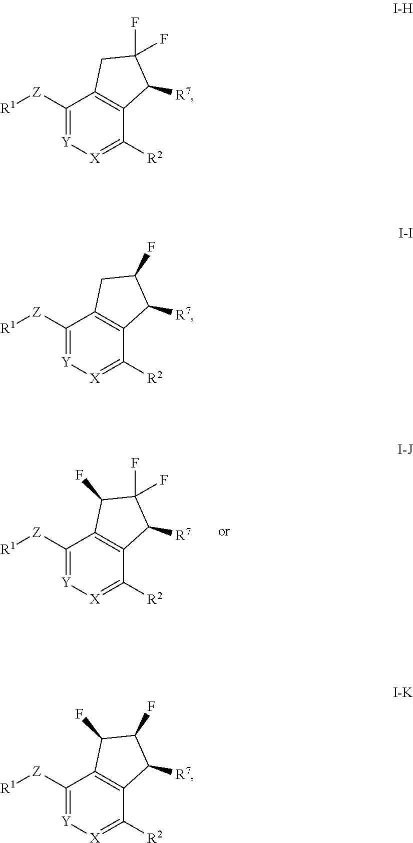

or a pharmaceutically acceptable salt or prodrug thereof, wherein:

X is selected from CR³ and N;

Y is selected from CR⁴ and N;

Z is selected from —O—, —S—, —S(O)—, —S(O)₂—, —C(O)—, —C(HR⁵)—, —N(R⁶)—, C₁-C₃ alkylene, C₁-C₃ heteroalkylene, C₁-C₃ alkenylene or absent;

R¹ is selected from C₁₋₆ alkyl, C₃₋₁₂ carbocycle and 3- to 12-membered heterocycle, each of which is optionally substituted with one or more R²⁰;

R², R³, R⁴, R⁵ and R⁷ are each independently selected from hydrogen and R²⁰;

R⁶ is selected from R²¹;

R²⁰ is independently selected at each occurrence from:
halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, and ═N(R²¹);
C₁₋₁₀ alkyl, C₂₋₁₀ alkenyl, and C₂₋₁₀ alkynyl, each of which is independently optionally substituted at each occurrence with one or more substituents selected from R²⁴; and
C₃₋₁₂ carbocycle and 3- to 12-membered heterocycle, each of which is independently optionally substituted at each occurrence with one or more substituents selected from R²⁵;

R²¹ is independently selected at each occurrence from hydrogen; and C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;

R²² and R²³ are taken together with the nitrogen atom to which they are attached to form a heterocycle, optionally substituted with one or more R²⁰;

R²⁴ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₃₋₁₂ carbocycle, and 3- to 12-membered heterocycle, wherein each C₃₋₁₂ carbocycle and 3- to 12-membered heterocycle is independently optionally substituted with one or more substituents selected from R²⁵; and

R²⁵ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₁₋₆ alkyl, C₁₋₆ haloalkyl, C₂₋₆ alkenyl, and C₂₋₆ alkynyl.

In some embodiments, for a compound of Formula I-H, I-I, I-J or I-K, R⁷ is selected from hydrogen, —OH, C₁₋₆ alkoxy, —N(R²¹)₂ and —NR²²R²³. In some embodiments, R⁷ is selected from —OH and —N(R²¹)₂. In some embodiments, R⁷ is —OH.

In some embodiments, for a compound of Formula I-C, I-D, I-E, I-F, I-G, I-H, I-I, I-J or I-K, R⁷ is selected from —OR²¹ and —N(R²¹)₂, such as —OH and —NH₂. In some embodiments, R⁷ is OH.

In some embodiments, for a compound of Formula I, I, I-C, I-D, I-E, I-F, I-G, I-H, I-I, I-J or I-K, R¹ is selected from C₆₋₁₀ aryl, 5- to 8-membered heteroaryl, C₃₋₈ cycloalkyl and 3- to 8-membered heterocycloalkyl. In some embodiments, R¹ is selected from phenyl and pyridyl. In some embodiments, R¹ is phenyl. In some embodiments, R¹ is selected from C₃₋₈ cycloalkyl and 3- to 8-membered heterocycloalkyl.

In some embodiments, for a compound of Formula I, I, I-C, I-D, I-E, I-F, I-G, I-H, I-I, I-J or I-K, R¹ is

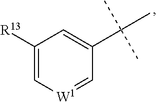

wherein W¹ is N or CR¹⁴; and R¹³ and R¹⁴ are independently selected from hydrogen and R²⁰. In some embodiments, R¹⁴ is selected from hydrogen, halogen, —CN, C₁₋₆ alkyl and C₁₋₆ alkoxy. In some embodiments, R¹⁴ is selected from halogen, —CN, C₁₋₆ alkyl and C₁₋₆ alkoxy. In some embodiments, R¹⁴ is selected from halogen and —CN. In some embodiments, R¹⁴ is —F. In some embodiments, R¹³ is selected from halogen, —CN, C₁₋₆ alkyl and C₁₋₆ alkoxy. In some embodiments, R¹³ is selected from halogen and —CN. In some embodiments, R¹³ is —CN. In some embodiments, R¹³ is —CN and R¹⁴ is —F.

In some embodiments, for a compound of Formula I′, I, I-C, I-D, I-E, I-F, I-G, I-H, I-I, I-J or I-K, R¹ is

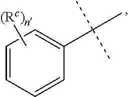

wherein R^{c} is independently selected at each occurrence from hydrogen and R²⁰; and n′ is 0, 1, 2, 3 or 4. In some embodiments, R^{c} is independently selected at each occurrence from hydrogen, halogen, —CN, C₁₋₆ alkyl and C₁₋₆ alkoxy. In some embodiments, R is selected from halogen, —CN, C₁₋₆ alkyl and C₁₋₆ alkoxy. In some embodiments, R^{c} is selected from halogen and —CN. In some embodiments, R^{c} is —F and —CN. In some embodiments, n′ is 1, 2 or 3. In some embodiments, n′ is 2. In some embodiments, n′ is 2 and R^{c} is selected from halogen and —CN.

In some embodiments, for a compound of Formula I′, I, I-C, I-D, I-E, I-F, I-G, I-H, I-I, I-J or I-K, R¹ is bicyclic heteroaryl. In some embodiments, R¹ is selected from:

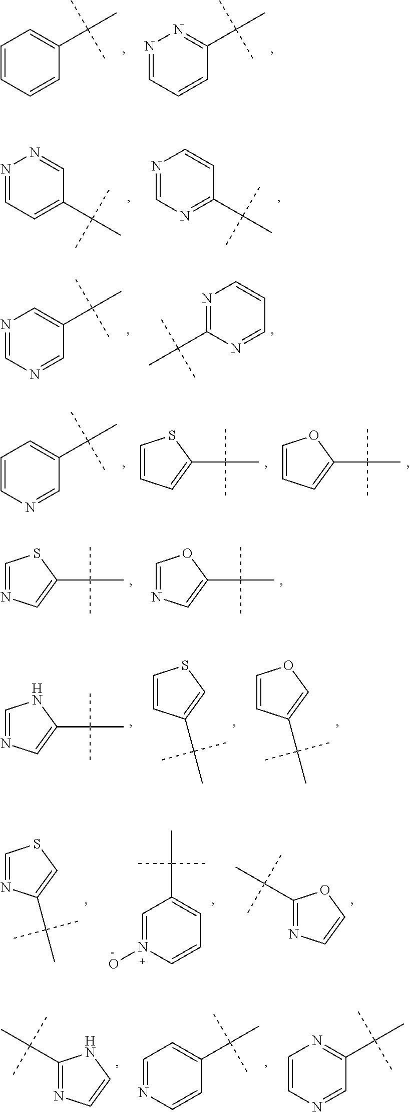

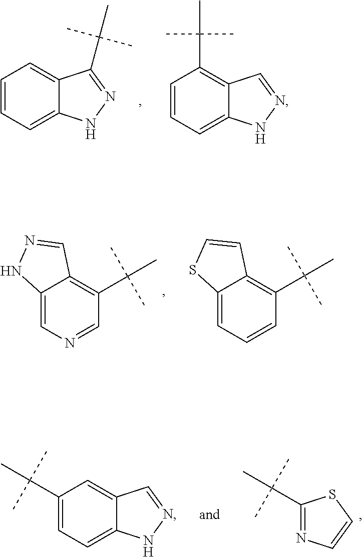

wherein the rings specified for R are optionally substituted with one or more R²⁰. In some embodiments, R¹ is selected from

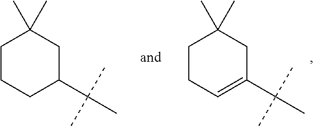

wherein the rings specified for R¹ are optionally substituted with one or more R²⁰.

In some embodiments, for a compound of Formula I′, I, I-C, I-D, I-E, I-F, I-G, I-H, I-I, I-J or I-K, R¹ is cycloalkyl. In some embodiments, R¹ is heterocycloalkyl. In some embodiments, R¹ is selected from C₃-C₆ cycloalkyl and 3- to 6-membered heterocycloalkyl. In some embodiments, R¹ is cyclobutyl. In some embodiments, said heterocycloalkyl, cycloalkyl and cyclobutyl are optionally substituted with one or more R²⁰. In some embodiments, R¹ is acyl or cyano. In some embodiments, R¹ is acetyl. In some embodiments, R¹ is alkyl. In some embodiments, the alkyl is substituted with one or more R²⁰. In some embodiments, the alkyl is substituted with at least one fluoro. In some embodiments, R¹ is heteroalkyl. In some embodiments, R¹ is selected from the group consisting of

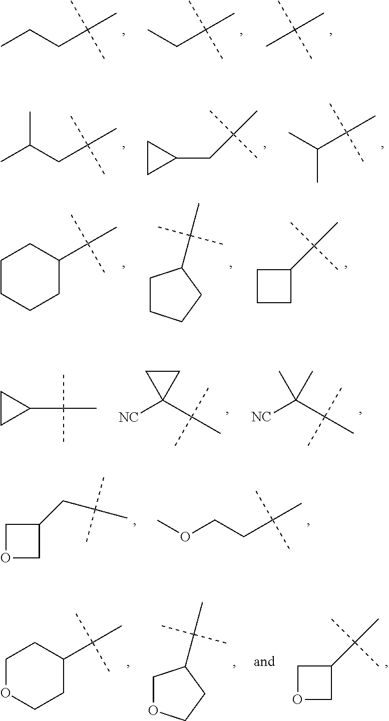

wherein each of the members are optionally substituted with one or more R²⁰.

In some embodiments, for a compound of Formula I′, I, I-C, I-D, I-E, I-F, I-G, I-H, I-I, I-J or I-K, R is substituted with one or more R²⁰. In some embodiments, R is substituted with two or more R²⁰. In some embodiments, R is substituted with three or more R²⁰. In some embodiments, R is substituted with two R²⁰ substituents. In some embodiments, R is substituted with one or more substituents selected from halogen, —CN, C₁₋₆ alkyl and C₁₋₆ alkoxy. In some embodiments, R is substituted with one or more substituents selected from halogen and —CN. In some embodiments, R is substituted with one or more substituents selected from —F and —CN. In some embodiments, R is substituted with halogen and —CN. In some embodiments, R is substituted with —F and —CN.

In some embodiments, for a compound of Formula I′, I, I-A, I-B, I-C, I-D, I-E, I-F, I-G, I-H, I-I, I-J or I-K, R² is selected from —CN, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, C₁₋₁₀ fluoroalkyl, C₃₋₁₂ carbocycle and 3- to 12-membered heterocycle. In some embodiments, R² is selected from —CN, —S(═O)R²¹, —S(═O)₂R²¹, —S(═O)(═NR²¹)R²¹, —S(═O)₂N(R²)₂, —S(═O)₂NR²²R²³, —NR²¹S(═O)₂R²¹ and C₁₋₁₀ fluoroalkyl. In some embodiments, R² is selected from —S(═O)₂CH₃, —S(═O)₂CHF₂, —S(═O)(═N—CN)CH₃ and CF₃. In some embodiments, R² is C₁₋₄ fluoroalkyl. In some embodiments, R² is —S(═O)₂R²¹. In some embodiments, R² is —S(═O)₂R²¹, wherein R² is C₁₋₄ fluoroalkyl. In some embodiments, R² is —S(═O)₂R²¹, wherein R²¹ is selected from C₁₋₁₀ alkyl or C₃₋₁₀ cycloalkyl. In some embodiments, R² is C₁₋₄ alkyl, optionally substituted with one or more fluorines. Suitable examples of fluorine-substituted C₁₋₄ alkyl include, but are not limited to, —CH₂F, —CHF₂, —CF₃, —CH₂CF₃, —CH₂CHF₂, —CH₂CH₂F, —CHFCH₃ and —CF₂CH₃. In some embodiments, R¹ is methyl, optionally substituted with one or more fluorines. In some embodiments, R² is —S(═O)(═NR²¹)R²¹, wherein each R²¹ is independently selected from hydrogen, —CN, C₁₋₆ alkyl and C₃₋₁₀ cycloalkyl. In some embodiments, R²¹ is C₁₋₄ alkyl, optionally substituted with one or more fluorines. In some embodiments, R² is —S(═O)₂N(R²¹)₂, wherein each R²¹ is independently selected from hydrogen, C₁₋₆ alkyl, 3- to 6-membered heteroalkyl, C₃₋₁₀ cycloalkyl or 3- to 10-membered heterocycloalkyl, wherein at least one R²¹ is hydrogen. In some embodiments, one R²¹ is hydrogen and the other R²¹ is C₁₋₄ alkyl. In some embodiments, R² is selected from —CN, —CF₃, —S(═O)CH₃, —S(═O)₂CH₃, —S(═O)₂CH₂F, —S(═O)₂CHF₂, —S(═O)₂CF₃, —S(═O)₂NH₂, —S(═O)₂NHCH₃, —S(═O)(═NH)CH₃, —S(═O)(═NH)CH₂F, —S(═O)(═NH)CHF₂, —S(═O)(═NH)CF₃, —S(═O)(═N—CN)CH₃, —S(═O)(═N—CN)CH₂F, —S(═O)(═N—CN)CHF₂ and —S(═O)(═N—CN)CF₃. In some embodiments, R² is selected from C₆₋₁₀ aryl and 5- to 8-membered heteroaryl, such as 5-membered heteroaryl. In some embodiments, R² is selected from C₃₋₁₀ cycloalkyl, C₆₋₁₀ aryl and 5- to 8-membered heteroaryl. In some embodiments, R² is selected from C₃₋₁₀ cycloalkyl, C₆₋₁₀ aryl and 5- to 8-membered heteroaryl, optionally substituted with one or more substituents selected from halogen, —CN, —OH, C₁₋₆ alkyl and C₁₋₆ haloalkyl.

In some embodiments, for a compound of Formula I′, I, I-A, I-B, I-C, I-D, I-E, I-F, I-G, I-H, I-I, I-J or I-K, R³, R⁴, R⁵ and R⁶ are independently selected from hydrogen, C₁₋₁₀ alkyl and C₁₋₁₀ alkoxy. In some embodiments, R³, R⁴, R⁵ and R⁶ are independently selected from hydrogen, C₁₋₄ alkyl and C₁₋₄ alkoxy. In some embodiments, R³ is hydrogen. In some embodiments, R⁴ is hydrogen. In some embodiments, R⁵ is hydrogen. In some embodiments, R⁶ is hydrogen. In some embodiments, R³ is methyl. In some embodiments, R⁴ is methyl. In some embodiments, R⁵ is methyl. In some embodiments, R⁶ is methyl.

In some embodiments, for a compound of Formula I′, I, I-A, I-B, I-C, I-D, I-E, I-F, I-G, I-H, I-I, I-J or I-K, X is N and Y is CR⁴. In some embodiments, X is CR³ and Y is N. In some embodiments, X is N and Y is N. In some embodiments, X is CR³ and Y is CR⁴. In some embodiments, X is CR³; Y is CR⁴; and R³ and R⁴ are independently selected from hydrogen, halogen, —CN, C₁₋₄ alkyl and C₁₋₄ alkoxy. In some embodiments, X is CR³; Y is CR⁴; and R³ and R⁴ are each hydrogen.

In some embodiments, for a compound of Formula I, I, I-A, I-B, I-C, I-D, I-E, I-F, I-G, I-H, I-I, I-J or I-K, Z is —O—, —S—, —S(O)—, —S(O)₂—, —S(O₂)N(R⁶)—, —C(O)—, —C(O)O—, —C(HR⁵)—, —N(R⁶)—, —C(O)N(R⁶)—, alkylene, alkenylene, alkynylene, heteroalkylene, or absent. In some embodiments, Z is —O—, —S—, —S(O)—, —S(O)₂—, —C(O)—, —C(HR⁵)—, —N(R⁶)—, C₁-C₃ alkylene, C₁-C₃ heteroalkylene, C₁-C₃ alkenylene or absent. In some embodiments, Z is —O—. In some embodiments, Z is —S—. In some embodiments, Z is —C(HR5)-. In some embodiments, Z is —N(R⁶). In some embodiments, Z is absent.

In some embodiments, for a compound of Formula I′, I, I-C, I-D, I-E, I-F, I-G, I-H, I-I, I-J or I-K:

X is CH;

Y is CH;

Z is —O—;

R¹ is phenyl, pyridyl or C₃₋₆ cycloalkyl, each of which is optionally substituted with one or more R²⁰; and

R² is selected from —CN, —S(═O)R²¹, —S(═O)₂R²¹, —S(═O)(═NR²¹)R²¹, —S(═O)₂N(R²¹)₂, —S(═O)₂NR¹²R²³, —NR²¹S(═O)₂R²¹, C₁₋₁₀ fluoroalkyl, C₃₋₁₂ carbocycle and 3- to 12-membered heterocycle, wherein said C₃₋₁₂ carbocycle and 3- to 12-membered heterocycle are optionally substituted with one or more R²⁰.

In some embodiments, the HIF-2α inhibitor is

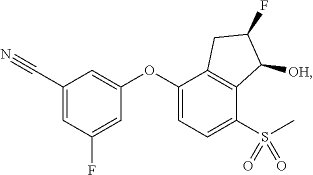 [SMILES: CS(=O)(=O)c1ccc(Oc2cc(F)cc(C#N)c2)c2c1[C@H](O)[C@H](F)C2]

or a pharmaceutically acceptable salt thereof. In some embodiments, the HIF-2α inhibitor is selected from the group consisting of:

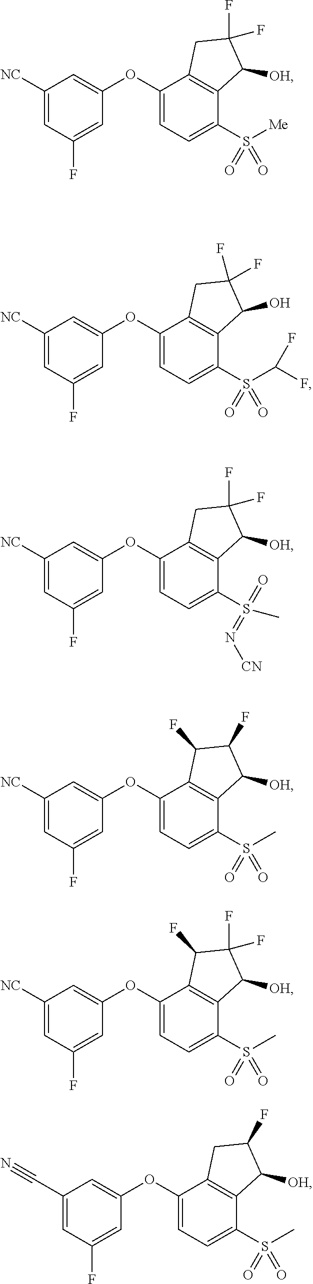

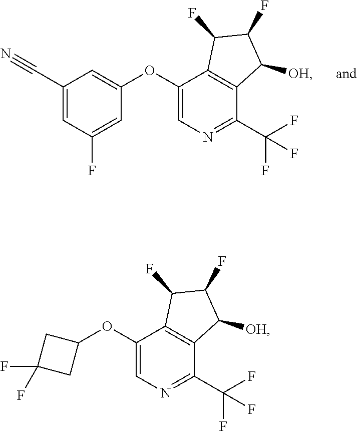 [SMILES: N#Cc1cc(F)cc(Oc2cnc(C(F)(F)F)c3c2[C@@H](F)[C@@H](F)[C@H]3O)c1 ; "and" ; O[C@H]1c2c(C(F)(F)F)ncc(OC3CC(F)(F)C3)c2[C@@H](F)[C@H]1F]

or a pharmaceutically acceptable salt thereof.

In certain aspects, the HIF-2α inhibitor is a compound of Formula II:

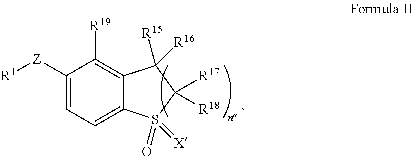

or a pharmaceutically acceptable salt or prodrug thereof, wherein:

Z is selected from —O—, —S—, —S(O)—, —S(O)₂—, —C(O)—, —C(HR⁵)—, —N(R⁶)—, C₁-C₃ alkylene, C₁-C₃ heteroalkylene, C₁-C₃ alkenylene or absent;

R¹ is selected from C₁₋₆ alkyl, C₃₋₁₂ carbocycle and 3- to 12-membered heterocycle, each of which is optionally substituted with one or more R²⁰;

R⁵ is selected from hydrogen and R²⁰;

R⁶ is selected from R²¹;

R¹⁵ is selected from hydrogen, —OH, and —N(R²¹)₂;

R¹⁶ is selected from hydrogen, deuterium and C₁₋₆ alkyl, wherein said C₁₋₆ alkyl is optionally substituted with one or more R²⁰; or R¹⁵ and R¹⁶ in combination form oxo or methylene;

R¹⁷ and R¹⁸ are independently selected from hydrogen and halogen; and C₁₋₆ alkyl, 2- to 6-membered heteroalkyl and C₃₋₁₀ cycloalkyl, each of which is optionally substituted with one or more R²⁰; or R¹ and R¹⁸ and the carbon to which they are attached form C₃-C₈ cycloalkyl or C₅-C₈ heterocycloalkyl, each of which is optionally substituted with one or more R²⁰;

R¹⁹ is selected from hydrogen, halogen, —CN, —NO₂, —C(O)R²¹, —C(O)OR²¹, —OC(O)R²¹, —OC(O)N(R²¹)₂, —OC(O)NR²²R²³, —NR²¹C(O)R²¹ and —S(═O)₂R²¹; and C₁₋₆ alkyl, 2- to 6-membered heteroalkyl, C₁₋₁₀ alkenyl, and C₁₋₁₀ alkynyl, each of which is optionally substituted with one or more R²⁰;

X′ is O or NR^{18′}, wherein R¹⁸ is selected from the group consisting of hydrogen, C₁₋₆ alkyl and —CN;

n″ is 1 or 2;

R²⁰ is independently selected at each occurrence from:
halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, and ═N(R²¹);
C₁₋₁₀ alkyl, C₂₋₁₀ alkenyl, and C₂₋₁₀ alkynyl, each of which is independently optionally substituted at each occurrence with one or more substituents selected from R²⁴; and
C₃₋₁₂ carbocycle and 3- to 12-membered heterocycle, each of which is independently optionally substituted at each occurrence with one or more substituents selected from R²⁵;

R²¹ is independently selected at each occurrence from hydrogen; and C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;

R²² and R²³ are taken together with the nitrogen atom to which they are attached to form a heterocycle, optionally substituted with one or more R²⁰;

R²⁴ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₃₋₁₂ carbocycle, and 3- to 12-membered heterocycle, wherein each C₃₋₁₂ carbocycle and 3- to 12-membered heterocycle is independently optionally substituted with one or more substituents selected from R²⁵; and

R²⁵ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₁₋₆ alkyl, C₁₋₆ haloalkyl, C₂₋₆ alkenyl, and C₂₋₆ alkynyl.

In some embodiments, for a compound of Formula II, R¹⁵ is selected from —OH, and —N(R²¹)₂. In some embodiments, R¹⁵ is —OH. In some embodiments, R¹⁵ is —N(R²¹)₂. In some embodiments, R¹⁵ is selected from —OH and —NH₂. In some embodiments, R¹⁵ is hydrogen. In some embodiments, R¹⁶ is selected from hydrogen and deuterium. In some embodiments, R¹⁶ is C₁₋₄ alkyl. In some embodiments, R¹⁶ is hydrogen. In some embodiments, R¹⁵ is selected from —OH and —NH₂ and R¹⁶ is hydrogen.

In some embodiments, for a compound of Formula II, each of R¹⁷ and R¹⁸ is independently hydrogen or —F. In some embodiments, each of R¹⁷ and R¹⁸ is hydrogen. In some embodiments, each of R¹⁷ and R¹⁸ is —F. In some embodiments, at least one of R¹⁷ and R¹⁸ is —F. In some embodiments, n″ is 1.

In certain aspects, the HIF-2α inhibitor is a compound of Formula II-A:

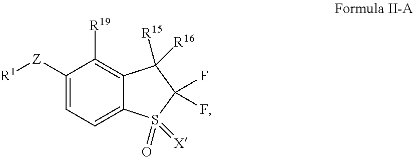

or a pharmaceutically acceptable salt or prodrug thereof, wherein:

Z is selected from —O—, —S—, —S(O)—, —S(O)₂—, —C(O)—, —C(HR⁵)—, —N(R⁶)—, C₁-C₃ alkylene, C₁-C₃ heteroalkylene, C₁-C₃ alkenylene or absent;

R¹ is selected from C₁₋₆ alkyl, C₃₋₁₂ carbocycle and 3- to 12-membered heterocycle, each of which is optionally substituted with one or more R²⁰;

R⁵ is selected from hydrogen and R²⁰;

R⁶ is selected from R²¹;

R¹⁵ is selected from hydrogen, —OH, and —N(R²¹)₂;

R¹⁶ is selected from hydrogen, deuterium and C₁₋₆ alkyl, wherein said C₁₋₆ alkyl is optionally substituted with one or more R²⁰; or R¹⁵ and R¹⁶ in combination form oxo or methylene;

R¹⁹ is selected from hydrogen, halogen, —CN, —NO₂, —C(O)R²¹, —C(O)OR²¹, —OC(O)R²¹, —OC(O)N(R²¹)₂, —OC(O)NR²²R²³, —NR²¹C(O)R²¹ and —S(═O)₂R²¹; and C₁₋₆ alkyl, 2- to 6-membered heteroalkyl, C₁₋₁₀ alkenyl, and C₁₋₁₀ alkynyl, each of which is optionally substituted with one or more R²⁰;

X′ is O or NR^{18′}, wherein R¹⁸ is selected from the group consisting of hydrogen, C₁₋₆ alkyl and —CN;

R²⁰ is independently selected at each occurrence from:
halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, and ═N(R²¹);
C₁₋₁₀ alkyl, C₂₋₁₀ alkenyl, and C₂₋₁₀ alkynyl, each of which is independently optionally substituted at each occurrence with one or more substituents selected from R²⁴; and
C₃₋₁₂ carbocycle and 3- to 12-membered heterocycle, each of which is independently optionally substituted at each occurrence with one or more substituents selected from R²⁵;

R²¹ is independently selected at each occurrence from hydrogen; and C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;

R²² and R²³ are taken together with the nitrogen atom to which they are attached to form a heterocycle, optionally substituted with one or more R²⁰;

R²⁴ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₃₋₁₂ carbocycle, and 3- to 12-membered heterocycle, wherein each C₃₋₁₂ carbocycle and 3- to 12-membered heterocycle is independently optionally substituted with one or more substituents selected from R²⁵; and

R²⁵ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₁₋₆ alkyl, C₁₋₆ haloalkyl, C₂₋₆ alkenyl, and C₂₋₆ alkynyl.

In some embodiments, for a compound of Formula II-A, R¹ is selected from —OH, and —N(R²¹)₂. In some embodiments, R¹⁵ is —OH. In some embodiments, R¹⁵ is —N(R²¹)₂. In some embodiments, R¹⁵ is selected from —OH and —NH₂. In some embodiments, R¹⁵ is hydrogen. In some embodiments, R¹⁶ is selected from hydrogen and deuterium. In some embodiments, R¹⁶ is C₁₋₄ alkyl. In some embodiments, R¹⁶ is hydrogen. In some embodiments, R¹⁵ is selected from —OH and —NH₂ and R¹⁶ is hydrogen.

In certain aspects, the HIF-2α inhibitor is a compound of Formula II-B:

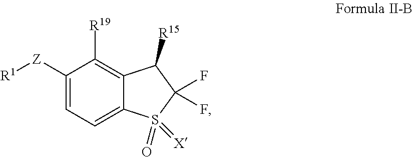

or a pharmaceutically acceptable salt or prodrug thereof, wherein:

Z is selected from —O—, —S—, —S(O)—, —S(O)₂—, —C(O)—, —C(HR⁵)—, —N(R⁶)—, C₁-C₃ alkylene, C₁-C₃ heteroalkylene, C₁-C₃ alkenylene or absent;

R¹ is selected from C₁₋₆ alkyl, C₃₋₁₂ carbocycle and 3- to 12-membered heterocycle, each of which is optionally substituted with one or more R²⁰;

R⁵ is selected from hydrogen and R²⁰;

R⁶ is selected from R²¹;

R¹⁵ is selected from hydrogen, —OH, and —N(R²¹)₂;

R¹⁹ is selected from hydrogen, halogen, —CN, —NO₂, —C(O)R²¹, —C(O)OR²¹, —OC(O)R²¹, —OC(O)N(R²¹)₂, —OC(O)NR²²R²³, —NR²¹C(O)R²¹ and —S(═O)₂R²¹; and C₁₋₆ alkyl, 2- to 6-membered heteroalkyl, C₁₋₁₀ alkenyl, and C₁₋₁₀ alkynyl, each of which is optionally substituted with one or more R²⁰;

X′ is O or NR^{18′}, wherein R¹⁸ is selected from the group consisting of hydrogen, C₁₋₆ alkyl and —CN;

R²⁰ is independently selected at each occurrence from:
halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)R²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, and ═N(R²¹);
C₁₋₁₀ alkyl, C₂₋₁₀ alkenyl, and C₂₋₁₀ alkynyl, each of which is independently optionally substituted at each occurrence with one or more substituents selected from R²⁴; and
C₃₋₁₂ carbocycle and 3- to 12-membered heterocycle, each of which is independently optionally substituted at each occurrence with one or more substituents selected from R²⁵;

R²¹ is independently selected at each occurrence from hydrogen; and C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;

R²² and R²³ are taken together with the nitrogen atom to which they are attached to form a heterocycle, optionally substituted with one or more R²⁰;

R²⁵ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₃₋₁₂ carbocycle, and 3- to 12-membered heterocycle, wherein each C₃₋₁₂ carbocycle and 3- to 12-membered heterocycle is independently optionally substituted with one or more substituents selected from R²⁵; and

R²⁵ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₁₋₆ alkyl, C₁₋₆ haloalkyl, C₂₋₆ alkenyl, and C₂₋₆ alkynyl.

In some embodiments, for a compound of Formula II-B, R¹ is selected from —OH, and —N(R²¹)₂. In some embodiments, R¹⁵ is —OH. In some embodiments, R¹⁵ is —N(R²¹)₂. In some embodiments, R¹⁵ is selected from —OH and —NH₂. In some embodiments, R¹⁵ is hydrogen.

In some embodiments, for a compound of Formula II, II-A or II-B, R¹ is further selected from —C(O)R²¹ and —CN; and C₁₋₆ alkyl, 2- to 6-membered heteroalkyl, C₃₋₁₂ cycloalkyl, C₃₋₁₂ cycloalkenyl, 3- to 12-membered heterocycloalkyl, C₃₋₁₀ aryl and 3- to 10-membered heteroaryl, each of which is optionally substituted with one or more R²⁰.

In some embodiments, for a compound of Formula II, II-A or II-B, R¹ is selected from C₆₋₁₀ aryl, 5- to 8-membered heteroaryl, C₃₋₈ cycloalkyl and 3- to 8-membered heterocycloalkyl. In some embodiments, R¹ is selected from phenyl and pyridyl. In some embodiments, R¹ is phenyl. In some embodiments, R¹ is selected from C₃₋₈ cycloalkyl and 3- to 8-membered heterocycloalkyl.

In some embodiments, for a compound of Formula II, II-A or II-B, R¹ is

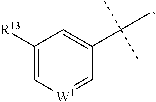

wherein W¹ is N or CR¹⁴; and R¹³ and R¹⁴ are independently selected from hydrogen and R²⁰. In some embodiments, R¹⁴ is selected from hydrogen, halogen, —CN, C₁₋₆ alkyl and C₁₋₆ alkoxy. In some embodiments, R¹⁴ is selected from halogen, —CN, C₁₋₆ alkyl and C₁₋₆ alkoxy. In some embodiments, R¹⁴ is selected from halogen and —CN. In some embodiments, R¹⁴ is —F. In some embodiments, R¹³ is selected from halogen, —CN, C₁₋₆ alkyl and C₁₋₆ alkoxy. In some embodiments, R¹³ is selected from halogen and —CN. In some embodiments, R¹³ is —CN. In some embodiments, R¹³ is —CN and R¹⁴ is —F.

In some embodiments, for a compound of Formula II, II-A or II-B, R¹ is

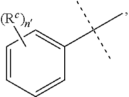

wherein R^{c} is independently selected at each occurrence from hydrogen and R²⁰; and n′ is 0, 1, 2, 3 or 4. In some embodiments, R^{c} is independently selected at each occurrence from hydrogen, halogen, —CN, C₁₋₆ alkyl and C₁₋₆ alkoxy. In some embodiments, R is selected from halogen, —CN, C₁₋₆ alkyl and C₁₋₆ alkoxy. In some embodiments, R^{c} is selected from halogen and —CN. In some embodiments, R^{c} is —F and —CN. In some embodiments, n′ is 1, 2 or 3. In some embodiments, n′ is 2. In some embodiments, n′ is 2 and R^{c} is selected from halogen and —CN.

In some embodiments, for a compound of Formula II, II-A or II-B, R¹ is

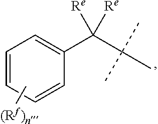

wherein each of R^{e} is independently hydrogen or C₁₋₄ alkyl, or two R^{e}s and the carbon atom to which they are attached form a 4- to 8-membered cyclic moiety; each of R^{f} is independently selected from the group consisting of halogen, —CN, C₁₋₆ alkoxy and C₁₋₆ alkyl; and n′″ is 0, 1, 2, 3 or 4. In some embodiments, the 4- to 8-membered cyclic moiety is an all carbon or heterocyclic ring system.

In some embodiments, for a compound of Formula II, II-A or II-B, R¹ is bicyclic heteroaryl. In some embodiments R¹ is selected from:

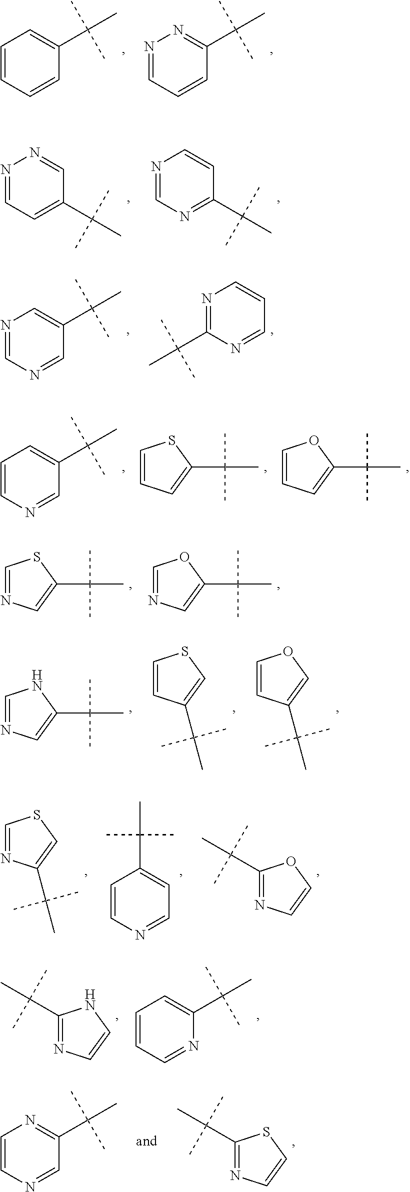

wherein the rings specified for R¹ are optionally substituted with one or more R²⁰. In some embodiments, R¹ is selected from

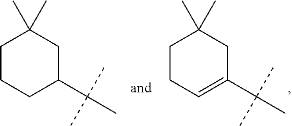

wherein the rings specified for R¹ are optionally substituted with one or more R²⁰.

In some embodiments, for a compound of Formula II, II-A or II-B, R¹ is cycloalkyl. In some embodiments, R¹ is heterocycloalkyl. In some embodiments, R¹ is selected from C₃-C₆ cycloalkyl and 3- to 6-membered heterocycloalkyl. In some embodiments, R¹ is cyclobutyl. In some embodiments, said heterocycloalkyl, cycloalkyl and cyclobutyl are optionally substituted with one or more R²⁰. In some embodiments, R¹ is acyl or cyano. In some embodiments, R¹ is acetyl. In some embodiments, R¹ is alkyl. In some embodiments, the alkyl is substituted with one or more R²⁰. In some embodiments, the alkyl is substituted with at least one fluoro. In some embodiments, R¹ is heteroalkyl. In some embodiments, R¹ is selected from the group consisting of:

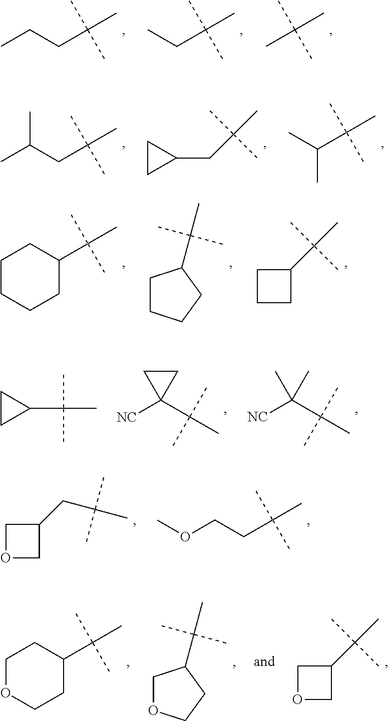

wherein each of the members are optionally substituted with one or more R²⁰.

In some embodiments, for a compound of Formula II, II-A or II-B, R¹ is substituted with one or more R²⁰. In some embodiments, R¹ is substituted with two or more R²⁰. In some embodiments, R¹ is substituted with three or more R²⁰. In some embodiments, R is substituted with two R²⁰ substituents. In some embodiments, R¹ is substituted with one or more substituents selected from halogen, —CN, C₁₋₆ alkyl and C₁₋₆ alkoxy. In some embodiments, R¹ is substituted with one or more substituents selected from halogen and —CN. In some embodiments, R¹ is substituted with one or more substituents selected from —F and —CN. In some embodiments, R¹ is substituted with halogen and —CN. In some embodiments, R¹ is substituted with —F and —CN.

In some embodiments, for a compound of Formula II, II-A or II-B, R¹⁹ is selected from halogen, —CN, —NO₂, —C(O)R²¹, —C(O)OR²¹, —OC(O)R²¹, —OC(O)N(R²¹)₂, —OC(O)NR²²R²³, —NR²¹C(O)R²¹ and —S(═O)₂R²¹; and C₁₋₆ alkyl, 2- to 6-membered heteroalkyl, C₁₋₁₀ alkenyl, and C₁₋₁₀ alkynyl, each of which is optionally substituted with one or more R²⁰. In some embodiments, R¹⁹ is selected from halogen, —CN, and —NO₂; and C₁₋₆ alkyl, 2- to 6-membered heteroalkyl, C₁₋₁₀ alkenyl, and C₁₋₁₀ alkynyl, each of which is optionally substituted with one or more R²⁰. In some embodiments, R¹⁹ is selected from halogen, —CN and C₁₋₆ alkyl, wherein said C₁₋₆ alkyl is optionally substituted with one or more R²⁰. In some embodiments, R¹⁹ is selected from halogen and C₁₋₆ alkyl, wherein said C₁₋₆ alkyl is optionally substituted with one or more R²⁰, such as C₁₋₆ alkyl optionally substituted with one or more halogens. In some embodiments, R¹⁹ is selected from halogen, —CN and C₁₋₆ fluoroalkyl. In some embodiments, R¹⁹ is C₁₋₆ fluoroalkyl. Exemplary C₁₋₆ fluoroalkyl groups include —CH₂F, —CHF₂, —CF₃, and —CF₂CH₃.

In some embodiments, for a compound of Formula II, II-A or II-B, X′ is O or NR^{18′}, wherein R¹⁸ is selected from the group consisting of hydrogen, C₁₋₆ alkyl and —CN. In some embodiments, X′ is O.

In some embodiments, for a compound of Formula II, II-A or II-B, Z is —O—, —S—, —S(O)—, —S(O)₂—, —S(O₂)N(R⁶)—, —C(O)—, —C(O)O—, —C(HR)—, —N(R⁶)—, —C(O)N(R⁶)—, alkylene, alkenylene, alkynylene, heteroalkylene, or absent. In some embodiments, Z is —O—, —S—, —S(O)—, —S(O)₂—, —C(O)—, —C(HR)—, —N(R⁶)—, C₁-C₃ alkylene, C₁-C₃ heteroalkylene, C₁-C₃ alkenylene or absent. In some embodiments, Z is —O—. In some embodiments, Z is —S—. In some embodiments, Z is —C(HR5)-. In some embodiments, Z is —N(R⁶). In some embodiments, Z is absent.

In some embodiments, for a compound of Formula II, II-A or II-B:

Z is —O—;

X′ is O;

R¹ is phenyl, pyridyl or C₃₋₆ cycloalkyl, each of which is optionally substituted with one or more R²⁰; and

R¹⁹ is selected from halogen, —CN, and —NO₂; and C₁₋₆ alkyl, 2- to 6-membered heteroalkyl, C₁₋₁₀ alkenyl, and C₁₋₁₀ alkynyl, each of which is optionally substituted with one or more R²⁰.

In some embodiments, a compound of Formula I′, I, I-A, I-B, I-C, I-D, I-E, I-F, I-G, I-H, I-I, I-J, I-K, II, II-A or II-B is provided as a substantially pure stereoisomer. The stereoisomer may be provided in at least 90% diastereomeric excess. In some embodiments, a compound of Formula I′, I, I-A, I-B, I-C, I-D, I-E, I-F, I-G, I-H, I-I, I-J, I-K, II, II-A or II-B may have an diastereomeric excess of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or even higher. In some embodiments, a compound of Formula I′, I, I-A, I-B, I-C, I-D, I-E, I-F, I-G, I-H, I-I, I-J, I-K, II, II-A or II-B may have an diastereomeric excess of about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99%. The stereoisomer may be provided in at least 90% enantiomeric excess. In some embodiments, a compound of Formula I′, I, I-A, I-B, I-C, I-D, I-E, I-F, I-G, I-H, I-I, I-J, I-K, II, II-A or II-B may have an enantiomeric excess of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or even higher. In some embodiments, a compound of Formula I′, I, I-A, I-B, I-C, I-D, I-E, I-F, I-G, I-H, I-I, I-J, I-K, II, II-A or II-B may have an enantiomeric excess of about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99%.

In another aspect, the present disclosure provides a compound or pharmaceutically acceptable salt or prodrug thereof, selected from the group consisting of the compounds given in Table 1 and Table 2.

The chemical entities described herein can be synthesized according to one or more illustrative schemes herein and/or techniques known in the art, including those described in U.S. Pub. Nos. 2016/0251307 and 2018/0042884, each of which is incorporated herein by reference in its entirety. Materials used herein are either commercially available or prepared by synthetic methods generally known in the art. These schemes are not limited to the compounds listed in the examples or by any particular substituents, which are employed for illustrative purposes. Although various steps are described and depicted in Schemes 1-5, the steps in some cases may be performed in a different order than the order shown in Schemes 1-5. Various modifications to these synthetic reaction schemes may be made and will be suggested to one skilled in the art having referred to the disclosure contained in this Application. Numberings or R groups in each scheme do not necessarily correspond to that of the claims or other schemes or tables herein.

Unless specified to the contrary, the reactions described herein take place at atmospheric pressure, generally within a temperature range from −10° C. to 200° C. Further, except as otherwise specified, reaction times and conditions are intended to be approximate, e.g., taking place at about atmospheric pressure within a temperature range of about −10° C. to about 110° C. over a period of about 1 to about 24 hours; reactions left to run overnight average a period of about 16 hours.

In general, compounds of the invention may be prepared by the following reaction schemes:

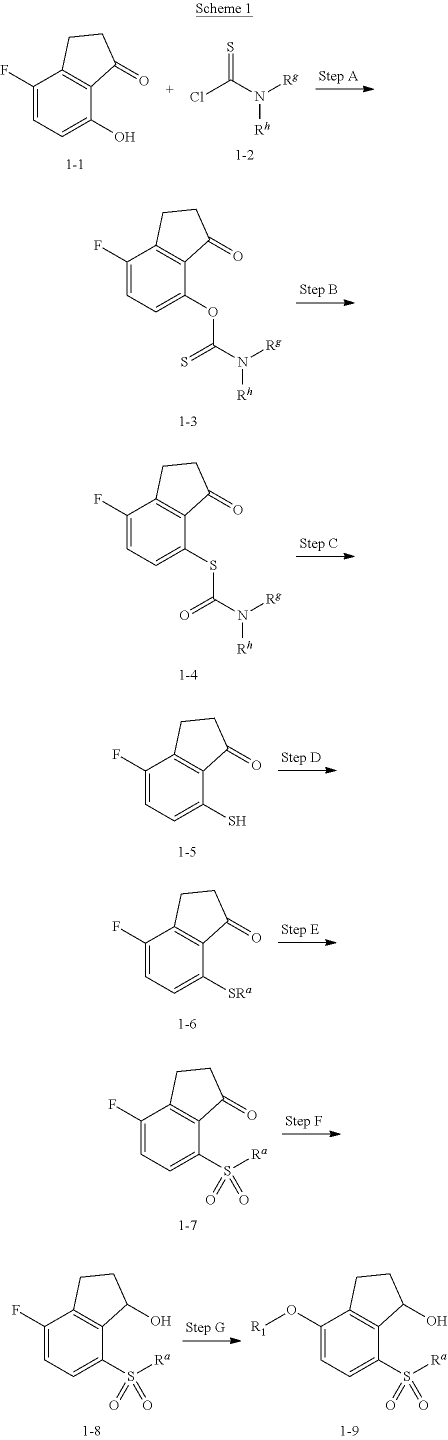

In some embodiments, a compound of Formula 1-9 can be prepared according to steps outlined in Scheme 1. The synthesis starts with phenol 1-1. Reaction of 1-1 with chloride 1-2 (wherein R^{g} and R^{h} are independently alkyl) provides intermediate 1-3. The reaction may be carried out in a suitable organic solvent in the presence of a base. Suitable bases for the reaction include, but are not limited to, organic bases, for example, triethylamine, N,N-diisopropylethylamine, 1,4-diazabicyclo[2.2.2]octane, and inorganic bases, for example, sodium hydroxide, cesium carbonate, cesium bicarbonate, sodium carbonate, and potassium carbonate. A compound of Formula 1-3 is then subjected to a rearrangement reaction to give a compound of Formula 1-4. Elevated temperature may be needed for the rearrangement to occur. The temperature may be in a range of 100° C. to 300° C. In some embodiments, the temperature is in a range of 180° C. to 240° C. Hydrolysis of a compound of Formula 1-4 provides thiophenol 1-5, which is alkylated to provide a compound if Formula 1-6. A variety of alkyl groups may be introduced in Step D. In some embodiments, R^{a} is a C₁-C₄ alkyl. In a further embodiment, R^{a} is a C₁-C₄ fluoroalkyl. Oxidation of a compound of Formula 1-6 may be accomplished by a variety of methods known in the art, including, but not limited to, RuCl₃ catalyzed oxidation in the presence of NaIO₄, oxidation with m-chloroperoxybenzoic acid (mCPBA) and oxidation with Oxone®. Ketone 1-7 is then reduced to give alcohol 1-8, which then undergoes a nucleophilic aromatic substitution (SNAr) reaction with a suitable substrate R¹OH to give a compound of Formula 1-9. Temperatures for carrying out the SNAr reaction may depend on the reactivity of both R¹OH and/or compound 1-8. The reaction may be carried out in a temperature range from about room temperature to 200° C. In some embodiments, the temperature range is from room temperature to 60° C. In some other embodiments, the temperature range is from 60° C. to 100° C. In some other embodiments, the temperature range is from 100° C. to 200° C.

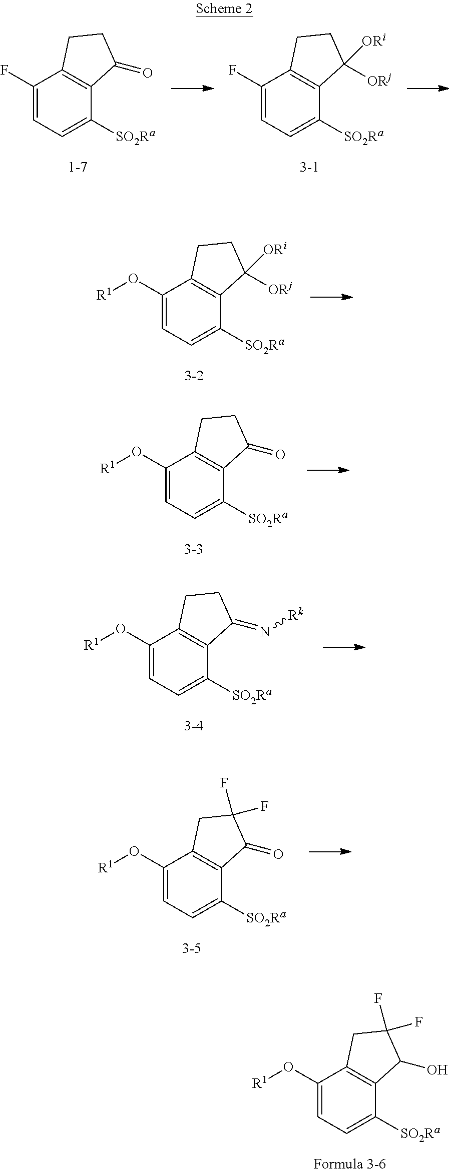

In some embodiments, a compound of Formula 3-6 may be prepared according to Scheme 2. The ketone in 1-7 is protected as a ketal to give a compound of Formula 3-1, wherein each of R¹ and R^{j} is independently an alkyl group. In addition, R and R may optionally be connected to form a cyclic ketal. Exemplary structures of ketal 3-1 include, but are not limited to, the following:

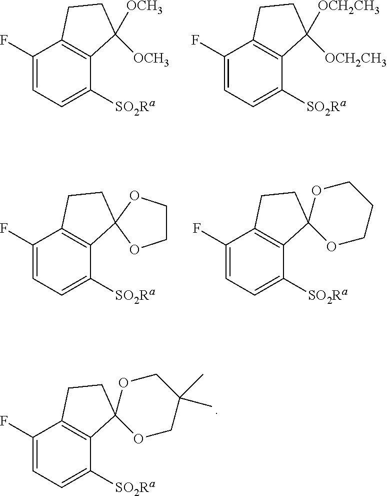

A compound of Formula 3-1 and a suitable R¹OH may undergo a nucleophilic aromatic substitution reaction (SNAr) to give biaryl ether 3-2. As described in Step G of Scheme 1, the reaction temperature of the SNAr reaction may depend on the reactivity of the aryl halide (i.e. compound 3-1) and/or R¹OH. Ketone 3-3, resulting from the deprotection of ketal 3-2, is condensed with an amine to form imine 3-4, wherein R^{k} is alkyl. The imine functional group in a compound of Formula 3-4 may exist as a mixture of E and Z isomers. Fluorination of 3-4 can be accomplished with a fluorinating reagent, for example, 1-(chloromethyl)-4-fluoro-1,4-diazoniabicyclo[2.2.2]octane ditetrafluoroborate, to give difluoroketone 3-5 after acid hydrolysis. Finally, reduction of ketone 3-5 with a hydride donor gives a compound of Formula 3-6.

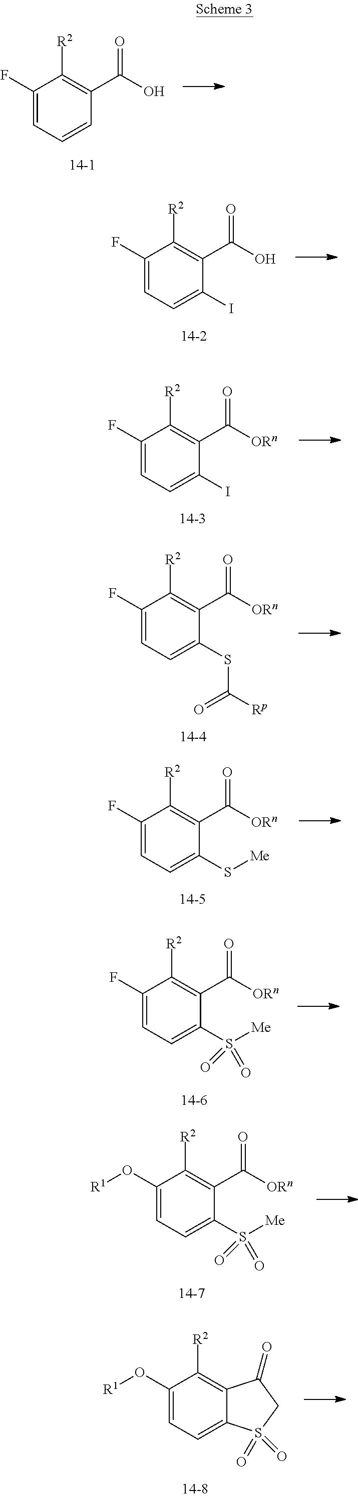

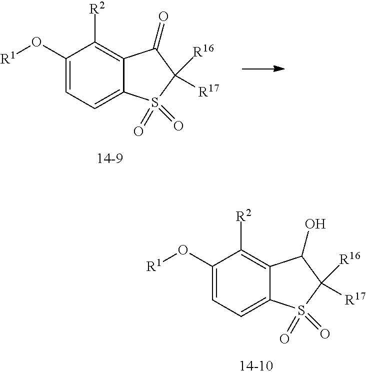

In some embodiments, a compound of Formula 14-10 can be prepared according to steps outlined in Scheme 3, wherein R¹ is alkyl, heteroalkyl, cycloalkyl, heterocycloalkyl, aryl or heteroaryl; R² is halo, cyano, alkyl, alkenyl or alkynyl; and R¹⁶ and R¹ are fluoro or alkyl, or R¹⁶ and R¹⁷ and the carbon to which they are attached form C₃-C₈ cycloalkyl or C₅-C₈ heterocycloalkyl. The synthesis commences with compounds of Formula 14-1. Orthoiodination of 14-1 provides compound 14-2. The reaction may be carried out in a suitable organic solvent in the presence of iodine and a palladium catalyst at an elevated temperature, if needed. After esterification of 14-2, the resulting ester 14-3 may undergo a transition-metal catalyzed coupling reaction with a thioate, e.g., potassium ethanethioate or sodium ethanethioate, to give compounds of Formula 14-4. Suitable transition-metal catalysts include, but are not limited to, Pd(PPh₃)₄, Pd₂(dba)₃ chloroform complex or Pd(OAc)₂, in the presence or absence of a suitable ligand. Hydrolysis of a compound of Formula 14-4 followed by alkylation of the resulting thiophenol intermediate with an alkyl halide, e.g., methyl iodide, gives a compound of Formula 14-5. The hydrolysis and alkylation may be carried out in a one-pot procedure without purification. In some embodiments, this is carried out by treating a compound of Formula 14-4 with a carbonate base in a suitable solvent at or near room temperature for a period ranging from 0.1 to 24 hours, followed by addition of an alkyl halide. Carbonate bases include, but are not limited to, sodium carbonate, potassium carbonate, cesium carbonate, potassium bicarbonate and cesium bicarbonate. Oxidation of a compound of Formula 14-5 to give a compound of Formula 14-6 may be accomplished by a variety of methods known in the art, including, but not limited to, RuCl₃ catalyzed oxidation in the presence of NaIO₄, oxidation with m-chloroperoxybenzoic acid (mCPBA), and oxidation with Oxone®. A compound of Formula 14-6 is then subjected to a nucleophilic aromatic substitution (SNAr) reaction with R¹OH (wherein R¹ is alkyl, aryl or heteroaryl) to give a compound of Formula 14-7. Temperature for carrying out the SNAr reaction may depend on the reactivity of both R¹OH and/or a compound of Formula 14-6. The reaction may be carried out at a temperature ranging from −10° C. to 200° C. In some embodiments, the temperature range is from 30° C. to 120° C. In some other embodiments, the temperature range is from 0° C. to room temperature. Cyclization of a compound of Formula 14-7 may be effected with a base, e.g., sodium hydride, in a suitable solvent to yield a compound of Formula 14-8. After the cyclization, a variety of R¹⁶ and R¹⁷ groups may be introduced. In some embodiments, a compound of Formula 14-8 is difluorinated to give a compound of Formula 14-9, formed by treatment with a fluorinating agent, e.g., 1-(chloromethyl)-4-fluoro-1,4-diazo niabicyclo[2.2.2]octane ditetrafluoroborate (Selectfluor®), in the presence of suitable base, e.g., sodium carbonate. Reduction of a compound of Formula 14-9 yields a compound of Formula 14-10. In some embodiments, the reduction is carried out with a hydride, e.g., sodium borohydride and sodium triacetoxyborohydride, to give a racemic mixture. In some embodiments, an asymmetric reduction is carried out to give an enantiomer having an enantiomeric execess as disclosed herein.

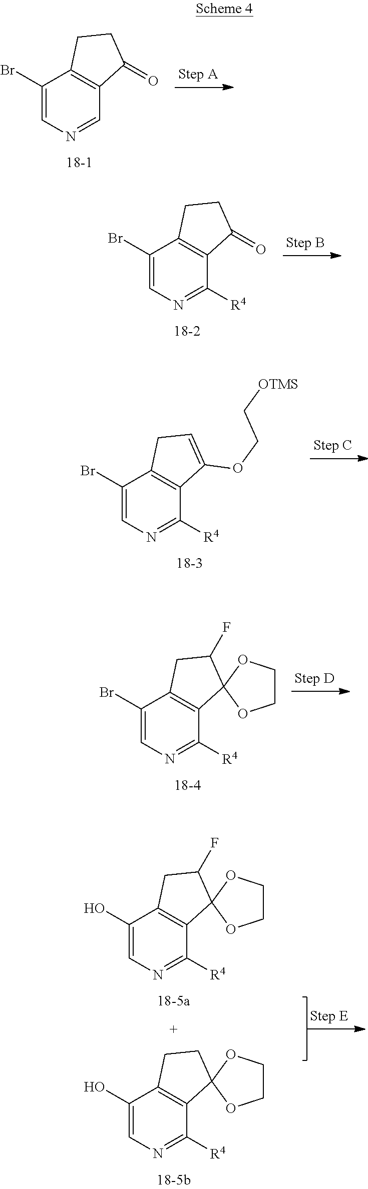

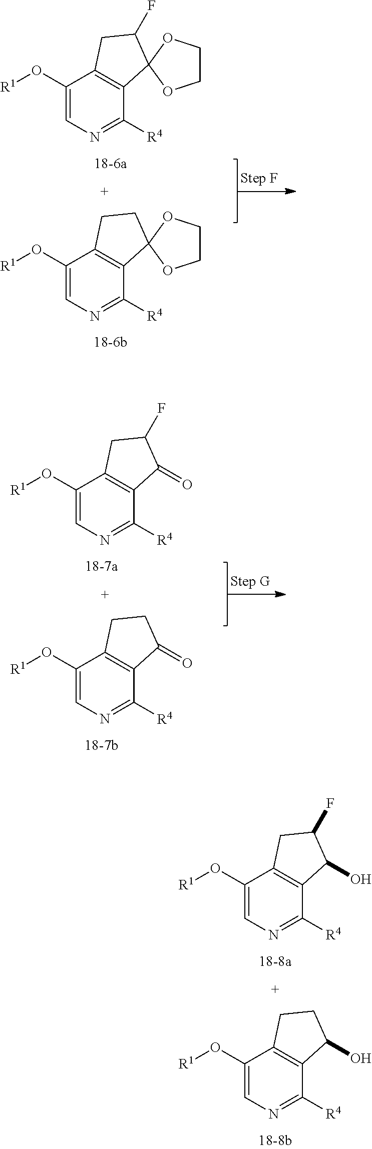

In some embodiments, compounds of Formula 18-8a and 18-8b may be prepared according to Scheme 4. For example, pyridine 18-1 may be converted to alkylaryl derivative 18-2 in Step A, wherein R⁴ is, for example, trifluoromethyl. The ketone may be converted to protected enol ether 18-3, then fluorinated to give fluoroketal 18-4. Treatment of a compound of Formula 18-4 with a suitable hydroxide source gives a mixture of phenols 18-5a and 18-5b. The phenols can undergo an SNAr reaction with a suitable halide to give aryl ethers of Formulae 18-6a and 18-6b, which may be deprotected to give the resultant ketones. In some embodiments, a compound of Formula 18-7 is reduced with a hydride source to give a racemic mixture. In other embodiments, an asymmetric reduction is carried out, affording alcohols 18-8a and 18-8b, separable by methods known to one skilled in the art, such as, for example, conventional column chromatography.

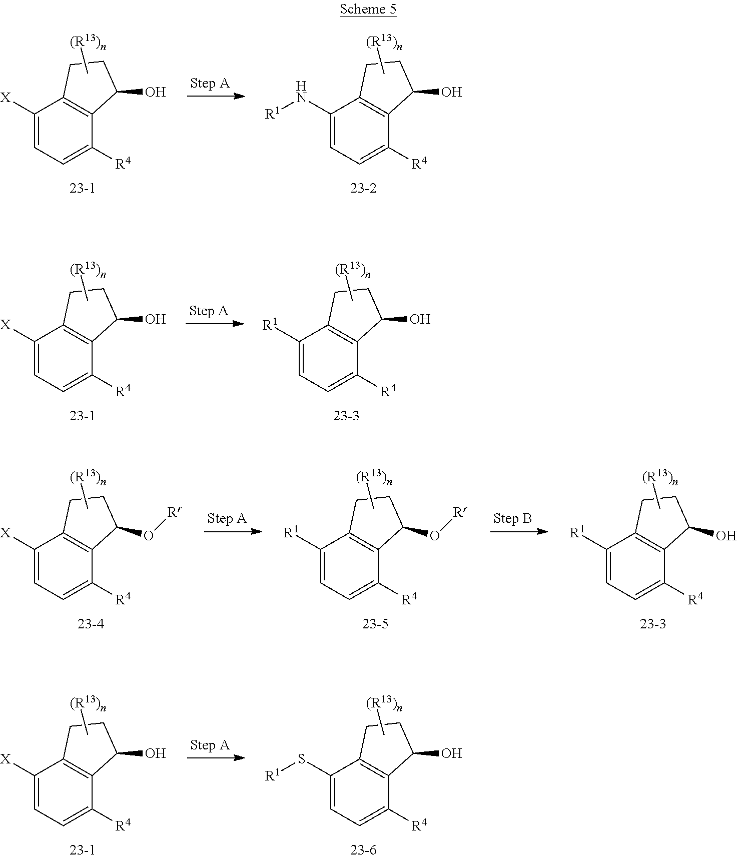

In some embodiments, R can be coupled to a compound of Formula 23-1 or 23-4 via a reaction scheme represented generally in Scheme 5. In some embodiments, wherein Z is —N(R⁸), an aryl halide of Formula 23-1 is coupled to a suitably substituted amine, i.e. NHR¹R⁸, via a Buchwald-Hartwig amination to give a compound of Formula 23-2. In a further embodiment, Step A is a cross coupling reaction, including, but not limited to, a Stille, Negishi or Suzuki reaction, wherein an aryl halide of Formula 23-1 is combined with an appropriate reactant containing R and a suitable catalyst to afford a compound of Formula 23-3. In other embodiments, a compound of Formula 23-4 undergoes an SNAr reaction and a subsequent deprotection to give a compound of Formula 23-3. R in a compound of Formula 23-5 may be, for example, morpholine, wherein a C—N bond connects said morpholine to the aryl ring. In still other embodiments, Z is —S—, and R¹S— is attached to a compound of Formula 23-1 via an SNAr reaction to give a compound of Formula 23-6.

In some other embodiments, a compound of a formula given in Table 1 or Table 2 is synthesized according to one of the general routes outlined in Schemes 1-5, Examples 1-8 or by methods generally known in the art.

TABLE 1

| No. | Structure |
|---|---|
| 1 | 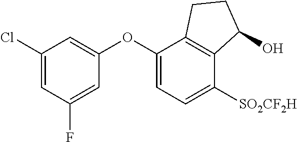 |
| | m/z: 393 (M + H) |
| 2 | 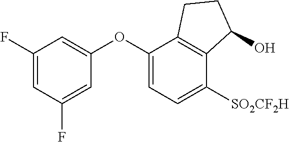 |
| | m/z: 377 (M + H) |
| 3 | 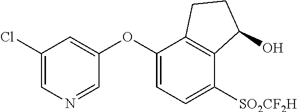 |
| | m/z: 376, 378 (M + H) |
| 4 | 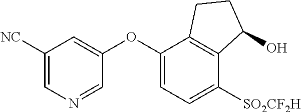 |
| | m/z: 367 (M + H) |
| 5 | 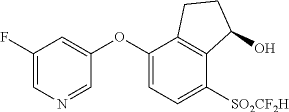 |
| | m/z: 360 (M + H) |
| 6 | 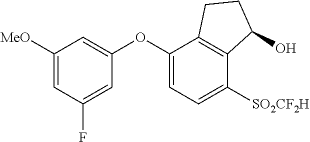 |
| | m/z: 433 (M − H + 46) |
| 7 | 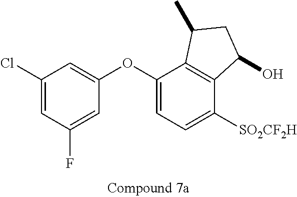 |
| | 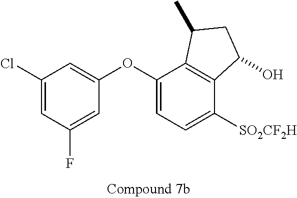 |
| | m/z: 7a, 429, 431 (M + Na); |
| | 7b, 429, 431 (M + Na) |
| 8 | 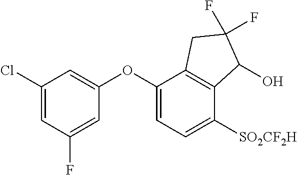 |
| | m/z: 429, 431 (M + H) |
| 9 | 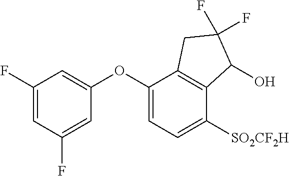 |
| | m/z: 413 (M + H) |
| 10 | 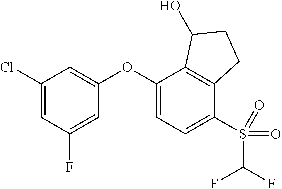 [SMILES: O=S(=O)(c1ccc(Oc2cc(F)cc(Cl)c2)c2c1CCC2O)C(F)F] |
| | m/z: 375, 377 (M − OH) |
| 11 | 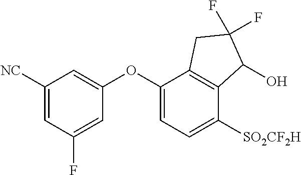 |
| | m/z: 437 (M + NH₄) |
| 12 | 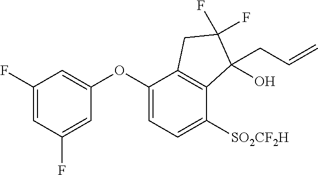 |
| | m/z: 451 (M − H) |
| 13 | 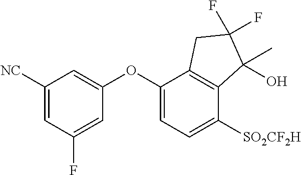 |
| | m/z: 451 (M + NH₄) |
| 14 | 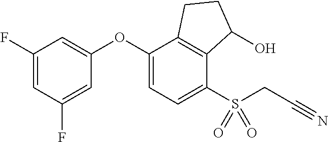 [SMILES: N#CCS(=O)(=O)c1ccc(Oc2cc(F)cc(F)c2)c2c1C(O)CC2] |
| | m/z: 364 (M − H) |
| 15 | 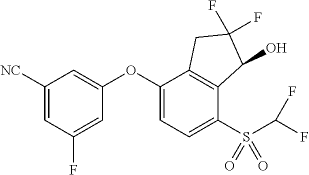 [SMILES: N#Cc1cc(F)cc(Oc2ccc(S(=O)(=O)C(F)F)c3c2CC(F)(F)[C@H]3O)c1] |
| | m/z: 420 (M + H) |
| 16 | 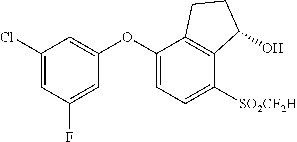 |
| | m/z: 437/439 (M − H + 46) |
| 17 | 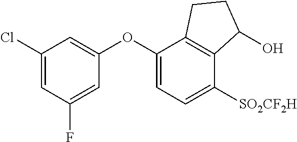 |
| | m/z: 393 (M + H) |
| 18 | 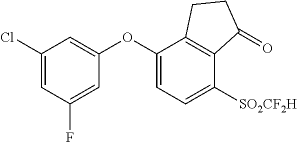 |
| | m/z: 391, 393 (M + H) |
| 19 | 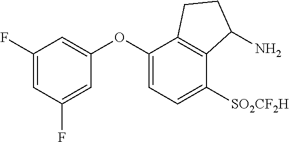 |
| | m/z: 376 (M + H) |
| 20 | 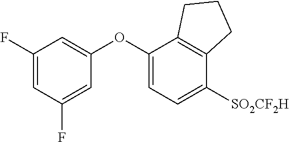 |
| | m/z: 361 (M + H) |
| 21 | 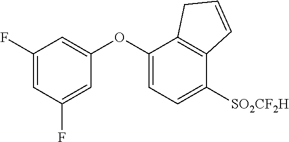 |
| | m/z: 359 (M + H) |
| 22 | 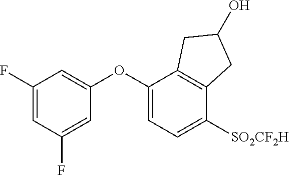 |
| | m/z: 421 (M − H + 46) |
| 23 | 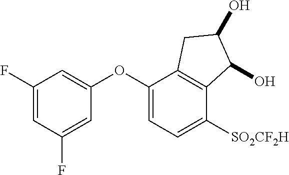 |
| | m/z: 437 (M − H + 46) |
| 24 | 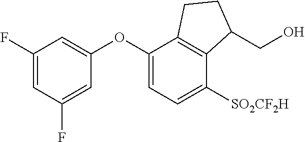 |
| | m/z: 391 (M + H) |
| 25 | 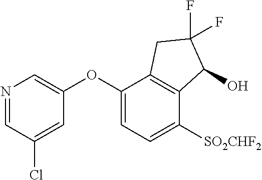 |
| | m/z: 410, 412 (M + H) |
| 26 | 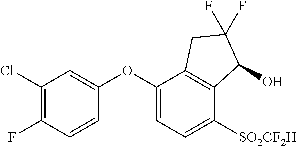 |
| | m/z: 473, 475 (M − H + 46) |
| 27 | 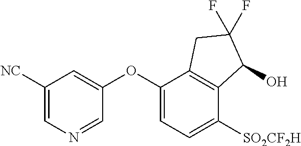 |
| | m/z: 403 (M + H) |
| 28 | 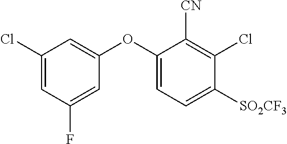 |
| | m/z: 412, 414 (M − H) |
| 29 | 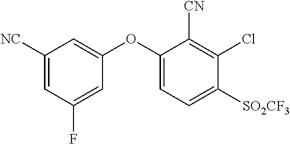 |
| | m/z: 403/405 (M − H) |
| 30 | 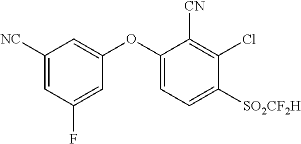 |
| | m/z: 385, 387 (M − H) |
| 31 | 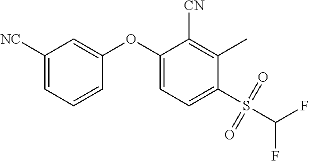 [SMILES: Cc1c(S(=O)(=O)C(F)F)ccc(Oc2cccc(C#N)c2)c1C#N] |
| | m/z: [M − H] 374 |
| 32 | 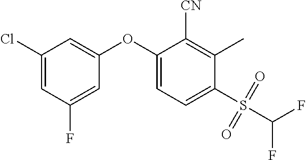 [SMILES: Cc1c(S(=O)(=O)C(F)F)ccc(Oc2cc(F)cc(Cl)c2)c1C#N] |
| | m/z: [M − 1] 374 |
| 33 | 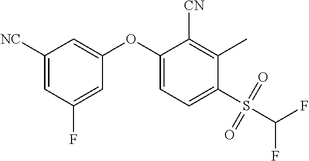 [SMILES: Cc1c(S(=O)(=O)C(F)F)ccc(Oc2cc(F)cc(C#N)c2)c1C#N] |
| | m/z: [M − H] 365 |
| 34 | 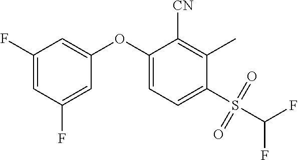 [SMILES: Cc1c(S(=O)(=O)C(F)F)ccc(Oc2cc(F)cc(F)c2)c1C#N] |
| | m/z: [M − H] 358 |
| 35 | 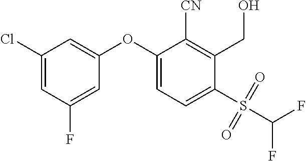 [SMILES: N#Cc1c(Oc2cc(F)cc(Cl)c2)ccc(S(=O)(=O)C(F)F)c1CO] |
| | m/z: [M − H] 391 |
| 36 | 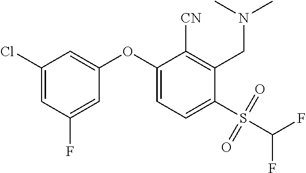 [SMILES: CN(C)Cc1c(S(=O)(=O)C(F)F)ccc(Oc2cc(F)cc(Cl)c2)c1C#N] |
| | m/z: [M + H] 419 |
| 37 | 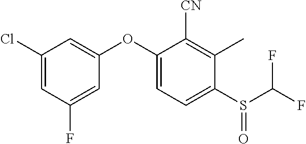 [SMILES: Cc1c(S(=O)C(F)F)ccc(Oc2cc(F)cc(Cl)c2)c1C#N] |
| | m/z: [M − H] 358 |
| 38 | 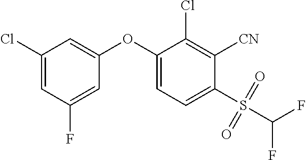 [SMILES: N#Cc1c(S(=O)(=O)C(F)F)ccc(Oc2cc(F)cc(Cl)c2)c1Cl] |
| | m/z: [M − H + 18] 413 |
| 39 | 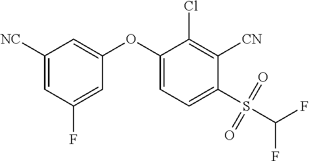 [SMILES: N#Cc1cc(F)cc(Oc2ccc(S(=O)(=O)C(F)F)c(C#N)c2Cl)c1] |
| | m/z: [M − H + 18] 404 |
| 40 | 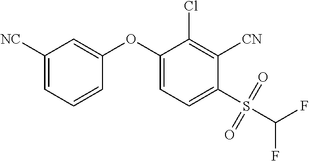 [SMILES: N#Cc1cccc(Oc2ccc(S(=O)(=O)C(F)F)c(C#N)c2Cl)c1] |
| | m/z: [M − H + 18] 386 |
| 41 | 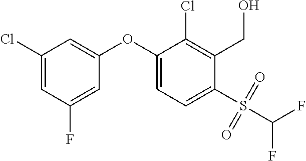 [SMILES: O=S(=O)(c1ccc(Oc2cc(F)cc(Cl)c2)c(Cl)c1CO)C(F)F] |
| | m/z: [M − H + 46] 445 |
| 42 | 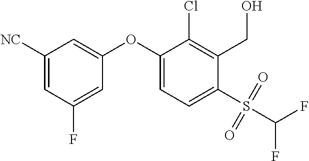 [SMILES: N#Cc1cc(F)cc(Oc2ccc(S(=O)(=O)C(F)F)c(CO)c2Cl)c1] |
| | m/z: [M − H + 46] 436 |
| 43 | 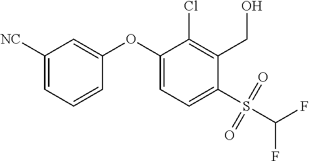 [SMILES: N#Cc1cccc(Oc2ccc(S(=O)(=O)C(F)F)c(CO)c2Cl)c1] |
| | m/z: [M − H + 46] 418 |
| 44 | 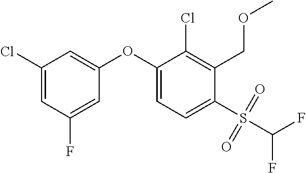 [SMILES: COCc1c(S(=O)(=O)C(F)F)ccc(Oc2cc(F)cc(Cl)c2)c1Cl] |
| | m/z: [M − H + 46] 445 |
| 45 | 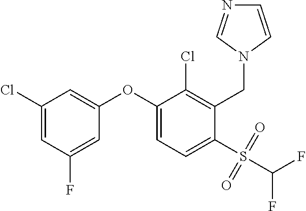 [SMILES: O=S(=O)(c1ccc(Oc2cc(F)cc(Cl)c2)c(Cl)c1Cn1ccnc1)C(F)F] |
| | m/z: [M + H] 451 |
| 46 | 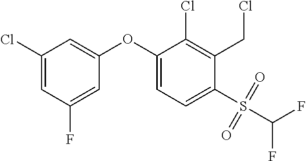 [SMILES: O=S(=O)(c1ccc(Oc2cc(F)cc(Cl)c2)c(Cl)c1CCl)C(F)F] |
| | m/z: [M − H + 46] 463 |
| 47 | 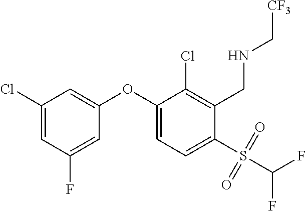 [SMILES: O=S(=O)(c1ccc(Oc2cc(F)cc(Cl)c2)c(Cl)c1CNCC(F)(F)F)C(F)F] |
| | m/z: [M + H] 482 |
| 48 | 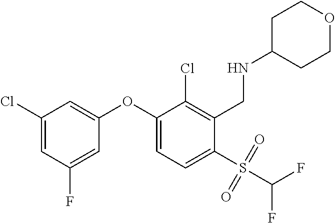 [SMILES: O=S(=O)(c1ccc(Oc2cc(F)cc(Cl)c2)c(Cl)c1CNC1CCOCC1)C(F)F] |
| | m/z: [M + H] 484 |
| 49 | 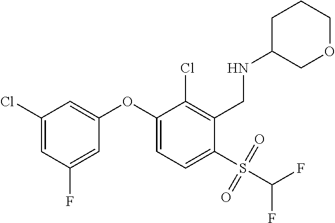 [SMILES: O=S(=O)(c1ccc(Oc2cc(F)cc(Cl)c2)c(Cl)c1CNC1CCCOC1)C(F)F] |
| | m/z: [M + H] 484 |
| 50 | 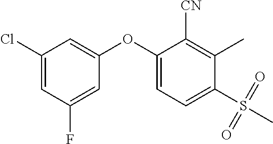 [SMILES: Cc1c(S(C)(=O)=O)ccc(Oc2cc(F)cc(Cl)c2)c1C#N] |
| | m/z: [M − H] 338 |
| 51 | 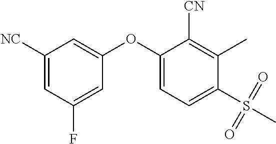 [SMILES: Cc1c(S(C)(=O)=O)ccc(Oc2cc(F)cc(C#N)c2)c1C#N] |
| | m/z: [M − H] 329 |
| 52 | 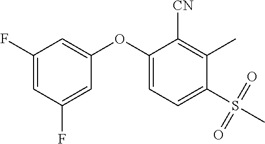 [SMILES: Cc1c(S(C)(=O)=O)ccc(Oc2cc(F)cc(F)c2)c1C#N] |
| | m/z: [M − H] 322 |
| 53 | 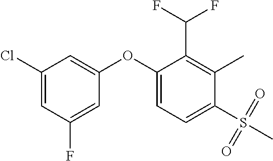 [SMILES: Cc1c(S(C)(=O)=O)ccc(Oc2cc(F)cc(Cl)c2)c1C(F)F] |
| | m/z: [M − H] 363 |
| 54 | 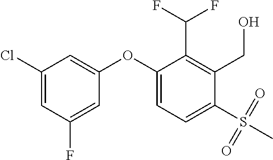 [SMILES: CS(=O)(=O)c1ccc(Oc2cc(F)cc(Cl)c2)c(C(F)F)c1CO] |
| | m/z: [M − H + 46] 425 |
| 55 | 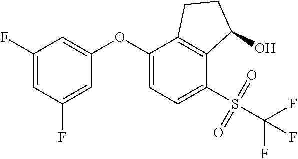 [SMILES: O=S(=O)(c1ccc(Oc2cc(F)cc(F)c2)c2c1[C@H](O)CC2)C(F)(F)F] |
| | m/z: [M − H] 393 |
| 56 | 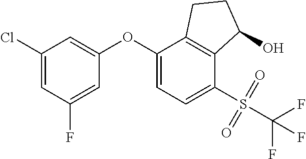 [SMILES: O=S(=O)(c1ccc(Oc2cc(F)cc(Cl)c2)c2c1[C@H](O)CC2)C(F)(F)F] |
| | m/z: [M − H + 46] 455 |
| 57 | 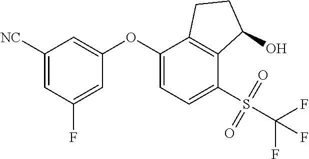 [SMILES: N#Cc1cc(F)cc(Oc2ccc(S(=O)(=O)C(F)(F)F)c3c2CC[C@H]3O)c1] |
| | m/z: [M − H + 46] 446 |
| 58 | 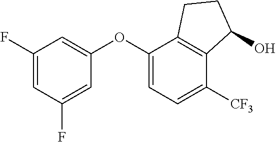 [SMILES: O[C@@H]1CCc2c(Oc3cc(F)cc(F)c3)ccc(C(F)(F)F)c21] |
| | m/z: [M − H] 329 |
| 59 | 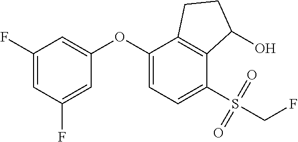 [SMILES: O=S(=O)(CF)c1ccc(Oc2cc(F)cc(F)c2)c2c1C(O)CC2] |
| | m/z: [M − H + 46] 403 |
| 60 | 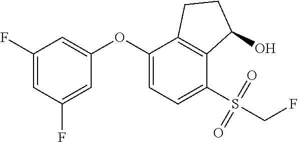 [SMILES: O=S(=O)(CF)c1ccc(Oc2cc(F)cc(F)c2)c2c1[C@H](O)CC2] |
| | m/z: [M − H + 46] 403 |
| 61 | 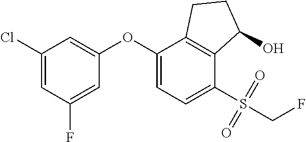 [SMILES: O=S(=O)(CF)c1ccc(Oc2cc(F)cc(Cl)c2)c2c1[C@H](O)CC2] |
| | m/z: [M − H + 46] 419 |
| 62 | 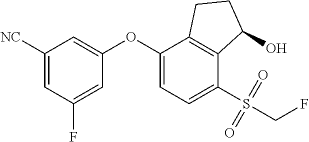 [SMILES: N#Cc1cc(F)cc(Oc2ccc(S(=O)(=O)CF)c3c2CC[C@H]3O)c1] |
| | m/z: [M − H + 46] 410 |
| 63 | 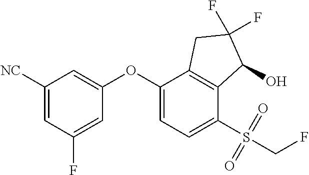 [SMILES: N#Cc1cc(F)cc(Oc2ccc(S(=O)(=O)CF)c3c2CC(F)(F)[C@H]3O)c1] |
| | m/z: [M − H + 46] 446 |
| 64 | 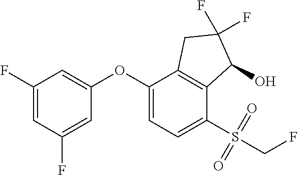 [SMILES: O=S(=O)(CF)c1ccc(Oc2cc(F)cc(F)c2)c2c1[C@H](O)C(F)(F)C2] |
| | m/z: [M − H + 46] 439 |
| 65 | 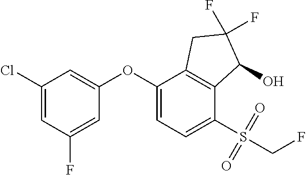 [SMILES: O=S(=O)(CF)c1ccc(Oc2cc(F)cc(Cl)c2)c2c1[C@H](O)C(F)(F)C2] |
| | m/z: [M − H + 46] 455 |
| 66 | 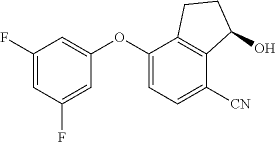 [SMILES: N#Cc1ccc(Oc2cc(F)cc(F)c2)c2c1[C@H](O)CC2] |
| | m/z: [M − H] 286 |
| 67 | 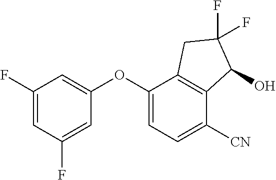 [SMILES: N#Cc1ccc(Oc2cc(F)cc(F)c2)c2c1[C@H](O)C(F)(F)C2] |
| | m/z: [M − H + 46] 368 |
| 68 | 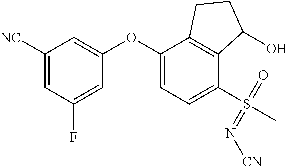 [SMILES: CS(=O)(=NC#N)c1ccc(Oc2cc(F)cc(C#N)c2)c2c1C(O)CC2] |
| | m/z: [M + H] 372 |
| 69 | 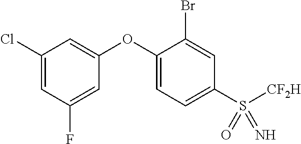 [SMILES: N=S(=O)(c1ccc(Oc2cc(F)cc(Cl)c2)c(Br)c1)C(F)F] |
| | m/z: 414, 416, 418 (M + H) |
| 70 | 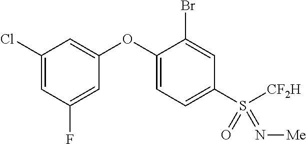 [SMILES: CN=S(=O)(c1ccc(Oc2cc(F)cc(Cl)c2)c(Br)c1)C(F)F] |
| | m/z: 428, 430, 432 (M + H) |
| 71 | 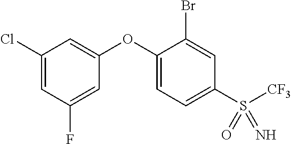 [SMILES: N=S(=O)(c1ccc(Oc2cc(F)cc(Cl)c2)c(Br)c1)C(F)(F)F] |
| | m/z: 430, 432, 434 (M − H) |
| 72 | 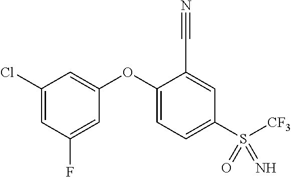 [SMILES: N#Cc1cc(S(=N)(=O)C(F)(F)F)ccc1Oc1cc(F)cc(Cl)c1] |
| | m/z: 377, 379 (M − H) |
| 73 | 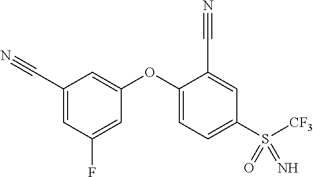 [SMILES: N#Cc1cc(F)cc(Oc2ccc(S(=N)(=O)C(F)(F)F)cc2C#N)c1] |
| | m/z: 368 (M − H) |
| 74 | 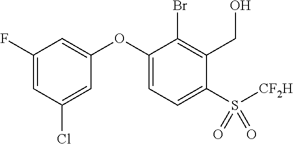 [SMILES: O=S(=O)(c1ccc(Oc2cc(F)cc(Cl)c2)c(Br)c1CO)C(F)F] |
| | m/z: 462, 464, 466 (M + NH₄) |
| 75 | 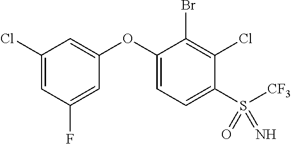 [SMILES: N=S(=O)(c1ccc(Oc2cc(F)cc(Cl)c2)c(Br)c1Cl)C(F)(F)F] |
| | m/z: 464, 466, 468 (M − H) |
| 76 | 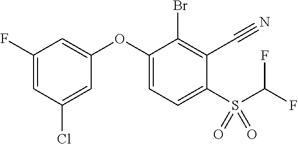 [SMILES: N#Cc1c(S(=O)(=O)C(F)F)ccc(Oc2cc(F)cc(Cl)c2)c1Br] |
| | m/z: 441, 443, 445 (M + H) |
| 77 | 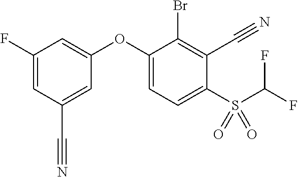 [SMILES: N#Cc1cc(F)cc(Oc2ccc(S(=O)(=O)C(F)F)c(C#N)c2Br)c1] |
| | m/z: 432, 434 (M + H) |
| 78 | 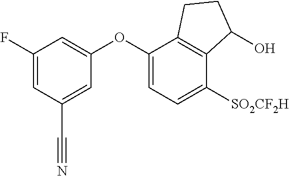 |
| | m/z: 401 (M + NH₄) |
| 79 | 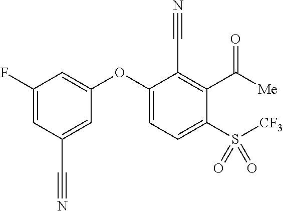 [SMILES: CC(=O)c1c(S(=O)(=O)C(F)(F)F)ccc(Oc2cc(F)cc(C#N)c2)c1C#N] |
| 80 | 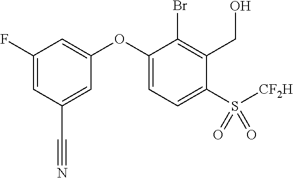 [SMILES: N#Cc1cc(F)cc(Oc2ccc(S(=O)(=O)C(F)F)c(CO)c2Br)c1] |
| | m/z: 453, 455 (M + NH₄) |
| 81 | 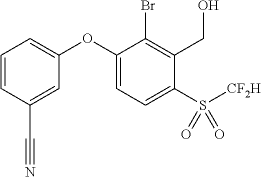 [SMILES: N#Cc1cccc(Oc2ccc(S(=O)(=O)C(F)F)c(CO)c2Br)c1] |
| | m/z: 435, 437 (M + NH₄) |
| 82 | 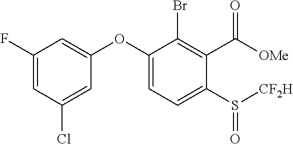 [SMILES: COC(=O)c1c(S(=O)C(F)F)ccc(Oc2cc(F)cc(Cl)c2)c1Br] |
| | m/z: 457, 459, 461 (M + H) |
| 83 | 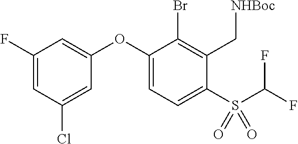 [SMILES: CC(C)(C)OC(=O)NCc1c(S(=O)(=O)C(F)F)ccc(Oc2cc(F)cc(Cl)c2)c1Br] |
| | m/z: 488, 490, 492 [MH⁺ − C₄H₈] |
| 84 | 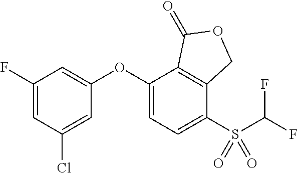 [SMILES: O=C1OCc2c(S(=O)(=O)C(F)F)ccc(Oc3cc(F)cc(Cl)c3)c21] |
| | m/z: 393, 395 (M + H) |
| 85 | 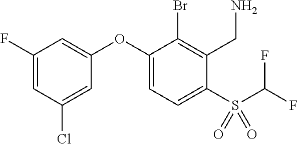 [SMILES: NCc1c(S(=O)(=O)C(F)F)ccc(Oc2cc(F)cc(Cl)c2)c1Br] |
| | m/z: 444, 446, 448 (M + H) |
| 86 | 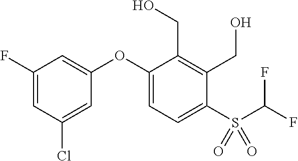 [SMILES: O=S(=O)(c1ccc(Oc2cc(F)cc(Cl)c2)c(CO)c1CO)C(F)F] |
| | m/z: 486, 488, 490 (M + H) |
| 87 | 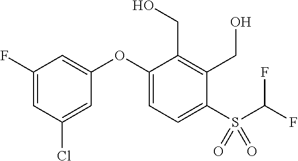 [SMILES: O=S(=O)(c1ccc(Oc2cc(F)cc(Cl)c2)c(CO)c1CO)C(F)F] |
| | m/z: 379, 381 (M + H − 16) |
| 88 | 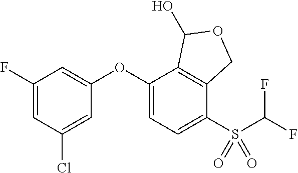 [SMILES: O=S(=O)(c1ccc(Oc2cc(F)cc(Cl)c2)c2c1COC2O)C(F)F] |
| | m/z: 377, 379 (M + H − 16) |
| 89 | 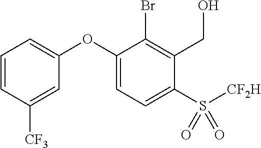 [SMILES: O=S(=O)(c1ccc(Oc2cccc(C(F)(F)F)c2)c(Br)c1CO)C(F)F] |
| | m/z: 478, 480 (M + NH₄) |
| 90 | 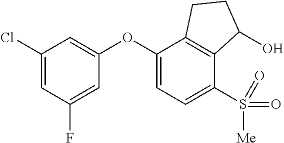 [SMILES: CS(=O)(=O)c1ccc(Oc2cc(F)cc(Cl)c2)c2c1C(O)CC2] |
| | m/z: 339, 341 (M + H − 16) |
| 91 | 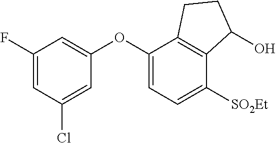 |
| | m/z: 353, 355 (M − OH) |
| 92 | 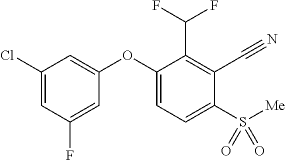 [SMILES: CS(=O)(=O)c1ccc(Oc2cc(F)cc(Cl)c2)c(C(F)F)c1C#N] |
| | m/z: 393, 395 (M + NH₄) |
| 93 | 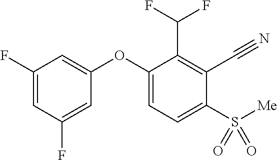 [SMILES: CS(=O)(=O)c1ccc(Oc2cc(F)cc(F)c2)c(C(F)F)c1C#N] |
| | m/z: 377 (M + NH₄) |
| 94 | 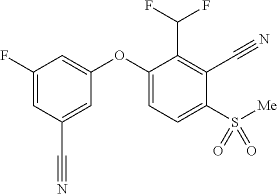 [SMILES: CS(=O)(=O)c1ccc(Oc2cc(F)cc(C#N)c2)c(C(F)F)c1C#N] |
| | m/z: 384 (M + NH₄) |
| 95 | 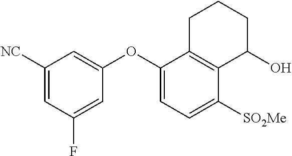 [SMILES: CS(=O)(=O)c1ccc(Oc2cc(F)cc(C#N)c2)c2c1C(O)CCC2] |
| | m/z: 379 (M + NH₄) |
| 96 | 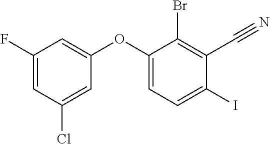 [SMILES: N#Cc1c(I)ccc(Oc2cc(F)cc(Cl)c2)c1Br] |
| 97 | 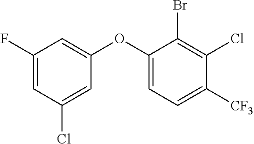 [SMILES: Fc1cc(Cl)cc(Oc2ccc(C(F)(F)F)c(Cl)c2Br)c1] |
| 98 | 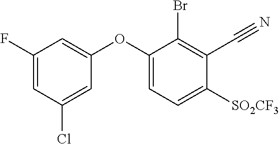 |
| | m/z: (M − H) 456, 458 |
| 99 | 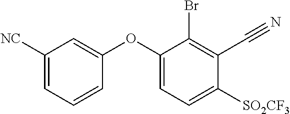 |
| | m/z: (M + NH₄) 448 |
| 100 | 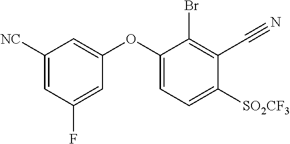 |
| | m/z: (M + NH₄) 466 |
| 101 | 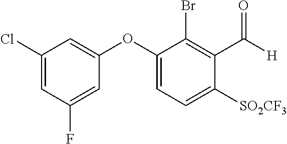 |
| 102 | 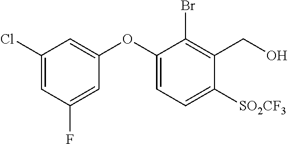 |
| 103 | 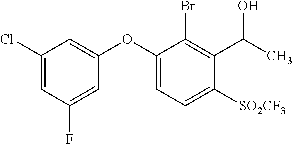 |
| | m/z: (M − H) 475, 477 |
| 104 | 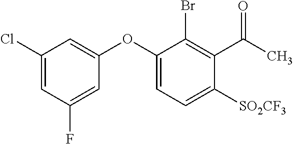 |
| | m/z: (M + H) 474.8/476.7 |
| 105 | 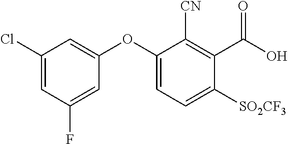 |
| | m/z: (M + H) 424, 426 |
| 106 | 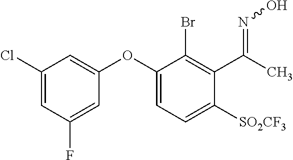 |
| | m/z: (M + H) 492, 494 |
| 107 | 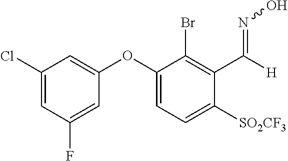 |
| | m/z: (M + H) 476, 478 |
| 108 | 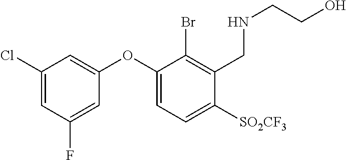 |
| | m/z: (M + H) 506, 508 |
| 109 | 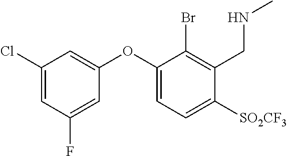 |
| | m/z: (M + H) 476, 478 |
| 110 | 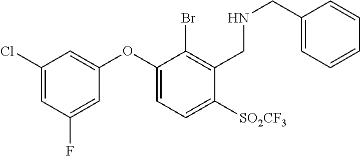 |
| | m/z: (M + H) 552, 554 |
| 111 | 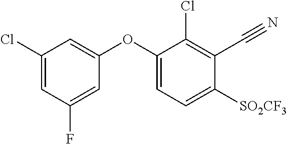 |
| | m/z: (M + NH₄) 431, 433 |
| 112 | 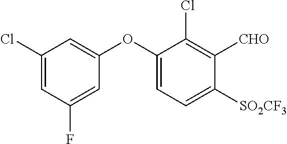 |
| 113 | 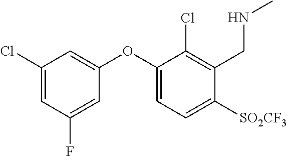 |
| | m/z: (M + H) 432, 434 |
| 114 | 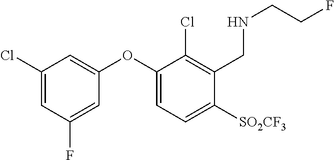 |
| | m/z: (M + H) 464, 466 |
| 115 | 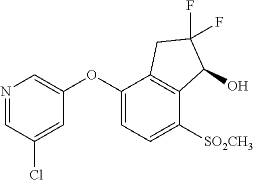 [SMILES: CS(=O)(=O)c1ccc(Oc2cncc(Cl)c2)c2c1[C@H](O)C(F)(F)C2] |
| | m/z: (M + H) 376, 378 |
| 116 | 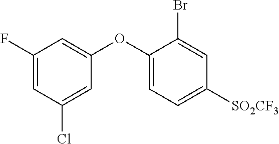 |
| | m/z: 431 (M − H) |
| 117 | 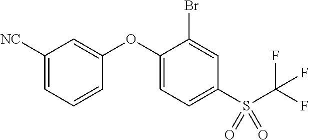 [SMILES: N#Cc1cccc(Oc2ccc(S(=O)(=O)C(F)(F)F)cc2Br)c1] |
| | m/z: 404 (M − H) |
| 118 | 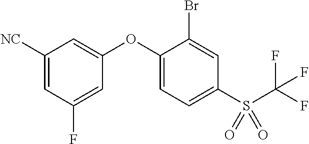 [SMILES: N#Cc1cc(F)cc(Oc2ccc(S(=O)(=O)C(F)(F)F)cc2Br)c1] |
| | m/z: 422 (M − H) |
| 119 | 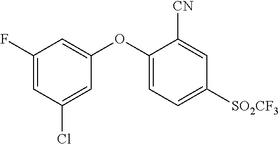 |
| | m/z: 379 (M − H) |
| 120 | 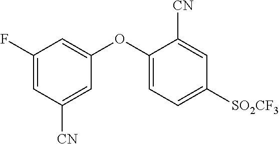 |
| | m/z: 369 (M − H) |
| 121 | 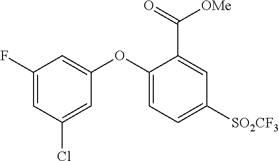 |
| | m/z: 411 (M − H) |
| 122 | 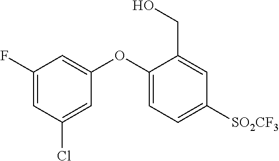 |
| | m/z: 383 (M − H) |
| 123 | 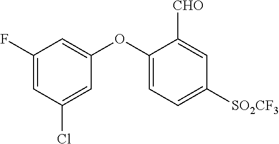 |
| 124 | 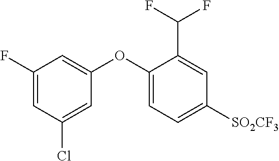 |
| 125 | 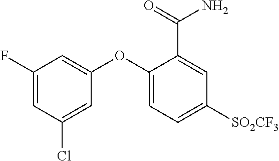 |
| | m/z: 396 (M − H) |
| 126 | 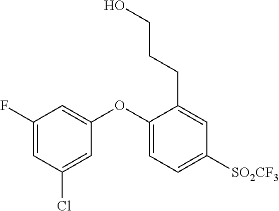 |
| | m/z: 457 (M + HCO₂⁻) |
| 127 | 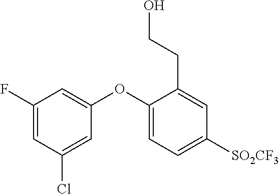 |
| | m/z: 397 (M − H) |
| 128 | 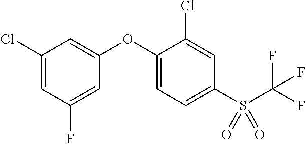 [SMILES: O=S(=O)(c1ccc(Oc2cc(F)cc(Cl)c2)c(Cl)c1)C(F)(F)F] |
| | m/z: 387 (M − H) |
| 129 | 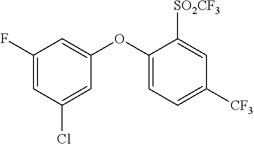 |
| | m/z: 421 (M − H) |
| 130 | 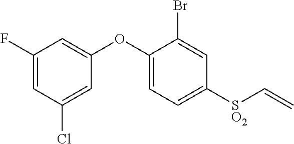 [SMILES: C=CS(=O)(=O)c1ccc(Oc2cc(F)cc(Cl)c2)c(Br)c1] |
| | m/z: 389 (M − H) |
| 131 | 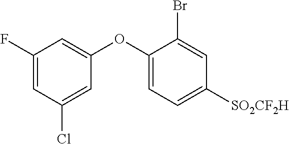 |
| | m/z: 431 (M − H) |
| 132 | 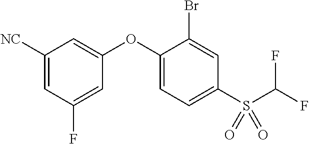 [SMILES: N#Cc1cc(F)cc(Oc2ccc(S(=O)(=O)C(F)F)cc2Br)c1] |
| | m/z: 404 (M − H) |
| 133 | 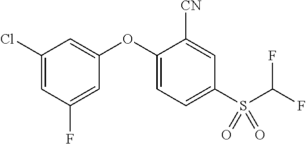 [SMILES: N#Cc1cc(S(=O)(=O)C(F)F)ccc1Oc1cc(F)cc(Cl)c1] |
| | m/z: 360 (M − H) |
| 134 | 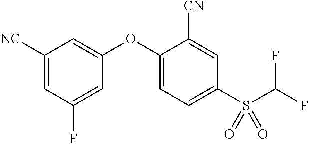 [SMILES: N#Cc1cc(F)cc(Oc2ccc(S(=O)(=O)C(F)F)cc2C#N)c1] |
| | m/z: 351 (M − H) |
| 135 | 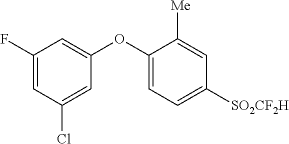 |
| | m/z: 349 (M − H) |
| 136 | 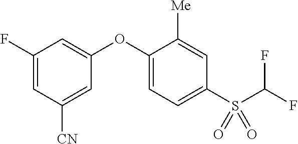 [SMILES: Cc1cc(S(=O)(=O)C(F)F)ccc1Oc1cc(F)cc(C#N)c1] |
| | m/z: 340 (M − H) |
| 137 | 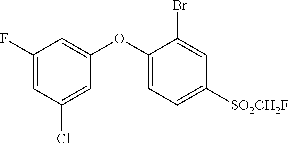 |
| | m/z: 395 (M − H) |
| 138 | 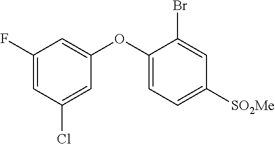 [SMILES: CS(=O)(=O)c1ccc(Oc2cc(F)cc(Cl)c2)c(Br)c1] |
| | m/z: 377 (M − H) |
| 139 | 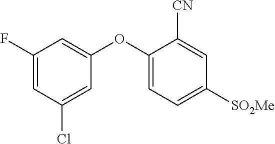 [SMILES: CS(=O)(=O)c1ccc(Oc2cc(F)cc(Cl)c2)c(C#N)c1] |
| | m/z: 370 (M + HCO₂⁻) |
| 140 | 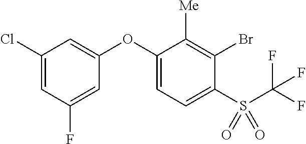 [SMILES: Cc1c(Oc2cc(F)cc(Cl)c2)ccc(S(=O)(=O)C(F)(F)F)c1Br] |
| | m/z: 445 (M − H) |
| 141 | 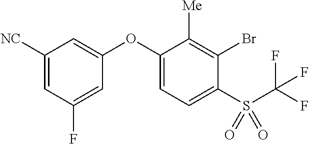 [SMILES: Cc1c(Oc2cc(F)cc(C#N)c2)ccc(S(=O)(=O)C(F)(F)F)c1Br] |
| | m/z: 438 (M + H) |
| 142 | 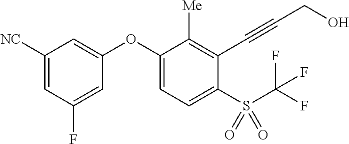 [SMILES: Cc1c(Oc2cc(F)cc(C#N)c2)ccc(S(=O)(=O)C(F)(F)F)c1C#CCO] |
| | m/z: 412 (M − H) |
| 143 | 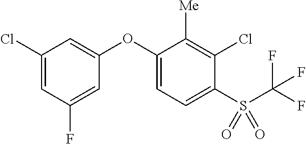 [SMILES: Cc1c(Oc2cc(F)cc(Cl)c2)ccc(S(=O)(=O)C(F)(F)F)c1Cl] |
| | m/z: 401 (M − H) |
| 144 | 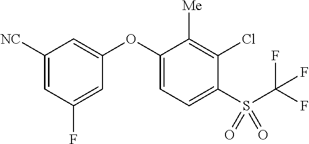 [SMILES: Cc1c(Oc2cc(F)cc(C#N)c2)ccc(S(=O)(=O)C(F)(F)F)c1Cl] |
| | m/z: 392 (M − H) |
| 145 | 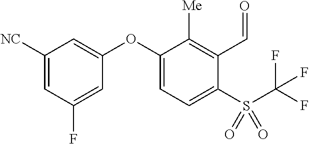 [SMILES: Cc1c(Oc2cc(F)cc(C#N)c2)ccc(S(=O)(=O)C(F)(F)F)c1C=O] |
| | m/z: 386 (M − H) |
| 146 | 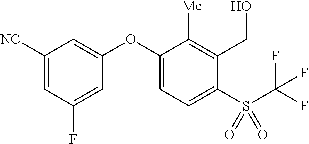 [SMILES: Cc1c(Oc2cc(F)cc(C#N)c2)ccc(S(=O)(=O)C(F)(F)F)c1CO] |
| | m/z: 434 (M + HCO₂⁻) |
| 147 | 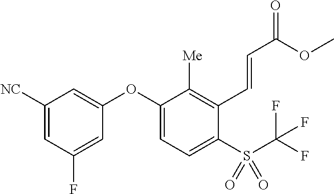 [SMILES: COC(=O)/C=C/c1c(S(=O)(=O)C(F)(F)F)ccc(Oc2cc(F)cc(C#N)c2)c1C] |
| | m/z: 442 (M − H) |
| 148 | 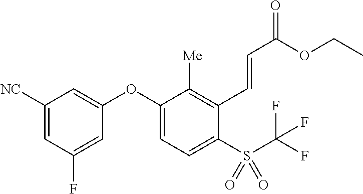 [SMILES: CCOC(=O)/C=C/c1c(S(=O)(=O)C(F)(F)F)ccc(Oc2cc(F)cc(C#N)c2)c1C] |
| | m/z: 458 (M + H) |
| 149 | 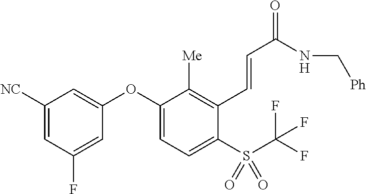 [SMILES: Cc1c(Oc2cc(F)cc(C#N)c2)ccc(S(=O)(=O)C(F)(F)F)c1/C=C/C(=O)NCc1ccccc1] |
| | m/z: 519 (M + H) |
| 150 | 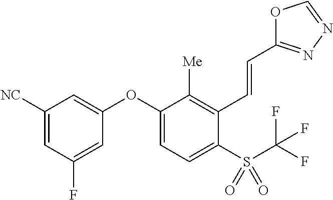 [SMILES: Cc1c(Oc2cc(F)cc(C#N)c2)ccc(S(=O)(=O)C(F)(F)F)c1/C=C/c1nnco1] |
| | m/z: 454 (M + H) |
| 151 | 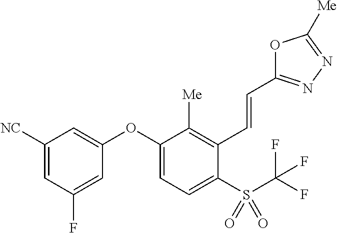 [SMILES: Cc1nnc(/C=C/c2c(S(=O)(=O)C(F)(F)F)ccc(Oc3cc(F)cc(C#N)c3)c2C)o1] |
| | m/z: 468 (M + H) |
| 152 | 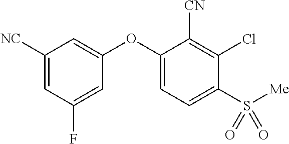 [SMILES: CS(=O)(=O)c1ccc(Oc2cc(F)cc(C#N)c2)c(C#N)c1Cl] |
| | m/z: 349 (M − H) |
| 153 | 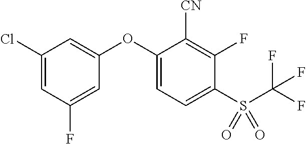 [SMILES: N#Cc1c(Oc2cc(F)cc(Cl)c2)ccc(S(=O)(=O)C(F)(F)F)c1F] |
| | m/z: 396 (M − H) |
| 154 | 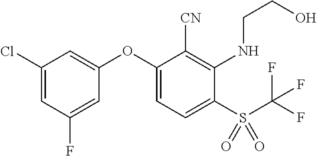 [SMILES: N#Cc1c(Oc2cc(F)cc(Cl)c2)ccc(S(=O)(=O)C(F)(F)F)c1NCCO] |
| | m/z: 437 (M − H) |
| 155 | 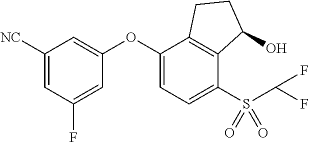 [SMILES: N#Cc1cc(F)cc(Oc2ccc(S(=O)(=O)C(F)F)c3c2CC[C@H]3O)c1] |
| | m/z: 428 (M + HCO₂⁻) |
| 156 | 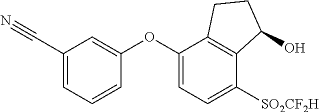 |
| | m/z: 410 (M + HCO₂⁻) |
| 157 | 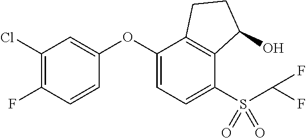 [SMILES: O=S(=O)(c1ccc(Oc2ccc(F)c(Cl)c2)c2c1[C@H](O)CC2)C(F)F] |
| | m/z: 437 (M + HCO₂⁻) |
| 158 | 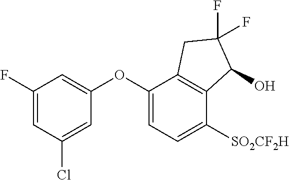 |
| | m/z: 473 (M + HCO₂⁻) |
| 159 | 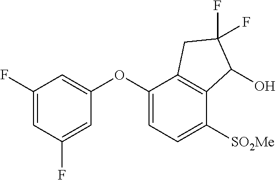 [SMILES: CS(=O)(=O)c1ccc(Oc2cc(F)cc(F)c2)c2c1C(O)C(F)(F)C2] |
| | m/z: 421 (M + HCO₂⁻) |
| 160 | 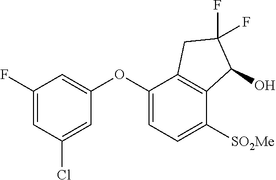 [SMILES: CS(=O)(=O)c1ccc(Oc2cc(F)cc(Cl)c2)c2c1[C@H](O)C(F)(F)C2] |
| | m/z: 393 (M + H) |
| 161 | 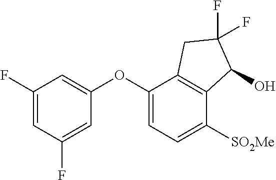 [SMILES: CS(=O)(=O)c1ccc(Oc2cc(F)cc(F)c2)c2c1[C@H](O)C(F)(F)C2] |
| | m/z: 421 (M + HCO₂⁻) |
| 162 | 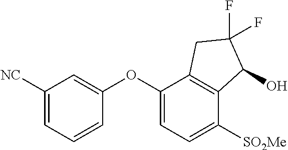 [SMILES: CS(=O)(=O)c1ccc(Oc2cccc(C#N)c2)c2c1[C@H](O)C(F)(F)C2] |
| | m/z: 410 (M + HCO₂⁻) |
| 163 | 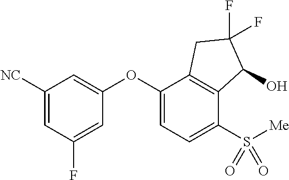 [SMILES: CS(=O)(=O)c1ccc(Oc2cc(F)cc(C#N)c2)c2c1[C@H](O)C(F)(F)C2] |
| | m/z: 428 (M + HCO₂⁻) |
| 164 | 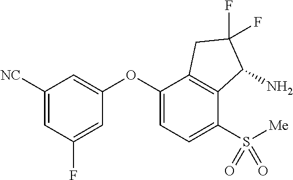 [SMILES: CS(=O)(=O)c1ccc(Oc2cc(F)cc(C#N)c2)c2c1[C@@H](N)C(F)(F)C2] |
| | m/z: 383 (M + H) |
| 165 | 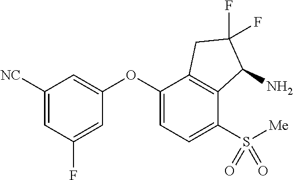 [SMILES: CS(=O)(=O)c1ccc(Oc2cc(F)cc(C#N)c2)c2c1[C@H](N)C(F)(F)C2] |
| | m/z: 383 (M + H) |
| 166 | 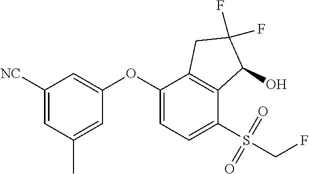 [SMILES: Cc1cc(C#N)cc(Oc2ccc(S(=O)(=O)CF)c3c2CC(F)(F)[C@H]3O)c1] |
| | m/z: [M − H + 46]: 442 |
| 167 | 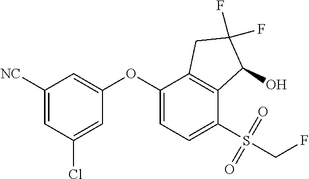 [SMILES: N#Cc1cc(Cl)cc(Oc2ccc(S(=O)(=O)CF)c3c2CC(F)(F)[C@H]3O)c1] |
| | m/z: [M − H + 46]: 462 |
| 168 | 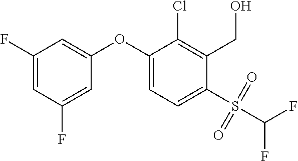 [SMILES: O=S(=O)(c1ccc(Oc2cc(F)cc(F)c2)c(Cl)c1CO)C(F)F] |
| | m/z: [M − H + 46]: 429 |
| 169 | 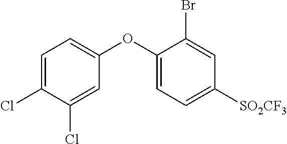 |
| 170 | 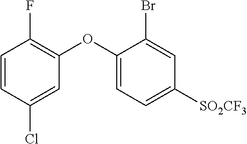 |
| 171 | 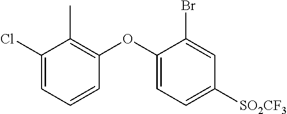 |
| 172 | 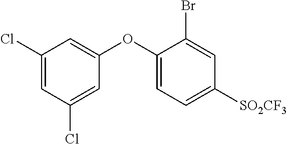 |
| 173 | 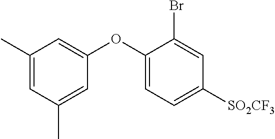 |
| 174 | 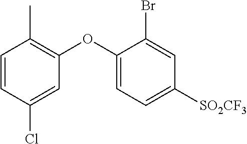 |
| 175 | 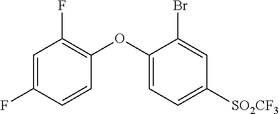 |
| 176 | 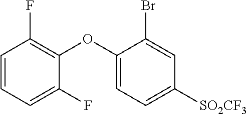 |
| 177 | 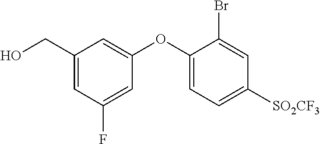 |
| 178 | 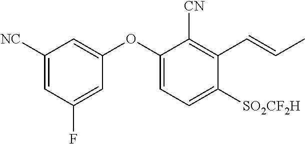 |
| | m/z: 391 (M − H) |
| 179 | 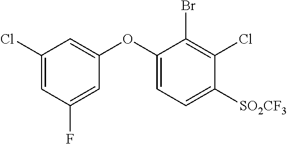 |
| 180 | 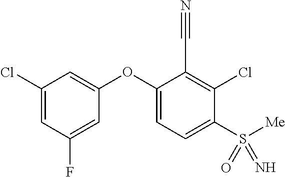 [SMILES: CS(=N)(=O)c1ccc(Oc2cc(F)cc(Cl)c2)c(C#N)c1Cl] |
| | m/z: 359, 361 (M + H) |
| 181 | 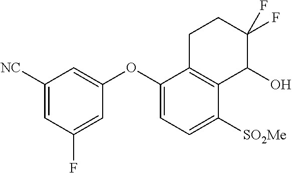 [SMILES: CS(=O)(=O)c1ccc(Oc2cc(F)cc(C#N)c2)c2c1C(O)C(F)(F)CC2] |
| 182 | 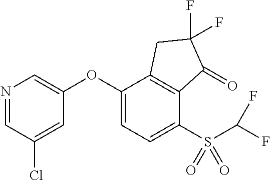 [SMILES: O=C1c2c(S(=O)(=O)C(F)F)ccc(Oc3cncc(Cl)c3)c2CC1(F)F] |
| | m/z: (M + H) 410/412 |
| 183 | 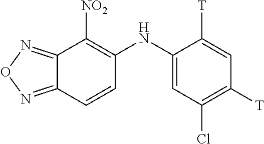 |
| | m/z: 478 mCi |
| 184 | 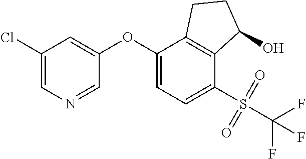 [SMILES: O=S(=O)(c1ccc(Oc2cncc(Cl)c2)c2c1[C@H](O)CC2)C(F)(F)F] |
| | m/z: [M + H] 394 |
| 185 | 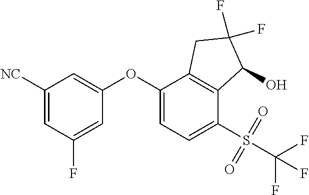 [SMILES: N#Cc1cc(F)cc(Oc2ccc(S(=O)(=O)C(F)(F)F)c3c2CC(F)(F)[C@H]3O)c1] |
| | m/z: [M − H] 436 |
| 186 | 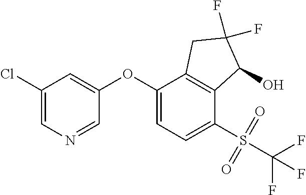 [SMILES: O=S(=O)(c1ccc(Oc2cncc(Cl)c2)c2c1[C@H](O)C(F)(F)C2)C(F)(F)F] |
| | m/z: [M + H] 430 |
| 187 | 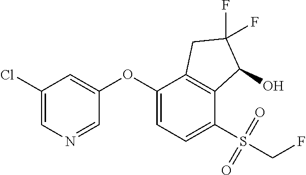 [SMILES: O=S(=O)(CF)c1ccc(Oc2cncc(Cl)c2)c2c1[C@H](O)C(F)(F)C2] |
| | m/z: [M + H] 394 |
| 188 | 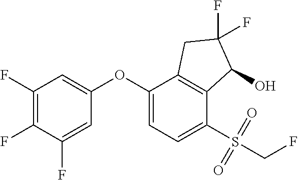 [SMILES: O=S(=O)(CF)c1ccc(Oc2cc(F)c(F)c(F)c2)c2c1[C@H](O)C(F)(F)C2] |
| | m/z: [M − H + 46] 457 |
| 189 | 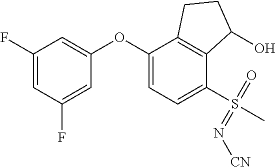 [SMILES: CS(=O)(=NC#N)c1ccc(Oc2cc(F)cc(F)c2)c2c1C(O)CC2] |
| | m/z: [M + H] 365 |
| 190 | 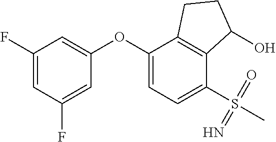 [SMILES: CS(=N)(=O)c1ccc(Oc2cc(F)cc(F)c2)c2c1C(O)CC2] |
| | m/z: [M + H] 340 |
| 191 | 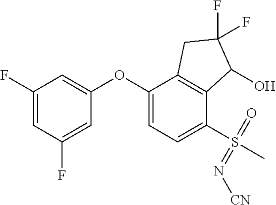 [SMILES: CS(=O)(=NC#N)c1ccc(Oc2cc(F)cc(F)c2)c2c1C(O)C(F)(F)C2] |
| | m/z: [M + H] 401 |
| 192 | 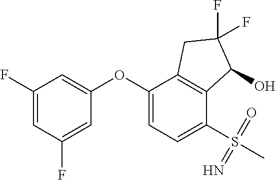 [SMILES: CS(=N)(=O)c1ccc(Oc2cc(F)cc(F)c2)c2c1[C@H](O)C(F)(F)C2] |
| | m/z: [M + H] 376 |
| 193 | 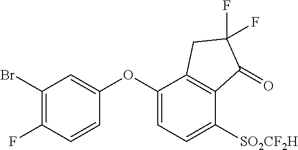 |
| 194 | 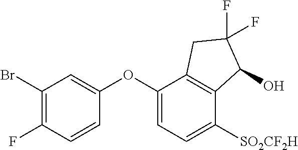 |
| | m/z: (M + HCOOH − H): 517, 519 |
| 195 | 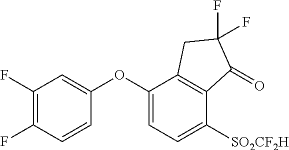 |
| 196 | 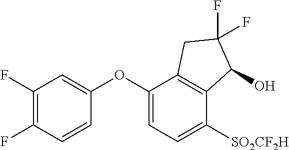 |
| | m/z: (M + HCOOH − H) 457 |
| 197 | 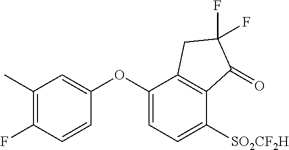 |
| 198 | 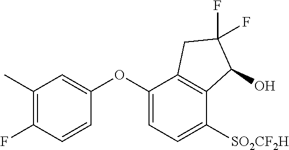 |
| | m/z: (M + HCOOH − H) 453 |
| 199 | 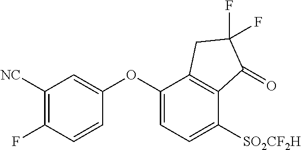 |
| 200 | 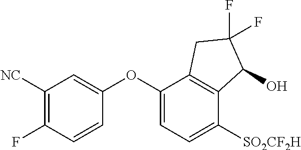 |
| | m/z: (M + HCOOH − H) 464 |
| 201 | 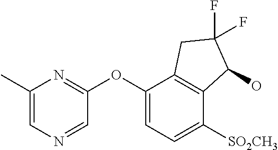 [SMILES: Cc1cncc(Oc2ccc(S(C)(=O)=O)c3c2CC(F)(F)[C@H]3O)n1] |
| | m/z: (M + HCOOH − H) 401 |
| 202 | 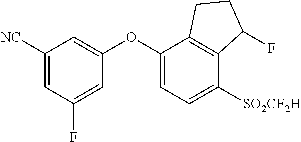 |
| | m/z: (M + HCOOH − H) 428 |
| 203 | 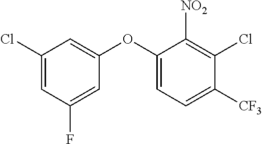 [SMILES: O=[N+]([O-])c1c(Oc2cc(F)cc(Cl)c2)ccc(C(F)(F)F)c1Cl] |
| 204 | 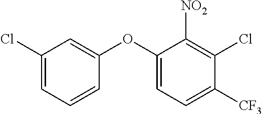 [SMILES: O=[N+]([O-])c1c(Oc2cccc(Cl)c2)ccc(C(F)(F)F)c1Cl] |
| | m/z: 350, 352, 354 (M − H) |
| 205 | 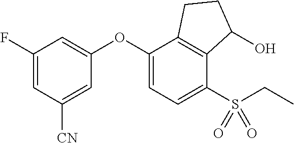 [SMILES: CCS(=O)(=O)c1ccc(Oc2cc(F)cc(C#N)c2)c2c1C(O)CC2] |
| 206 | 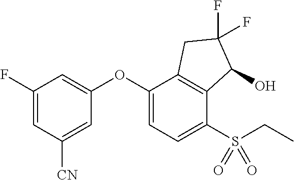 [SMILES: CCS(=O)(=O)c1ccc(Oc2cc(F)cc(C#N)c2)c2c1[C@H](O)C(F)(F)C2] |
| | m/z: 442 (M + HCO2) |
| 207 | 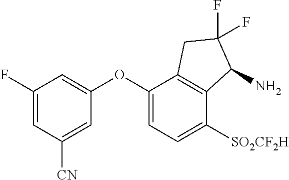 |
| | m/z: 419 (M + H) |
| 208 | 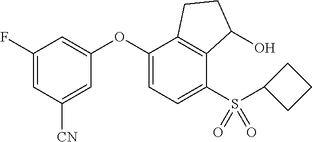 [SMILES: N#Cc1cc(F)cc(Oc2ccc(S(=O)(=O)C3CCC3)c3c2CCC3O)c1] |
| 209 | 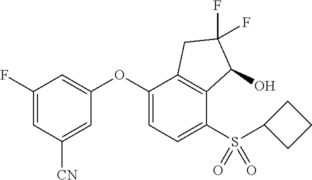 [SMILES: N#Cc1cc(F)cc(Oc2ccc(S(=O)(=O)C3CCC3)c3c2CC(F)(F)[C@H]3O)c1] |
| | m/z: 468 (M + HCO2) |
| 210 | 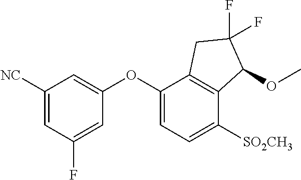 [SMILES: CO[C@H]1c2c(S(C)(=O)=O)ccc(Oc3cc(F)cc(C#N)c3)c2CC1(F)F] |
| | m/z: 398 (M + H) |
| 211 | 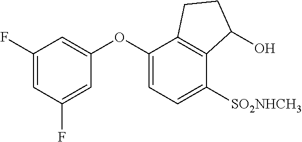 |
| | m/z: 354 (M − H) |
| 212 | 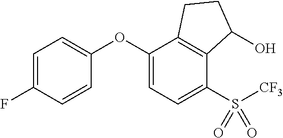 [SMILES: O=S(=O)(c1ccc(Oc2ccc(F)cc2)c2c1C(O)CC2)C(F)(F)F] |
| 213 | 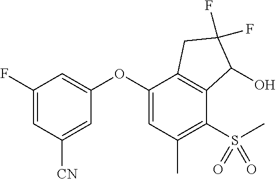 [SMILES: Cc1cc(Oc2cc(F)cc(C#N)c2)c2c(c1S(C)(=O)=O)C(O)C(F)(F)C2] |
| 214 | 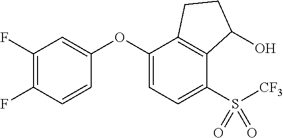 [SMILES: O=S(=O)(c1ccc(Oc2ccc(F)c(F)c2)c2c1C(O)CC2)C(F)(F)F] |
| 215 | 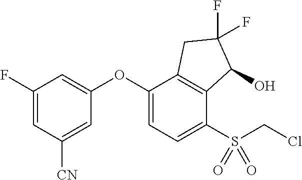 [SMILES: N#Cc1cc(F)cc(Oc2ccc(S(=O)(=O)CCl)c3c2CC(F)(F)[C@H]3O)c1] |
| 216 | 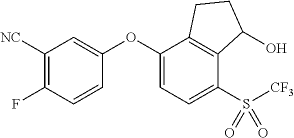 [SMILES: N#Cc1cc(Oc2ccc(S(=O)(=O)C(F)(F)F)c3c2CCC3O)ccc1F] |
| 217 | 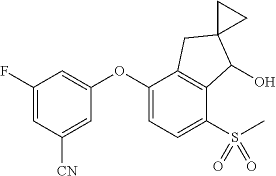 [SMILES: CS(=O)(=O)c1ccc(Oc2cc(F)cc(C#N)c2)c2c1C(O)C1(CC1)C2] |
| | m/z: 418 (M + HCO₂⁻) |
| 218 | 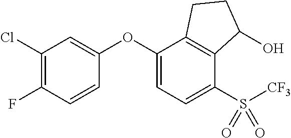 [SMILES: O=S(=O)(c1ccc(Oc2ccc(F)c(Cl)c2)c2c1C(O)CC2)C(F)(F)F] |
| 219 | 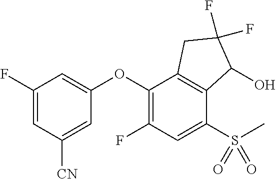 [SMILES: CS(=O)(=O)c1cc(F)c(Oc2cc(F)cc(C#N)c2)c2c1C(O)C(F)(F)C2] |
| 220 | 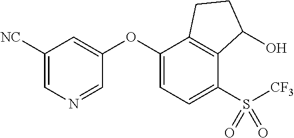 [SMILES: N#Cc1cncc(Oc2ccc(S(=O)(=O)C(F)(F)F)c3c2CCC3O)c1] |
| 221 | 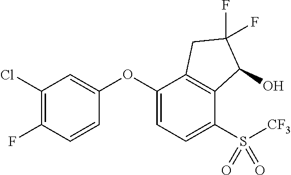 [SMILES: O=S(=O)(c1ccc(Oc2ccc(F)c(Cl)c2)c2c1[C@H](O)C(F)(F)C2)C(F)(F)F] |
| | m/z: 445, 447 (M − H) |
| 222 | 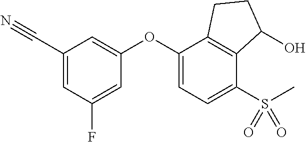 [SMILES: CS(=O)(=O)c1ccc(Oc2cc(F)cc(C#N)c2)c2c1C(O)CC2] |
| | m/z: 392 (M + HCO₂⁻) |
| 223 | 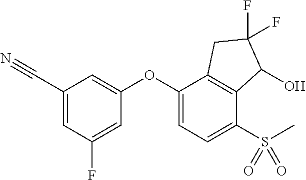 [SMILES: CS(=O)(=O)c1ccc(Oc2cc(F)cc(C#N)c2)c2c1C(O)C(F)(F)C2] |
| | m/z: 428 (M + HCO₂⁻) |
| 224 | 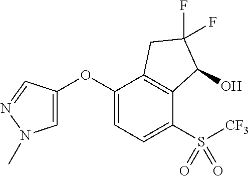 [SMILES: Cn1cc(Oc2ccc(S(=O)(=O)C(F)(F)F)c3c2CC(F)(F)[C@H]3O)cn1] |
| | m/z: 399 (M + H) |
| 225 | 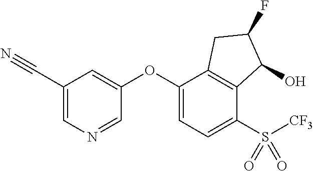 [SMILES: N#Cc1cncc(Oc2ccc(S(=O)(=O)C(F)(F)F)c3c2C[C@@H](F)[C@H]3O)c1] |
| | m/z: 401 (M − H) |
| 226 | 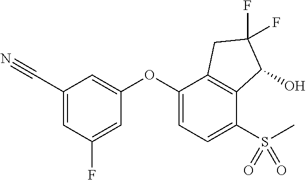 [SMILES: CS(=O)(=O)c1ccc(Oc2cc(F)cc(C#N)c2)c2c1[C@@H](O)C(F)(F)C2] |
| | m/z: 428 (M + HCO₂⁻) |
| 227 | 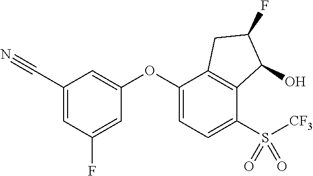 [SMILES: N#Cc1cc(F)cc(Oc2ccc(S(=O)(=O)C(F)(F)F)c3c2C[C@@H](F)[C@H]3O)c1] |
| | m/z: 428 (M + HCO₂⁻) |
| 228 | 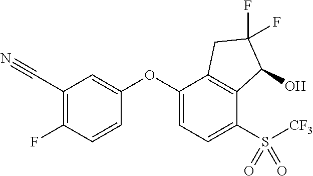 [SMILES: N#Cc1cc(Oc2ccc(S(=O)(=O)C(F)(F)F)c3c2CC(F)(F)[C@H]3O)ccc1F] |
| | m/z: 436 (M − H) |
| 229 | 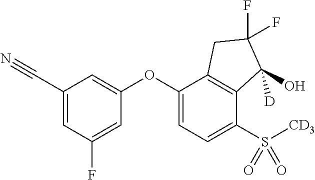 [SMILES: [2H]C([2H])([2H])S(=O)(=O)c1ccc(Oc2cc(F)cc(C#N)c2)c2c1[C@]([2H])(O)C(F)(F)C2] |
| | m/z: 432 (M + HCO₂⁻) |
| 230 | 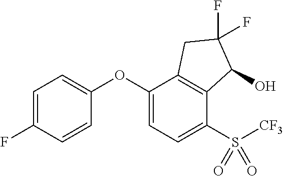 [SMILES: O=S(=O)(c1ccc(Oc2ccc(F)cc2)c2c1[C@H](O)C(F)(F)C2)C(F)(F)F] |
| | m/z: 411 (M − H) |
| 231 | 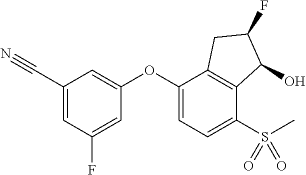 [SMILES: CS(=O)(=O)c1ccc(Oc2cc(F)cc(C#N)c2)c2c1[C@H](O)[C@H](F)C2] |
| | m/z: 383 (M + NH₄⁺) |
| 232 | 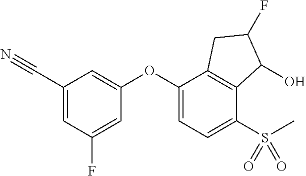 [SMILES: CS(=O)(=O)c1ccc(Oc2cc(F)cc(C#N)c2)c2c1C(O)C(F)C2] |
| | m/z: 383 (M + NH₄⁺) |
| 233 | 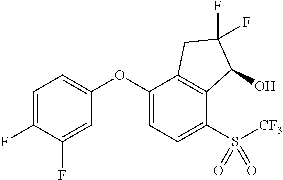 [SMILES: O=S(=O)(c1ccc(Oc2ccc(F)c(F)c2)c2c1[C@H](O)C(F)(F)C2)C(F)(F)F] |
| | m/z: 429 (M − H) |
| 234 | 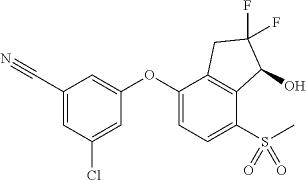 [SMILES: CS(=O)(=O)c1ccc(Oc2cc(Cl)cc(C#N)c2)c2c1[C@H](O)C(F)(F)C2] |
| | m/z: 444 (M + HCO₂⁻) |
| 235 | 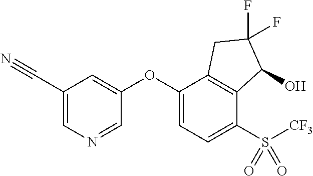 [SMILES: N#Cc1cncc(Oc2ccc(S(=O)(=O)C(F)(F)F)c3c2CC(F)(F)[C@H]3O)c1] |
| | m/z: 419 (M − H) |
| 236 | 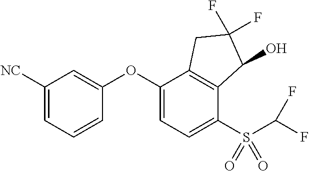 [SMILES: N#Cc1cccc(Oc2ccc(S(=O)(=O)C(F)F)c3c2CC(F)(F)[C@H]3O)c1] |
| | m/z: 419 (M + NH4) |
| 237 | 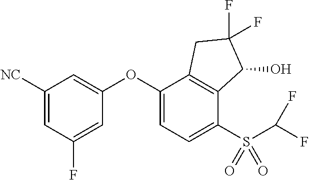 [SMILES: N#Cc1cc(F)cc(Oc2ccc(S(=O)(=O)C(F)F)c3c2CC(F)(F)[C@@H]3O)c1] |
| 238 | 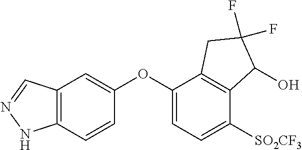 |
| | m/z: [M + H] 435 |
| 239 | 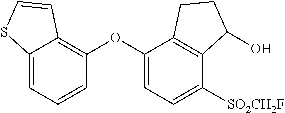 |
| | m/z: [M + formic acid] 459 |
| 240 | 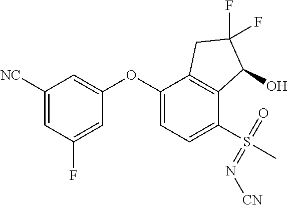 [SMILES: CS(=O)(=NC#N)c1ccc(Oc2cc(F)cc(C#N)c2)c2c1[C@H](O)C(F)(F)C2] |
| | m/z: [M + H] 408 |
| 241 | 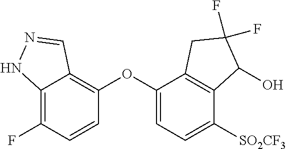 |
| | m/z: [M + 1] 453 |
| 242 | 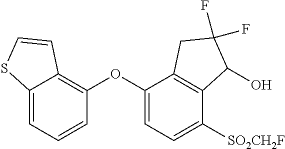 |
| | m/z: [M + formic acid] 459 |
| 243 | 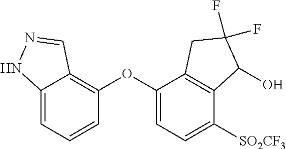 |
| | m/z: [M + H] 435 |
| 244 | 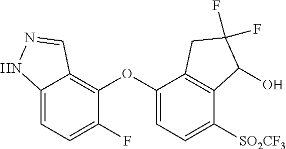 |
| | m/z: [M + H] 453 |
| 245 | 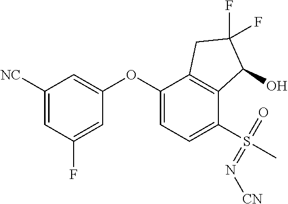 [SMILES: CS(=O)(=NC#N)c1ccc(Oc2cc(F)cc(C#N)c2)c2c1[C@H](O)C(F)(F)C2] |
| | m/z: [M + H] 408 |
| 246 | 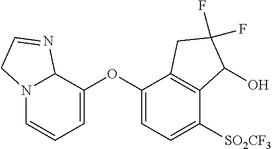 |
| | m/z: [M + H] 435 |
| 247 | 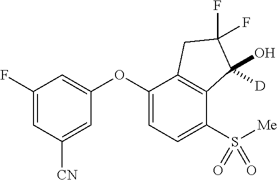 [SMILES: [2H][C@]1(O)c2c(S(C)(=O)=O)ccc(Oc3cc(F)cc(C#N)c3)c2CC1(F)F] |
| | m/z: (M + H) 385 |
| 248 | 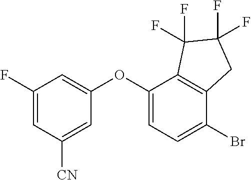 [SMILES: N#Cc1cc(F)cc(Oc2ccc(Br)c3c2C(F)(F)C(F)(F)C3)c1] |
| | m/z: (M − H) 402, 404 |
| 249 | 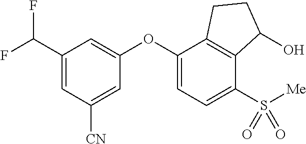 [SMILES: CS(=O)(=O)c1ccc(Oc2cc(C#N)cc(C(F)F)c2)c2c1C(O)CC2] |
| | m/z: (M + NH₄) 397 |
| 250 | 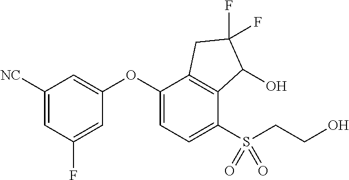 [SMILES: N#Cc1cc(F)cc(Oc2ccc(S(=O)(=O)CCO)c3c2CC(F)(F)C3O)c1] |
| | m/z: 414 (M + H) |
| 251 | 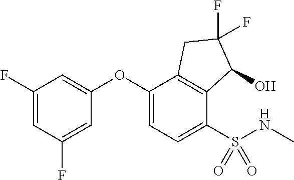 [SMILES: CNS(=O)(=O)c1ccc(Oc2cc(F)cc(F)c2)c2c1[C@H](O)C(F)(F)C2] |
| | m/z: 392 (M + H) |
| 252 | 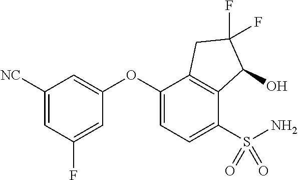 [SMILES: N#Cc1cc(F)cc(Oc2ccc(S(N)(=O)=O)c3c2CC(F)(F)[C@H]3O)c1] |
| | m/z: 383 (M − H) |
| 253 | 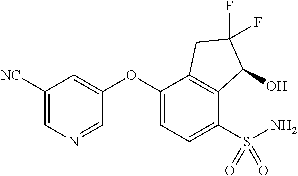 [SMILES: N#Cc1cncc(Oc2ccc(S(N)(=O)=O)c3c2CC(F)(F)[C@H]3O)c1] |
| | m/z: 368 (M + H) |
| 254 | 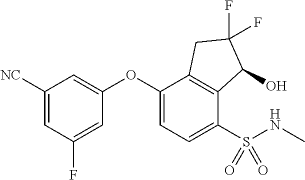 [SMILES: CNS(=O)(=O)c1ccc(Oc2cc(F)cc(C#N)c2)c2c1[C@H](O)C(F)(F)C2] |
| | m/z: 399 (M + H) |
| 255 | 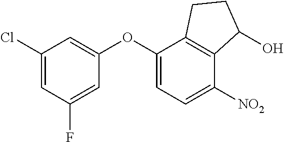 [SMILES: O=[N+]([O-])c1ccc(Oc2cc(F)cc(Cl)c2)c2c1C(O)CC2] |
| | m/z: 322 (M − H) |
| 256 | 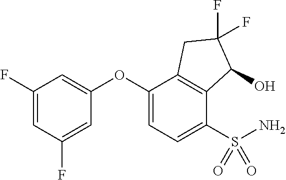 [SMILES: NS(=O)(=O)c1ccc(Oc2cc(F)cc(F)c2)c2c1[C@H](O)C(F)(F)C2] |
| | m/z: 378 (M + H) |
| 257 | 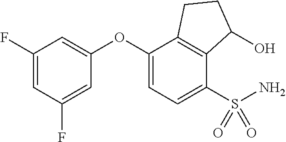 [SMILES: NS(=O)(=O)c1ccc(Oc2cc(F)cc(F)c2)c2c1C(O)CC2] |
| | m/z: 340 (M − H) |
| 258 | 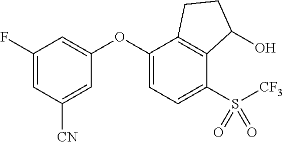 [SMILES: N#Cc1cc(F)cc(Oc2ccc(S(=O)(=O)C(F)(F)F)c3c2CCC3O)c1] |
| | m/z: (M − H) 399 |
| 259 | 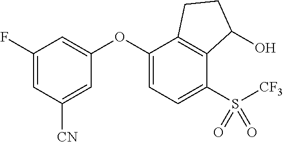 [SMILES: N#Cc1cc(F)cc(Oc2ccc(S(=O)(=O)C(F)(F)F)c3c2CCC3O)c1] |
| | m/z: (M − H) 399 |
| 260 | 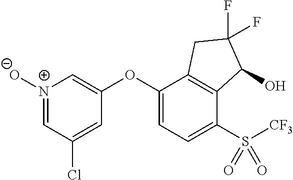 [SMILES: O=S(=O)(c1ccc(Oc2cc(Cl)c[n+]([O-])c2)c2c1[C@H](O)C(F)(F)C2)C(F)(F)F] |
| | m/z: (M + H) 446, 448 |
| 261 | 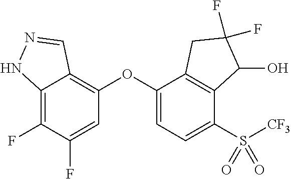 [SMILES: O=S(=O)(c1ccc(Oc2cc(F)c(F)c3[nH]ncc23)c2c1C(O)C(F)(F)C2)C(F)(F)F] |
| | m/z: (M + H) 471 |
| 262 | 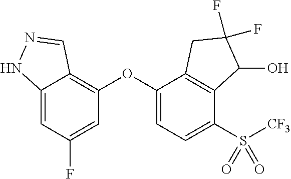 [SMILES: O=S(=O)(c1ccc(Oc2cc(F)cc3[nH]ncc23)c2c1C(O)C(F)(F)C2)C(F)(F)F] |
| | m/z: (M + H) 453 |
| 263 | 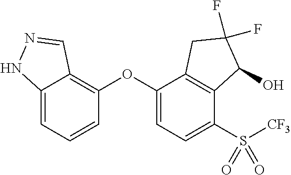 [SMILES: O=S(=O)(c1ccc(Oc2cccc3[nH]ncc23)c2c1[C@H](O)C(F)(F)C2)C(F)(F)F] |
| | m/z: (M + H) 435 |
| 264 | 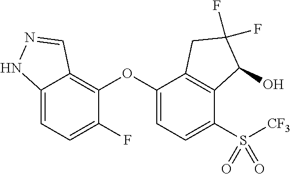 [SMILES: O=S(=O)(c1ccc(Oc2c(F)ccc3[nH]ncc23)c2c1[C@H](O)C(F)(F)C2)C(F)(F)F] |
| | m/z: (M + H) 453 |
| 265 | 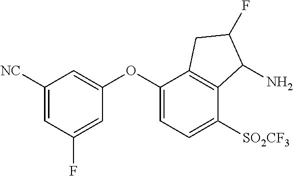 |
| | m/z: 419 (M + H) |
| 266 | 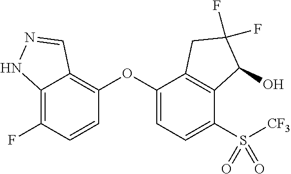 [SMILES: O=S(=O)(c1ccc(Oc2ccc(F)c3[nH]ncc23)c2c1[C@H](O)C(F)(F)C2)C(F)(F)F] |
| | m/z: (M + H) 453 |
| 267 | 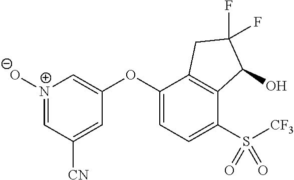 [SMILES: N#Cc1cc(Oc2ccc(S(=O)(=O)C(F)(F)F)c3c2CC(F)(F)[C@H]3O)c[n+]([O-])c1] |
| | m/z: (M − H) 435 |
| 268 | 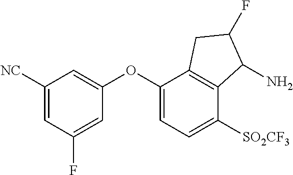 |
| | m/z: 419 (M + H) |
| 269 | 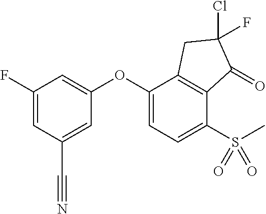 [SMILES: CS(=O)(=O)c1ccc(Oc2cc(F)cc(C#N)c2)c2c1C(=O)C(F)(Cl)C2] |
| | m/z: 398/400 (M + NH₄⁺) |
| 270 | 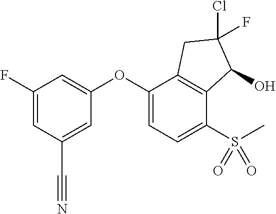 [SMILES: CS(=O)(=O)c1ccc(Oc2cc(F)cc(C#N)c2)c2c1[C@H](O)C(F)(Cl)C2] |
| | m/z: 417/419 (M + NH₄⁺) |
| 271 | 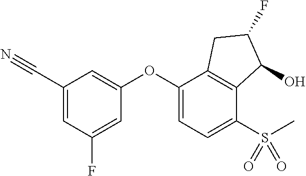 [SMILES: CS(=O)(=O)c1ccc(Oc2cc(F)cc(C#N)c2)c2c1[C@H](O)[C@@H](F)C2] |
| | m/z: 383 (M + NH₄⁺) |
| 272 | 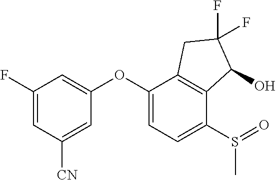 [SMILES: CS(=O)c1ccc(Oc2cc(F)cc(C#N)c2)c2c1[C@H](O)C(F)(F)C2] |
| | m/z: [M + H] 368 |
| 273 | 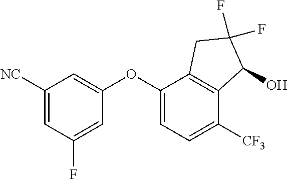 [SMILES: N#Cc1cc(F)cc(Oc2ccc(C(F)(F)F)c3c2CC(F)(F)[C@H]3O)c1] |
| | m/z: [M + formate − H] 418 |
| 274 | 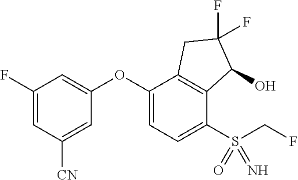 [SMILES: N#Cc1cc(F)cc(Oc2ccc(S(=N)(=O)CF)c3c2CC(F)(F)[C@H]3O)c1] |
| | m/z: (M + H) 401 |
| 275 | 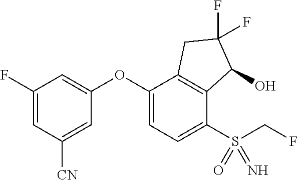 [SMILES: N#Cc1cc(F)cc(Oc2ccc(S(=N)(=O)CF)c3c2CC(F)(F)[C@H]3O)c1] |
| | m/z: (M + H) 401 |
| 276 | 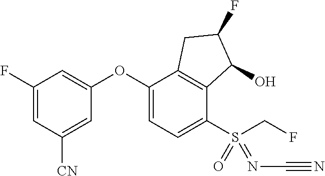 [SMILES: N#CN=S(=O)(CF)c1ccc(Oc2cc(F)cc(C#N)c2)c2c1[C@H](O)[C@H](F)C2] |
| | m/z: (M + H) 408 |
| 277 | 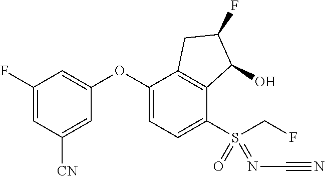 [SMILES: N#CN=S(=O)(CF)c1ccc(Oc2cc(F)cc(C#N)c2)c2c1[C@H](O)[C@H](F)C2] |
| | m/z: (M + H) 408 |
| 278 | 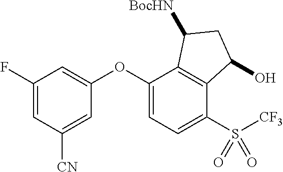 [SMILES: CC(C)(C)OC(=O)N[C@H]1C[C@@H](O)c2c(S(=O)(=O)C(F)(F)F)ccc(Oc3cc(F)cc(C#N)c3)c21] |
| | m/z: (M + Cl⁻) 551, 553 |
| 279 | 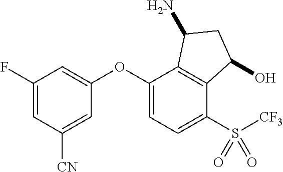 [SMILES: N#Cc1cc(F)cc(Oc2ccc(S(=O)(=O)C(F)(F)F)c3c2[C@@H](N)C[C@H]3O)c1] |
| | m/z: (M + H) 417 |
| 280 | 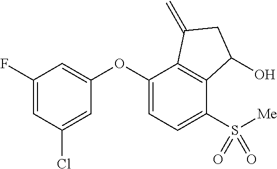 [SMILES: C=C1CC(O)c2c(S(C)(=O)=O)ccc(Oc3cc(F)cc(Cl)c3)c21] |
| | m/z: (M + H) 369, 371 |
| 281 | 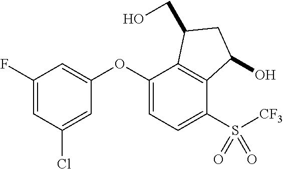 [SMILES: O=S(=O)(c1ccc(Oc2cc(F)cc(Cl)c2)c2c1[C@H](O)C[C@@H]2CO)C(F)(F)F] |
| | m/z: (M + H) 387, 389 |
| 282 | 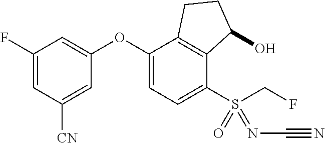 [SMILES: N#CN=S(=O)(CF)c1ccc(Oc2cc(F)cc(C#N)c2)c2c1[C@H](O)CC2] |
| | m/z: (M + H) 390 |
| 283 | 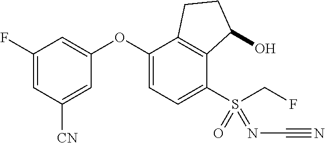 [SMILES: N#CN=S(=O)(CF)c1ccc(Oc2cc(F)cc(C#N)c2)c2c1[C@H](O)CC2] |
| | m/z: (M + H) 390 |
| 284 | 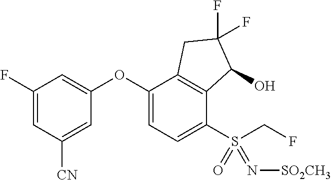 [SMILES: CS(=O)(=O)N=S(=O)(CF)c1ccc(Oc2cc(F)cc(C#N)c2)c2c1[C@H](O)C(F)(F)C2] |
| | m/z: (M + H) 479 |
| 285 | 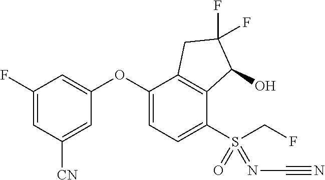 [SMILES: N#CN=S(=O)(CF)c1ccc(Oc2cc(F)cc(C#N)c2)c2c1[C@H](O)C(F)(F)C2] |
| | m/z: (M + H) 426 |
| 286 | 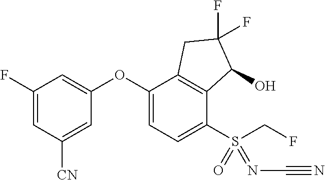 [SMILES: N#CN=S(=O)(CF)c1ccc(Oc2cc(F)cc(C#N)c2)c2c1[C@H](O)C(F)(F)C2] |
| | m/z: (M + H) 426 |
| 287 | 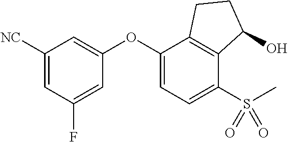 [SMILES: CS(=O)(=O)c1ccc(Oc2cc(F)cc(C#N)c2)c2c1[C@H](O)CC2] |
| | m/z: 392 (M + HCO₂⁻) |
| 288 | 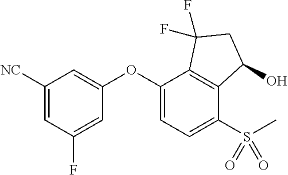 [SMILES: CS(=O)(=O)c1ccc(Oc2cc(F)cc(C#N)c2)c2c1[C@H](O)CC2(F)F] |
| | m/z: 428 (M + HCO₂⁻) |
| 289 | 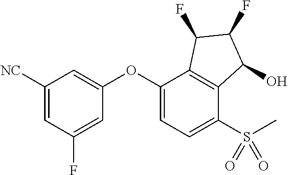 [SMILES: CS(=O)(=O)c1ccc(Oc2cc(F)cc(C#N)c2)c2c1[C@H](O)[C@H](F)[C@@H]2F] |
| | m/z: 384 (M + H) |
| 290 | 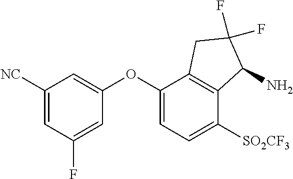 |
| | m/z: 437 (M + H) |
| 291 | 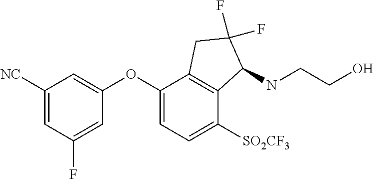 |
| | m/z: 481 (M + H) |
| 292 | 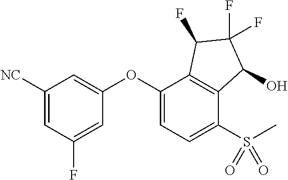 [SMILES: CS(=O)(=O)c1ccc(Oc2cc(F)cc(C#N)c2)c2c1[C@H](O)C(F)(F)[C@@H]2F] |
| | m/z: 419 (M + NH₄⁻) |
| 293 | 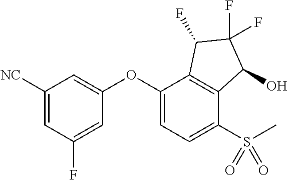 [SMILES: CS(=O)(=O)c1ccc(Oc2cc(F)cc(C#N)c2)c2c1[C@H](O)C(F)(F)[C@H]2F] |
| | m/z: 419 (M + NH₄⁻) |
| 294 | 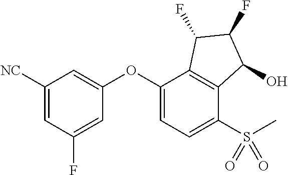 [SMILES: CS(=O)(=O)c1ccc(Oc2cc(F)cc(C#N)c2)c2c1[C@H](O)[C@H](F)[C@H]2F] |
| | m/z: 384 (M + H) |
| 295 | 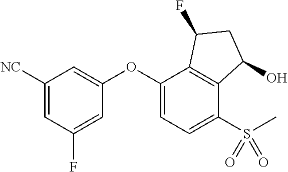 [SMILES: CS(=O)(=O)c1ccc(Oc2cc(F)cc(C#N)c2)c2c1[C@H](O)C[C@@H]2F] |
| | m/z: 383 (M + NH₄⁻) |
| 296 | 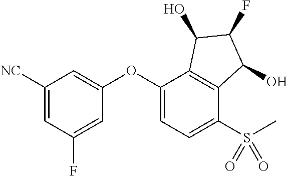 [SMILES: CS(=O)(=O)c1ccc(Oc2cc(F)cc(C#N)c2)c2c1[C@H](O)[C@H](F)[C@@H]2O] |
| | m/z: 399 (M + NH₄⁻) |
| 297 | 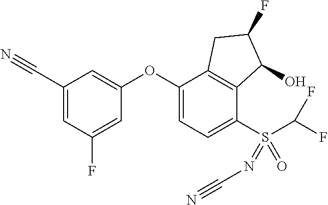 [SMILES: N#CN=S(=O)(c1ccc(Oc2cc(F)cc(C#N)c2)c2c1[C@H](O)[C@H](F)C2)C(F)F] |
| | m/z: 426 (M + H) |
| 298 | 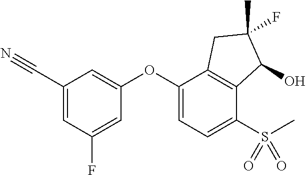 [SMILES: C[C@]1(F)Cc2c(Oc3cc(F)cc(C#N)c3)ccc(S(C)(=O)=O)c2[C@@H]1O] |
| | m/z: 397 (M + NH₄) |
| 299 | 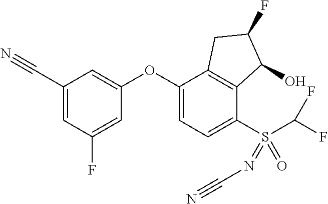 [SMILES: N#CN=S(=O)(c1ccc(Oc2cc(F)cc(C#N)c2)c2c1[C@H](O)[C@H](F)C2)C(F)F] |
| | m/z: 426 (M + H) |
| 300 | 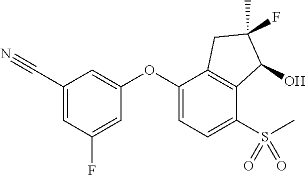 [SMILES: C[C@@]1(F)Cc2c(Oc3cc(F)cc(C#N)c3)ccc(S(C)(=O)=O)c2[C@@H]1O] |
| | m/z: 397 (M + NH₄) |
| 301 | 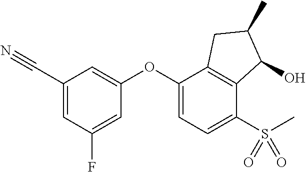 [SMILES: C[C@@H]1Cc2c(Oc3cc(F)cc(C#N)c3)ccc(S(C)(=O)=O)c2[C@@H]1O] |
| | m/z: 379 (M + NH₄) |
| 302 | 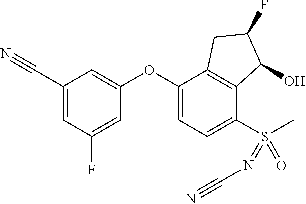 [SMILES: CS(=O)(=NC#N)c1ccc(Oc2cc(F)cc(C#N)c2)c2c1[C@H](O)[C@H](F)C2] |
| | m/z: 390 (M + H) |
| 303 | 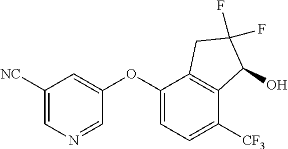 [SMILES: N#Cc1cncc(Oc2ccc(C(F)(F)F)c3c2CC(F)(F)[C@H]3O)c1] |
| | m/z: [M + H] = 357 |
| 304 | 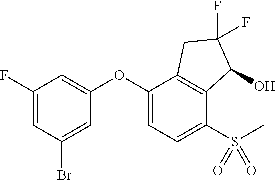 [SMILES: CS(=O)(=O)c1ccc(Oc2cc(F)cc(Br)c2)c2c1[C@H](O)C(F)(F)C2] |
| | m/z: 437, 439 (M + H⁺) |
| 305 | 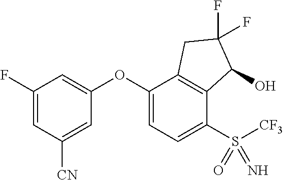 [SMILES: N#Cc1cc(F)cc(Oc2ccc(S(=N)(=O)C(F)(F)F)c3c2CC(F)(F)[C@H]3O)c1] |
| | m/z: (M + H) 437 |
| 306 | 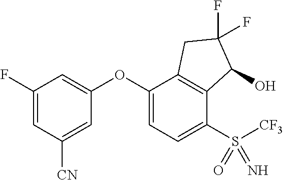 [SMILES: N#Cc1cc(F)cc(Oc2ccc(S(=N)(=O)C(F)(F)F)c3c2CC(F)(F)[C@H]3O)c1] |
| | m/z: (M + H) 437 |
| 307 | 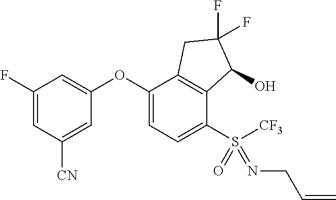 [SMILES: C=CCN=S(=O)(c1ccc(Oc2cc(F)cc(C#N)c2)c2c1[C@H](O)C(F)(F)C2)C(F)(F)F] |
| | m/z: (M + H) 477 |
| 308 | 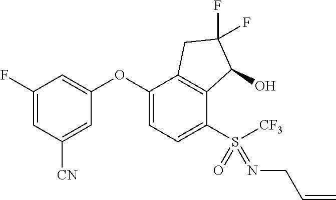 [SMILES: C=CCN=S(=O)(c1ccc(Oc2cc(F)cc(C#N)c2)c2c1[C@H](O)C(F)(F)C2)C(F)(F)F] |
| | m/z: (M + H) 477 |
| 309 | 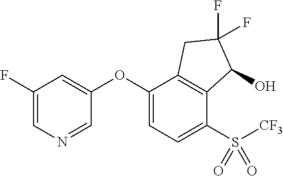 [SMILES: O=S(=O)(c1ccc(Oc2cncc(F)c2)c2c1[C@H](O)C(F)(F)C2)C(F)(F)F] |
| | m/z: (M + H) 414 |
| 310 | 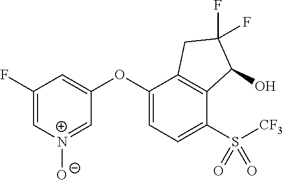 [SMILES: O=S(=O)(c1ccc(Oc2cc(F)c[n+]([O-])c2)c2c1[C@H](O)C(F)(F)C2)C(F)(F)F] |
| | m/z: (M + H) 430 |
| 311 | 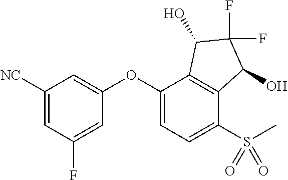 [SMILES: CS(=O)(=O)c1ccc(Oc2cc(F)cc(C#N)c2)c2c1[C@H](O)C(F)(F)[C@H]2O] |
| | m/z: 400 (M + H) |
| 312 | 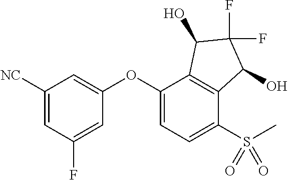 [SMILES: CS(=O)(=O)c1ccc(Oc2cc(F)cc(C#N)c2)c2c1[C@H](O)C(F)(F)[C@@H]2O] |
| | m/z: 400 (M + H) |
| 313 | 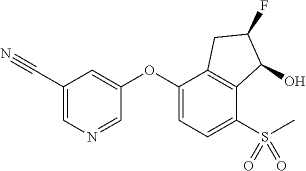 [SMILES: CS(=O)(=O)c1ccc(Oc2cncc(C#N)c2)c2c1[C@H](O)[C@H](F)C2] |
| | m/z: 349 (M + H) |
| 314 | 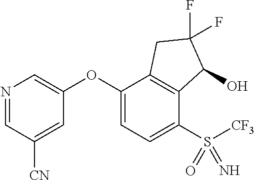 [SMILES: N#Cc1cncc(Oc2ccc(S(=N)(=O)C(F)(F)F)c3c2CC(F)(F)[C@H]3O)c1] |
| | m/z: (M + H) 420 |
| 315 | 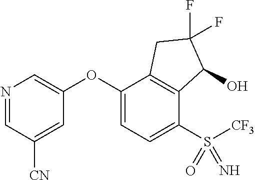 [SMILES: N#Cc1cncc(Oc2ccc(S(=N)(=O)C(F)(F)F)c3c2CC(F)(F)[C@H]3O)c1] |
| | m/z: (M + H) 420 |
| 316 | 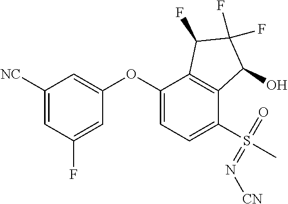 [SMILES: CS(=O)(=NC#N)c1ccc(Oc2cc(F)cc(C#N)c2)c2c1[C@H](O)C(F)(F)[C@@H]2F] |
| | m/z: [M + H] 426 |
| 317 | 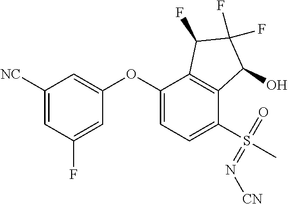 [SMILES: CS(=O)(=NC#N)c1ccc(Oc2cc(F)cc(C#N)c2)c2c1[C@H](O)C(F)(F)[C@@H]2F] |
| | m/z: [M + H] 426 |
| 318 | 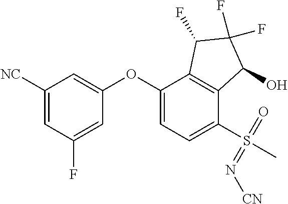 [SMILES: CS(=O)(=NC#N)c1ccc(Oc2cc(F)cc(C#N)c2)c2c1[C@H](O)C(F)(F)[C@H]2F] |
| | m/z: [M + H] 426 |
| 319 | 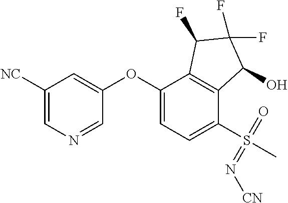 [SMILES: CS(=O)(=NC#N)c1ccc(Oc2cncc(C#N)c2)c2c1[C@H](O)C(F)(F)[C@@H]2F] |
| | m/z: [M + H] 409 |
| 320 | 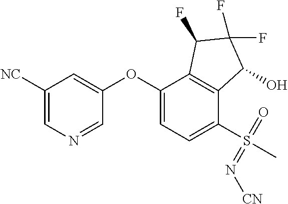 [SMILES: CS(=O)(=NC#N)c1ccc(Oc2cncc(C#N)c2)c2c1[C@@H](O)C(F)(F)[C@@H]2F] |
| | m/z: [M + H] 409 |
| 321 | 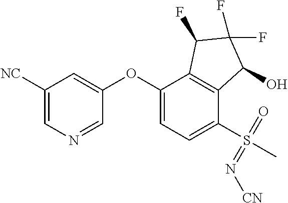 [SMILES: CS(=O)(=NC#N)c1ccc(Oc2cncc(C#N)c2)c2c1[C@H](O)C(F)(F)[C@@H]2F] |
| | m/z: [M + H] 409 |
| 322 | 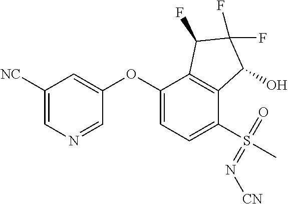 [SMILES: CS(=O)(=NC#N)c1ccc(Oc2cncc(C#N)c2)c2c1[C@@H](O)C(F)(F)[C@@H]2F] |
| | m/z: [M + H] 409 |
| 323 | 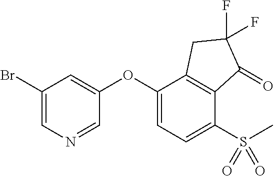 [SMILES: CS(=O)(=O)c1ccc(Oc2cncc(Br)c2)c2c1C(=O)C(F)(F)C2] |
| | m/z: 418, 420 (M + H) |
| 324 | 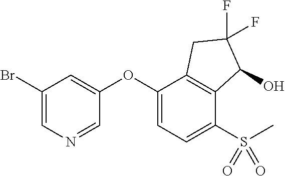 [SMILES: CS(=O)(=O)c1ccc(Oc2cncc(Br)c2)c2c1[C@H](O)C(F)(F)C2] |
| | m/z: 420, 422 (M + H) |
| 325 | 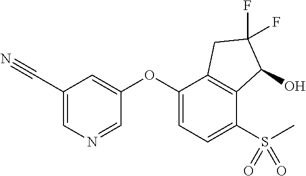 [SMILES: CS(=O)(=O)c1ccc(Oc2cncc(C#N)c2)c2c1[C@H](O)C(F)(F)C2] |
| | m/z: 367 (M + H) |
| 326 | 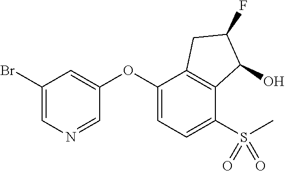 [SMILES: CS(=O)(=O)c1ccc(Oc2cncc(Br)c2)c2c1[C@H](O)[C@H](F)C2] |
| | m/z: 402, 404 (M + H) |
| 327 | 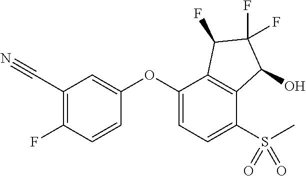 [SMILES: CS(=O)(=O)c1ccc(Oc2ccc(F)c(C#N)c2)c2c1[C@H](O)C(F)(F)[C@@H]2F] |
| | m/z: 402 (M + H) |
| 328 | 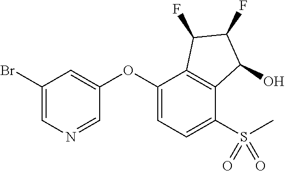 [SMILES: CS(=O)(=O)c1ccc(Oc2cncc(Br)c2)c2c1[C@H](O)[C@H](F)[C@@H]2F] |
| | m/z: 420, 422 (M + H) |
| 329 | 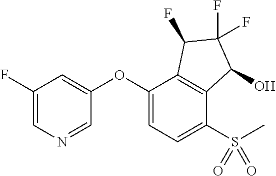 [SMILES: CS(=O)(=O)c1ccc(Oc2cncc(F)c2)c2c1[C@H](O)C(F)(F)[C@@H]2F] |
| | m/z: 378 (M + H) |
| 330 | 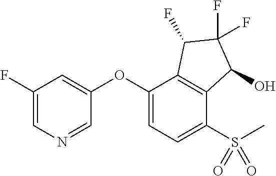 [SMILES: CS(=O)(=O)c1ccc(Oc2cncc(F)c2)c2c1[C@H](O)C(F)(F)[C@H]2F] |
| | m/z: 378 (M + H) |
| 331 | 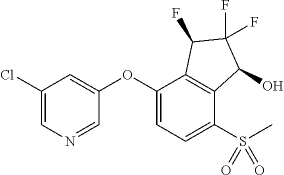 [SMILES: CS(=O)(=O)c1ccc(Oc2cncc(Cl)c2)c2c1[C@H](O)C(F)(F)[C@@H]2F] |
| | m/z: 394, 396 (M + H) |
| 332 | 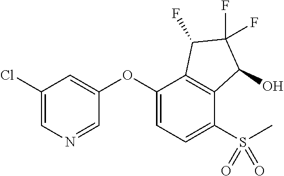 [SMILES: CS(=O)(=O)c1ccc(Oc2cncc(Cl)c2)c2c1[C@H](O)C(F)(F)[C@H]2F] |
| | m/z: 394, 396 (M + H) |
| 333 | 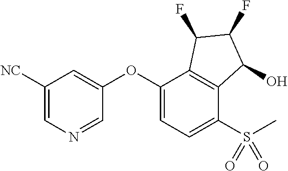 [SMILES: CS(=O)(=O)c1ccc(Oc2cncc(C#N)c2)c2c1[C@H](O)[C@H](F)[C@@H]2F] |
| | m/z: 367 (M + H) |
| 334 | 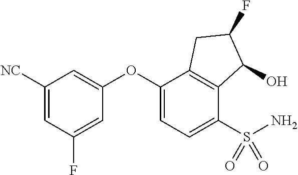 [SMILES: N#Cc1cc(F)cc(Oc2ccc(S(N)(=O)=O)c3c2C[C@@H](F)[C@H]3O)c1] |
| | m/z: 365 (M − H) |
| 335 | 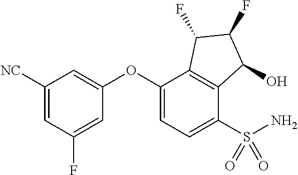 [SMILES: N#Cc1cc(F)cc(Oc2ccc(S(N)(=O)=O)c3c2[C@H](F)[C@@H](F)[C@H]3O)c1] |
| | m/z: 383 (M − H) |
| 336 | 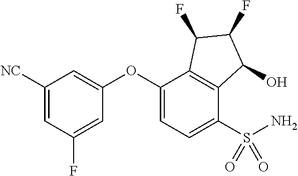 [SMILES: N#Cc1cc(F)cc(Oc2ccc(S(N)(=O)=O)c3c2[C@@H](F)[C@@H](F)[C@H]3O)c1] |
| | m/z: 383 (M − H) |
| 337 | 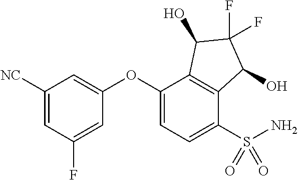 [SMILES: N#Cc1cc(F)cc(Oc2ccc(S(N)(=O)=O)c3c2[C@@H](O)C(F)(F)[C@H]3O)c1] |
| | m/z: 399 (M − H) |
| 338 | 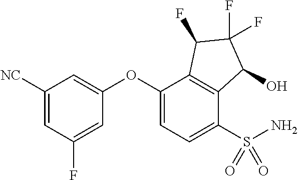 [SMILES: N#Cc1cc(F)cc(Oc2ccc(S(N)(=O)=O)c3c2[C@@H](F)C(F)(F)[C@H]3O)c1] |
| | m/z: 401 (M − H) |
| 339 | 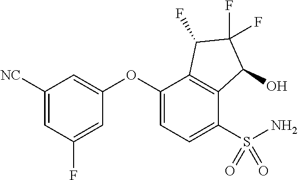 [SMILES: N#Cc1cc(F)cc(Oc2ccc(S(N)(=O)=O)c3c2[C@H](F)C(F)(F)[C@H]3O)c1] |
| | m/z: 401 (M − H) |
| 340 | 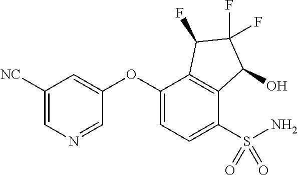 [SMILES: N#Cc1cncc(Oc2ccc(S(N)(=O)=O)c3c2[C@@H](F)C(F)(F)[C@H]3O)c1] |
| | m/z: 384 (M − H) |
| 341 | 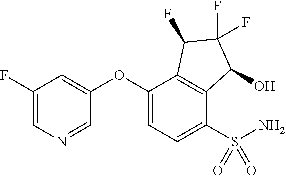 [SMILES: NS(=O)(=O)c1ccc(Oc2cncc(F)c2)c2c1[C@H](O)C(F)(F)[C@@H]2F] |
| | m/z: 377 (M − H) |
| 342 | 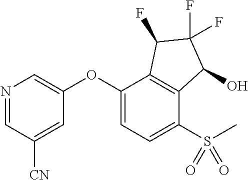 [SMILES: CS(=O)(=O)c1ccc(Oc2cncc(C#N)c2)c2c1[C@H](O)C(F)(F)[C@@H]2F] |
| | m/z: (M + H) 385 |
| 343 | 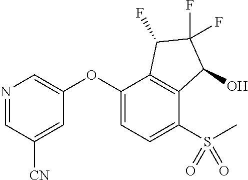 [SMILES: CS(=O)(=O)c1ccc(Oc2cncc(C#N)c2)c2c1[C@H](O)C(F)(F)[C@H]2F] |
| | m/z: (M + H) 385 |
| 344 | 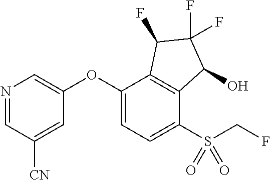 [SMILES: N#Cc1cncc(Oc2ccc(S(=O)(=O)CF)c3c2[C@@H](F)C(F)(F)[C@H]3O)c1] |
| | m/z: (M + H) 403 |
| 345 | 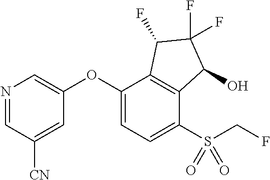 [SMILES: N#Cc1cncc(Oc2ccc(S(=O)(=O)CF)c3c2[C@H](F)C(F)(F)[C@H]3O)c1] |
| | m/z: (M + H) 403 |
| 346 | 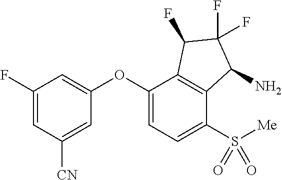 [SMILES: CS(=O)(=O)c1ccc(Oc2cc(F)cc(C#N)c2)c2c1[C@H](N)C(F)(F)[C@@H]2F] |
| | m/z: (M + H) 401 |
| 347 | 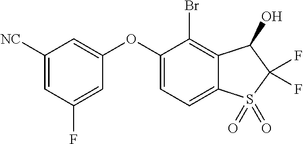 [SMILES: N#Cc1cc(F)cc(Oc2ccc3c(c2Br)[C@@H](O)C(F)(F)S3(=O)=O)c1] |
| 348 | 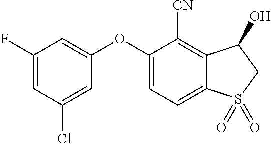 [SMILES: N#Cc1c(Oc2cc(F)cc(Cl)c2)ccc2c1[C@@H](O)CS2(=O)=O] |
| | m/z: 352, 354 (M − H) |
| 349 | 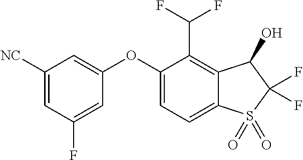 [SMILES: N#Cc1cc(F)cc(Oc2ccc3c(c2C(F)F)[C@@H](O)C(F)(F)S3(=O)=O)c1] |
| | m/z: 404 (M − H) |
| 350 | 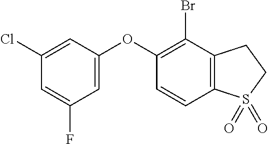 [SMILES: O=S1(=O)CCc2c1ccc(Oc1cc(F)cc(Cl)c1)c2Br] |
| | m/z: 391, 393, 395 (M + H) |
| 351 | 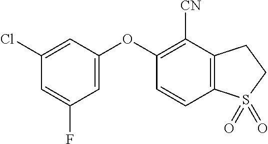 [SMILES: N#Cc1c(Oc2cc(F)cc(Cl)c2)ccc2c1CCS2(=O)=O] |
| | m/z: 336, 338 (M + H) |
| 352 | 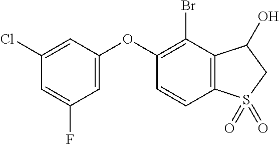 [SMILES: O=S1(=O)CC(O)c2c1ccc(Oc1cc(F)cc(Cl)c1)c2Br] |
| | m/z: 424, 426, 428 (M + NH₄) |
| 353 | 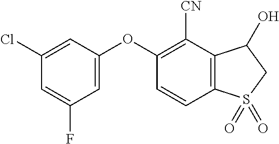 [SMILES: N#Cc1c(Oc2cc(F)cc(Cl)c2)ccc2c1C(O)CS2(=O)=O] |
| | m/z: 352, 354 (M − H) |
| 354 | 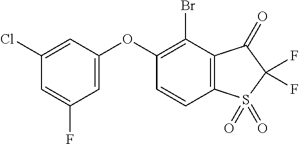 [SMILES: O=C1c2c(ccc(Oc3cc(F)cc(Cl)c3)c2Br)S(=O)(=O)C1(F)F] |
| 355 | 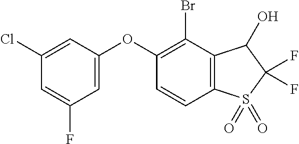 [SMILES: O=S1(=O)c2ccc(Oc3cc(F)cc(Cl)c3)c(Br)c2C(O)C1(F)F] |
| | m/z: 425, 427, 429 (M − OH) |
| 356 | 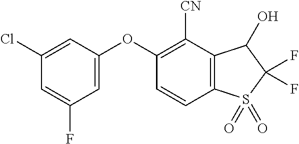 [SMILES: N#Cc1c(Oc2cc(F)cc(Cl)c2)ccc2c1C(O)C(F)(F)S2(=O)=O] |
| | m/z: 388, 390 (M − H) |
| 357 | 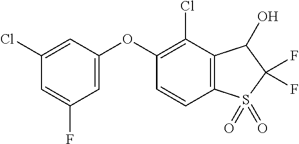 [SMILES: O=S1(=O)c2ccc(Oc3cc(F)cc(Cl)c3)c(Cl)c2C(O)C1(F)F] |
| | m/z: 397, 399 (M − H) |
| 358 | 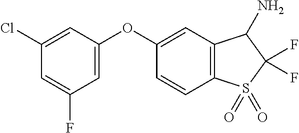 [SMILES: NC1c2cc(Oc3cc(F)cc(Cl)c3)ccc2S(=O)(=O)C1(F)F] |
| | m/z: 364, 366 (M + H) |
| 359 | 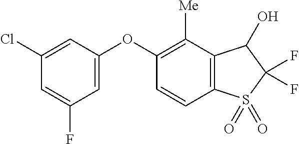 [SMILES: Cc1c(Oc2cc(F)cc(Cl)c2)ccc2c1C(O)C(F)(F)S2(=O)=O] |
| | m/z: 377, 379 (M − H) |
| 360 | 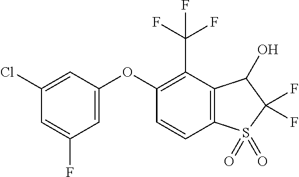 [SMILES: O=S1(=O)c2ccc(Oc3cc(F)cc(Cl)c3)c(C(F)(F)F)c2C(O)C1(F)F] |
| | m/z: 431, 433 (M − H) |
| 361 | 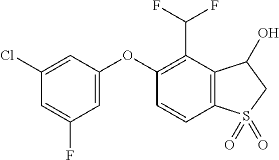 [SMILES: O=S1(=O)CC(O)c2c1ccc(Oc1cc(F)cc(Cl)c1)c2C(F)F] |
| | m/z: 377, 379 (M − H) |
| 362 | 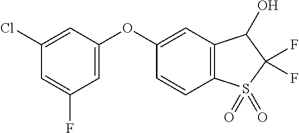 [SMILES: O=S1(=O)c2ccc(Oc3cc(F)cc(Cl)c3)cc2C(O)C1(F)F] |
| | m/z: 363, 365 (M − H) |
| 363 | 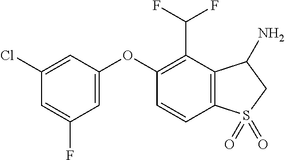 [SMILES: NC1CS(=O)(=O)c2ccc(Oc3cc(F)cc(Cl)c3)c(C(F)F)c21] |
| | m/z: 378, 380 (M + H) |
| 364 | 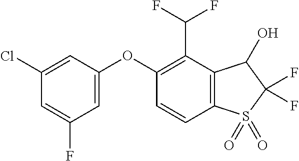 [SMILES: O=S1(=O)c2ccc(Oc3cc(F)cc(Cl)c3)c(C(F)F)c2C(O)C1(F)F] |
| | m/z: 413, 415 (M − H) |
| 365 | 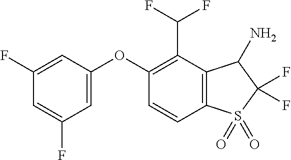 [SMILES: NC1c2c(ccc(Oc3cc(F)cc(F)c3)c2C(F)F)S(=O)(=O)C1(F)F] |
| | m/z: 398 (M + H) |
| 366 | 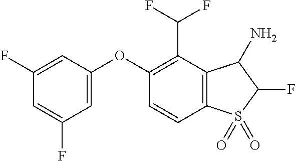 [SMILES: NC1c2c(ccc(Oc3cc(F)cc(F)c3)c2C(F)F)S(=O)(=O)C1F] |
| | m/z: 380 (M + H) |
| 367 | 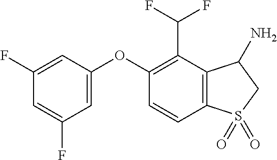 [SMILES: NC1CS(=O)(=O)c2ccc(Oc3cc(F)cc(F)c3)c(C(F)F)c21] |
| | m/z: 362 (M + H) |
| 368 | 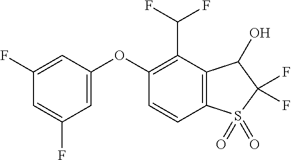 [SMILES: O=S1(=O)c2ccc(Oc3cc(F)cc(F)c3)c(C(F)F)c2C(O)C1(F)F] |
| | m/z: 397 (M − H) |
| 369 | 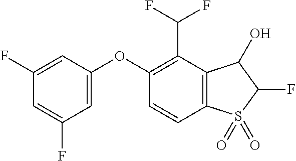 [SMILES: O=S1(=O)c2ccc(Oc3cc(F)cc(F)c3)c(C(F)F)c2C(O)C1F] |
| | m/z: 379 (M − H) |
| 370 | 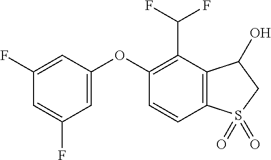 [SMILES: O=S1(=O)CC(O)c2c1ccc(Oc1cc(F)cc(F)c1)c2C(F)F] |
| | m/z: 363 (M + H) |
| 371 | 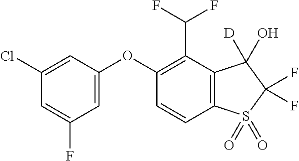 [SMILES: [2H]C1(O)c2c(ccc(Oc3cc(F)cc(Cl)c3)c2C(F)F)S(=O)(=O)C1(F)F] |
| | m/z: 414, 416 (M − H) |
| 372 | 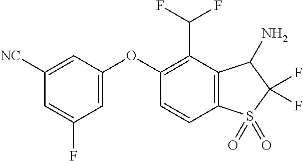 [SMILES: N#Cc1cc(F)cc(Oc2ccc3c(c2C(F)F)C(N)C(F)(F)S3(=O)=O)c1] |
| | m/z: 405 (M + H) |
| 373 | 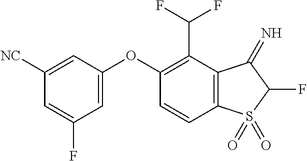 [SMILES: N#Cc1cc(F)cc(Oc2ccc3c(c2C(F)F)C(=N)C(F)S3(=O)=O)c1] |
| | m/z: 385 (M + H) |
| 374 | 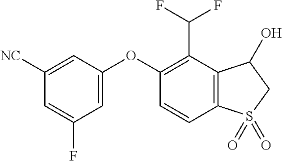 [SMILES: N#Cc1cc(F)cc(Oc2ccc3c(c2C(F)F)C(O)CS3(=O)=O)c1] |
| | m/z: 370 (M − H) |
| 375 | 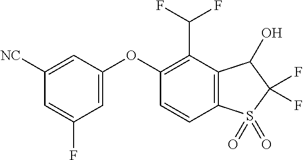 [SMILES: N#Cc1cc(F)cc(Oc2ccc3c(c2C(F)F)C(O)C(F)(F)S3(=O)=O)c1] |
| | m/z: 404 (M − H) |
| 376 | 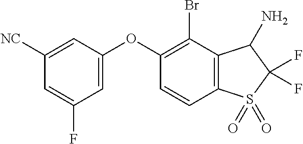 [SMILES: N#Cc1cc(F)cc(Oc2ccc3c(c2Br)C(N)C(F)(F)S3(=O)=O)c1] |
| | m/z: 426, 428 (M + H) |
| 377 | 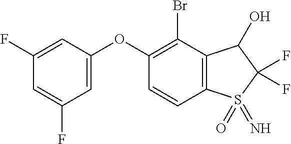 [SMILES: N=S1(=O)c2ccc(Oc3cc(F)cc(F)c3)c(Br)c2C(O)C1(F)F] |
| | m/z: 426, 428 (M + H) |
| 378 | 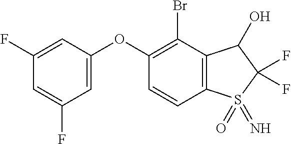 [SMILES: N=S1(=O)c2ccc(Oc3cc(F)cc(F)c3)c(Br)c2C(O)C1(F)F] |
| | m/z: 426, 428 (M + H) |
| 379 | 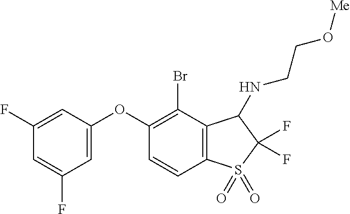 [SMILES: COCCNC1c2c(ccc(Oc3cc(F)cc(F)c3)c2Br)S(=O)(=O)C1(F)F] |
| | m/z: 484, 486 (M + H) |
| 380 | 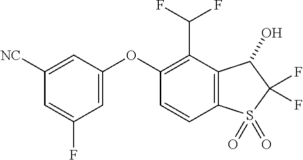 [SMILES: N#Cc1cc(F)cc(Oc2ccc3c(c2C(F)F)[C@H](O)C(F)(F)S3(=O)=O)c1] |
| | m/z: 404 (M − H) |
| 381 | 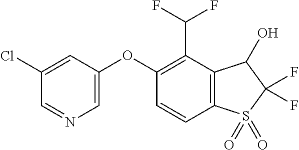 [SMILES: O=S1(=O)c2ccc(Oc3cncc(Cl)c3)c(C(F)F)c2C(O)C1(F)F] |
| | m/z: 398, 400 (M + H) |
| 382 | 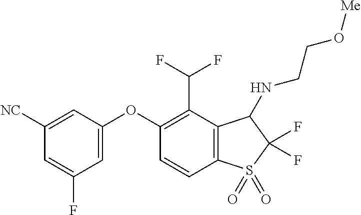 [SMILES: COCCNC1c2c(ccc(Oc3cc(F)cc(C#N)c3)c2C(F)F)S(=O)(=O)C1(F)F] |
| | m/z: 463 (M + H) |
| 383 | 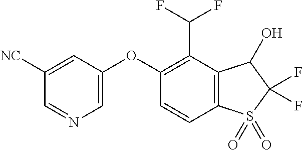 [SMILES: N#Cc1cncc(Oc2ccc3c(c2C(F)F)C(O)C(F)(F)S3(=O)=O)c1] |
| | m/z: 389 (M + H) |
| 384 | 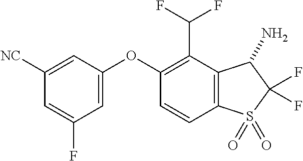 [SMILES: N#Cc1cc(F)cc(Oc2ccc3c(c2C(F)F)[C@H](N)C(F)(F)S3(=O)=O)c1] |
| | m/z: 405 (M + H) |
| 385 | 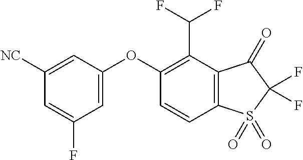 [SMILES: N#Cc1cc(F)cc(Oc2ccc3c(c2C(F)F)C(=O)C(F)(F)S3(=O)=O)c1] |
| 386 | 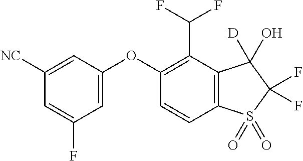 [SMILES: [2H]C1(O)c2c(ccc(Oc3cc(F)cc(C#N)c3)c2C(F)F)S(=O)(=O)C1(F)F] |
| | m/z: 405 (M − H) |
| 387 | 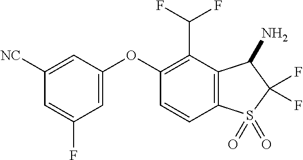 [SMILES: N#Cc1cc(F)cc(Oc2ccc3c(c2C(F)F)[C@@H](N)C(F)(F)S3(=O)=O)c1] |
| | m/z: 405 (M + H) |
| 388 | 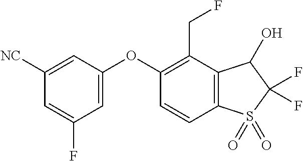 [SMILES: N#Cc1cc(F)cc(Oc2ccc3c(c2CF)C(O)C(F)(F)S3(=O)=O)c1] |
| | m/z: 405 (M + NH₄) |
| 389 | 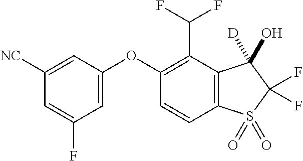 [SMILES: [2H][C@@]1(O)c2c(ccc(Oc3cc(F)cc(C#N)c3)c2C(F)F)S(=O)(=O)C1(F)F] |
| | m/z: 405 (M − H) |
| 390 | 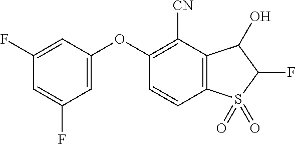 [SMILES: N#Cc1c(Oc2cc(F)cc(F)c2)ccc2c1C(O)C(F)S2(=O)=O] |
| | m/z: 373 (M + NH₄) |
| 391 | 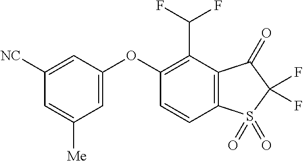 [SMILES: Cc1cc(C#N)cc(Oc2ccc3c(c2C(F)F)C(=O)C(F)(F)S3(=O)=O)c1] |
| 392 | 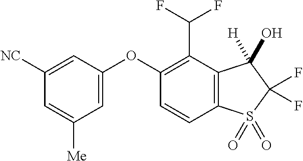 [SMILES: Cc1cc(C#N)cc(Oc2ccc3c(c2C(F)F)[C@@H](O)C(F)(F)S3(=O)=O)c1] |
| | m/z: 419 (M + NH₄) |
| 393 | 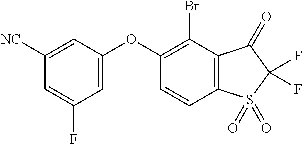 [SMILES: N#Cc1cc(F)cc(Oc2ccc3c(c2Br)C(=O)C(F)(F)S3(=O)=O)c1] |
| | m/z: 467, 469 (M + H2O + NH4) |
| 394 | 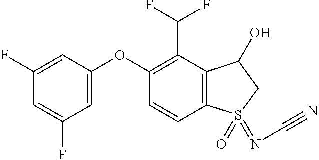 [SMILES: N#CN=S1(=O)CC(O)c2c1ccc(Oc1cc(F)cc(F)c1)c2C(F)F] |
| | m/z: 387 (M + H) |
| 395 | 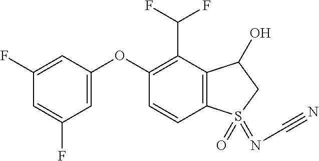 [SMILES: N#CN=S1(=O)CC(O)c2c1ccc(Oc1cc(F)cc(F)c1)c2C(F)F] |
| | m/z: 387 (M + H) |
| 396 | 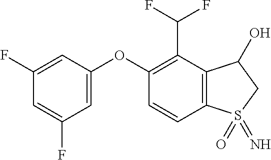 [SMILES: N=S1(=O)CC(O)c2c1ccc(Oc1cc(F)cc(F)c1)c2C(F)F] |
| | m/z: 362 (M + H) |
| 397 | 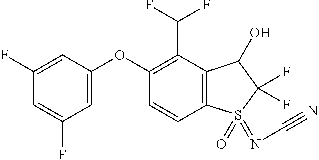 [SMILES: N#CN=S1(=O)c2ccc(Oc3cc(F)cc(F)c3)c(C(F)F)c2C(O)C1(F)F] |
| | m/z: 423 (M + H) |
| 398 | 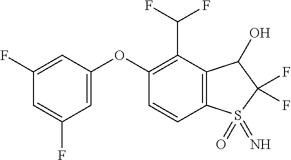 [SMILES: N=S1(=O)c2ccc(Oc3cc(F)cc(F)c3)c(C(F)F)c2C(O)C1(F)F] |
| | m/z: 398 (M + H) |
| 399 | 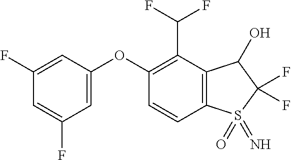 [SMILES: N=S1(=O)c2ccc(Oc3cc(F)cc(F)c3)c(C(F)F)c2C(O)C1(F)F] |
| | m/z: 398 (M + H) |
| 400 | 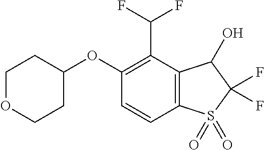 [SMILES: O=S1(=O)c2ccc(OC3CCOCC3)c(C(F)F)c2C(O)C1(F)F] |
| | m/z: 387 (M + H) |
| 401 | 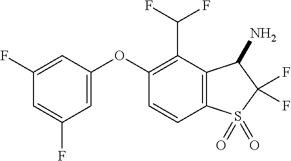 [SMILES: N[C@@H]1c2c(ccc(Oc3cc(F)cc(F)c3)c2C(F)F)S(=O)(=O)C1(F)F] |
| | m/z: 398 (M + H) |
| 402 | 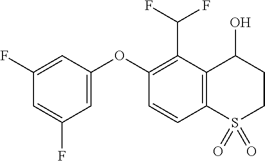 [SMILES: O=S1(=O)CCC(O)c2c1ccc(Oc1cc(F)cc(F)c1)c2C(F)F] |
| | m/z: 394 (M + NH₄) |
| 403 | 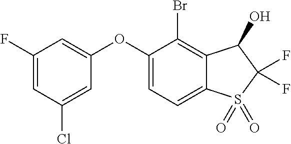 [SMILES: O=S1(=O)c2ccc(Oc3cc(F)cc(Cl)c3)c(Br)c2[C@@H](O)C1(F)F] |
| | m/z: 441, 443, 445 (M − H) |
| 404 | 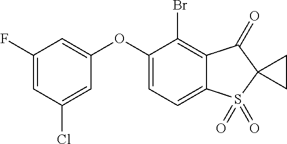 [SMILES: O=C1c2c(ccc(Oc3cc(F)cc(Cl)c3)c2Br)S(=O)(=O)C12CC2] |
| | m/z: 475, 477, 479 (M + HCO2-) |
| 405 | 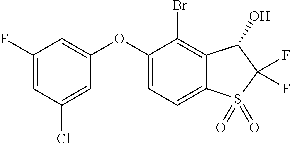 [SMILES: O=S1(=O)c2ccc(Oc3cc(F)cc(Cl)c3)c(Br)c2[C@H](O)C1(F)F] |
| | m/z: 441, 443, 445 (M − H) |
| 406 | 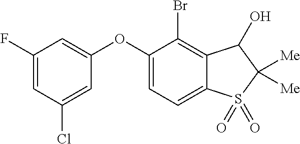 [SMILES: CC1(C)C(O)c2c(ccc(Oc3cc(F)cc(Cl)c3)c2Br)S1(=O)=O] |
| | m/z: 479, 481, 483 (M + HCO₂⁻) |
| 407 | 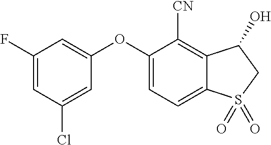 [SMILES: N#Cc1c(Oc2cc(F)cc(Cl)c2)ccc2c1[C@H](O)CS2(=O)=O] |
| | m/z: 352, 354 (M − H) |
| 408 | 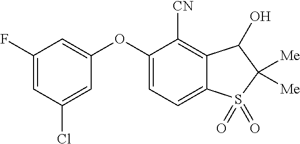 [SMILES: CC1(C)C(O)c2c(ccc(Oc3cc(F)cc(Cl)c3)c2C#N)S1(=O)=O] |
| | m/z: 380, 382 (M − H) |
| 409 | 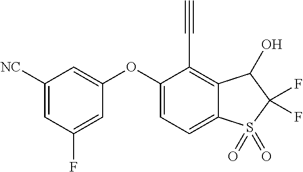 [SMILES: C#Cc1c(Oc2cc(F)cc(C#N)c2)ccc2c1C(O)C(F)(F)S2(=O)=O] |
| | m/z: 397 (M + NH₄) |
| 410 | 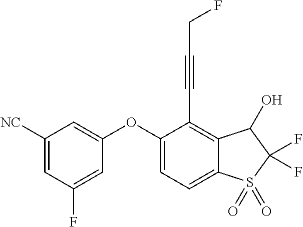 [SMILES: N#Cc1cc(F)cc(Oc2ccc3c(c2C#CCF)C(O)C(F)(F)S3(=O)=O)c1] |
| | m/z: 410 (M − H) |
| 411 | 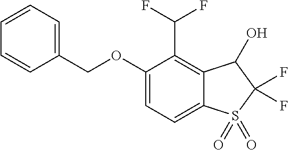 [SMILES: O=S1(=O)c2ccc(OCc3ccccc3)c(C(F)F)c2C(O)C1(F)F] |
| | m/z: 394 (M + NH₄) |
| 412 | 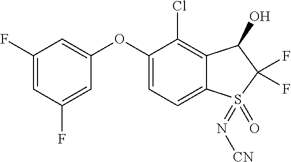 [SMILES: N#CN=S1(=O)c2ccc(Oc3cc(F)cc(F)c3)c(Cl)c2[C@@H](O)C1(F)F] |
| | m/z: 407, 409 (M + H) |
| 413 | 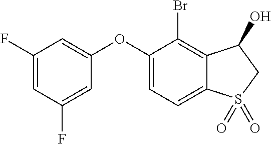 [SMILES: O=S1(=O)C[C@H](O)c2c1ccc(Oc1cc(F)cc(F)c1)c2Br] |
| 414 | 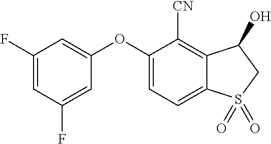 [SMILES: N#Cc1c(Oc2cc(F)cc(F)c2)ccc2c1[C@@H](O)CS2(=O)=O] |
| 415 | 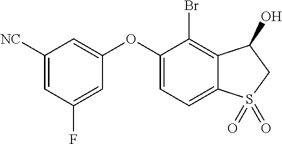 [SMILES: N#Cc1cc(F)cc(Oc2ccc3c(c2Br)[C@@H](O)CS3(=O)=O)c1] |
| 416 | 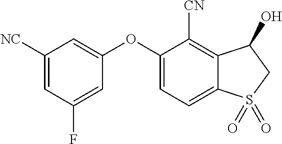 [SMILES: N#Cc1cc(F)cc(Oc2ccc3c(c2C#N)[C@@H](O)CS3(=O)=O)c1] |
| 417 | 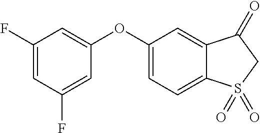 [SMILES: O=C1CS(=O)(=O)c2ccc(Oc3cc(F)cc(F)c3)cc21] |
| 418 | 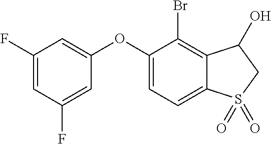 [SMILES: O=S1(=O)CC(O)c2c1ccc(Oc1cc(F)cc(F)c1)c2Br] |
| 419 | 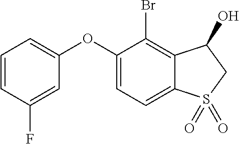 [SMILES: O=S1(=O)C[C@H](O)c2c1ccc(Oc1cccc(F)c1)c2Br] |
| 420 | 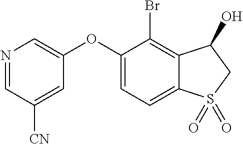 [SMILES: N#Cc1cncc(Oc2ccc3c(c2Br)[C@@H](O)CS3(=O)=O)c1] |
| 421 | 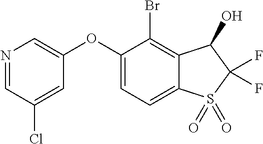 [SMILES: O=S1(=O)c2ccc(Oc3cncc(Cl)c3)c(Br)c2[C@@H](O)C1(F)F] |
| 422 | 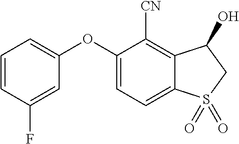 [SMILES: N#Cc1c(Oc2cccc(F)c2)ccc2c1[C@@H](O)CS2(=O)=O] |
| 423 | 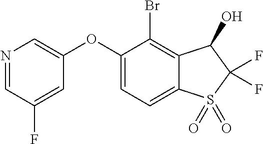 [SMILES: O=S1(=O)c2ccc(Oc3cncc(F)c3)c(Br)c2[C@@H](O)C1(F)F] |
| 424 | 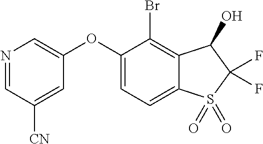 [SMILES: N#Cc1cncc(Oc2ccc3c(c2Br)[C@@H](O)C(F)(F)S3(=O)=O)c1] |
| 425 | 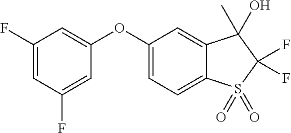 [SMILES: CC1(O)c2cc(Oc3cc(F)cc(F)c3)ccc2S(=O)(=O)C1(F)F] |
| | m/z: 407 (M + HCOOH − H) |
| 426 | 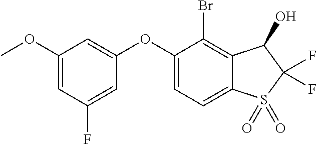 [SMILES: COc1cc(F)cc(Oc2ccc3c(c2Br)[C@@H](O)C(F)(F)S3(=O)=O)c1] |
| | m/z: 437, 439 (M − H) |
| 427 | 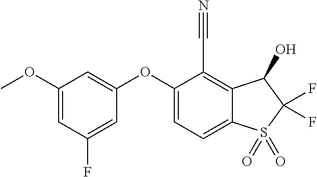 [SMILES: COc1cc(F)cc(Oc2ccc3c(c2C#N)[C@@H](O)C(F)(F)S3(=O)=O)c1] |
| | m/z: 384 (M − H) |
| 428 | 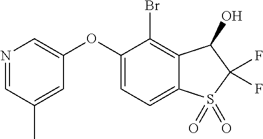 [SMILES: Cc1cncc(Oc2ccc3c(c2Br)[C@@H](O)C(F)(F)S3(=O)=O)c1] |
| 429 | 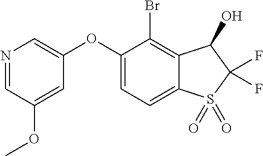 [SMILES: COc1cncc(Oc2ccc3c(c2Br)[C@@H](O)C(F)(F)S3(=O)=O)c1] |
| 430 | 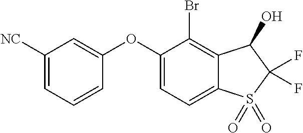 [SMILES: N#Cc1cccc(Oc2ccc3c(c2Br)[C@@H](O)C(F)(F)S3(=O)=O)c1] |
| 431 | 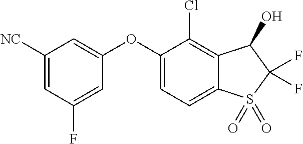 [SMILES: N#Cc1cc(F)cc(Oc2ccc3c(c2Cl)[C@@H](O)C(F)(F)S3(=O)=O)c1] |
| | m/z: 388, 390 (M − H) |
| 432 | 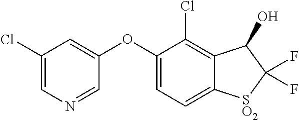 [SMILES: O=S1(=O)c2ccc(Oc3cncc(Cl)c3)c(Cl)c2[C@@H](O)C1(F)F] |
| 433 | 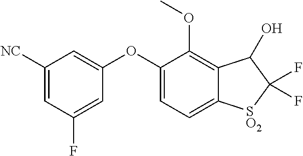 [SMILES: COc1c(Oc2cc(F)cc(C#N)c2)ccc2c1C(O)C(F)(F)S2(=O)=O] |
| | m/z: 430 (M + HCOOH − H) |
| 434 | 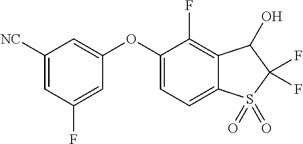 [SMILES: N#Cc1cc(F)cc(Oc2ccc3c(c2F)C(O)C(F)(F)S3(=O)=O)c1] |
| | m/z: 418 (M + HCOOH − H) |
| 435 | 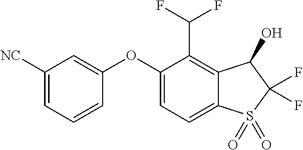 [SMILES: N#Cc1cccc(Oc2ccc3c(c2C(F)F)[C@@H](O)C(F)(F)S3(=O)=O)c1] |
| 436 | 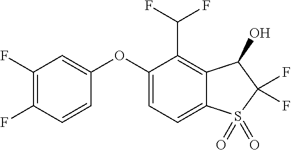 [SMILES: O=S1(=O)c2ccc(Oc3ccc(F)c(F)c3)c(C(F)F)c2[C@@H](O)C1(F)F] |
| 437 | 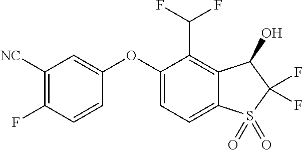 [SMILES: N#Cc1cc(Oc2ccc3c(c2C(F)F)[C@@H](O)C(F)(F)S3(=O)=O)ccc1F] |
| 438 | 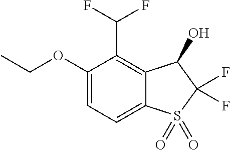 [SMILES: CCOc1ccc2c(c1C(F)F)[C@@H](O)C(F)(F)S2(=O)=O] |
| 439 | 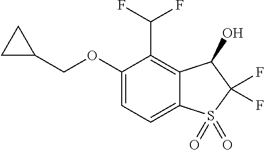 [SMILES: O=S1(=O)c2ccc(OCC3CC3)c(C(F)F)c2[C@@H](O)C1(F)F] |
| 440 | 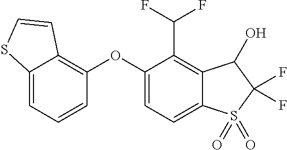 [SMILES: O=S1(=O)c2ccc(Oc3cccc4sccc34)c(C(F)F)c2C(O)C1(F)F] |
| 441 | 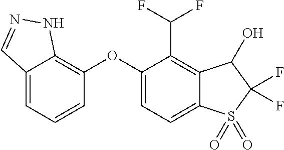 [SMILES: O=S1(=O)c2ccc(Oc3cccc4cn[nH]c34)c(C(F)F)c2C(O)C1(F)F] |
| 442 | 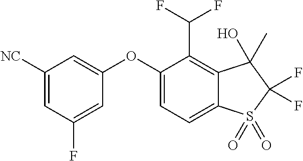 [SMILES: CC1(O)c2c(ccc(Oc3cc(F)cc(C#N)c3)c2C(F)F)S(=O)(=O)C1(F)F] |
| | m/z: 418 (M − H) |
| 443 | 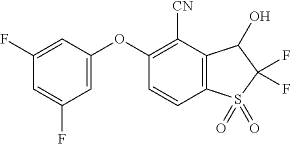 [SMILES: N#Cc1c(Oc2cc(F)cc(F)c2)ccc2c1C(O)C(F)(F)S2(=O)=O] |
| | m/z: 372 (M − H) |
| 444 | 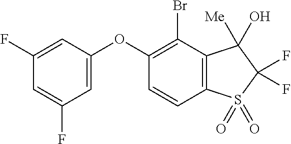 [SMILES: CC1(O)c2c(ccc(Oc3cc(F)cc(F)c3)c2Br)S(=O)(=O)C1(F)F] |
| | m/z: 439, 441 (M − H) |
| 445 | 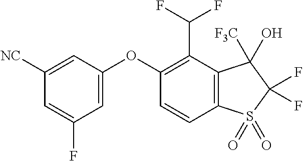 [SMILES: N#Cc1cc(F)cc(Oc2ccc3c(c2C(F)F)C(O)(C(F)(F)F)C(F)(F)S3(=O)=O)c1] |
| | m/z: 472 (M − H) |
| 446 | 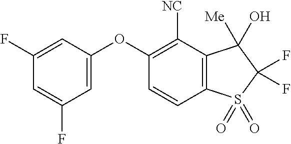 [SMILES: CC1(O)c2c(ccc(Oc3cc(F)cc(F)c3)c2C#N)S(=O)(=O)C1(F)F] |
| | m/z: 386 (M − H) |
| 447 | 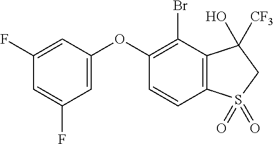 [SMILES: O=S1(=O)CC(O)(C(F)(F)F)c2c1ccc(Oc1cc(F)cc(F)c1)c2Br] |
| | m/z: 457, 459 (M − H) |
| 448 | 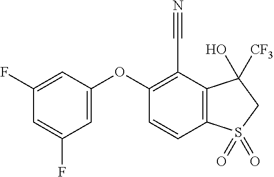 [SMILES: N#Cc1c(Oc2cc(F)cc(F)c2)ccc2c1C(O)(C(F)(F)F)CS2(=O)=O] |
| | m/z: 404 (M − H) |
| 449 | 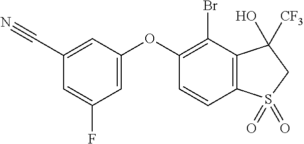 [SMILES: N#Cc1cc(F)cc(Oc2ccc3c(c2Br)C(O)(C(F)(F)F)CS3(=O)=O)c1] |
| | m/z: 464, 466 (M − H) |
| 450 | 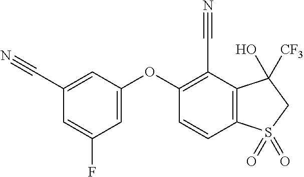 [SMILES: N#Cc1cc(F)cc(Oc2ccc3c(c2C#N)C(O)(C(F)(F)F)CS3(=O)=O)c1] |
| | m/z: 411 (M − H) |
| 451 | 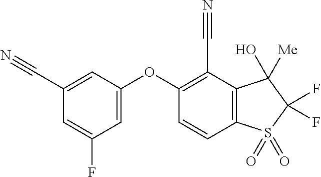 [SMILES: CC1(O)c2c(ccc(Oc3cc(F)cc(C#N)c3)c2C#N)S(=O)(=O)C1(F)F] |
| | m/z: 393 (M − H) |
| 452 | 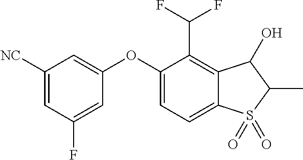 [SMILES: CC1C(O)c2c(ccc(Oc3cc(F)cc(C#N)c3)c2C(F)F)S1(=O)=O] |
| | m/z: 401 (M + NH₄⁺) |
| 453 | 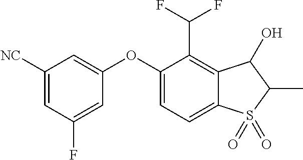 [SMILES: CC1C(O)c2c(ccc(Oc3cc(F)cc(C#N)c3)c2C(F)F)S1(=O)=O] |
| | m/z: 401 (M + NH₄⁺) |
| 454 | 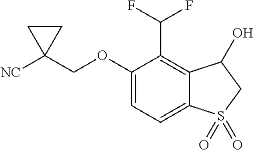 [SMILES: N#CC1(COc2ccc3c(c2C(F)F)C(O)CS3(=O)=O)CC1] |
| | m/z: 347 (M + NH₄⁺) |
| 455 | 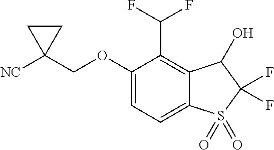 [SMILES: N#CC1(COc2ccc3c(c2C(F)F)C(O)C(F)(F)S3(=O)=O)CC1] |
| | m/z: 410 (M + HCO₂⁻) |
| 456 | 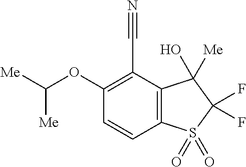 [SMILES: CC(C)Oc1ccc2c(c1C#N)C(C)(O)C(F)(F)S2(=O)=O] |
| | m/z: 316 (M − H) |
| 457 | 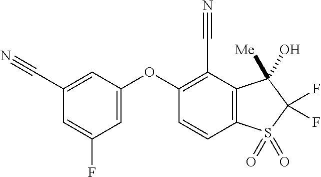 [SMILES: C[C@]1(O)c2c(ccc(Oc3cc(F)cc(C#N)c3)c2C#N)S(=O)(=O)C1(F)F] |
| | m/z: 393 (M − H) |
| 458 | 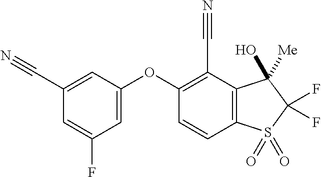 [SMILES: C[C@@]1(O)c2c(ccc(Oc3cc(F)cc(C#N)c3)c2C#N)S(=O)(=O)C1(F)F] |
| | m/z: 393 (M − H) |
| 459 | 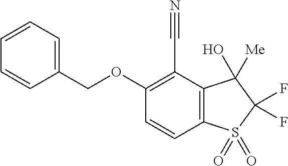 [SMILES: CC1(O)c2c(ccc(OCc3ccccc3)c2C#N)S(=O)(=O)C1(F)F] |
| | m/z: 410 (M + HCO2-) |
| 460 | 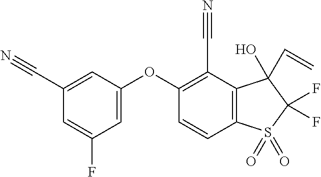 [SMILES: C=CC1(O)c2c(ccc(Oc3cc(F)cc(C#N)c3)c2C#N)S(=O)(=O)C1(F)F] |
| | m/z: 405 (M − H) |
| 461 | 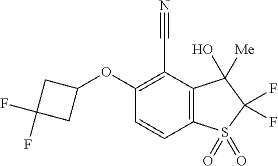 [SMILES: CC1(O)c2c(ccc(OC3CC(F)(F)C3)c2C#N)S(=O)(=O)C1(F)F] |
| | m/z: 364 (M − H) |
| 462 | 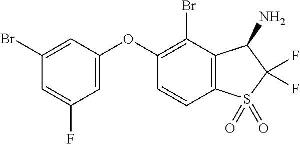 [SMILES: N[C@@H]1c2c(ccc(Oc3cc(F)cc(Br)c3)c2Br)S(=O)(=O)C1(F)F] |
| | m/z: 486, 488, 490 (M + H) |
| 463 | 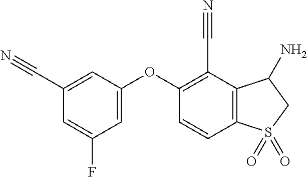 [SMILES: N#Cc1cc(F)cc(Oc2ccc3c(c2C#N)C(N)CS3(=O)=O)c1] |
| | m/z: 342 (M − H) |
| 464 | 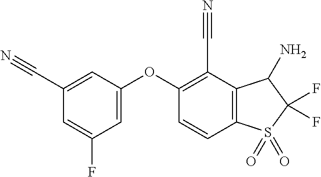 [SMILES: N#Cc1cc(F)cc(Oc2ccc3c(c2C#N)C(N)C(F)(F)S3(=O)=O)c1] |
| | m/z: 380 (M + H) |
| 465 | 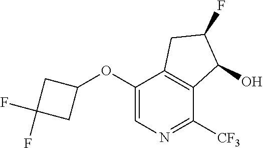 [SMILES: O[C@H]1c2c(C(F)(F)F)ncc(OC3CC(F)(F)C3)c2C[C@H]1F] |
| | m/z: (M + H) 328 |
| 466 | 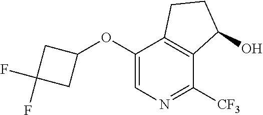 [SMILES: O[C@@H]1CCc2c(OC3CC(F)(F)C3)cnc(C(F)(F)F)c21] |
| | m/z: (M + H) 310 |
| 467 | 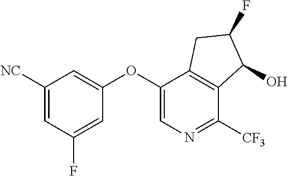 [SMILES: N#Cc1cc(F)cc(Oc2cnc(C(F)(F)F)c3c2C[C@@H](F)[C@H]3O)c1] |
| | m/z: (M + H) 357 |
| 468 | 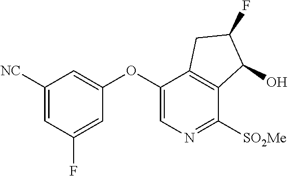 [SMILES: CS(=O)(=O)c1ncc(Oc2cc(F)cc(C#N)c2)c2c1[C@H](O)[C@H](F)C2] |
| | m/z: (M + H) 367 |
| 469 | 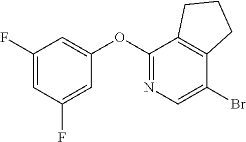 [SMILES: Fc1cc(F)cc(Oc2ncc(Br)c3c2CCC3)c1] |
| | m/z: 326/328 (M + H) |
| 470 | 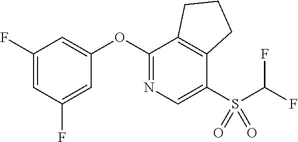 [SMILES: O=S(=O)(c1cnc(Oc2cc(F)cc(F)c2)c2c1CCC2)C(F)F] |
| | m/z: 362 (M + H) |
| 471 | 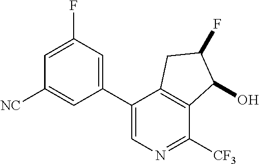 [SMILES: N#Cc1cc(F)cc(-c2cnc(C(F)(F)F)c3c2C[C@@H](F)[C@H]3O)c1] |
| | m/z: (M + H) 341 |
| 472 | 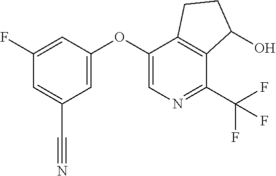 [SMILES: N#Cc1cc(F)cc(Oc2cnc(C(F)(F)F)c3c2CCC3O)c1] |
| | m/z: 339 (M + H) |
| 473 | 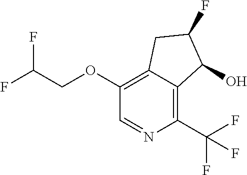 [SMILES: O[C@H]1c2c(C(F)(F)F)ncc(OCC(F)F)c2C[C@H]1F] |
| | m/z: 302 (M + H) |
| 474 | 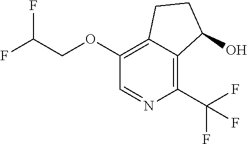 [SMILES: O[C@@H]1CCc2c(OCC(F)F)cnc(C(F)(F)F)c21] |
| | m/z: 284 (M + H) |
| 475 | 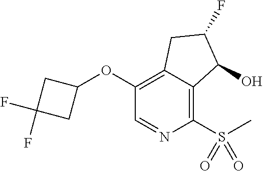 [SMILES: CS(=O)(=O)c1ncc(OC2CC(F)(F)C2)c2c1[C@H](O)[C@@H](F)C2] |
| | m/z: 338 (M + H) |
| 476 | 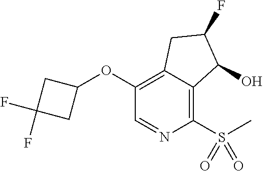 [SMILES: CS(=O)(=O)c1ncc(OC2CC(F)(F)C2)c2c1[C@H](O)[C@H](F)C2] |
| | m/z: 338 (M + H) |
| 477 | 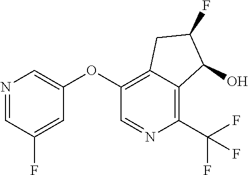 [SMILES: O[C@H]1c2c(C(F)(F)F)ncc(Oc3cncc(F)c3)c2C[C@H]1F] |
| | m/z: 333 (M + H) |
| 478 | 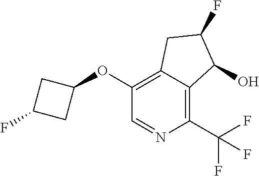 [SMILES: O[C@H]1c2c(C(F)(F)F)ncc(O[C@H]3C[C@H](F)C3)c2C[C@H]1F] |
| | m/z: 310 (M + H) |
| 479 | 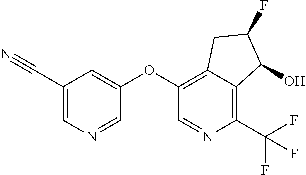 [SMILES: N#Cc1cncc(Oc2cnc(C(F)(F)F)c3c2C[C@@H](F)[C@H]3O)c1] |
| | m/z: 340 (M + H) |
| 480 | 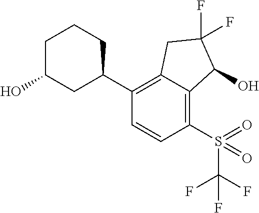 [SMILES: O=S(=O)(c1ccc([C@@H]2CCC[C@@H](O)C2)c2c1[C@H](O)C(F)(F)C2)C(F)(F)F] |
| | m/z: (M + HCO₂⁻) 445 |
| 481 | 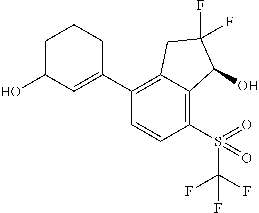 [SMILES: O=S(=O)(c1ccc(C2=CC(O)CCC2)c2c1[C@H](O)C(F)(F)C2)C(F)(F)F] |
| | m/z: (M − OH) 381 |
| 482 | 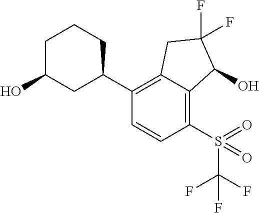 [SMILES: O=S(=O)(c1ccc([C@@H]2CCC[C@H](O)C2)c2c1[C@H](O)C(F)(F)C2)C(F)(F)F] |
| | m/z: (M + HCO₂⁻) 445 |
| 483 | 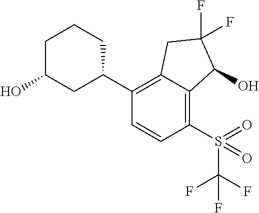 [SMILES: O=S(=O)(c1ccc([C@H]2CCC[C@@H](O)C2)c2c1[C@H](O)C(F)(F)C2)C(F)(F)F] |
| | m/z: (M + HCO₂⁻) 445 |
| 484 | 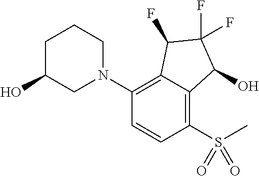 [SMILES: CS(=O)(=O)c1ccc(N2CCC[C@H](O)C2)c2c1[C@H](O)C(F)(F)[C@@H]2F] |
| | m/z: 366 (M + H) |
| 485 | 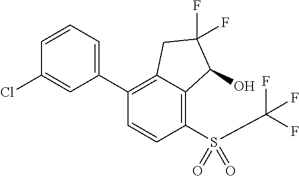 [SMILES: O=S(=O)(c1ccc(-c2cccc(Cl)c2)c2c1[C@H](O)C(F)(F)C2)C(F)(F)F] |
| | m/z: (M − H) 411/413 |
| 486 | 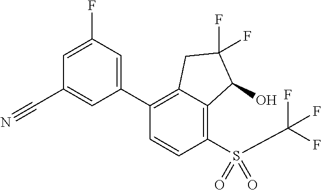 [SMILES: N#Cc1cc(F)cc(-c2ccc(S(=O)(=O)C(F)(F)F)c3c2CC(F)(F)[C@H]3O)c1] |
| | m/z: (M − H) 420 |
| 487 | 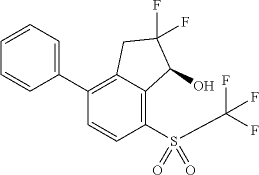 [SMILES: O=S(=O)(c1ccc(-c2ccccc2)c2c1[C@H](O)C(F)(F)C2)C(F)(F)F] |
| | m/z: (M − H) 377 |
| 488 | 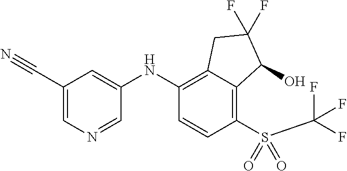 [SMILES: N#Cc1cncc(Nc2ccc(S(=O)(=O)C(F)(F)F)c3c2CC(F)(F)[C@H]3O)c1] |
| | m/z: (M + H) 420 |
| 489 | 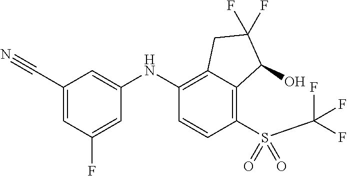 [SMILES: N#Cc1cc(F)cc(Nc2ccc(S(=O)(=O)C(F)(F)F)c3c2CC(F)(F)[C@H]3O)c1] |
| | m/z: (M − H) 435 |
| 490 | 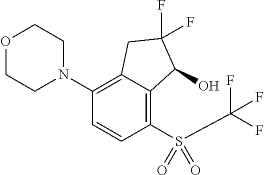 [SMILES: O=S(=O)(c1ccc(N2CCOCC2)c2c1[C@H](O)C(F)(F)C2)C(F)(F)F] |
| | m/z: (M + H) 388 |
| 491 | 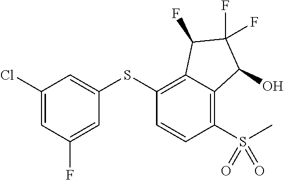 [SMILES: CS(=O)(=O)c1ccc(Sc2cc(F)cc(Cl)c2)c2c1[C@H](O)C(F)(F)[C@@H]2F] |
| | m/z: (M + Na) 449 |
| 492 | 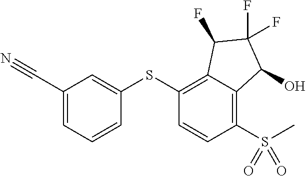 [SMILES: CS(=O)(=O)c1ccc(Sc2cccc(C#N)c2)c2c1[C@H](O)C(F)(F)[C@@H]2F] |
| | m/z: (M + Na) 422 |
| 493 | 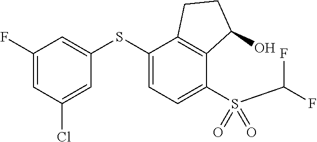 [SMILES: O=S(=O)(c1ccc(Sc2cc(F)cc(Cl)c2)c2c1[C@H](O)CC2)C(F)F] |
| | m/z: 453/455 (M + HCO₂⁻) |
| 494 | 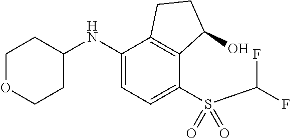 [SMILES: O=S(=O)(c1ccc(NC2CCOCC2)c2c1[C@H](O)CC2)C(F)F] |
| | m/z: 346 (M − H) |
| 495 | 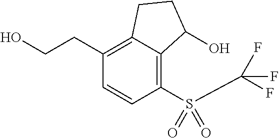 [SMILES: O=S(=O)(c1ccc(CCO)c2c1C(O)CC2)C(F)(F)F] |
| | m/z: 309 (M − H) |
| 496 | 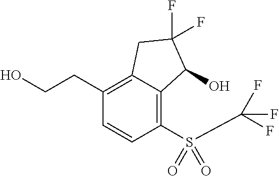 [SMILES: O=S(=O)(c1ccc(CCO)c2c1[C@H](O)C(F)(F)C2)C(F)(F)F] |
| | m/z: 345 (M − H) |
| 497 | 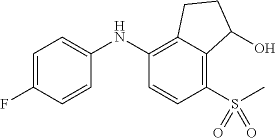 [SMILES: CS(=O)(=O)c1ccc(Nc2ccc(F)cc2)c2c1C(O)CC2] |
| | m/z: 320 (M − H) |
| 498 | 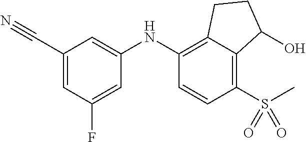 [SMILES: CS(=O)(=O)c1ccc(Nc2cc(F)cc(C#N)c2)c2c1C(O)CC2] |
| | m/z: 345 (M − H) |
| 499 | 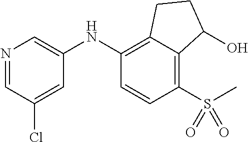 [SMILES: CS(=O)(=O)c1ccc(Nc2cncc(Cl)c2)c2c1C(O)CC2] |
| | m/z: 337 (M − H) |
| 500 | 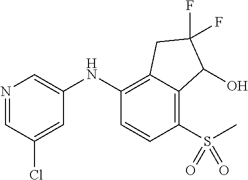 [SMILES: CS(=O)(=O)c1ccc(Nc2cncc(Cl)c2)c2c1C(O)C(F)(F)C2] |
| | m/z: 373 (M − H) |
| 501 | 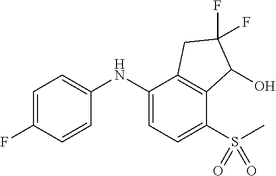 [SMILES: CS(=O)(=O)c1ccc(Nc2ccc(F)cc2)c2c1C(O)C(F)(F)C2] |
| | m/z: 356 (M − H) |
| 502 | 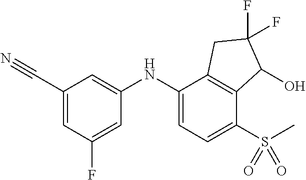 [SMILES: CS(=O)(=O)c1ccc(Nc2cc(F)cc(C#N)c2)c2c1C(O)C(F)(F)C2] |
| | m/z: 381 (M − H) |
| 503 | 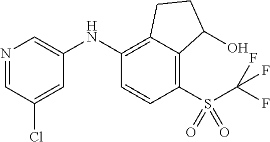 [SMILES: O=S(=O)(c1ccc(Nc2cncc(Cl)c2)c2c1C(O)CC2)C(F)(F)F] |
| | m/z: 391 (M − H) |
| 504 | 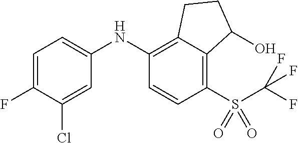 [SMILES: O=S(=O)(c1ccc(Nc2ccc(F)c(Cl)c2)c2c1C(O)CC2)C(F)(F)F] |
| | m/z: 408 (M − H) |
| 505 | 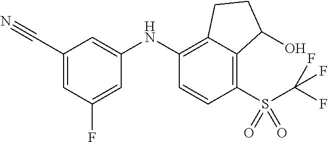 [SMILES: N#Cc1cc(F)cc(Nc2ccc(S(=O)(=O)C(F)(F)F)c3c2CCC3O)c1] |
| | m/z: 399 (M − H) |
| 506 | 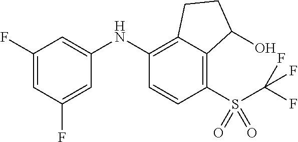 [SMILES: O=S(=O)(c1ccc(Nc2cc(F)cc(F)c2)c2c1C(O)CC2)C(F)(F)F] |
| | m/z: 392 (M − H) |
| 507 | 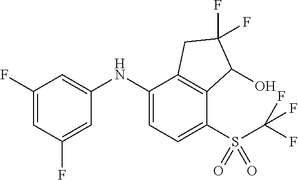 [SMILES: O=S(=O)(c1ccc(Nc2cc(F)cc(F)c2)c2c1C(O)C(F)(F)C2)C(F)(F)F] |
| 508 | 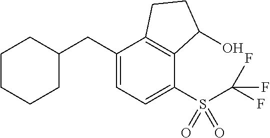 [SMILES: O=S(=O)(c1ccc(CC2CCCCC2)c2c1C(O)CC2)C(F)(F)F] |
| | m/z: 361 (M − H) |
| 509 | 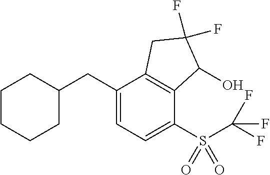 [SMILES: O=S(=O)(c1ccc(CC2CCCCC2)c2c1C(O)C(F)(F)C2)C(F)(F)F] |
| | m/z: 397 (M − H) |
| 510 | 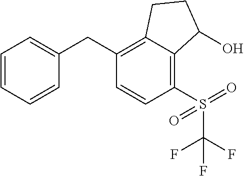 [SMILES: O=S(=O)(c1ccc(Cc2ccccc2)c2c1C(O)CC2)C(F)(F)F] |
| | m/z: 355 (M − H) |
| 511 | 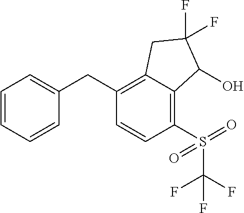 [SMILES: O=S(=O)(c1ccc(Cc2ccccc2)c2c1C(O)C(F)(F)C2)C(F)(F)F] |
| | m/z: 391 (M − H) |
| 512 | 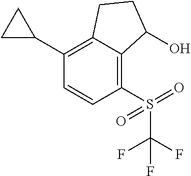 [SMILES: O=S(=O)(c1ccc(C2CC2)c2c1C(O)CC2)C(F)(F)F] |
| 513 | 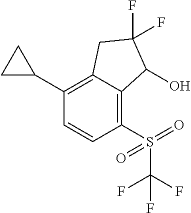 [SMILES: O=S(=O)(c1ccc(C2CC2)c2c1C(O)C(F)(F)C2)C(F)(F)F] |
| | m/z: 341 (M − H) |
| 514 | 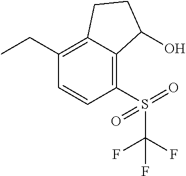 [SMILES: CCc1ccc(S(=O)(=O)C(F)(F)F)c2c1CCC2O] |
| 515 | 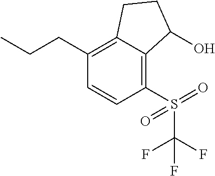 [SMILES: CCCc1ccc(S(=O)(=O)C(F)(F)F)c2c1CCC2O] |
| | m/z: 307 (M − H) |
| 516 | 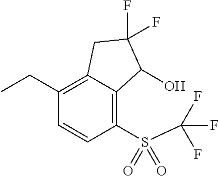 [SMILES: CCc1ccc(S(=O)(=O)C(F)(F)F)c2c1CC(F)(F)C2O] |
| | m/z: 329 (M − H) |
| 517 | 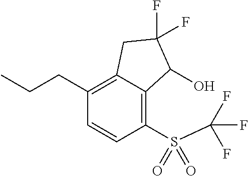 [SMILES: CCCc1ccc(S(=O)(=O)C(F)(F)F)c2c1CC(F)(F)C2O] |
| | m/z: 343 (M − H) |
| 518 | 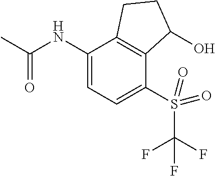 [SMILES: CC(=O)Nc1ccc(S(=O)(=O)C(F)(F)F)c2c1CCC2O] |
| | m/z: 322 (M − H) |
| 519 | 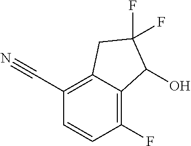 [SMILES: N#Cc1ccc(F)c2c1CC(F)(F)C2O] |
| | m/z: 214 (M + H) |
| 520 | 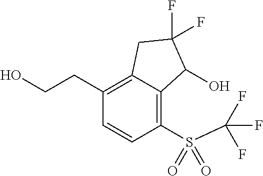 [SMILES: O=S(=O)(c1ccc(CCO)c2c1C(O)C(F)(F)C2)C(F)(F)F] |
| | m/z: 345 (M − H) |
| 521 | 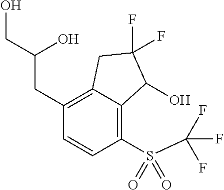 [SMILES: O=S(=O)(c1ccc(CC(O)CO)c2c1C(O)C(F)(F)C2)C(F)(F)F] |
| | m/z: 375 (M − H) |
| 522 | 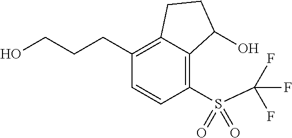 [SMILES: O=S(=O)(c1ccc(CCCO)c2c1C(O)CC2)C(F)(F)F] |
| | m/z: 323 (M − H) |
| 523 | 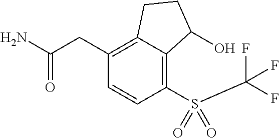 [SMILES: NC(=O)Cc1ccc(S(=O)(=O)C(F)(F)F)c2c1CCC2O] |
| | m/z: 322 (M − H) |
| 524 | 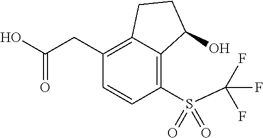 [SMILES: O=C(O)Cc1ccc(S(=O)(=O)C(F)(F)F)c2c1CC[C@H]2O] |
| | m/z: 323 (M − H) |
| 525 | 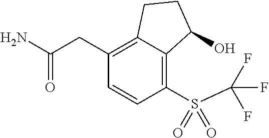 [SMILES: NC(=O)Cc1ccc(S(=O)(=O)C(F)(F)F)c2c1CC[C@H]2O] |
| 526 | 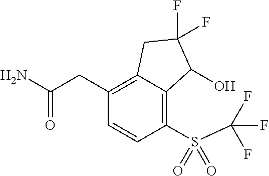 [SMILES: NC(=O)Cc1ccc(S(=O)(=O)C(F)(F)F)c2c1CC(F)(F)C2O] |
| | m/z: 360 (M + H) |
| 527 | 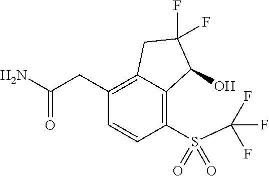 [SMILES: NC(=O)Cc1ccc(S(=O)(=O)C(F)(F)F)c2c1CC(F)(F)[C@H]2O] |
| | m/z: 360 (M + H) |
| 528 | 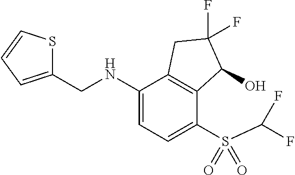 [SMILES: O=S(=O)(c1ccc(NCc2cccs2)c2c1[C@H](O)C(F)(F)C2)C(F)F] |
| 529 | 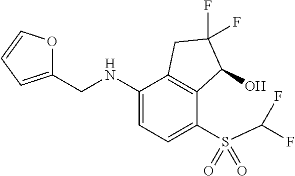 [SMILES: O=S(=O)(c1ccc(NCc2ccco2)c2c1[C@H](O)C(F)(F)C2)C(F)F] |
| 530 | 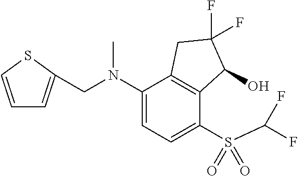 [SMILES: CN(Cc1cccs1)c1ccc(S(=O)(=O)C(F)F)c2c1CC(F)(F)[C@H]2O] |
| 531 | 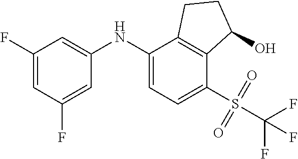 [SMILES: O=S(=O)(c1ccc(Nc2cc(F)cc(F)c2)c2c1[C@H](O)CC2)C(F)(F)F] |
| | m/z: [M − H]⁻ 392 |
| 532 | 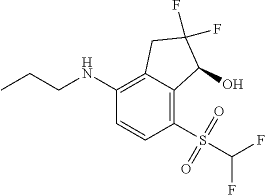 [SMILES: CCCNc1ccc(S(=O)(=O)C(F)F)c2c1CC(F)(F)[C@H]2O] |
| | m/z: [M + H]⁺ 343 |
| 533 | 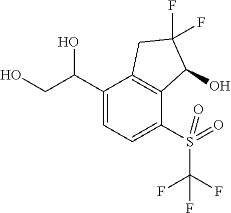 [SMILES: O=S(=O)(c1ccc(C(O)CO)c2c1[C@H](O)C(F)(F)C2)C(F)(F)F] |
| 534 | 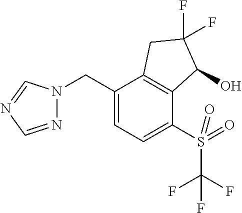 [SMILES: O=S(=O)(c1ccc(Cn2cncn2)c2c1[C@H](O)C(F)(F)C2)C(F)(F)F] |
| | m/z: [M − H]⁻ 382 |
| 535 | 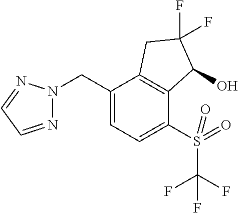 [SMILES: O=S(=O)(c1ccc(Cn2nccn2)c2c1[C@H](O)C(F)(F)C2)C(F)(F)F] |
| | m/z: [M − H]⁻ 382 |
| 536 | 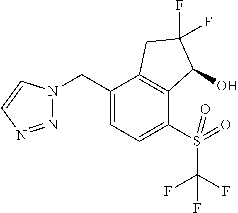 [SMILES: O=S(=O)(c1ccc(Cn2ccnn2)c2c1[C@H](O)C(F)(F)C2)C(F)(F)F] |
| | m/z: [M − H]⁻ 382 |
| 537 | 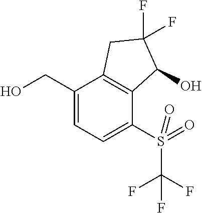 [SMILES: O=S(=O)(c1ccc(CO)c2c1[C@H](O)C(F)(F)C2)C(F)(F)F] |
| | m/z: [M − H]⁻ 331 |
| 538 | 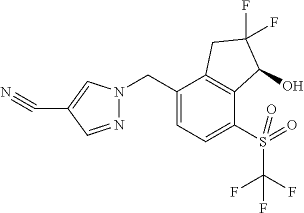 [SMILES: N#Cc1cnn(Cc2ccc(S(=O)(=O)C(F)(F)F)c3c2CC(F)(F)[C@H]3O)c1] |
| | m/z: [M − H]⁻ 406 |
| 539 | 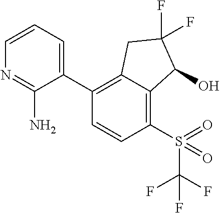 [SMILES: Nc1ncccc1-c1ccc(S(=O)(=O)C(F)(F)F)c2c1CC(F)(F)[C@H]2O] |
| | m/z: [M + H]⁺ 395 |
| 540 | 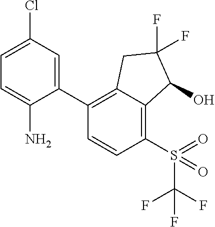 [SMILES: Nc1ccc(Cl)cc1-c1ccc(S(=O)(=O)C(F)(F)F)c2c1CC(F)(F)[C@H]2O] |
| | m/z: [M + H]⁺ 428/430 |
| 541 | 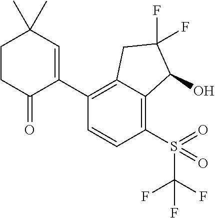 [SMILES: CC1(C)C=C(c2ccc(S(=O)(=O)C(F)(F)F)c3c2CC(F)(F)[C@H]3O)C(=O)CC1] |
| | m/z: [M + H]⁺ 425 |
| 542 | 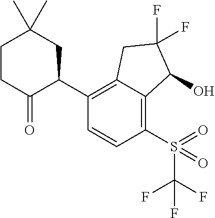 [SMILES: CC1(C)CCC(=O)[C@H](c2ccc(S(=O)(=O)C(F)(F)F)c3c2CC(F)(F)[C@H]3O)C1] |
| | m/z: [M + NH₄]⁺ 444 |
| 543 | 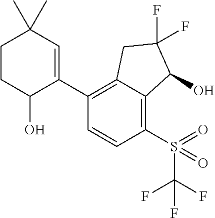 [SMILES: CC1(C)C=C(c2ccc(S(=O)(=O)C(F)(F)F)c3c2CC(F)(F)[C@H]3O)C(O)CC1] |
| | m/z: [M − OH]⁺ 409 |
| 544 | 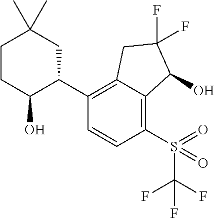 [SMILES: CC1(C)CC[C@H](O)[C@@H](c2ccc(S(=O)(=O)C(F)(F)F)c3c2CC(F)(F)[C@H]3O)C1] |
| | m/z: [M + Cl]⁻ 463/465 |
| 545 | 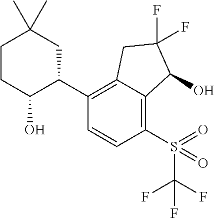 [SMILES: CC1(C)CC[C@@H](O)[C@@H](c2ccc(S(=O)(=O)C(F)(F)F)c3c2CC(F)(F)[C@H]3O)C1] |
| | m/z: [M + Cl]⁻ 463/465 |
| 546 | 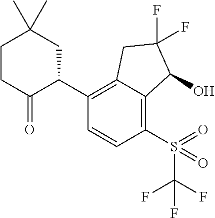 [SMILES: CC1(C)CCC(=O)[C@@H](c2ccc(S(=O)(=O)C(F)(F)F)c3c2CC(F)(F)[C@H]3O)C1] |
| | m/z: [M + NH₄]⁺ 444 |
| 547 | 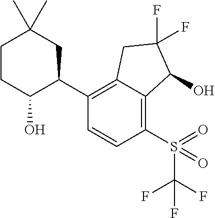 [SMILES: CC1(C)CC[C@@H](O)[C@H](c2ccc(S(=O)(=O)C(F)(F)F)c3c2CC(F)(F)[C@H]3O)C1] |
| | m/z: [M + Cl]⁻ 463/465 |
| 548 | 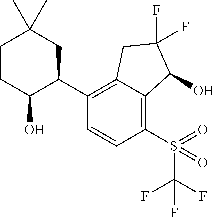 [SMILES: CC1(C)CC[C@H](O)[C@H](c2ccc(S(=O)(=O)C(F)(F)F)c3c2CC(F)(F)[C@H]3O)C1] |
| | m/z: [M + NH4]⁺ 446 |
| 549 | 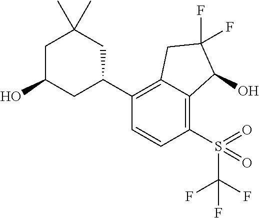 [SMILES: CC1(C)C[C@H](O)C[C@@H](c2ccc(S(=O)(=O)C(F)(F)F)c3c2CC(F)(F)[C@H]3O)C1] |
| | m/z: [M + NH4]⁺ 446 |
| 550 | 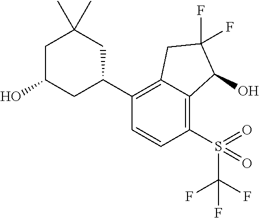 [SMILES: CC1(C)C[C@@H](O)C[C@@H](c2ccc(S(=O)(=O)C(F)(F)F)c3c2CC(F)(F)[C@H]3O)C1] |
| | m/z: [M + NH4]⁺ 446 |
| 551 | 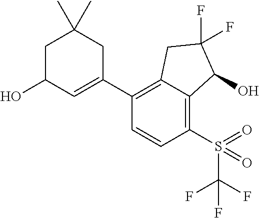 [SMILES: CC1(C)CC(c2ccc(S(=O)(=O)C(F)(F)F)c3c2CC(F)(F)[C@H]3O)=CC(O)C1] |
| | m/z: [M − OH]⁺ 409 |
| 552 | 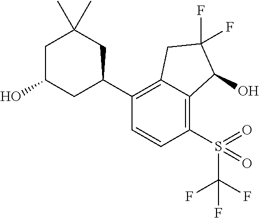 [SMILES: CC1(C)C[C@@H](O)C[C@H](c2ccc(S(=O)(=O)C(F)(F)F)c3c2CC(F)(F)[C@H]3O)C1] |
| | m/z: [M + NH4]⁺ 446 |
| 553 | 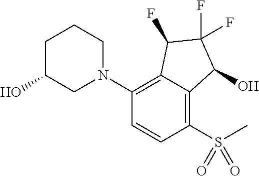 [SMILES: CS(=O)(=O)c1ccc(N2CCC[C@@H](O)C2)c2c1[C@H](O)C(F)(F)[C@@H]2F] |
| | m/z: [M + H]⁺ 366 |
| 554 | 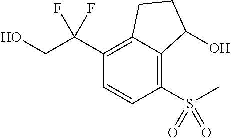 [SMILES: CS(=O)(=O)c1ccc(C(F)(F)CO)c2c1C(O)CC2] |
| | m/z: [M + NH₄]⁺ 310 |
| 555 | 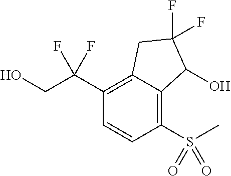 [SMILES: CS(=O)(=O)c1ccc(C(F)(F)CO)c2c1C(O)C(F)(F)C2] |
| | m/z: [M + NH₄]⁺ 346 |
| 556 | 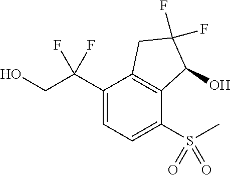 [SMILES: CS(=O)(=O)c1ccc(C(F)(F)CO)c2c1[C@H](O)C(F)(F)C2] |
| | m/z: [M + NH₄]⁺ 346 |
| 557 | 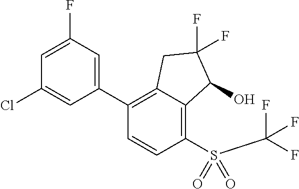 [SMILES: O=S(=O)(c1ccc(-c2cc(F)cc(Cl)c2)c2c1[C@H](O)C(F)(F)C2)C(F)(F)F] |
| | m/z: [M + Na]⁺ 454 |
| 558 | 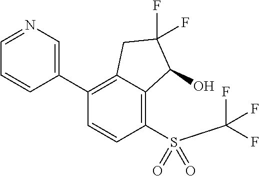 [SMILES: O=S(=O)(c1ccc(-c2cccnc2)c2c1[C@H](O)C(F)(F)C2)C(F)(F)F] |
| | m/z: [M + H]⁺ 380 |
| 559 | 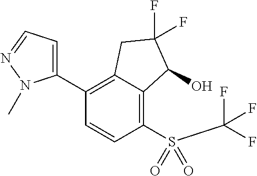 [SMILES: Cn1nccc1-c1ccc(S(=O)(=O)C(F)(F)F)c2c1CC(F)(F)[C@H]2O] |
| | m/z: [M + H]⁺ 383 |
| 560 | 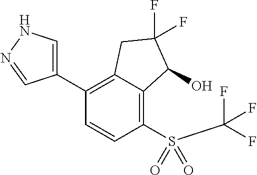 [SMILES: O=S(=O)(c1ccc(-c2cn[nH]c2)c2c1[C@H](O)C(F)(F)C2)C(F)(F)F] |
| | m/z: [M + H]⁺ 369 |
| 561 | 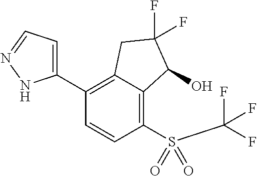 [SMILES: O=S(=O)(c1ccc(-c2ccn[nH]2)c2c1[C@H](O)C(F)(F)C2)C(F)(F)F] |
| | m/z: [M + H]⁺ 369 |
| 562 | 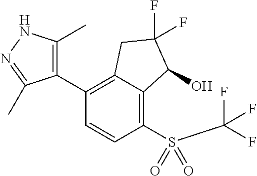 [SMILES: Cc1n[nH]c(C)c1-c1ccc(S(=O)(=O)C(F)(F)F)c2c1CC(F)(F)[C@H]2O] |
| | m/z: [M + H]⁺ 397 |
| 563 | 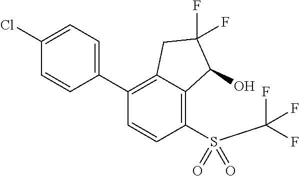 [SMILES: O=S(=O)(c1ccc(-c2ccc(Cl)cc2)c2c1[C@H](O)C(F)(F)C2)C(F)(F)F] |
| | m/z: [M + Na]⁺ 436/438 |
| 564 | 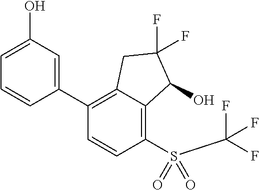 [SMILES: O=S(=O)(c1ccc(-c2cccc(O)c2)c2c1[C@H](O)C(F)(F)C2)C(F)(F)F] |
| | m/z: [M − H]⁻ 393 |
| 565 | 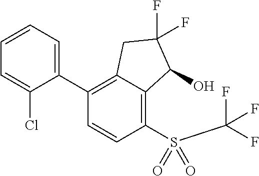 [SMILES: O=S(=O)(c1ccc(-c2ccccc2Cl)c2c1[C@H](O)C(F)(F)C2)C(F)(F)F] |
| | m/z: [M + Na]⁺ 436/438 |
| 566 | 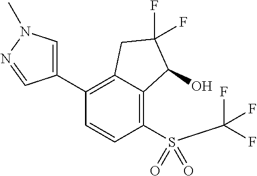 [SMILES: Cn1cc(-c2ccc(S(=O)(=O)C(F)(F)F)c3c2CC(F)(F)[C@H]3O)cn1] |
| | m/z: [M + H]⁺ 383 |
| 567 | 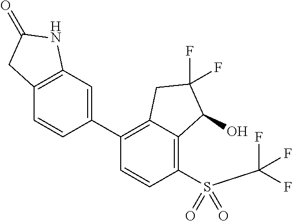 [SMILES: O=C1Cc2ccc(-c3ccc(S(=O)(=O)C(F)(F)F)c4c3CC(F)(F)[C@H]4O)cc2N1] |
| | m/z: [M + H]⁺ 434 |
| 568 | 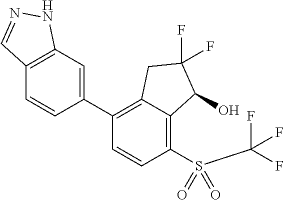 [SMILES: O=S(=O)(c1ccc(-c2ccc3cn[nH]c3c2)c2c1[C@H](O)C(F)(F)C2)C(F)(F)F] |
| | m/z: [M + H]⁺ 419 |
| 569 | 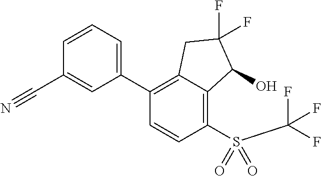 [SMILES: N#Cc1cccc(-c2ccc(S(=O)(=O)C(F)(F)F)c3c2CC(F)(F)[C@H]3O)c1] |
| | m/z: [M − H]⁻ 402 |
| 570 | 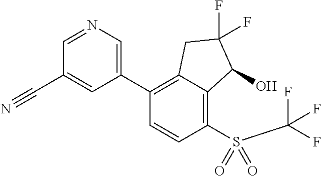 [SMILES: N#Cc1cncc(-c2ccc(S(=O)(=O)C(F)(F)F)c3c2CC(F)(F)[C@H]3O)c1] |
| | m/z: [M + H]⁺ 405 |
| 571 | 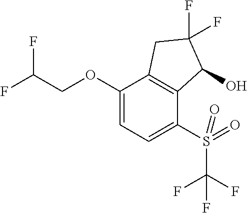 [SMILES: O=S(=O)(c1ccc(OCC(F)F)c2c1[C@H](O)C(F)(F)C2)C(F)(F)F] |
| | m/z: (M − H) 381 |
| 572 | 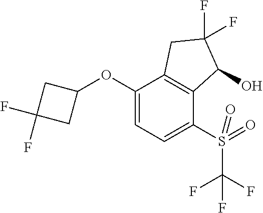 [SMILES: O=S(=O)(c1ccc(OC2CC(F)(F)C2)c2c1[C@H](O)C(F)(F)C2)C(F)(F)F] |
| | m/z: (M − H) 407 |
| 573 | 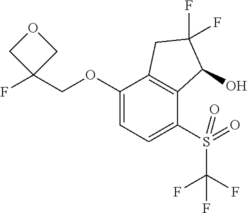 [SMILES: O=S(=O)(c1ccc(OCC2(F)COC2)c2c1[C@H](O)C(F)(F)C2)C(F)(F)F] |
| | m/z: (M − H) 405 |
| 574 | 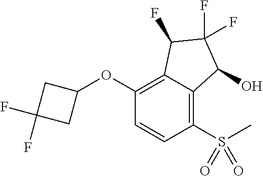 [SMILES: CS(=O)(=O)c1ccc(OC2CC(F)(F)C2)c2c1[C@H](O)C(F)(F)[C@@H]2F] |
| | m/z: 373 (M + H) |
| 575 | 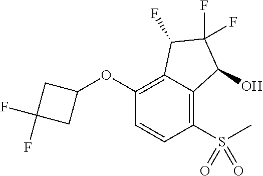 [SMILES: CS(=O)(=O)c1ccc(OC2CC(F)(F)C2)c2c1[C@H](O)C(F)(F)[C@H]2F] |
| | m/z: 373 (M + H) |
| 576 | 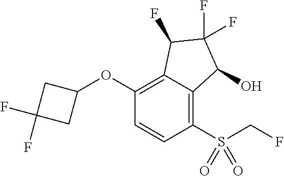 [SMILES: O=S(=O)(CF)c1ccc(OC2CC(F)(F)C2)c2c1[C@H](O)C(F)(F)[C@@H]2F] |
| | m/z: (M + H) 391 |
| 577 | 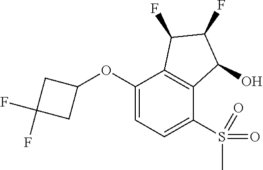 [SMILES: CS(=O)(=O)c1ccc(OC2CC(F)(F)C2)c2c1[C@H](O)[C@H](F)[C@@H]2F] |
| | m/z: (M + NH4) 372 |
| 578 | 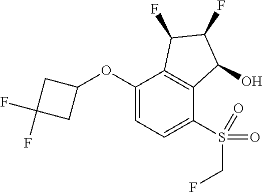 [SMILES: O=S(=O)(CF)c1ccc(OC2CC(F)(F)C2)c2c1[C@H](O)[C@H](F)[C@@H]2F] |
| | m/z: (M + NH4) 390 |
| 579 | 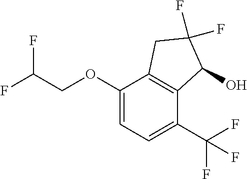 [SMILES: O[C@H]1c2c(C(F)(F)F)ccc(OCC(F)F)c2CC1(F)F] |
| | m/z: [M − H + formate] 363 |
| 580 | 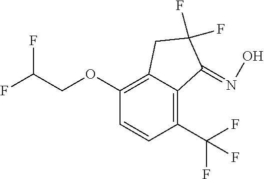 [SMILES: O/N=C1/c2c(C(F)(F)F)ccc(OCC(F)F)c2CC1(F)F] |
| | m/z: [M + H] 332 |
| 581 | 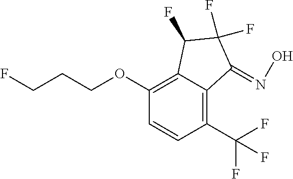 [SMILES: O/N=C1/c2c(C(F)(F)F)ccc(OCCCF)c2[C@@H](F)C1(F)F] |
| 582 | 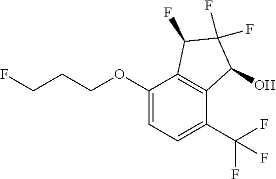 [SMILES: O[C@H]1c2c(C(F)(F)F)ccc(OCCCF)c2[C@@H](F)C1(F)F] |
| 583 | 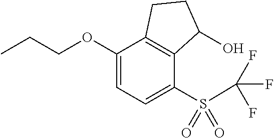 [SMILES: CCCOc1ccc(S(=O)(=O)C(F)(F)F)c2c1CCC2O] |
| 584 | 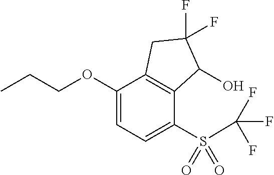 [SMILES: CCCOc1ccc(S(=O)(=O)C(F)(F)F)c2c1CC(F)(F)C2O] |
| | m/z: 359 (M − H) |
| 585 | 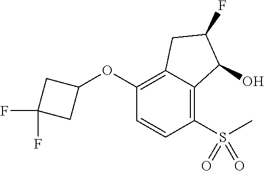 [SMILES: CS(=O)(=O)c1ccc(OC2CC(F)(F)C2)c2c1[C@H](O)[C@H](F)C2] |
| | m/z: 337 (M + H) |
| 586 | 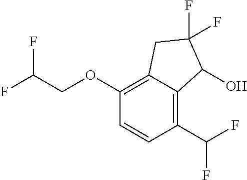 [SMILES: OC1c2c(C(F)F)ccc(OCC(F)F)c2CC1(F)F] |
| | m/z: 345 (M − H + HCOOH) |
| 587 | 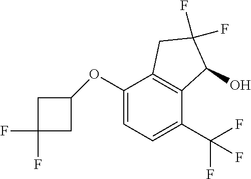 [SMILES: O[C@H]1c2c(C(F)(F)F)ccc(OC3CC(F)(F)C3)c2CC1(F)F] |
| | m/z: 389 (M − H + HCOOH) |
| 588 | 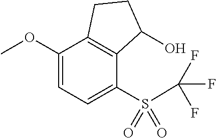 [SMILES: COc1ccc(S(=O)(=O)C(F)(F)F)c2c1CCC2O] |
| 589 | 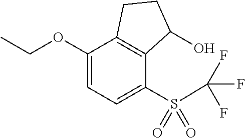 [SMILES: CCOc1ccc(S(=O)(=O)C(F)(F)F)c2c1CCC2O] |
| 590 | 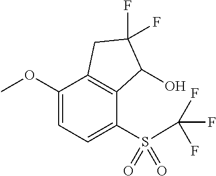 [SMILES: COc1ccc(S(=O)(=O)C(F)(F)F)c2c1CC(F)(F)C2O] |
| | m/z: 331 (M − H) |
| 591 | 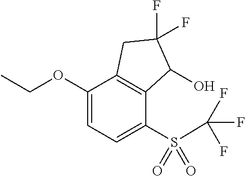 [SMILES: CCOc1ccc(S(=O)(=O)C(F)(F)F)c2c1CC(F)(F)C2O] |
| | m/z: 345 (M − H) |
| 592 | 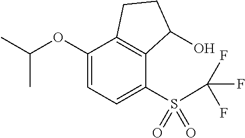 [SMILES: CC(C)Oc1ccc(S(=O)(=O)C(F)(F)F)c2c1CCC2O] |
| 593 | 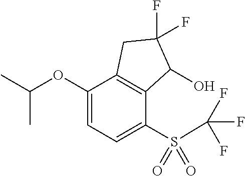 [SMILES: CC(C)Oc1ccc(S(=O)(=O)C(F)(F)F)c2c1CC(F)(F)C2O] |
| | m/z: 359 (M − H) |
| 594 | 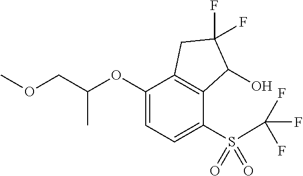 [SMILES: COCC(C)Oc1ccc(S(=O)(=O)C(F)(F)F)c2c1CC(F)(F)C2O] |
| | m/z: 389 (M − H) |
| 595 | 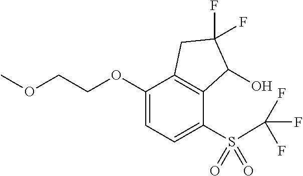 [SMILES: COCCOc1ccc(S(=O)(=O)C(F)(F)F)c2c1CC(F)(F)C2O] |
| | m/z: 375 (M − H) |
| 596 | 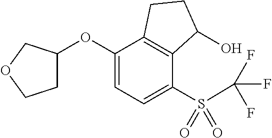 [SMILES: O=S(=O)(c1ccc(OC2CCOC2)c2c1C(O)CC2)C(F)(F)F] |
| 597 | 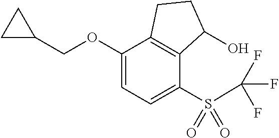 [SMILES: O=S(=O)(c1ccc(OCC2CC2)c2c1C(O)CC2)C(F)(F)F] |
| 598 | 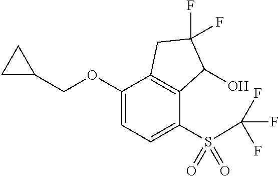 [SMILES: O=S(=O)(c1ccc(OCC2CC2)c2c1C(O)C(F)(F)C2)C(F)(F)F] |
| | m/z: 371 (M − H) |
| 599 | 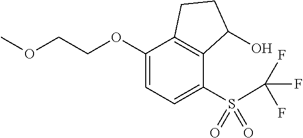 [SMILES: COCCOc1ccc(S(=O)(=O)C(F)(F)F)c2c1CCC2O] |
| 600 | 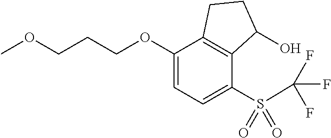 [SMILES: COCCCOc1ccc(S(=O)(=O)C(F)(F)F)c2c1CCC2O] |
| | m/z: 372 (M + NH₄) |
| 601 | 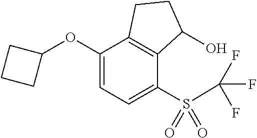 [SMILES: O=S(=O)(c1ccc(OC2CCC2)c2c1C(O)CC2)C(F)(F)F] |
| 602 | 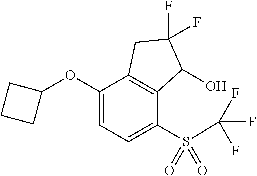 [SMILES: O=S(=O)(c1ccc(OC2CCC2)c2c1C(O)C(F)(F)C2)C(F)(F)F] |
| | m/z: 371 (M − H) |
| 603 | 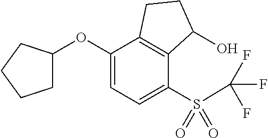 [SMILES: O=S(=O)(c1ccc(OC2CCCC2)c2c1C(O)CC2)C(F)(F)F] |
| 604 | 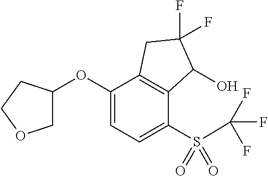 [SMILES: O=S(=O)(c1ccc(OC2CCOC2)c2c1C(O)C(F)(F)C2)C(F)(F)F] |
| | m/z: 387 (M − H) |
| 605 | 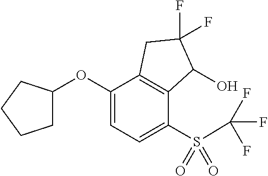 [SMILES: O=S(=O)(c1ccc(OC2CCCC2)c2c1C(O)C(F)(F)C2)C(F)(F)F] |
| | m/z: 385 (M − H) |
| 606 | 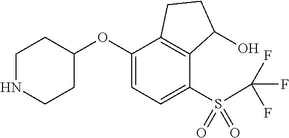 [SMILES: O=S(=O)(c1ccc(OC2CCNCC2)c2c1C(O)CC2)C(F)(F)F] |
| 607 | 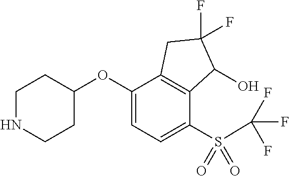 [SMILES: O=S(=O)(c1ccc(OC2CCNCC2)c2c1C(O)C(F)(F)C2)C(F)(F)F] |
| | m/z: 402 (M + H) |
| 608 | 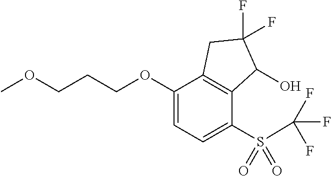 [SMILES: COCCCOc1ccc(S(=O)(=O)C(F)(F)F)c2c1CC(F)(F)C2O] |
| | m/z: 389 (M − H) |
| 609 | 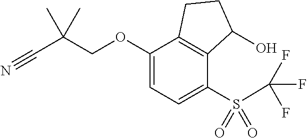 [SMILES: CC(C)(C#N)COc1ccc(S(=O)(=O)C(F)(F)F)c2c1CCC2O] |
| 610 | 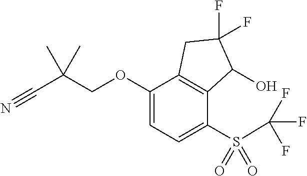 [SMILES: CC(C)(C#N)COc1ccc(S(=O)(=O)C(F)(F)F)c2c1CC(F)(F)C2O] |
| | m/z: 398 (M − H) |
| 611 | 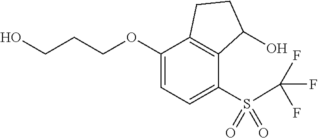 [SMILES: O=S(=O)(c1ccc(OCCCO)c2c1C(O)CC2)C(F)(F)F] |
| 612 | 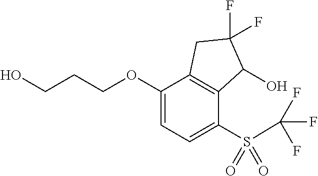 [SMILES: O=S(=O)(c1ccc(OCCCO)c2c1C(O)C(F)(F)C2)C(F)(F)F] |
| | m/z: 377 (M + H) |
| 613 | 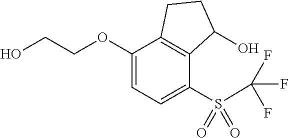 [SMILES: O=S(=O)(c1ccc(OCCO)c2c1C(O)CC2)C(F)(F)F] |
| | m/z: 371 (M − H + HCOOH) |
| 614 | 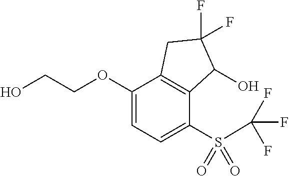 [SMILES: O=S(=O)(c1ccc(OCCO)c2c1C(O)C(F)(F)C2)C(F)(F)F] |
| | m/z: 363 (M + H) |
| 615 | 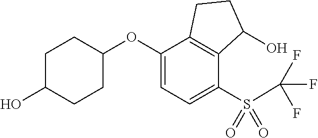 [SMILES: O=S(=O)(c1ccc(OC2CCC(O)CC2)c2c1C(O)CC2)C(F)(F)F] |
| | m/z: 425 (M − H + HCOOH) |
| 616 | 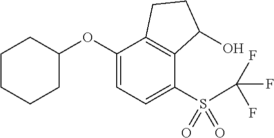 [SMILES: O=S(=O)(c1ccc(OC2CCCCC2)c2c1C(O)CC2)C(F)(F)F] |
| | m/z: 382 (M + NH₄⁺) |
| 617 | 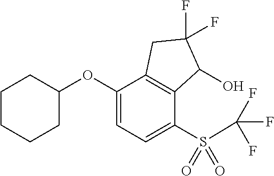 [SMILES: O=S(=O)(c1ccc(OC2CCCCC2)c2c1C(O)C(F)(F)C2)C(F)(F)F] |
| | m/z: 399 (M − H) |
| 618 | 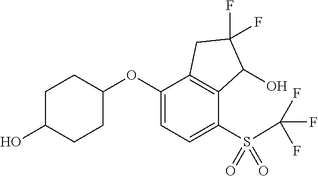 [SMILES: O=S(=O)(c1ccc(OC2CCC(O)CC2)c2c1C(O)C(F)(F)C2)C(F)(F)F] |
| | m/z: 415 (M − H) |
| 619 | 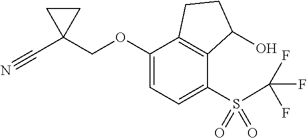 [SMILES: N#CC1(COc2ccc(S(=O)(=O)C(F)(F)F)c3c2CCC3O)CC1] |
| | m/z: 406 (M − H + HCOOH) |
| 620 | 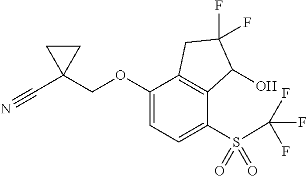 [SMILES: N#CC1(COc2ccc(S(=O)(=O)C(F)(F)F)c3c2CC(F)(F)C3O)CC1] |
| | m/z: 398 (M + H) |
| 621 | 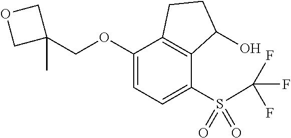 [SMILES: CC1(COc2ccc(S(=O)(=O)C(F)(F)F)c3c2CCC3O)COC1] |
| | m/z: 411 (M − H + HCOOH) |
| 622 | 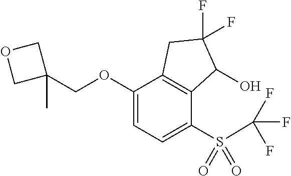 [SMILES: CC1(COc2ccc(S(=O)(=O)C(F)(F)F)c3c2CC(F)(F)C3O)COC1] |
| | m/z: 401 (M − H) |
| 623 | 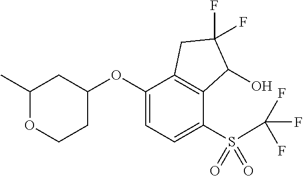 [SMILES: CC1CC(Oc2ccc(S(=O)(=O)C(F)(F)F)c3c2CC(F)(F)C3O)CCO1] |
| | m/z: 415 (M − H) |
| 624 | 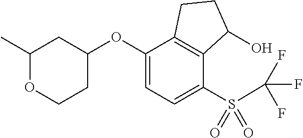 [SMILES: CC1CC(Oc2ccc(S(=O)(=O)C(F)(F)F)c3c2CCC3O)CCO1] |
| | m/z: 425 (M − H + HCOOH) |
| 625 | 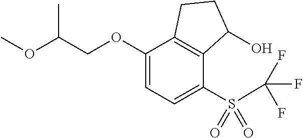 [SMILES: COC(C)COc1ccc(S(=O)(=O)C(F)(F)F)c2c1CCC2O] |
| | m/z: 372 (M + NH₄) |
| 626 | 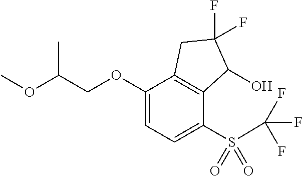 [SMILES: COC(C)COc1ccc(S(=O)(=O)C(F)(F)F)c2c1CC(F)(F)C2O] |
| | m/z: 389 (M − H) |
| 627 | 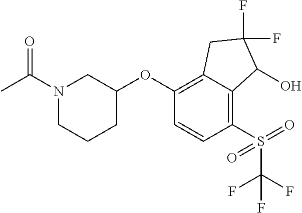 [SMILES: CC(=O)N1CCCC(Oc2ccc(S(=O)(=O)C(F)(F)F)c3c2CC(F)(F)C3O)C1] |
| | m/z: 444 (M + H) |
| 628 | 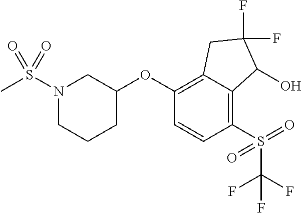 [SMILES: CS(=O)(=O)N1CCCC(Oc2ccc(S(=O)(=O)C(F)(F)F)c3c2CC(F)(F)C3O)C1] |
| | m/z: 480 (M + H) |
| 629 | 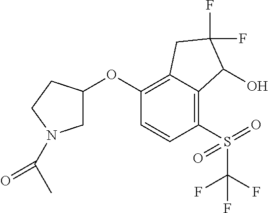 [SMILES: CC(=O)N1CCC(Oc2ccc(S(=O)(=O)C(F)(F)F)c3c2CC(F)(F)C3O)C1] |
| | m/z: 430 (M + H) |
| 630 | 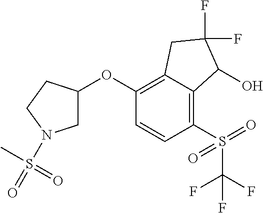 [SMILES: CS(=O)(=O)N1CCC(Oc2ccc(S(=O)(=O)C(F)(F)F)c3c2CC(F)(F)C3O)C1] |
| | m/z: 466 (M + H) |
| 631 | 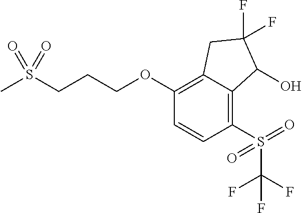 [SMILES: CS(=O)(=O)CCCOc1ccc(S(=O)(=O)C(F)(F)F)c2c1CC(F)(F)C2O] |
| | m/z: 439 (M + H) |
| 632 | 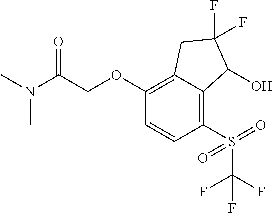 [SMILES: CN(C)C(=O)COc1ccc(S(=O)(=O)C(F)(F)F)c2c1CC(F)(F)C2O] |
| | m/z: 404 (M + H) |
| 633 | 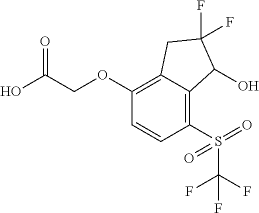 [SMILES: O=C(O)COc1ccc(S(=O)(=O)C(F)(F)F)c2c1CC(F)(F)C2O] |
| | m/z: 377 (M + H) |
| 634 | 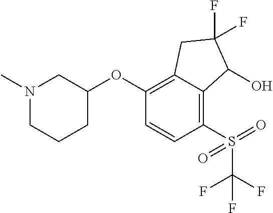 [SMILES: CN1CCCC(Oc2ccc(S(=O)(=O)C(F)(F)F)c3c2CC(F)(F)C3O)C1] |
| | m/z: 416 (M + H) |
| 635 | 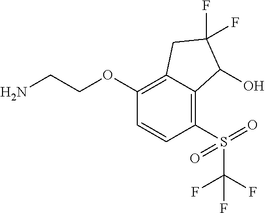 [SMILES: NCCOc1ccc(S(=O)(=O)C(F)(F)F)c2c1CC(F)(F)C2O] |
| | m/z: 362 (M + H) |
| 636 | 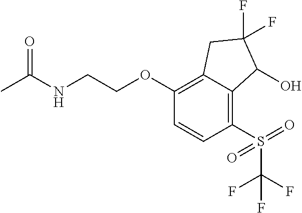 [SMILES: CC(=O)NCCOc1ccc(S(=O)(=O)C(F)(F)F)c2c1CC(F)(F)C2O] |
| | m/z: 404 (M + H) |
| 637 | 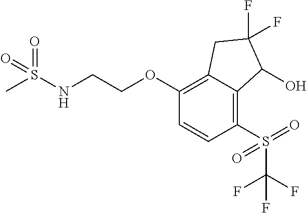 [SMILES: CS(=O)(=O)NCCOc1ccc(S(=O)(=O)C(F)(F)F)c2c1CC(F)(F)C2O] |
| | m/z: 440 (M + H) |
| 638 | 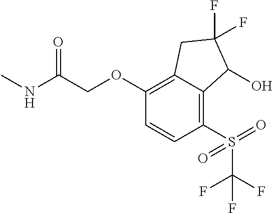 [SMILES: CNC(=O)COc1ccc(S(=O)(=O)C(F)(F)F)c2c1CC(F)(F)C2O] |
| | m/z: 390 (M + H) |
| 639 | 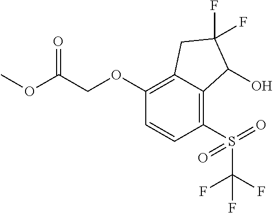 [SMILES: COC(=O)COc1ccc(S(=O)(=O)C(F)(F)F)c2c1CC(F)(F)C2O] |
| | m/z: 389 (M − H) |
| 640 | 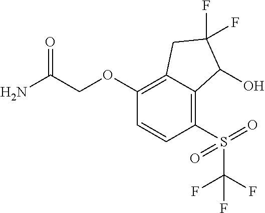 [SMILES: NC(=O)COc1ccc(S(=O)(=O)C(F)(F)F)c2c1CC(F)(F)C2O] |
| | m/z: 374 (M − H) |
| 641 | 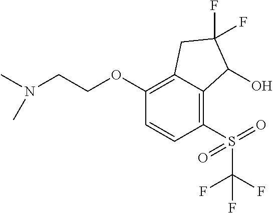 [SMILES: CN(C)CCOc1ccc(S(=O)(=O)C(F)(F)F)c2c1CC(F)(F)C2O] |
| | m/z: 390 (M + H) |
| 642 | 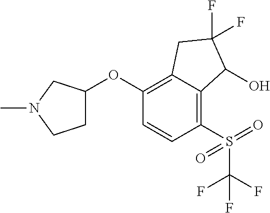 [SMILES: CN1CCC(Oc2ccc(S(=O)(=O)C(F)(F)F)c3c2CC(F)(F)C3O)C1] |
| 643 | 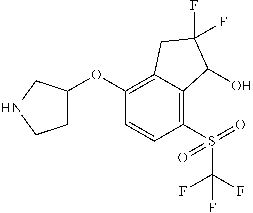 [SMILES: O=S(=O)(c1ccc(OC2CCNC2)c2c1C(O)C(F)(F)C2)C(F)(F)F] |
| | m/z: 388 (M + H) |
| 644 | 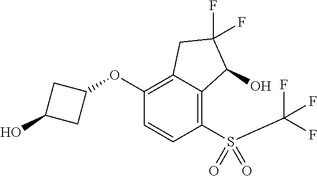 |
| | m/z: 433 (M − H + HCOOH) |
| 645 | 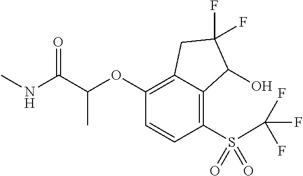 [SMILES: CNC(=O)C(C)Oc1ccc(S(=O)(=O)C(F)(F)F)c2c1CC(F)(F)C2O] |
| | m/z: 402 (M − H) |
| 646 | 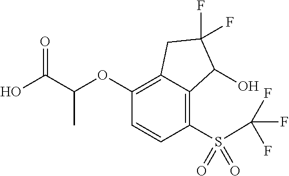 [SMILES: CC(Oc1ccc(S(=O)(=O)C(F)(F)F)c2c1CC(F)(F)C2O)C(=O)O] |
| | m/z: 389 (M − H) |
| 647 | 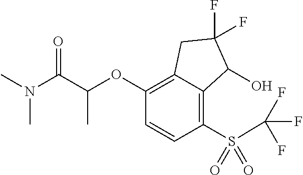 [SMILES: CC(Oc1ccc(S(=O)(=O)C(F)(F)F)c2c1CC(F)(F)C2O)C(=O)N(C)C] |
| | m/z: 418 (M + H) |
| 648 | 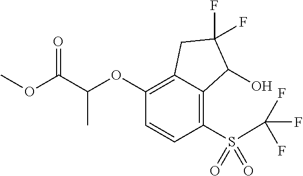 [SMILES: COC(=O)C(C)Oc1ccc(S(=O)(=O)C(F)(F)F)c2c1CC(F)(F)C2O] |
| 649 | 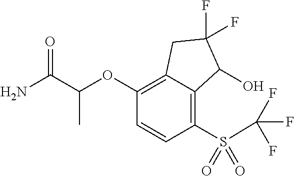 [SMILES: CC(Oc1ccc(S(=O)(=O)C(F)(F)F)c2c1CC(F)(F)C2O)C(N)=O] |
| | m/z: 388 (M − H) |
| 650 | 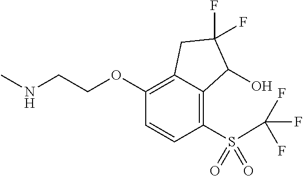 [SMILES: CNCCOc1ccc(S(=O)(=O)C(F)(F)F)c2c1CC(F)(F)C2O] |
| | m/z: 376 (M + H) |
| 651 | 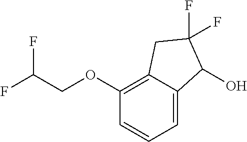 [SMILES: OC1c2cccc(OCC(F)F)c2CC1(F)F] |
| 652 | 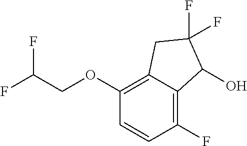 [SMILES: OC1c2c(F)ccc(OCC(F)F)c2CC1(F)F] |
| | m/z: 313 (M − H + HCOOH) |
| 653 | 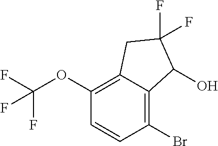 [SMILES: OC1c2c(Br)ccc(OC(F)(F)F)c2CC1(F)F] |
| 654 | 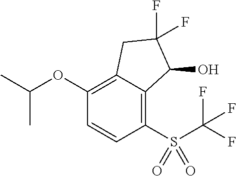 [SMILES: CC(C)Oc1ccc(S(=O)(=O)C(F)(F)F)c2c1CC(F)(F)[C@H]2O] |
| | m/z: 359 (M − H) |
| 655 | 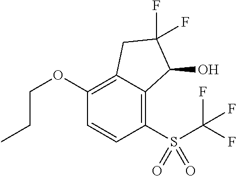 [SMILES: CCCOc1ccc(S(=O)(=O)C(F)(F)F)c2c1CC(F)(F)[C@H]2O] |
| | m/z: 359 (M − H) |
| 656 | 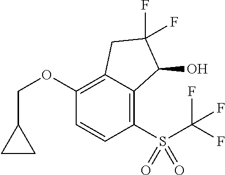 [SMILES: O=S(=O)(c1ccc(OCC2CC2)c2c1[C@H](O)C(F)(F)C2)C(F)(F)F] |
| | m/z: 371 (M − H) |
| 657 | 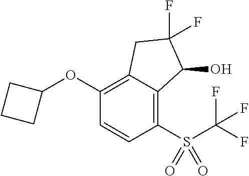 [SMILES: O=S(=O)(c1ccc(OC2CCC2)c2c1[C@H](O)C(F)(F)C2)C(F)(F)F] |
| | m/z: 371 (M − H) |
| 658 | 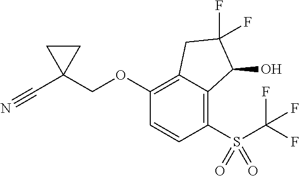 [SMILES: N#CC1(COc2ccc(S(=O)(=O)C(F)(F)F)c3c2CC(F)(F)[C@H]3O)CC1] |
| | m/z: 442 (M + HCO₂⁻) |
| 659 | 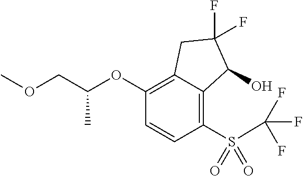 [SMILES: COC[C@@H](C)Oc1ccc(S(=O)(=O)C(F)(F)F)c2c1CC(F)(F)[C@H]2O] |
| | m/z: 389 (M − H) |
| 660 | 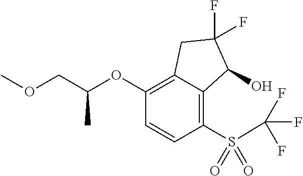 [SMILES: COC[C@H](C)Oc1ccc(S(=O)(=O)C(F)(F)F)c2c1CC(F)(F)[C@H]2O] |
| | m/z: 389 (M − H) |
| 661 | 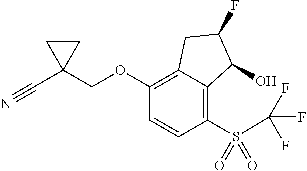 [SMILES: N#CC1(COc2ccc(S(=O)(=O)C(F)(F)F)c3c2C[C@@H](F)[C@H]3O)CC1] |
| | m/z: 424 (M + HCO₂⁻) |
| 662 | 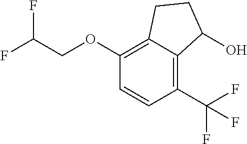 [SMILES: OC1CCc2c(OCC(F)F)ccc(C(F)(F)F)c21] |
| | m/z: 265 (M − OH) |
| 663 | 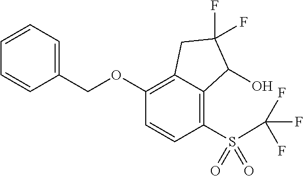 [SMILES: O=S(=O)(c1ccc(OCc2ccccc2)c2c1C(O)C(F)(F)C2)C(F)(F)F] |
| | m/z: (M + NH4) 426 |
| 664 | 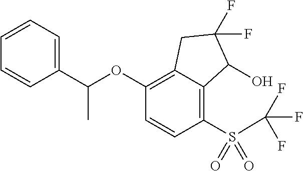 [SMILES: CC(Oc1ccc(S(=O)(=O)C(F)(F)F)c2c1CC(F)(F)C2O)c1ccccc1] |
| | m/z: (M − H) 421 |
| 665 | 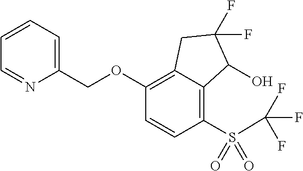 [SMILES: O=S(=O)(c1ccc(OCc2ccccn2)c2c1C(O)C(F)(F)C2)C(F)(F)F] |
| | m/z: (M + H) 410 |
| 666 | 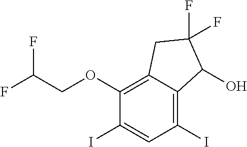 [SMILES: OC1c2c(I)cc(I)c(OCC(F)F)c2CC1(F)F] |
| 667 | 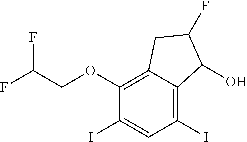 [SMILES: OC1c2c(I)cc(I)c(OCC(F)F)c2CC1F] |
| | m/z: (M − OH) 467 |
| 668 | 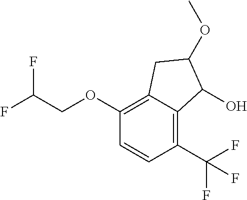 [SMILES: COC1Cc2c(OCC(F)F)ccc(C(F)(F)F)c2C1O] |
| | m/z: (M + NH4) 330 |
| 669 | 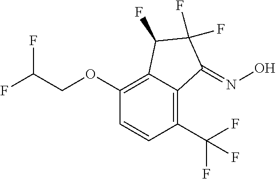 [SMILES: O/N=C1/c2c(C(F)(F)F)ccc(OCC(F)F)c2[C@@H](F)C1(F)F] |
| | m/z: (M + H) 350 |
| 670 | 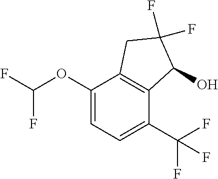 [SMILES: O[C@H]1c2c(C(F)(F)F)ccc(OC(F)F)c2CC1(F)F] |
| 671 | 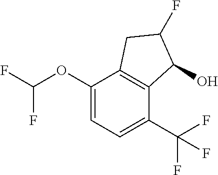 [SMILES: O[C@H]1c2c(C(F)(F)F)ccc(OC(F)F)c2CC1F] |
| 672 | 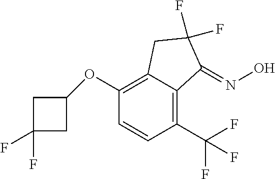 [SMILES: O/N=C1/c2c(C(F)(F)F)ccc(OC3CC(F)(F)C3)c2CC1(F)F] |
| | m/z: (M + H) 358 |
| 673 | 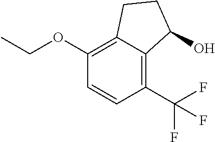 [SMILES: CCOc1ccc(C(F)(F)F)c2c1CC[C@H]2O] |
| | m/z: [M − OH]⁺ 229 |
| 674 | 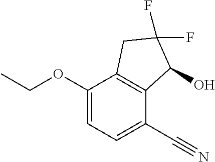 [SMILES: CCOc1ccc(C#N)c2c1CC(F)(F)[C@H]2O] |
| | m/z: [M − H + HCOOH]⁻ 284 |
| 675 | 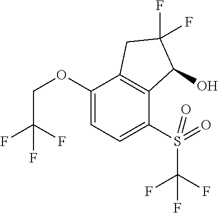 [SMILES: O=S(=O)(c1ccc(OCC(F)(F)F)c2c1[C@H](O)C(F)(F)C2)C(F)(F)F] |
| | m/z: [M − H + HCOOH]⁻ 445 |
| 676 | 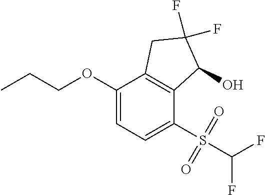 [SMILES: CCCOc1ccc(S(=O)(=O)C(F)F)c2c1CC(F)(F)[C@H]2O] |
| | m/z: [M + NH4]⁺ 360 |
| 677 | 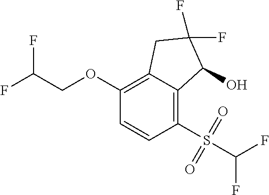 [SMILES: O=S(=O)(c1ccc(OCC(F)F)c2c1[C@H](O)C(F)(F)C2)C(F)F] |
| | m/z: [M + NH4]⁺ 382 |
| 678 | 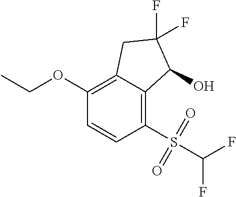 [SMILES: CCOc1ccc(S(=O)(=O)C(F)F)c2c1CC(F)(F)[C@H]2O] |
| | m/z: [M + NH4]⁺ 346 |
| 679 | 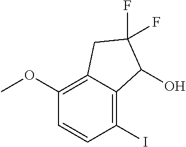 |
| 680 | 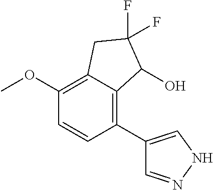 [SMILES: COc1ccc(-c2cn[nH]c2)c2c1CC(F)(F)C2O] |
| | m/z: [M + Na]⁺ 289 |
| 681 | 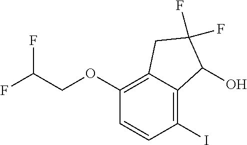 [SMILES: OC1c2c(I)ccc(OCC(F)F)c2CC1(F)F] |
| 682 | 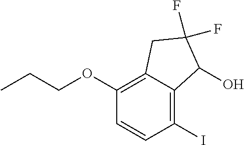 [SMILES: CCCOc1ccc(I)c2c1CC(F)(F)C2O] |
| 683 | 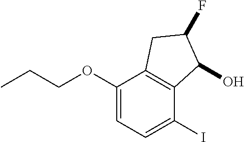 [SMILES: CCCOc1ccc(I)c2c1C[C@@H](F)[C@H]2O] |
| 684 | 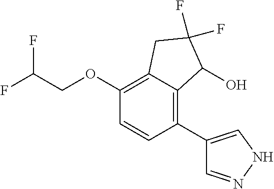 [SMILES: OC1c2c(-c3cn[nH]c3)ccc(OCC(F)F)c2CC1(F)F] |
| | m/z: [M + H]⁺ 317 |
| 685 | 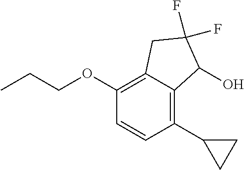 [SMILES: CCCOc1ccc(C2CC2)c2c1CC(F)(F)C2O] |
| | m/z: [M + NH4]⁻ 251 |
| 686 | 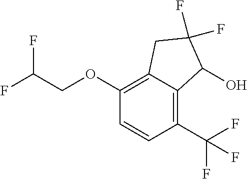 [SMILES: OC1c2c(C(F)(F)F)ccc(OCC(F)F)c2CC1(F)F] |
| | m/z: [M − H + HCOOH]⁻ 363 |
| 687 | 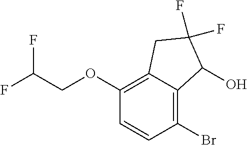 [SMILES: OC1c2c(Br)ccc(OCC(F)F)c2CC1(F)F] |
| 688 | 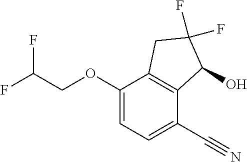 [SMILES: N#Cc1ccc(OCC(F)F)c2c1[C@H](O)C(F)(F)C2] |
| | m/z: [M + H]⁺ 276 |
| 689 | 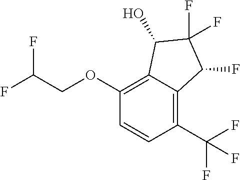 [SMILES: O[C@H]1c2c(OCC(F)F)ccc(C(F)(F)F)c2[C@@H](F)C1(F)F] |
| 690 | 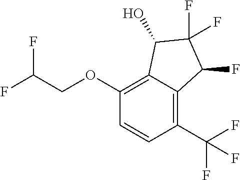 [SMILES: O[C@H]1c2c(OCC(F)F)ccc(C(F)(F)F)c2[C@H](F)C1(F)F] |
| 691 | 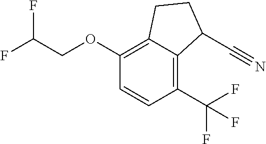 [SMILES: N#CC1CCc2c(OCC(F)F)ccc(C(F)(F)F)c21] |
| | m/z: [M − H]⁻ 290 |
| 692 | 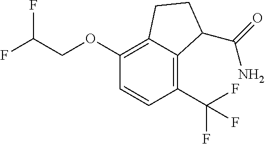 [SMILES: NC(=O)C1CCc2c(OCC(F)F)ccc(C(F)(F)F)c21] |
| | m/z: [M + H]⁺ 310 |
| 693 | 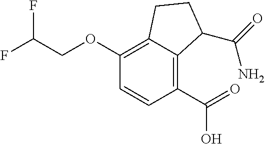 [SMILES: NC(=O)C1CCc2c(OCC(F)F)ccc(C(=O)O)c21] |
| | m/z: [M + H]⁺ 286 |
| 694 | 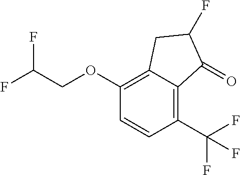 [SMILES: O=C1c2c(C(F)(F)F)ccc(OCC(F)F)c2CC1F] |
| | m/z: [M − H + HCOOH]⁻ 343 |
| 695 | 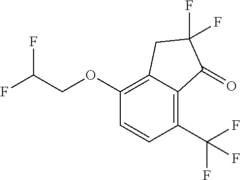 [SMILES: O=C1c2c(C(F)(F)F)ccc(OCC(F)F)c2CC1(F)F] |
| | m/z: [M + H]⁺ 317 |
| 696 | 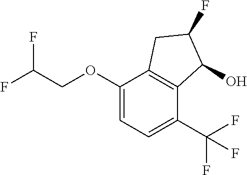 [SMILES: O[C@H]1c2c(C(F)(F)F)ccc(OCC(F)F)c2C[C@H]1F] |
| 697 | 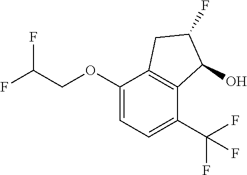 [SMILES: O[C@H]1c2c(C(F)(F)F)ccc(OCC(F)F)c2C[C@@H]1F] |
| 698 | 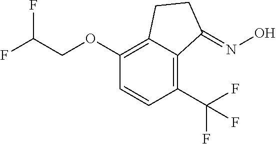 [SMILES: O/N=C1\CCc2c(OCC(F)F)ccc(C(F)(F)F)c21] |
| 699 | 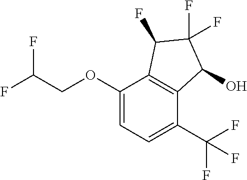 [SMILES: O[C@H]1c2c(C(F)(F)F)ccc(OCC(F)F)c2[C@@H](F)C1(F)F] |
| | m/z: [M − H + HCOOH]⁻ 381 |
| 700 | 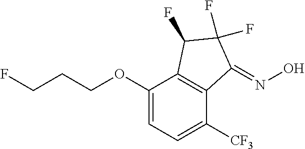 [SMILES: O/N=C1/c2c(C(F)(F)F)ccc(OCCCF)c2[C@@H](F)C1(F)F] |
| | m/z: [M − H]⁻ 344 |
| 701 | 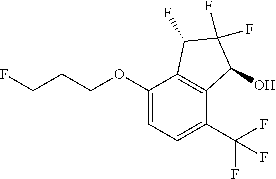 [SMILES: O[C@H]1c2c(C(F)(F)F)ccc(OCCCF)c2[C@H](F)C1(F)F] |
| | m/z: [M − H + HCOOH]⁻ 377 |
| 702 | 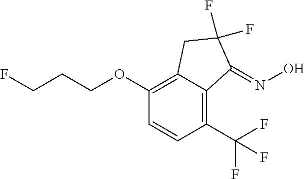 [SMILES: O/N=C1/c2c(C(F)(F)F)ccc(OCCCF)c2CC1(F)F] |
| | m/z: [M + H]⁺ 328 |
| 703 | 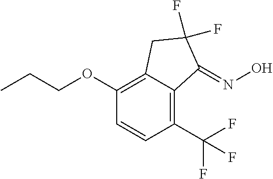 [SMILES: CCCOc1ccc(C(F)(F)F)c2c1CC(F)(F)/C2=N\O] |
| | m/z: [M + H]⁺ 310 |
| 704 | 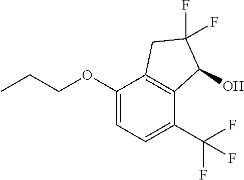 [SMILES: CCCOc1ccc(C(F)(F)F)c2c1CC(F)(F)[C@H]2O] |
| | m/z: [M + Na]⁺ 320 |
| 705 | 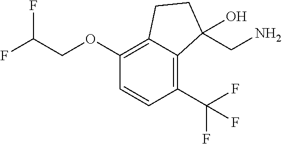 [SMILES: NCC1(O)CCc2c(OCC(F)F)ccc(C(F)(F)F)c21] |
| | m/z: [M + H]⁺ 312 |
| 706 | 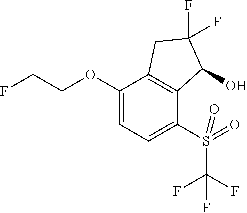 [SMILES: O=S(=O)(c1ccc(OCCF)c2c1[C@H](O)C(F)(F)C2)C(F)(F)F] |
| | m/z: 363 (M − H)⁻ |
| 707 | 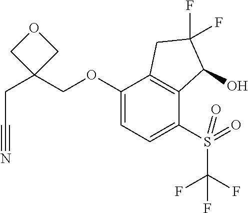 [SMILES: N#CCC1(COc2ccc(S(=O)(=O)C(F)(F)F)c3c2CC(F)(F)[C@H]3O)COC1] |
| | m/z: 472 (M + HCO₂)⁻ |
| 708 | 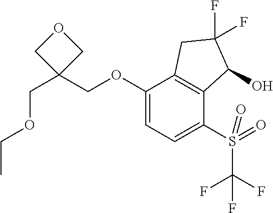 [SMILES: CCOCC1(COc2ccc(S(=O)(=O)C(F)(F)F)c3c2CC(F)(F)[C@H]3O)COC1] |
| | m/z: 491 (M + HCO₂)⁻ |
| 709 | 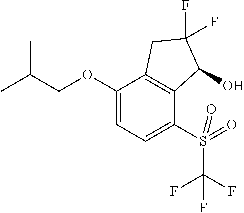 [SMILES: CC(C)COc1ccc(S(=O)(=O)C(F)(F)F)c2c1CC(F)(F)[C@H]2O] |
| | m/z: 373 (M − H)⁻ |
| 710 | 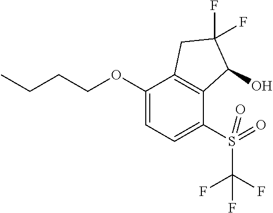 [SMILES: CCCCOc1ccc(S(=O)(=O)C(F)(F)F)c2c1CC(F)(F)[C@H]2O] |
| | m/z: 373 (M − H)⁻ |
| 711 | 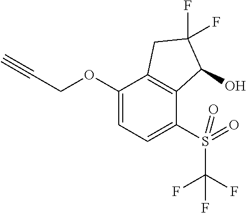 [SMILES: C#CCOc1ccc(S(=O)(=O)C(F)(F)F)c2c1CC(F)(F)[C@H]2O] |
| | m/z: 355 (M − H)⁻ |
| 712 | 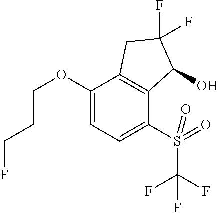 [SMILES: O=S(=O)(c1ccc(OCCCF)c2c1[C@H](O)C(F)(F)C2)C(F)(F)F] |
| | m/z: 377 (M − H)⁻ |
| 713 | 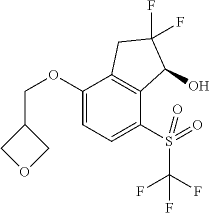 [SMILES: O=S(=O)(c1ccc(OCC2COC2)c2c1[C@H](O)C(F)(F)C2)C(F)(F)F] |
| | m/z: 387 (M − H)⁻ |
| 714 | 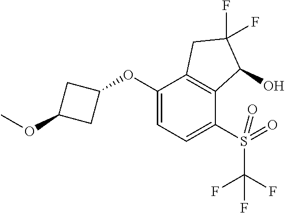 [SMILES: CO[C@H]1C[C@H](Oc2ccc(S(=O)(=O)C(F)(F)F)c3c2CC(F)(F)[C@H]3O)C1] |
| | m/z: 401 (M − H)⁻ |
| 715 | 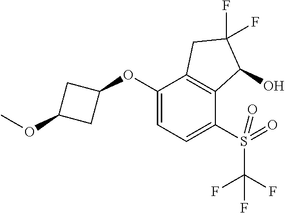 [SMILES: CO[C@H]1C[C@@H](Oc2ccc(S(=O)(=O)C(F)(F)F)c3c2CC(F)(F)[C@H]3O)C1] |
| | m/z: 401 (M − H)⁻ |
| 716 | 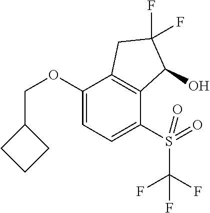 [SMILES: O=S(=O)(c1ccc(OCC2CCC2)c2c1[C@H](O)C(F)(F)C2)C(F)(F)F] |
| | m/z: 385 (M − H)⁻ |
| 717 | 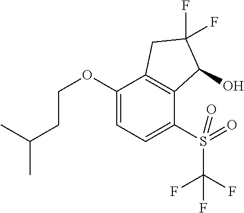 [SMILES: CC(C)CCOc1ccc(S(=O)(=O)C(F)(F)F)c2c1CC(F)(F)[C@H]2O] |
| | m/z: 387 (M − H)⁻ |
| 718 | 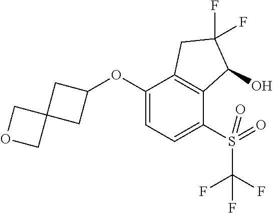 [SMILES: O=S(=O)(c1ccc(OC2CC3(COC3)C2)c2c1[C@H](O)C(F)(F)C2)C(F)(F)F] |
| | m/z: 413 (M − H)⁻ |
| 719 | 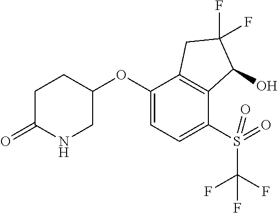 [SMILES: O=C1CCC(Oc2ccc(S(=O)(=O)C(F)(F)F)c3c2CC(F)(F)[C@H]3O)CN1] |
| | m/z: 416 (M + H)⁺ |
| 720 | 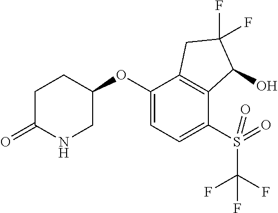 [SMILES: O=C1CC[C@@H](Oc2ccc(S(=O)(=O)C(F)(F)F)c3c2CC(F)(F)[C@H]3O)CN1] |
| | m/z: 416 (M + H)⁺ |
| 721 | 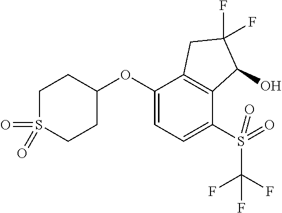 [SMILES: O=S1(=O)CCC(Oc2ccc(S(=O)(=O)C(F)(F)F)c3c2CC(F)(F)[C@H]3O)CC1] |
| | m/z: 495 (M + HCO₂)⁻ |
| 722 | 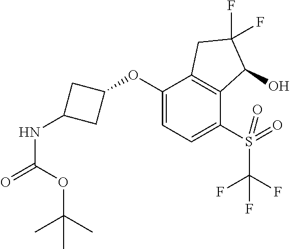 |
| | m/z: 388 (M + H − CO₂—C₄H₈)⁺ |
| 723 | 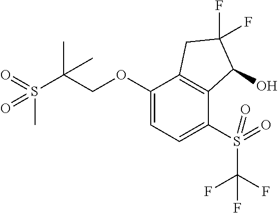 [SMILES: CC(C)(COc1ccc(S(=O)(=O)C(F)(F)F)c2c1CC(F)(F)[C@H]2O)S(C)(=O)=O] |
| | m/z: 451 (M − H)⁻ |
| 724 | 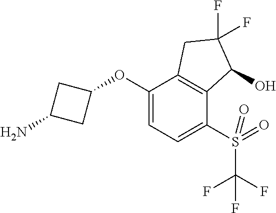 [SMILES: N[C@H]1C[C@@H](Oc2ccc(S(=O)(=O)C(F)(F)F)c3c2CC(F)(F)[C@H]3O)C1] |
| | m/z: 388 (M + H)⁺ |
| 725 | 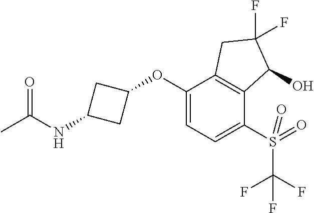 [SMILES: CC(=O)N[C@H]1C[C@@H](Oc2ccc(S(=O)(=O)C(F)(F)F)c3c2CC(F)(F)[C@H]3O)C1] |
| | m/z: 430 (M + H)⁺ |
| 726 | 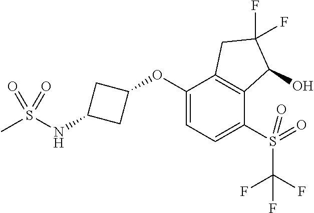 [SMILES: CS(=O)(=O)N[C@H]1C[C@@H](Oc2ccc(S(=O)(=O)C(F)(F)F)c3c2CC(F)(F)[C@H]3O)C1] |
| | m/z: 510 (M + HCO₂)⁻ |
| 727 | 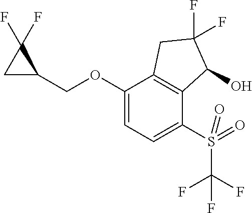 [SMILES: O=S(=O)(c1ccc(OC[C@H]2CC2(F)F)c2c1[C@H](O)C(F)(F)C2)C(F)(F)F] |
| | m/z: 407 (M − H)⁻ |
| 728 | 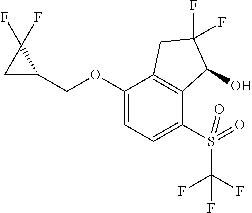 [SMILES: O=S(=O)(c1ccc(OC[C@@H]2CC2(F)F)c2c1[C@H](O)C(F)(F)C2)C(F)(F)F] |
| | m/z: 407 (M − H) |
| 729 | 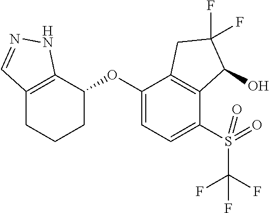 [SMILES: O=S(=O)(c1ccc(O[C@@H]2CCCc3cn[nH]c32)c2c1[C@H](O)C(F)(F)C2)C(F)(F)F] |
| | m/z: 439 (M + H)⁺ |
| 730 | 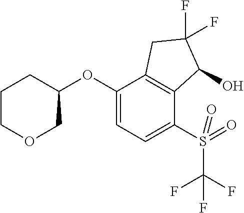 [SMILES: O=S(=O)(c1ccc(O[C@@H]2CCCOC2)c2c1[C@H](O)C(F)(F)C2)C(F)(F)F] |
| | m/z: 401 (M − H)⁻ |
| 731 | 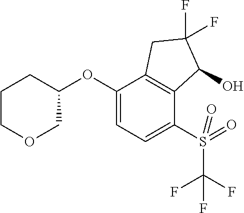 [SMILES: O=S(=O)(c1ccc(O[C@H]2CCCOC2)c2c1[C@H](O)C(F)(F)C2)C(F)(F)F] |
| | m/z: 401 (M − H)⁻ |
| 732 | 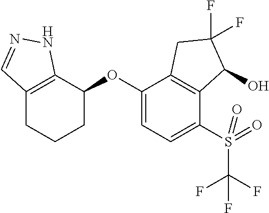 [SMILES: O=S(=O)(c1ccc(O[C@H]2CCCc3cn[nH]c32)c2c1[C@H](O)C(F)(F)C2)C(F)(F)F] |
| | m/z: 439 (M + H)⁺ |
| 733 | 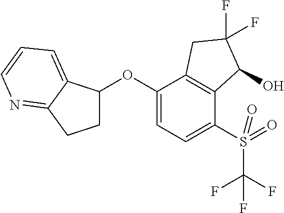 [SMILES: O=S(=O)(c1ccc(OC2CCc3ncccc32)c2c1[C@H](O)C(F)(F)C2)C(F)(F)F] |
| | m/z: 436 (M + H)⁺ |
| 734 | 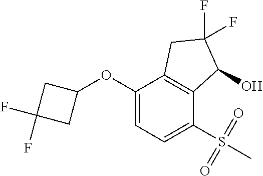 [SMILES: CS(=O)(=O)c1ccc(OC2CC(F)(F)C2)c2c1[C@H](O)C(F)(F)C2] |
| | m/z: 355 (M + H)⁺ |
| 735 | 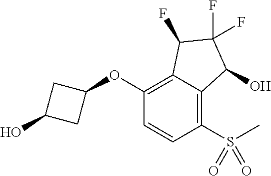 [SMILES: CS(=O)(=O)c1ccc(O[C@H]2C[C@@H](O)C2)c2c1[C@H](O)C(F)(F)[C@@H]2F] |
| | m/z: 353 (M + H)⁺ |
| 736 | 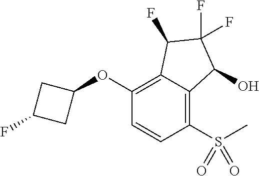 [SMILES: CS(=O)(=O)c1ccc(O[C@H]2C[C@H](F)C2)c2c1[C@H](O)C(F)(F)[C@@H]2F] |
| | m/z: 355 (M + H)⁺ |
| 737 | 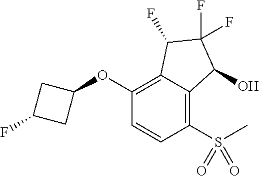 [SMILES: CS(=O)(=O)c1ccc(O[C@H]2C[C@H](F)C2)c2c1[C@H](O)C(F)(F)[C@H]2F] |
| | m/z: 355 (M + H)⁺ |
| 738 | 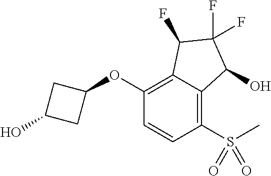 [SMILES: CS(=O)(=O)c1ccc(O[C@H]2C[C@H](O)C2)c2c1[C@H](O)C(F)(F)[C@@H]2F] |
| | m/z: 353 (M + H)⁺ |
| 739 | 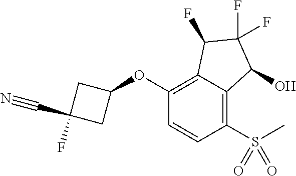 [SMILES: CS(=O)(=O)c1ccc(O[C@H]2C[C@@](F)(C#N)C2)c2c1[C@H](O)C(F)(F)[C@@H]2F] |
| | m/z: 402 (M + Na)⁺ |
| 740 | 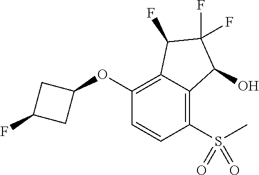 [SMILES: CS(=O)(=O)c1ccc(O[C@H]2C[C@@H](F)C2)c2c1[C@H](O)C(F)(F)[C@@H]2F] |
| | m/z: 355 (M + H)⁺ |
| 741 | 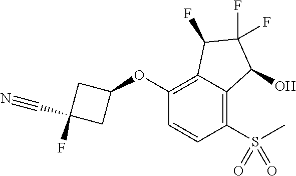 [SMILES: CS(=O)(=O)c1ccc(O[C@H]2C[C@](F)(C#N)C2)c2c1[C@H](O)C(F)(F)[C@@H]2F] |
| | m/z: 380 (M + H)⁺ |
| 742 | 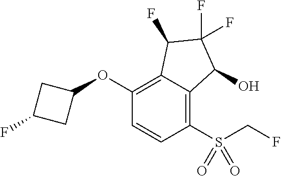 |
| | m/z: 390 (M + NH₄)⁺ |
| 743 | 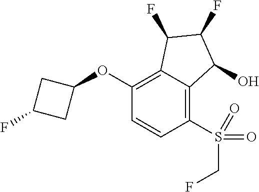 [SMILES: O=S(=O)(CF)c1ccc(O[C@H]2C[C@H](F)C2)c2c1[C@H](O)[C@H](F)[C@@H]2F] |
| | m/z: 372 (M + NH₄)⁺ |
| 744 | 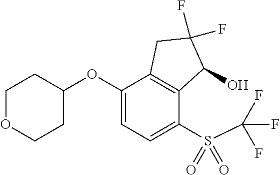 [SMILES: O=S(=O)(c1ccc(OC2CCOCC2)c2c1[C@H](O)C(F)(F)C2)C(F)(F)F] |
| | m/z: [M + NH₄]⁺ 420 |
| 745 | 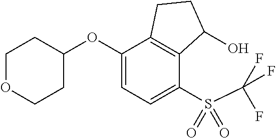 [SMILES: O=S(=O)(c1ccc(OC2CCOCC2)c2c1C(O)CC2)C(F)(F)F] |
| | m/z: [M + NH₄]⁺ 384 |
| 746 | 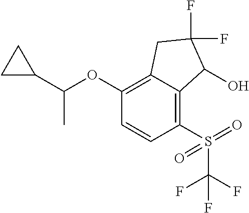 [SMILES: CC(Oc1ccc(S(=O)(=O)C(F)(F)F)c2c1CC(F)(F)C2O)C1CC1] |
| | m/z: [M + NH₄]⁺ 404 |
| 747 | 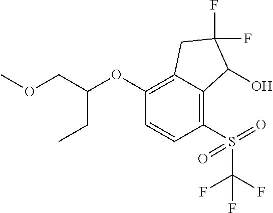 [SMILES: CCC(COC)Oc1ccc(S(=O)(=O)C(F)(F)F)c2c1CC(F)(F)C2O] |
| | m/z: [M + NH₄]⁺ 422 |
| 748 | 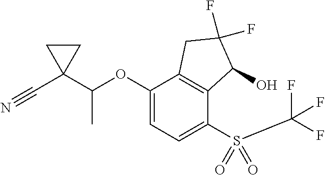 [SMILES: CC(Oc1ccc(S(=O)(=O)C(F)(F)F)c2c1CC(F)(F)[C@H]2O)C1(C#N)CC1] |
| | m/z: [M + NH₄]⁺ 429 |
| 749 | 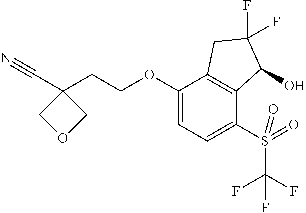 [SMILES: N#CC1(CCOc2ccc(S(=O)(=O)C(F)(F)F)c3c2CC(F)(F)[C@H]3O)COC1] |
| | m/z: [M + NH₄]⁺ 445 |
| 750 | 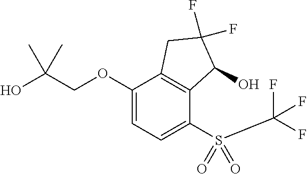 [SMILES: CC(C)(O)COc1ccc(S(=O)(=O)C(F)(F)F)c2c1CC(F)(F)[C@H]2O] |
| | m/z: [M + NH₄]⁺ 408 |
| 751 | 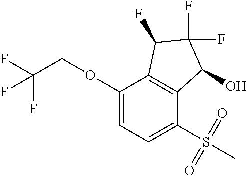 [SMILES: CS(=O)(=O)c1ccc(OCC(F)(F)F)c2c1[C@H](O)C(F)(F)[C@@H]2F] |
| | m/z: [M + NH₄]⁺ 382 |
| 752 | 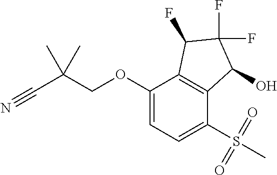 [SMILES: CC(C)(C#N)COc1ccc(S(C)(=O)=O)c2c1[C@@H](F)C(F)(F)[C@H]2O] |
| | m/z: [M + NH₄]⁺ 381 |
| 753 | 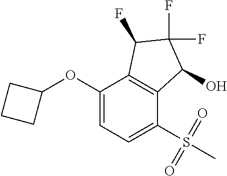 [SMILES: CS(=O)(=O)c1ccc(OC2CCC2)c2c1[C@H](O)C(F)(F)[C@@H]2F] |
| | m/z: [M + NH₄]⁺ 354 |
| 754 | 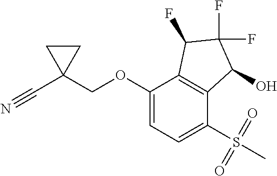 [SMILES: CS(=O)(=O)c1ccc(OCC2(C#N)CC2)c2c1[C@H](O)C(F)(F)[C@@H]2F] |
| | m/z: [M + NH₄]⁺ 379 |
| 755 | 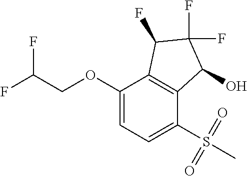 [SMILES: CS(=O)(=O)c1ccc(OCC(F)F)c2c1[C@H](O)C(F)(F)[C@@H]2F] |
| | m/z: [M + NH₄]⁺ 364 |
| 756 | 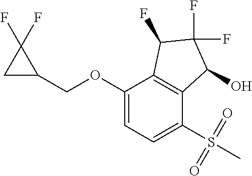 [SMILES: CS(=O)(=O)c1ccc(OCC2CC2(F)F)c2c1[C@H](O)C(F)(F)[C@@H]2F] |
| | m/z: [M + NH₄]⁺ 390 |
| 757 | 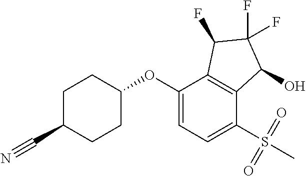 [SMILES: CS(=O)(=O)c1ccc(O[C@H]2CC[C@H](C#N)CC2)c2c1[C@H](O)C(F)(F)[C@@H]2F] |
| | m/z: [M + NH₄]⁺ 407 |
| 758 | 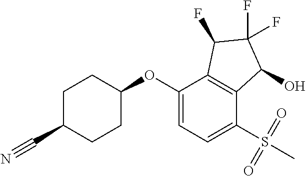 [SMILES: CS(=O)(=O)c1ccc(O[C@H]2CC[C@@H](C#N)CC2)c2c1[C@H](O)C(F)(F)[C@@H]2F] |
| | m/z: [M + NH₄]⁺ 407 |
| 759 | 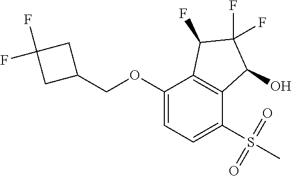 [SMILES: CS(=O)(=O)c1ccc(OCC2CC(F)(F)C2)c2c1[C@H](O)C(F)(F)[C@@H]2F] |
| | m/z: [M + NH₄]⁺ 404 |
| 760 | 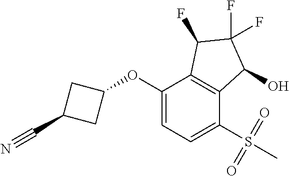 [SMILES: CS(=O)(=O)c1ccc(O[C@H]2C[C@H](C#N)C2)c2c1[C@H](O)C(F)(F)[C@@H]2F] |
| | m/z: [M + NH₄]⁺ 379 |
| 761 | 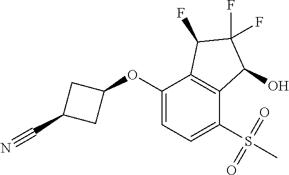 [SMILES: CS(=O)(=O)c1ccc(O[C@H]2C[C@@H](C#N)C2)c2c1[C@H](O)C(F)(F)[C@@H]2F] |
| | m/z: [M + NH₄]⁺ 379 |
| 762 | 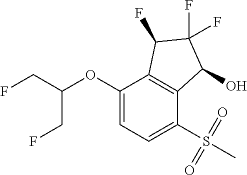 [SMILES: CS(=O)(=O)c1ccc(OC(CF)CF)c2c1[C@H](O)C(F)(F)[C@@H]2F] |
| | m/z: [M + NH₄]⁺ 378 |
| 763 | 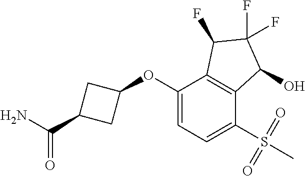 [SMILES: CS(=O)(=O)c1ccc(O[C@H]2C[C@@H](C(N)=O)C2)c2c1[C@H](O)C(F)(F)[C@@H]2F] |
| | m/z: [M + NH₄]⁺ 397 |
| 764 | 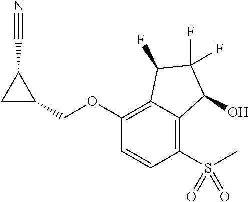 [SMILES: CS(=O)(=O)c1ccc(OC[C@@H]2C[C@@H]2C#N)c2c1[C@H](O)C(F)(F)[C@@H]2F] |
| | m/z: [M + NH₄]⁺ 379 |
| 765 | 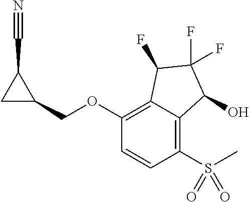 [SMILES: CS(=O)(=O)c1ccc(OC[C@H]2C[C@H]2C#N)c2c1[C@H](O)C(F)(F)[C@@H]2F] |
| | m/z: [M + NH₄]⁺ 379 |
| 766 | 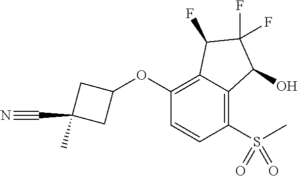 |
| | m/z: [M + NH₄]⁺ 393 |
| 767 | 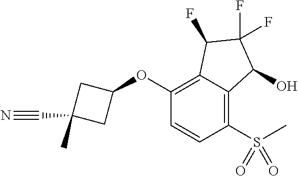 [SMILES: CS(=O)(=O)c1ccc(O[C@H]2C[C@](C)(C#N)C2)c2c1[C@H](O)C(F)(F)[C@@H]2F] |
| | m/z: [M + NH₄]⁺ 393 |
| 768 | 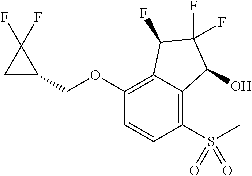 [SMILES: CS(=O)(=O)c1ccc(OC[C@@H]2CC2(F)F)c2c1[C@H](O)C(F)(F)[C@@H]2F] |
| | m/z: [M + NH₄]⁺ 390 |
| 769 | 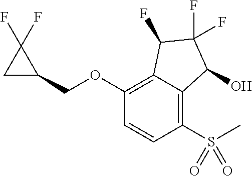 [SMILES: CS(=O)(=O)c1ccc(OC[C@H]2CC2(F)F)c2c1[C@H](O)C(F)(F)[C@@H]2F] |
| | m/z: [M + NH₄]⁺ 390 |
| 770 | 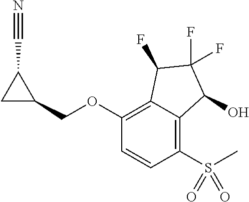 [SMILES: CS(=O)(=O)c1ccc(OC[C@H]2C[C@@H]2C#N)c2c1[C@H](O)C(F)(F)[C@@H]2F] |
| | m/z: [M + NH₄]⁺ 379 |
| 771 | 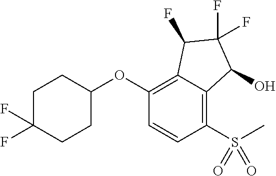 [SMILES: CS(=O)(=O)c1ccc(OC2CCC(F)(F)CC2)c2c1[C@H](O)C(F)(F)[C@@H]2F] |
| | m/z: [M + NH₄]⁺ 418 |
| 772 | 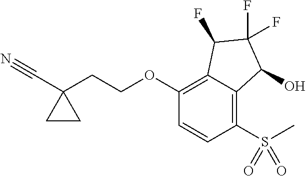 [SMILES: CS(=O)(=O)c1ccc(OCCC2(C#N)CC2)c2c1[C@H](O)C(F)(F)[C@@H]2F] |
| | m/z: [M + NH₄]⁺ 393 |
| 773 | 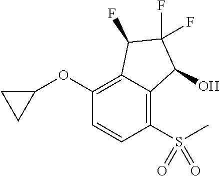 [SMILES: CS(=O)(=O)c1ccc(OC2CC2)c2c1[C@H](O)C(F)(F)[C@@H]2F] |
| | m/z: [M + NH₄]⁺ 340 |
| 774 | 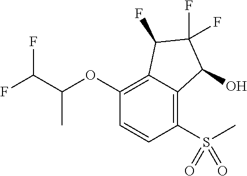 [SMILES: CC(Oc1ccc(S(C)(=O)=O)c2c1[C@@H](F)C(F)(F)[C@H]2O)C(F)F] |
| | m/z: [M + NH₄]⁺ 378 |
| 775 | 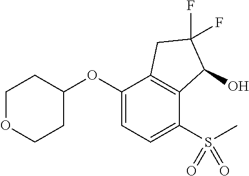 [SMILES: CS(=O)(=O)c1ccc(OC2CCOCC2)c2c1[C@H](O)C(F)(F)C2] |
| | m/z: [M + NH₄]⁺ 366 |
| 776 | 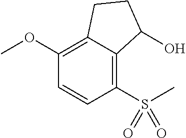 |
| | m/z: [M + NH₄]⁺ 260 |
| 777 | 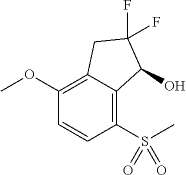 |
| | m/z: [M + NH₄]⁺ 296 |
| 778 | 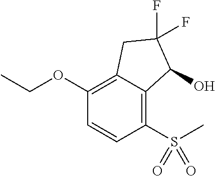 [SMILES: CCOc1ccc(S(C)(=O)=O)c2c1CC(F)(F)[C@H]2O] |
| | m/z: [M + NH₄]⁺ 310 |
| 779 | 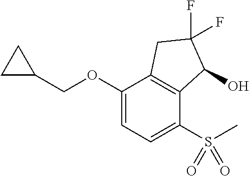 [SMILES: CS(=O)(=O)c1ccc(OCC2CC2)c2c1[C@H](O)C(F)(F)C2] |
| | m/z: [M + NH₄]⁺ 336 |
| 780 | 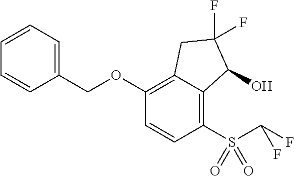 [SMILES: O=S(=O)(c1ccc(OCc2ccccc2)c2c1[C@H](O)C(F)(F)C2)C(F)F] |
| | m/z: [M + NH₄]⁺ 408 |
| 781 | 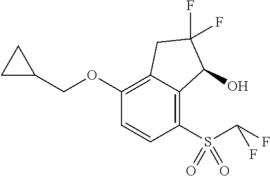 [SMILES: O=S(=O)(c1ccc(OCC2CC2)c2c1[C@H](O)C(F)(F)C2)C(F)F] |
| | m/z: [M + NH₄]⁺ 372 |
| 782 | 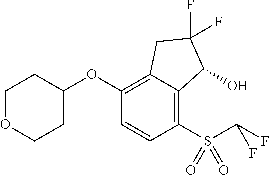 [SMILES: O=S(=O)(c1ccc(OC2CCOCC2)c2c1[C@@H](O)C(F)(F)C2)C(F)F] |
| 783 | 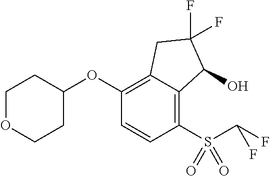 [SMILES: O=S(=O)(c1ccc(OC2CCOCC2)c2c1[C@H](O)C(F)(F)C2)C(F)F] |
| | m/z: [M + NH₄]⁺ 402 |
| 784 | 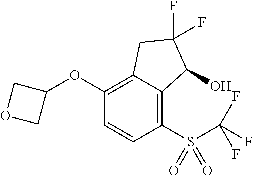 [SMILES: O=S(=O)(c1ccc(OC2COC2)c2c1[C@H](O)C(F)(F)C2)C(F)(F)F] |
| | m/z: [M + NH₄]⁺ 392 |
| 785 | 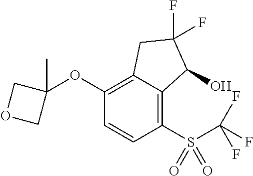 [SMILES: CC1(Oc2ccc(S(=O)(=O)C(F)(F)F)c3c2CC(F)(F)[C@H]3O)COC1] |
| | m/z: [M + NH₄]⁺ 406 |
| 786 | 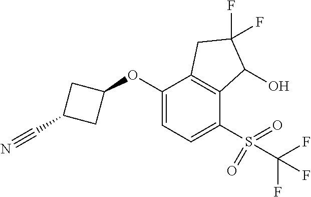 [SMILES: N#C[C@H]1C[C@H](Oc2ccc(S(=O)(=O)C(F)(F)F)c3c2CC(F)(F)C3O)C1] |
| | m/z: [M + NH₄]⁺ 415 |
| 787 | 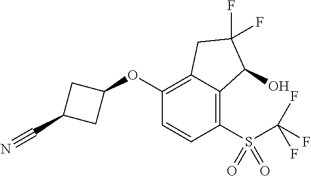 [SMILES: N#C[C@H]1C[C@@H](Oc2ccc(S(=O)(=O)C(F)(F)F)c3c2CC(F)(F)[C@H]3O)C1] |
| | m/z: [M + NH₄]⁺ 415 |
| 788 | 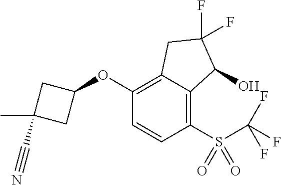 [SMILES: C[C@]1(C#N)C[C@H](Oc2ccc(S(=O)(=O)C(F)(F)F)c3c2CC(F)(F)[C@H]3O)C1] |
| | m/z: [M + NH₄]⁺ 429 |
| 789 | 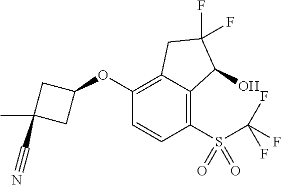 [SMILES: C[C@]1(C#N)C[C@@H](Oc2ccc(S(=O)(=O)C(F)(F)F)c3c2CC(F)(F)[C@H]3O)C1] |
| | m/z: [M + NH₄]⁺ 429 |
| 790 | 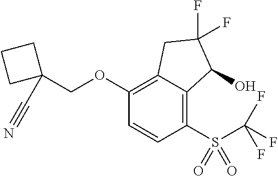 [SMILES: N#CC1(COc2ccc(S(=O)(=O)C(F)(F)F)c3c2CC(F)(F)[C@H]3O)CCC1] |
| | m/z: [M + NH₄]⁺ 429 |
| 791 | 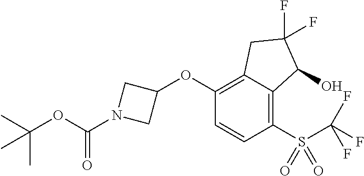 [SMILES: CC(C)(C)OC(=O)N1CC(Oc2ccc(S(=O)(=O)C(F)(F)F)c3c2CC(F)(F)[C@H]3O)C1] |
| | m/z: [M + HCl—H]⁻ 508 |
| 792 | 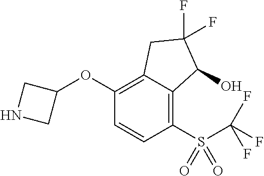 [SMILES: O=S(=O)(c1ccc(OC2CNC2)c2c1[C@H](O)C(F)(F)C2)C(F)(F)F] |
| | m/z: [M + H]⁺ 374 |
| 793 | 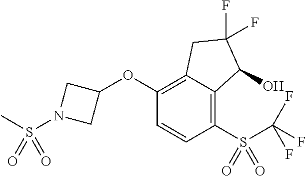 [SMILES: CS(=O)(=O)N1CC(Oc2ccc(S(=O)(=O)C(F)(F)F)c3c2CC(F)(F)[C@H]3O)C1] |
| | m/z: [M + NH₄]⁺ 469 |
| 794 | 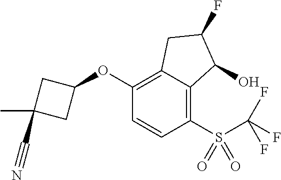 [SMILES: C[C@]1(C#N)C[C@@H](Oc2ccc(S(=O)(=O)C(F)(F)F)c3c2C[C@@H](F)[C@H]3O)C1] |
| | m/z: [M + NH₄]⁺ 411 |
| 795 | 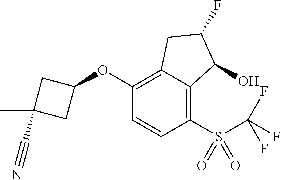 [SMILES: C[C@]1(C#N)C[C@H](Oc2ccc(S(=O)(=O)C(F)(F)F)c3c2C[C@H](F)[C@H]3O)C1] |
| | m/z: [M + NH₄]⁺ 411 |
| 796 | 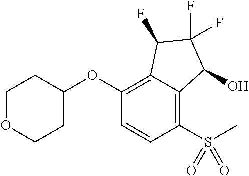 [SMILES: CS(=O)(=O)c1ccc(OC2CCOCC2)c2c1[C@H](O)C(F)(F)[C@@H]2F] |
| | m/z: [M + NH₄]⁺ 384 |
| 797 | 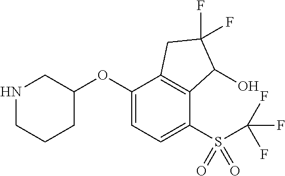 [SMILES: O=S(=O)(c1ccc(OC2CCCNC2)c2c1C(O)C(F)(F)C2)C(F)(F)F] |
| | m/z: 402 (M + H) |
| 798 | 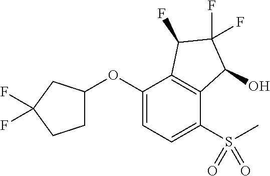 [SMILES: CS(=O)(=O)c1ccc(OC2CCC(F)(F)C2)c2c1[C@H](O)C(F)(F)[C@@H]2F] |
| | m/z: [M + NH₄]⁺ 404 |
| 799 | 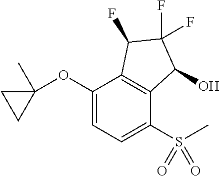 [SMILES: CC1(Oc2ccc(S(C)(=O)=O)c3c2[C@@H](F)C(F)(F)[C@H]3O)CC1] |
| | m/z: [M + NH₄]⁺ 354 |
| 800 | 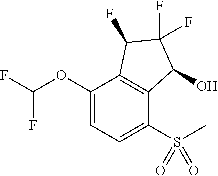 [SMILES: CS(=O)(=O)c1ccc(OC(F)F)c2c1[C@H](O)C(F)(F)[C@@H]2F] |
| | m/z: [M + NH₄]⁺ 350 |
| 801 | 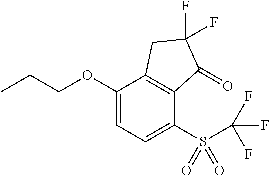 [SMILES: CCCOc1ccc(S(=O)(=O)C(F)(F)F)c2c1CC(F)(F)C2=O] |
| 802 | 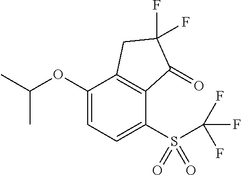 [SMILES: CC(C)Oc1ccc(S(=O)(=O)C(F)(F)F)c2c1CC(F)(F)C2=O] |
| 803 | 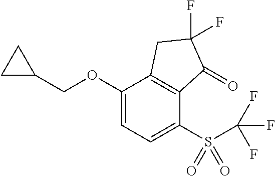 [SMILES: O=C1c2c(S(=O)(=O)C(F)(F)F)ccc(OCC3CC3)c2CC1(F)F] |
| 804 | 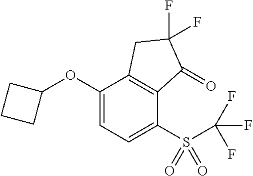 [SMILES: O=C1c2c(S(=O)(=O)C(F)(F)F)ccc(OC3CCC3)c2CC1(F)F] |
| 805 | 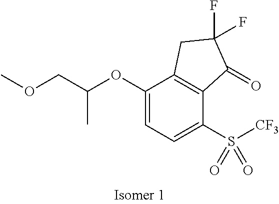 [SMILES: COCC(C)Oc1ccc(S(=O)(=O)C(F)(F)F)c2c1CC(F)(F)C2=O] |
| 806 | 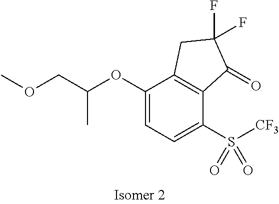 [SMILES: COCC(C)Oc1ccc(S(=O)(=O)C(F)(F)F)c2c1CC(F)(F)C2=O] |
| 807 | 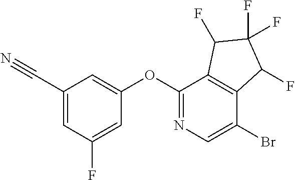 [SMILES: N#Cc1cc(F)cc(Oc2ncc(Br)c3c2C(F)C(F)(F)C3F)c1] |
| | m/z: 405/407 (M + H) |
| 808 | 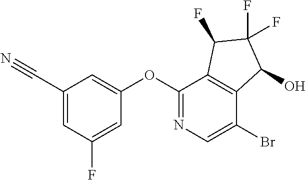 [SMILES: N#Cc1cc(F)cc(Oc2ncc(Br)c3c2[C@@H](F)C(F)(F)[C@H]3O)c1] |
| | m/z: 403/405 (M + H) |
| 809 | 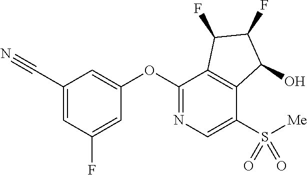 [SMILES: CS(=O)(=O)c1cnc(Oc2cc(F)cc(C#N)c2)c2c1[C@H](O)[C@H](F)[C@@H]2F] |
| | m/z: 385 (M + H) |
| 810 | 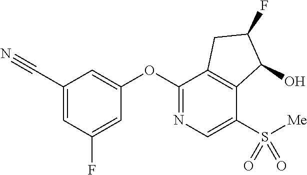 [SMILES: CS(=O)(=O)c1cnc(Oc2cc(F)cc(C#N)c2)c2c1[C@H](O)[C@H](F)C2] |
| | m/z: 367 (M + H) |
| 811 | 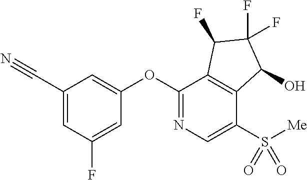 [SMILES: CS(=O)(=O)c1cnc(Oc2cc(F)cc(C#N)c2)c2c1[C@H](O)C(F)(F)[C@@H]2F] |
| | m/z: 403 (M + H) |
| 812 | 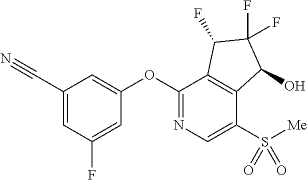 [SMILES: CS(=O)(=O)c1cnc(Oc2cc(F)cc(C#N)c2)c2c1[C@H](O)C(F)(F)[C@H]2F] |
| | m/z: 403 (M + H) |
| 813 | 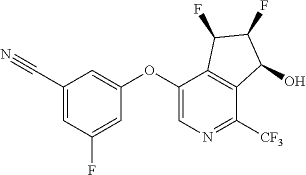 [SMILES: N#Cc1cc(F)cc(Oc2cnc(C(F)(F)F)c3c2[C@@H](F)[C@@H](F)[C@H]3O)c1] |
| | m/z: 375 (M + H) |
| 814 | 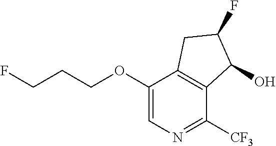 [SMILES: O[C@H]1c2c(C(F)(F)F)ncc(OCCCF)c2C[C@H]1F] |
| | m/z: 298 (M + H) |
| 815 | 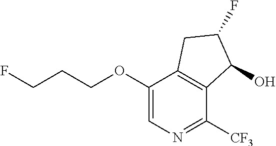 [SMILES: O[C@H]1c2c(C(F)(F)F)ncc(OCCCF)c2C[C@@H]1F] |
| | m/z: 298 (M + H) |
| 816 | 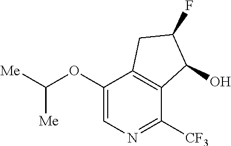 [SMILES: CC(C)Oc1cnc(C(F)(F)F)c2c1C[C@@H](F)[C@H]2O] |
| | m/z: 280 (M + H) |
| 817 | 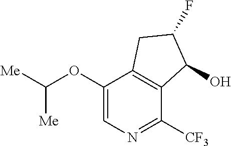 [SMILES: CC(C)Oc1cnc(C(F)(F)F)c2c1C[C@H](F)[C@H]2O] |
| | m/z: 280 (M + H) |
| 818 | 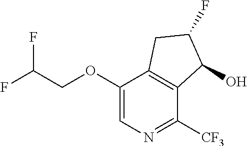 [SMILES: O[C@H]1c2c(C(F)(F)F)ncc(OCC(F)F)c2C[C@@H]1F] |
| | m/z: 302 (M + H) |
| 819 | 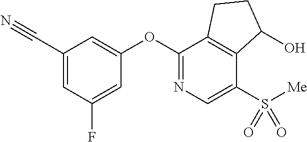 [SMILES: CS(=O)(=O)c1cnc(Oc2cc(F)cc(C#N)c2)c2c1C(O)CC2] |
| | m/z: 349 (M + H) |
| 820 | 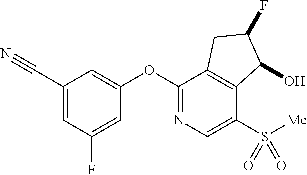 [SMILES: CS(=O)(=O)c1cnc(Oc2cc(F)cc(C#N)c2)c2c1[C@H](O)[C@H](F)C2] |
| | m/z: 367 (M + H) |
| 821 | 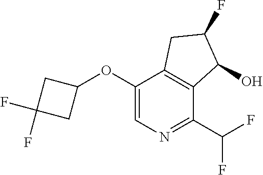 [SMILES: O[C@H]1c2c(C(F)F)ncc(OC3CC(F)(F)C3)c2C[C@H]1F] |
| | m/z: 310 (M + H) |
| 822 | 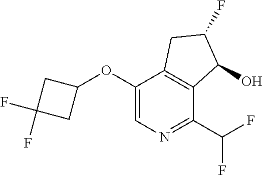 [SMILES: O[C@H]1c2c(C(F)F)ncc(OC3CC(F)(F)C3)c2C[C@@H]1F] |
| | m/z: 310 (M + H) |
| 823 | 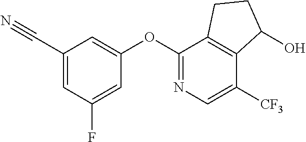 [SMILES: N#Cc1cc(F)cc(Oc2ncc(C(F)(F)F)c3c2CCC3O)c1] |
| | m/z: 339 (M + H) |
| 824 | 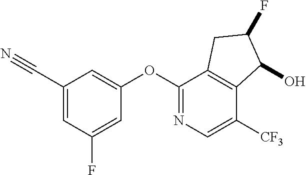 [SMILES: N#Cc1cc(F)cc(Oc2ncc(C(F)(F)F)c3c2C[C@@H](F)[C@H]3O)c1] |
| | m/z: 357 (M + H) |
| 825 | 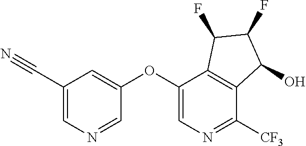 [SMILES: N#Cc1cncc(Oc2cnc(C(F)(F)F)c3c2[C@@H](F)[C@@H](F)[C@H]3O)c1] |
| | m/z: 358 (M + H) |
| 826 | 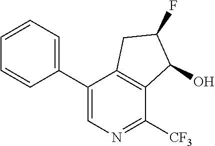 [SMILES: O[C@H]1c2c(C(F)(F)F)ncc(-c3ccccc3)c2C[C@H]1F] |
| | m/z: 298 (M + H) |
| 827 | 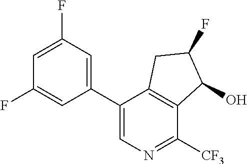 [SMILES: O[C@H]1c2c(C(F)(F)F)ncc(-c3cc(F)cc(F)c3)c2C[C@H]1F] |
| | m/z: 334 (M + H) |
| 828 | 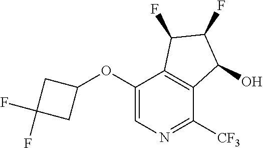 [SMILES: O[C@H]1c2c(C(F)(F)F)ncc(OC3CC(F)(F)C3)c2[C@@H](F)[C@H]1F] |
| | m/z: 346 (M + H) |
| 829 | 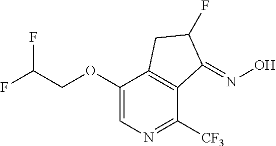 [SMILES: O/N=C1/c2c(C(F)(F)F)ncc(OCC(F)F)c2CC1F] |
| | m/z: 315 (M + H) |
| 830 | 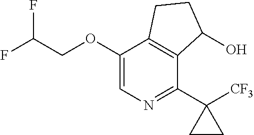 [SMILES: OC1CCc2c(OCC(F)F)cnc(C3(C(F)(F)F)CC3)c21] |
| | m/z: 324 (M + H) |
| 831 | 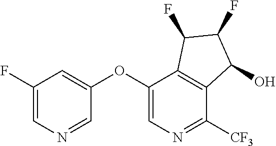 [SMILES: O[C@H]1c2c(C(F)(F)F)ncc(Oc3cncc(F)c3)c2[C@@H](F)[C@H]1F] |
| | m/z: 351 (M + H) |
| 832 | 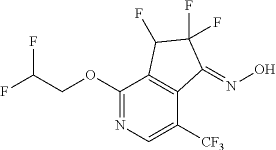 [SMILES: O/N=C1/c2c(C(F)(F)F)cnc(OCC(F)F)c2C(F)C1(F)F] |
| | m/z: 351 (M + H) |
| 833 | 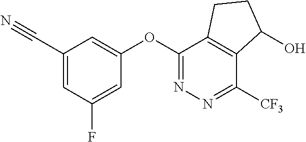 [SMILES: N#Cc1cc(F)cc(Oc2nnc(C(F)(F)F)c3c2CCC3O)c1] |
| | m/z: 340 (M + H) |

TABLE 2

| No. | Structure |
|---|---|
| II-1 | 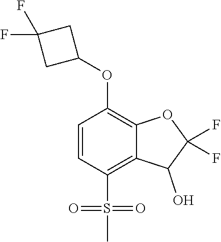 [SMILES: CS(=O)(=O)c1ccc(OC2CC(F)(F)C2)c2c1C(O)C(F)(F)O2] |
| II-2 | 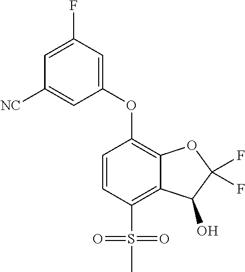 [SMILES: CS(=O)(=O)c1ccc(Oc2cc(F)cc(C#N)c2)c2c1[C@H](O)C(F)(F)O2] |
| II-3 | 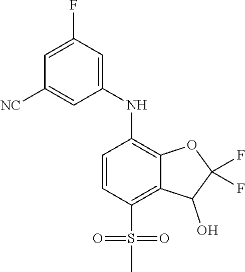 [SMILES: CS(=O)(=O)c1ccc(Nc2cc(F)cc(C#N)c2)c2c1C(O)C(F)(F)O2] |
| II-4 | 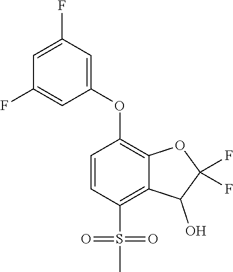 [SMILES: CS(=O)(=O)c1ccc(Oc2cc(F)cc(F)c2)c2c1C(O)C(F)(F)O2] |
| II-5 | 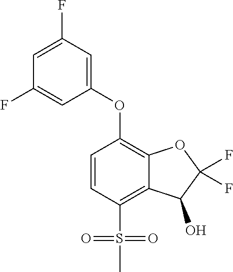 [SMILES: CS(=O)(=O)c1ccc(Oc2cc(F)cc(F)c2)c2c1[C@H](O)C(F)(F)O2] |
| II-6 | 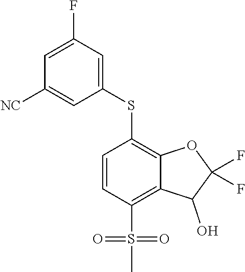 [SMILES: CS(=O)(=O)c1ccc(Sc2cc(F)cc(C#N)c2)c2c1C(O)C(F)(F)O2] |
| II-7 | 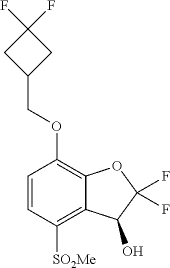 [SMILES: CS(=O)(=O)c1ccc(OCC2CC(F)(F)C2)c2c1[C@H](O)C(F)(F)O2] |
| II-8 | 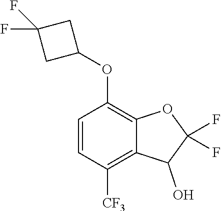 [SMILES: OC1c2c(C(F)(F)F)ccc(OC3CC(F)(F)C3)c2OC1(F)F] |
| II-9 | 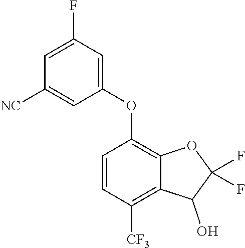 [SMILES: N#Cc1cc(F)cc(Oc2ccc(C(F)(F)F)c3c2OC(F)(F)C3O)c1] |
| II-10 | 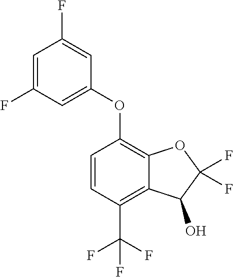 [SMILES: O[C@H]1c2c(C(F)(F)F)ccc(Oc3cc(F)cc(F)c3)c2OC1(F)F] |
| II-11 | 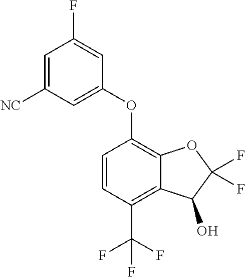 [SMILES: N#Cc1cc(F)cc(Oc2ccc(C(F)(F)F)c3c2OC(F)(F)[C@H]3O)c1] |
| II-12 | 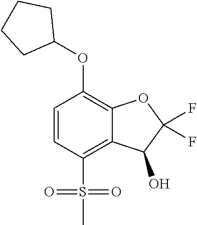 [SMILES: CS(=O)(=O)c1ccc(OC2CCCC2)c2c1[C@H](O)C(F)(F)O2] |
| II-13 | 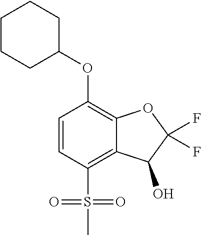 [SMILES: CS(=O)(=O)c1ccc(OC2CCCCC2)c2c1[C@H](O)C(F)(F)O2] |
| II-14 | 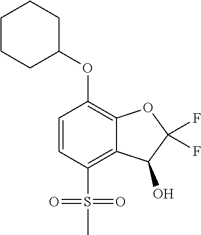 [SMILES: CS(=O)(=O)c1ccc(OC2CCCCC2)c2c1[C@H](O)C(F)(F)O2] |
| II-15 | 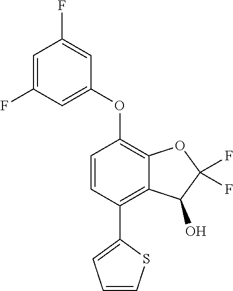 [SMILES: O[C@H]1c2c(-c3cccs3)ccc(Oc3cc(F)cc(F)c3)c2OC1(F)F] |
| II-16 | 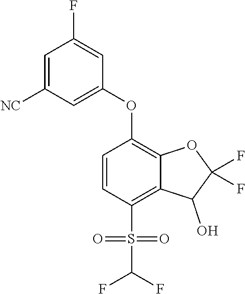 [SMILES: N#Cc1cc(F)cc(Oc2ccc(S(=O)(=O)C(F)F)c3c2OC(F)(F)C3O)c1] |
| II-17 | 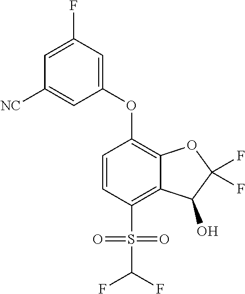 [SMILES: N#Cc1cc(F)cc(Oc2ccc(S(=O)(=O)C(F)F)c3c2OC(F)(F)[C@H]3O)c1] |
| II-18 | 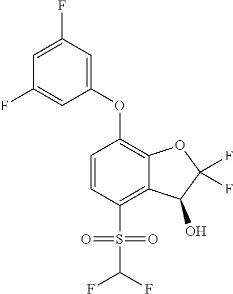 [SMILES: O=S(=O)(c1ccc(Oc2cc(F)cc(F)c2)c2c1[C@H](O)C(F)(F)O2)C(F)F] |
| II-19 | 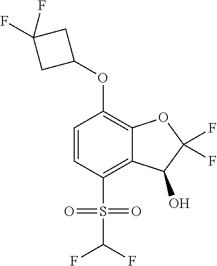 [SMILES: O=S(=O)(c1ccc(OC2CC(F)(F)C2)c2c1[C@H](O)C(F)(F)O2)C(F)F] |
| II-20 | 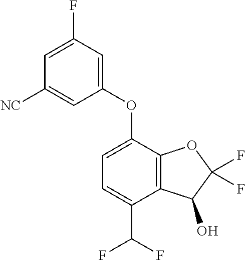 [SMILES: N#Cc1cc(F)cc(Oc2ccc(C(F)F)c3c2OC(F)(F)[C@H]3O)c1] |
| II-21 | 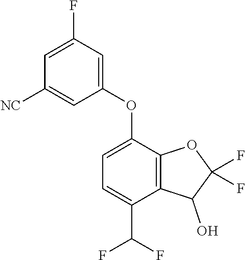 [SMILES: N#Cc1cc(F)cc(Oc2ccc(C(F)F)c3c2OC(F)(F)C3O)c1] |
| II-22 | 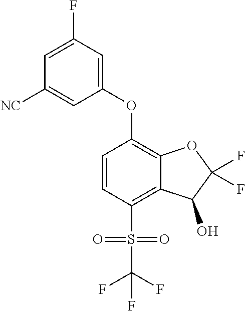 [SMILES: N#Cc1cc(F)cc(Oc2ccc(S(=O)(=O)C(F)(F)F)c3c2OC(F)(F)[C@H]3O)c1] |
| II-23 | 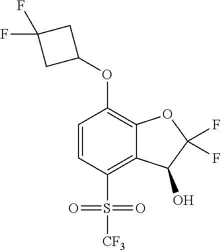 [SMILES: O=S(=O)(c1ccc(OC2CC(F)(F)C2)c2c1[C@H](O)C(F)(F)O2)C(F)(F)F] |
| II-24 | 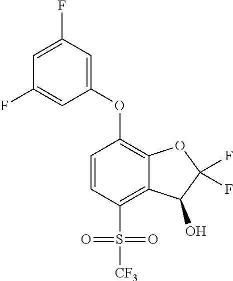 [SMILES: O=S(=O)(c1ccc(Oc2cc(F)cc(F)c2)c2c1[C@H](O)C(F)(F)O2)C(F)(F)F] |
| II-25k | 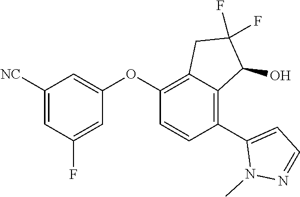 [SMILES: Cn1nccc1-c1ccc(Oc2cc(F)cc(C#N)c2)c2c1[C@H](O)C(F)(F)C2] |
| II-25l | 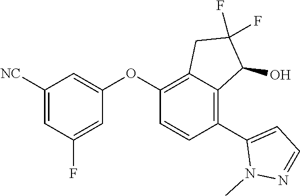 [SMILES: Cn1nccc1-c1ccc(Oc2cc(F)cc(C#N)c2)c2c1[C@H](O)C(F)(F)C2] |
| II-25m | 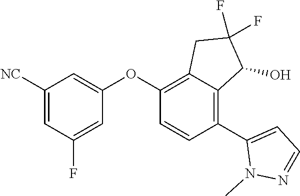 [SMILES: Cn1nccc1-c1ccc(Oc2cc(F)cc(C#N)c2)c2c1[C@@H](O)C(F)(F)C2] |
| II-26 | 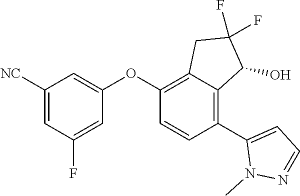 [SMILES: Cn1nccc1-c1ccc(Oc2cc(F)cc(C#N)c2)c2c1[C@@H](O)C(F)(F)C2] |
| II-27 | 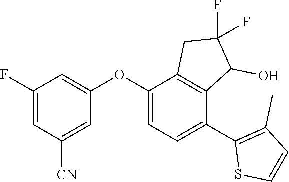 [SMILES: Cc1ccsc1-c1ccc(Oc2cc(F)cc(C#N)c2)c2c1C(O)C(F)(F)C2] |
| II-28 | 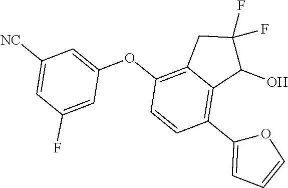 [SMILES: N#Cc1cc(F)cc(Oc2ccc(-c3ccco3)c3c2CC(F)(F)C3O)c1] |
| II-29 | 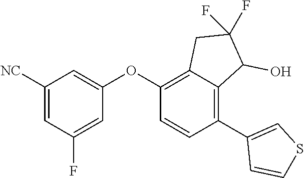 [SMILES: N#Cc1cc(F)cc(Oc2ccc(-c3ccsc3)c3c2CC(F)(F)C3O)c1] |
| II-30 | 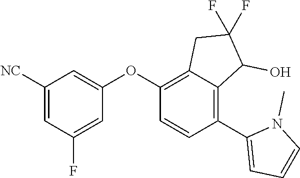 [SMILES: Cn1cccc1-c1ccc(Oc2cc(F)cc(C#N)c2)c2c1C(O)C(F)(F)C2] |
| II-31 | 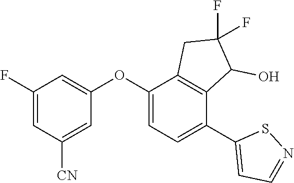 [SMILES: N#Cc1cc(F)cc(Oc2ccc(-c3ccns3)c3c2CC(F)(F)C3O)c1] |
| II-32 | 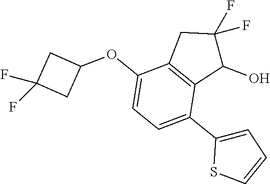 [SMILES: OC1c2c(-c3cccs3)ccc(OC3CC(F)(F)C3)c2CC1(F)F] |
| II-33 | 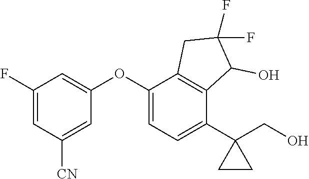 [SMILES: N#Cc1cc(F)cc(Oc2ccc(C3(CO)CC3)c3c2CC(F)(F)C3O)c1] |
| II-34 | 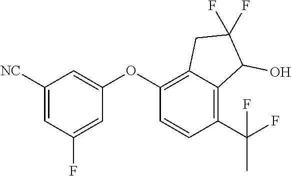 [SMILES: CC(F)(F)c1ccc(Oc2cc(F)cc(C#N)c2)c2c1C(O)C(F)(F)C2] |
| II-35 | 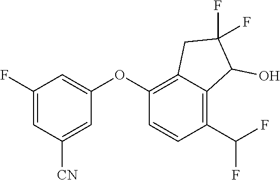 [SMILES: N#Cc1cc(F)cc(Oc2ccc(C(F)F)c3c2CC(F)(F)C3O)c1] |
| II-36 | 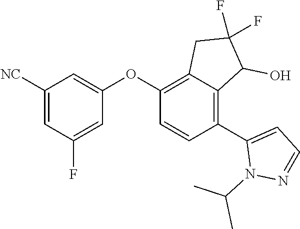 [SMILES: CC(C)n1nccc1-c1ccc(Oc2cc(F)cc(C#N)c2)c2c1C(O)C(F)(F)C2] |
| II-37 | 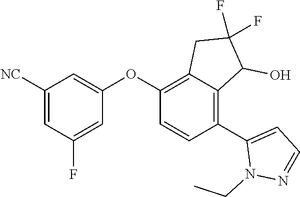 [SMILES: CCn1nccc1-c1ccc(Oc2cc(F)cc(C#N)c2)c2c1C(O)C(F)(F)C2] |
| II-38 | 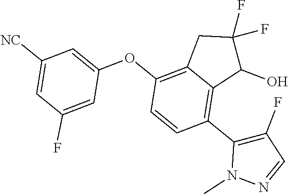 [SMILES: Cn1ncc(F)c1-c1ccc(Oc2cc(F)cc(C#N)c2)c2c1C(O)C(F)(F)C2] |
| II-39 | 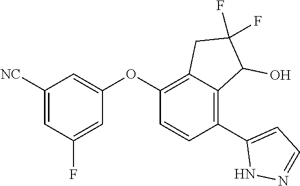 [SMILES: N#Cc1cc(F)cc(Oc2ccc(-c3ccn[nH]3)c3c2CC(F)(F)C3O)c1] |
| II-40 | 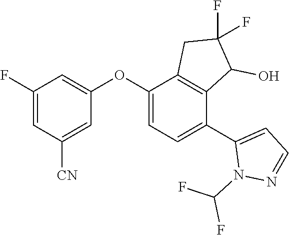 [SMILES: N#Cc1cc(F)cc(Oc2ccc(-c3ccnn3C(F)F)c3c2CC(F)(F)C3O)c1] |
| II-41 | 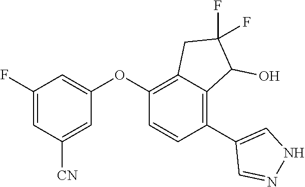 [SMILES: N#Cc1cc(F)cc(Oc2ccc(-c3cn[nH]c3)c3c2CC(F)(F)C3O)c1] |
| II-42 | 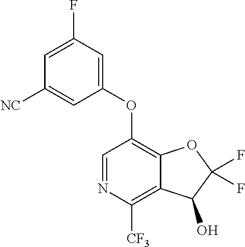 [SMILES: N#Cc1cc(F)cc(Oc2cnc(C(F)(F)F)c3c2OC(F)(F)[C@H]3O)c1] |
| II-43 | 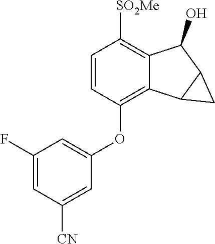 [SMILES: CS(=O)(=O)c1ccc(Oc2cc(F)cc(C#N)c2)c2c1[C@@H](O)C1CC21] |
| II-44 | 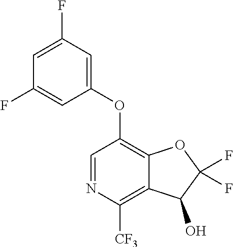 [SMILES: O[C@H]1c2c(C(F)(F)F)ncc(Oc3cc(F)cc(F)c3)c2OC1(F)F] |
| II-45 | 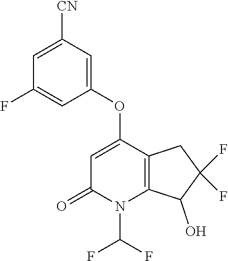 [SMILES: N#Cc1cc(F)cc(Oc2cc(=O)n(C(F)F)c3c2CC(F)(F)C3O)c1] |
| II-46 | 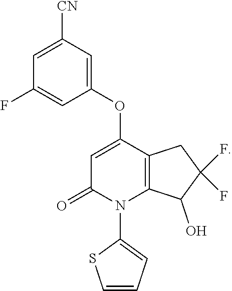 [SMILES: N#Cc1cc(F)cc(Oc2cc(=O)n(-c3cccs3)c3c2CC(F)(F)C3O)c1] |
| II-47 | 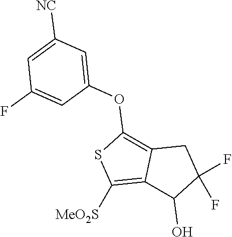 [SMILES: CS(=O)(=O)c1sc(Oc2cc(F)cc(C#N)c2)c2c1C(O)C(F)(F)C2] |
| II-48 | 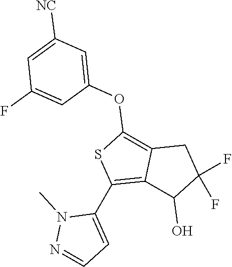 [SMILES: Cn1nccc1-c1sc(Oc2cc(F)cc(C#N)c2)c2c1C(O)C(F)(F)C2] |
| II-49 | 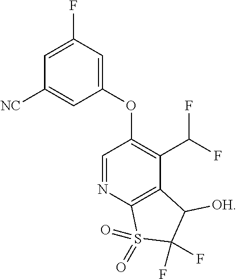 [SMILES: N#Cc1cc(F)cc(Oc2cnc3c(c2C(F)F)C(O)C(F)(F)S3(=O)=O)c1] |
| II-50 | 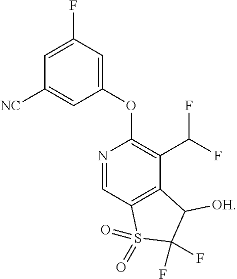 [SMILES: N#Cc1cc(F)cc(Oc2ncc3c(c2C(F)F)C(O)C(F)(F)S3(=O)=O)c1] |
| II-51 | 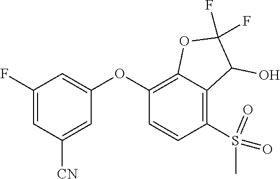 [SMILES: CS(=O)(=O)c1ccc(Oc2cc(F)cc(C#N)c2)c2c1C(O)C(F)(F)O2] |
| II-52 | 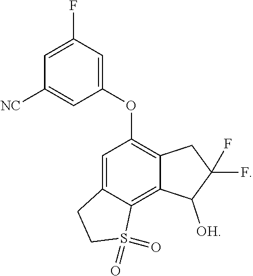 [SMILES: N#Cc1cc(F)cc(Oc2cc3c(c4c2CC(F)(F)C4O)S(=O)(=O)CC3)c1] |
| II-53 | 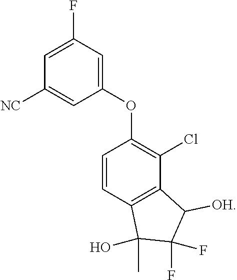 [SMILES: CC1(O)c2ccc(Oc3cc(F)cc(C#N)c3)c(Cl)c2C(O)C1(F)F] |
| II-54 | 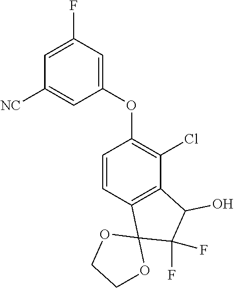 [SMILES: N#Cc1cc(F)cc(Oc2ccc3c(c2Cl)C(O)C(F)(F)C32OCCO2)c1] |
| II-55 | 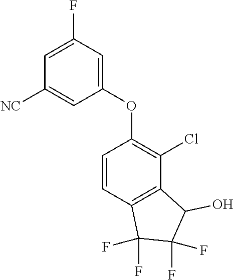 [SMILES: N#Cc1cc(F)cc(Oc2ccc3c(c2Cl)C(O)C(F)(F)C3(F)F)c1] |
| II-56 | 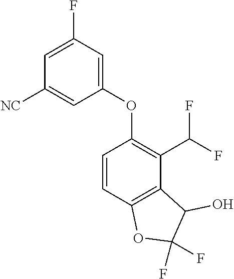 [SMILES: N#Cc1cc(F)cc(Oc2ccc3c(c2C(F)F)C(O)C(F)(F)O3)c1] |
| II-57 | 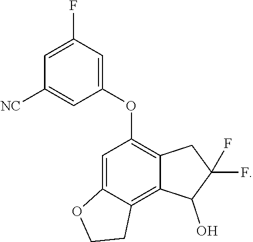 [SMILES: N#Cc1cc(F)cc(Oc2cc3c(c4c2CC(F)(F)C4O)CCO3)c1] |
| II-58 | 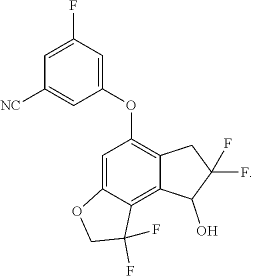 [SMILES: N#Cc1cc(F)cc(Oc2cc3c(c4c2CC(F)(F)C4O)C(F)(F)CO3)c1] |
| II-59 | 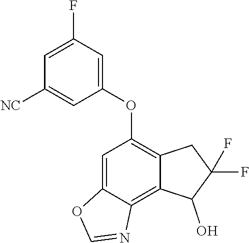 [SMILES: N#Cc1cc(F)cc(Oc2cc3ocnc3c3c2CC(F)(F)C3O)c1] |
| II-60 | 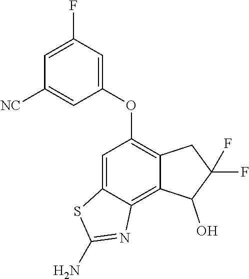 [SMILES: N#Cc1cc(F)cc(Oc2cc3sc(N)nc3c3c2CC(F)(F)C3O)c1] |

Pharmaceutical compositions: A composition of the present disclosure may be formulated in any suitable pharmaceutical formulation. A pharmaceutical composition of the present disclosure typically contains an active ingredient (e.g., a compound of Formula I′, I, I-A, I-B, I-C, I-D, I-E, I-F, I-G, I-H, I-I, I-J, I-K, II, II-A or II-B, or a pharmaceutically acceptable salt and/or coordination complex thereof), and one or more pharmaceutically acceptable excipients, carriers, including but not limited to, inert solid diluents and fillers, diluents, sterile aqueous solution and various organic solvents, permeation enhancers, solubilizers and adjuvants. A composition of the present disclosure may be formulated in any suitable pharmaceutical formulation. In some embodiments, the pharmaceutical acceptable carriers, excipients are selected from water, alcohol, glycerol, chitosan, alginate, chondroitin, Vitamin E, mineral oil, and dimethyl sulfoxide (DMSO). In some embodiments, the present disclosure provides a pharmaceutical composition comprising Compound 231 and a pharmaceutically acceptable carrier.

Pharmaceutical formulations may be provided in any suitable form, which may depend on the route of administration. In some embodiments, the pharmaceutical composition disclosed herein can be formulated in dosage form for administration to a subject. In some embodiments, the pharmaceutical composition is formulated for oral, intravenous, intraarterial, aerosol, parenteral, buccal, topical, transdermal, rectal, intramuscular, subcutaneous, intraosseous, intranasal, intrapulmonary, transmucosal, inhalation, and/or intraperitoneal administration. In some embodiments, the dosage form is formulated for oral intervention administration. For example, the pharmaceutical composition can be formulated in the form of a pill, a tablet, a capsule, an inhaler, a liquid suspension, a liquid emulsion, a gel, or a powder. In some embodiments, the pharmaceutical composition can be formulated as a unit dosage in liquid, gel, semi-liquid, semi-solid, foam, or solid form.

The amount of each compound administered will be dependent on the mammal being treated, the severity of the disorder or condition, the rate of administration, the disposition of the compound and the discretion of the prescribing physician. However, an effective dosage may be in the range of about 0.001 to about 100 mg per kg body weight per day, in single or divided doses. In some instances, dosage levels below the lower limit of the aforesaid range may be more than adequate, while in other cases still larger doses may be employed without causing any harmful side effect, e.g., by dividing such larger doses into several small doses for administration throughout the day.

In some embodiments, the disclosure provides a pharmaceutical composition comprising an amount of a HIF-2α inhibitor formulated for administration to a subject in need thereof. In some embodiments, the pharmaceutical composition comprises between about 0.0001-500 g, 0.001-250 g, 0.01-100 g, 0.1-50 g, or 1-10 g of HIF-2α inhibitor. In some embodiments, the pharmaceutical composition comprises about or more than about 0.0001 g, 0.001 g, 0.01 g, 0.1, 0.5 g, 1 g, 2 g, 3 g, 4 g, 5 g, 6 g, 7 g, 8 g, 9 g, 10 g, 15 g, 20 g, 25 g, 50 g, 100 g, 200 g, 250 g, 300 g, 350 g, 400 g, 450 g, 500 g, or more of a HIF-2α inhibitor. In some embodiments, the pharmaceutical composition comprises between 0.001-2 g of a HIF-2α inhibitor in a single dose. In some embodiments, the pharmaceutical composition comprises an amount between about 50-150 g of a HIF-2α inhibitor. In some embodiments, the therapeutic amount can be an amount between about 0.001-0.1 g of a HIF-2a inhibitor. In some embodiments, the therapeutic amount can be an amount between about 0.01-30 g of a HIF-2α inhibitor.

In some embodiments, a therapeutically effective amount of HIF-2α inhibitor, which can be a daily amount administered over the course of a period of treatment, can sufficiently provide any one or more of the therapeutic effects described herein. As an example, the therapeutic effective amount can be in the range of about 0.001-1000 mg/kg body weight, 0.01-500 mg/kg body weight, 0.01-100 mg/kg body weight, 0.01-30 mg/kg body weight, 0.1-200 mg/kg body weight, 3-200 mg/kg body weight, 5-500 mg/kg body weight, 10-100 mg/kg body weight, 10-1000 mg/kg body weight, 50-200 mg/kg body weight, 100-1000 mg/kg body weight, 200-500 mg/kg body weight, 250-350 mg/kg body weight, or 300-600 mg/kg body weight of a HIF-2α inhibitor. In some embodiments, the therapeutic amount can be about or more than about 0.001 mg/kg body weight, 0.01 mg/kg body weight, 0.1 mg/kg body weight, 0.5 mg/kg body weight, 1 mg/kg body weight, 2 mg/kg body weight, 3 mg/kg body weight, 4 mg/kg body weight, 5 mg/kg body weight, 6 mg/kg body weight, 7 mg/kg body weight, 8 mg/kg body weight, 9 mg/kg body weight, 10 mg/kg body weight, 15 mg/kg body weight, 20 mg/kg body weight, 25 mg/kg body weight, 50 mg/kg body weight, 100 mg/kg body weight, 200 mg/kg body weight, 250 mg/kg body weight, 300 mg/kg body weight, 350 mg/kg body weight, 400 mg/kg body weight, 450 mg/kg body weight, 500 mg/kg body weight, 600 mg/kg body weight, 800 mg/kg body weight, 1000 mg/kg body weight, or more of a HIF-2a inhibitor. In some embodiments, the effective amount is at least about 0.01 mg/kg body weight of a HIF-2α inhibitor. In some embodiments, the effective amount is an amount between about 0.01-30 mg/kg body weight of a HIF-2α inhibitor. In some embodiments, the therapeutic amount can be an amount between about 50-150 mg/kg body weight of a HIF-2α inhibitor.

In some embodiments, the composition is provided in one or more unit doses. For example, the composition can be administered in 1, 2, 3, 4, 5, 6, 7, 14, 30, 60, or more doses. Such amount can be administered each day, for example in individual doses administered once, twice, or three or more times a day. However, dosages stated herein on a per day basis should not be construed to require administration of the daily dose each and every day. For example, if one of the agents is provided in a suitably slow-release form, two or more daily dosage amounts can be administered at a lower frequency, e.g., as a depot every second day to once a month or even longer. Most typically and conveniently for the subject, a HIF-2α inhibitor can be administered once a day, for example in the morning, in the evening or during the day.

The unit doses can be administered simultaneously or sequentially. The composition can be administered for an extended treatment period. Illustratively, the treatment period can be at least about one month, for example at least about 3 months, at least about 6 months or at least about 1 year. In some cases, administration can continue for substantially the remainder of the life of the subject.

Pharmaceutical composition for oral administration: In some embodiments, the disclosure provides a pharmaceutical composition for oral administration containing at least one compound of the present disclosure and a pharmaceutical excipient suitable for oral administration. The composition may be in the form of a solid, liquid, gel, semi-liquid, or semi-solid. In some embodiments, the composition further comprises a second agent.

In some embodiments, the invention provides a solid pharmaceutical composition for oral administration containing: (i) a HIF-2α inhibitor; and (ii) a pharmaceutical excipient suitable for oral administration. In some embodiments, the composition further contains: (iii) a third agent or even a fourth agent. In some embodiments, each compound or agent is present in a therapeutically effective amount. In other embodiments, one or more compounds or agents is present in a sub-therapeutic amount, and the compounds or agents act synergistically to provide a therapeutically effective pharmaceutical composition.

Pharmaceutical compositions of the disclosure suitable for oral administration can be presented as discrete dosage forms, such as hard or soft capsules, cachets, troches, lozenges, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion, or dispersible powders or granules, or syrups or elixirs. Such dosage forms can be prepared by any of the methods of pharmacy, which typically include the step of bringing the active ingredient(s) into association with the carrier. In general, the composition are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient(s) in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This disclosure further encompasses anhydrous pharmaceutical composition and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the composition for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical composition and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical composition and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the composition of the disclosure to provide tablets that disintegrate when exposed to an aqueous environment. Too much of a disintegrant may produce tablets which may disintegrate in the bottle. Too little may be insufficient for disintegration to occur and may alter the rate and extent of release of the active ingredient(s) from the dosage form. A sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical composition and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical composition and dosage forms of the disclosure include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical composition and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group of glycerides, vegetable oils, hydrogenated vegetable oils, fatty acids, and sterols; polyoxyethylene sterols, derivatives, and analogues thereof, polyoxyethylated vitamins and derivatives thereof, polyoxyethylene-polyoxypropylene block copolymers; and mixtures thereof; polyethylene glycol sorbitan fatty acid esters and hydrophilic transesterification products of a polyol with at least one member of the group of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group of vegetable oils, hydrogenated vegetable oils, and triglycerides.

In one embodiment, the composition may include a solubilizer to ensure good solubilization and/or dissolution of the compound of the present disclosure and to minimize precipitation of the compound of the present disclosure. This can be especially important for composition for non-oral use, e.g., composition for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, F-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, F-caprolactone and isomers thereof, 6-valerolactone and isomers thereof, β-butyrolactone and isomers thereof, and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. If present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

Pharmaceutical composition for topical (e.g., transdermal) delivery. In some embodiments, the disclosure provides a pharmaceutical composition for transdermal delivery containing a compound of the present disclosure and a pharmaceutical excipient suitable for transdermal delivery. The composition may be in the form of a solid, liquid, gel, foam, semi-liquid, or semi-solid. In some embodiments, the composition further comprises a second agent. In some embodiments, the disclosure provides a pharmaceutical composition suitable for rectal administration, such as a suppository or an enema.

Composition of the present disclosure can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical composition also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Formulations for topical administration may include ointments, lotions, creams, gels (e.g., poloxamer gel), drops, suppositories, sprays, liquids and powders. Topical administration includes rectal administration, including, for example, suppositories and enemas. Conventional pharmaceutical carriers, aqueous, powder or oily bases, thickeners and the like may be necessary or desirable. The disclosed compositions can be administered, for example, in a microfiber, polymer (e.g., collagen), nanosphere, aerosol, lotion, cream, fabric, plastic, tissue engineered scaffold, matrix material, tablet, implanted container, powder, oil, resin, wound dressing, bead, microbead, slow release bead, capsule, injectables, intravenous drips, pump device, silicone implants, or any bio-engineered materials.

Pharmaceutical composition for injection: In some embodiments, the disclosure provides a pharmaceutical composition for injection containing a compound of the present disclosure and a pharmaceutical excipient suitable for injection. Components and amounts of agents in the composition are as described herein.

The forms in which the novel composition of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Other pharmaceutical compositions: Pharmaceutical compositions may also be prepared from composition described herein and one or more pharmaceutically acceptable excipients suitable for transdermal, inhalative, sublingual, buccal, rectal, intraosseous, intraocular, intranasal, epidural, or intraspinal administration. Preparations for such pharmaceutical composition are well-known in the art. See, e.g., See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

The compounds of the present invention can also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., “Methods in Cell Biology”, Volume XIV, ISBN: 978-0-12-564114-2, Academic Press, New York, N.W., p. 33 (1976) and Medina, Zhu, and Kairemo, “Targeted liposomal drug delivery in cancer”, Current Pharm. Des. 10: 2981-2989, 2004. For additional information regarding drug formulation and administration, see “Remington: The Science and Practice of Pharmacy,” Lippincott Williams & Wilkins, Philadelphia, ISBN-10: 0781746736, 21st Edition (2005).

When the method of the invention involves combination therapy, for example, wherein a secondary agent is co-administered with a HIF-2α inhibitor, the agents may be administered separately, at the same, or at different times of the day, or they may be administered in a single composition. In the combination therapies of the invention, each agent can be administered in an “immediate release” manner or in a “controlled release manner.” When the additional active agent is a corticosteroid, for instance, any dosage form containing both active agents, such as both the HIF-2α inhibitor and the corticosteroid, can provide for immediate release or controlled release of the corticosteroid, and either immediate release or controlled release of the HIF-2α inhibitor. In other formulations of the present disclosure, two or more additional active agents, which may or may not be in the same class of drug, can be present in combination, along with the HIF-2α inhibitor. In such a case, the effective amount of either or each individual additional active agent present will generally be reduced relative to the amount that would be required if only a single added agent were used.

### Examples

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Synthesis of 3-[(1S)-7-(difluoromethylsulfonyl)-2,2-difluoro-1-hydroxy-indan-4-yl]oxy-5-fluoro-benzonitrile (Compound 15)

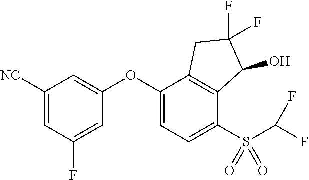 [SMILES: N#Cc1cc(F)cc(Oc2ccc(S(=O)(=O)C(F)F)c3c2CC(F)(F)[C@H]3O)c1]

Step A: Preparation of 3-((7-((difluoromethyl)sulfonyl)-2,3-dihydrospiro[indene-1,2′-[1,3]dioxolan]-4-yl)oxy)-5-fluorobenzonitrile: A mixture of 3-fluoro-5-hydroxy-benzonitrile (1.33 g, 9.7 mmol), 7′-(difluoromethylsulfonyl)-4′-fluoro-spiro[1,3-dioxolane-2,1′-indane] (1.0 g, 3.24 mmol), and cesium bicarbonate (1.26 g, 6.5 mmol) in 1-methyl-2-pyrrolidone (1.8 mL) was heated under N₂ at 110° C. (microwave) for 1 hour and 5 minutes. The reaction was repeated ten times. The reaction mixtures were combined, diluted with EtOAc, and washed twice with 1 N NaOH. The combined aqueous layer was extracted with EtOAc. The EtOAc extracts were combined and washed with brine, dried over Na₂SO₄, filtered, and concentrated to about 100 mL to give a suspension. The suspension was filtered to give 3-((7-((difluoromethyl)sulfonyl)-2,3-dihydrospiro[indene-1,2′-[1,3]dioxolan]-4-yl)oxy)-5-fluorobenzonitrile as an off-white solid (6.25 g). The filtrate was diluted with EtOAc, washed with brine (3×), dried over Na₂SO₄, filtered, and concentrated. The residue was purified by flash column chromatography on silica gel with EtOAc/hexane (0% to 40%) to give additional 3-((7-((difluoromethyl)sulfonyl)-2,2-difluoro-2,3-dihydrospiro[indene-1,2′-[1,3]dioxolan]-4-yl)oxy)-5-fluorobenzonitrile (3.3 g, 69% combined yield) as a white solid. LCMS ESI (+) m/z 426 (M+H).

Step B: Preparation of 3-((7-((difluoromethyl)sulfonyl)-1-oxo-2,3-dihydro-1H-inden-4-yl)oxy)-5-fluorobenzonitrile: A mixture of 3-((7-((difluoromethyl)sulfonyl)-2,3-dihydrospiro[indene-1,2′-[1,3]dioxolan]-4-yl)oxy)-5-fluorobenzonitrile (10.9 g, 25.6 mmol) and PPTS (667 mg, 2.66 mmol) in acetone (100 mL)/water (15 mL) was heated at 82° C. for 5 hours and then 75° C. overnight. The reaction mixture was cooled to room temperature, concentrated under reduced pressure, diluted with EtOAc, washed with saturated aqueous NaHCO₃, dried over MgSO₄, filtered, and concentrated. The residue was filtered and washed with water. The solid obtained was briefly dried under vacuum at 50° C. and then triturated with EtOAc/hexane to give 3-((7-((difluoromethyl)sulfonyl)-1-oxo-2,3-dihydro-1H-inden-4-yl)oxy)-5-fluorobenzonitrile (8 g). Flash column chromatography of the mother liquor on silica gel with EtOAc/hexane (0% to 80%) provided additional 3-((7-((difluoromethyl)sulfonyl)-1-oxo-2,3-dihydro-1H-inden-4-yl)oxy)-5-fluorobenzonitrile (1.3 g, combined 9.3 g, quant. yield). LCMS ESI (+) m/z 382 (M+H).

Step C: Preparation of (E, Z)-3-((1-(butylimino)-7-((difluoromethyl)sulfonyl)-2,3-dihydro-1H-inden-4-yl)oxy)-5-fluorobenzonitrile: A mixture of 3-((7-((difluoromethyl)sulfonyl)-1-oxo-2,3-dihydro-1H-inden-4-yl)oxy)-5-fluorobenzonitrile (1.42 g, 3.72 mmol), butylamine (6.0 mL) and 5 drops of trifluoroacetic acid (˜0.1 mL) in benzene (40 mL) was refluxed overnight with removal of water using a Dean-Stark trap. The reaction mixture was concentrated under reduced pressure, diluted with methyl tert-butyl ether, washed with saturated aqueous NaHCO₃ and brine, dried over Na₂SO₄, filtered, and concentrated. The residue was used in the next step without further purification.

Step D: Preparation of 3-((7-((difluoromethyl)sulfonyl)-2,2-difluoro-1-oxo-2,3-dihydro-1H-inden-4-yl)oxy)-5-fluorobenzonitrile: A mixture of (E, Z)-3-((1-(butylimino)-7-((difluoromethyl)sulfonyl)-2,3-dihydro-1H-inden-4-yl)oxy)-5-fluorobenzonitrile (1.29 g, 3 mmol, crude from step C), Selectfluor® (2.62 g, 7.4 mmol) and sodium sulfate (4 g, 28.2 mmol) under N₂ was heated at 82° C. for 4 hours. After cooling to room temperature, concentrated HCl (37%, 3 mL) was added. The mixture was stirred at room temperature for 15 minutes and then concentrated under reduced pressure. The residue was diluted with methyl t-butyl ether, washed with half saturated aqueous NaHCO₃ and then brine, dried over Na₂SO₄, filtered, and triturated with EtOAc/hexane to give 3-((7-((difluoromethyl)sulfonyl)-2,2-difluoro-1-oxo-2,3-dihydro-1H-inden-4-yl)oxy)-5-fluorobenzonitrile as an off-white solid (0.5 g). The mother liquor was purified by flash column chromatography with EtOAc/hexane (5% to 40%) to give additional 3-((7-((difluoromethyl)sulfonyl)-2,2-difluoro-1-oxo-2,3-dihydro-1H-inden-4-yl)oxy)-5-fluorobenzonitrile (0.13 g, 51% combined yield). LCMS ESI (+) m/z 418 (M+H) and 435 (M+NH₄).

Step E: Preparation of (S)-3-((7-((difluoromethyl)sulfonyl)-2,2-difluoro-1-hydroxy-2,3-dihydro-1H-inden-4-yl)oxy)-5-fluorobenzonitrile (Compound 15): An ice cold solution of RuCl(p-cymene)[(R,R)-Ts-DPEN] (0.6 mg) in dichloromethane (0.2 mL) was added by syringe under nitrogen to an ice cold solution of 3-[7-(difluoromethylsulfonyl)-2,2-difluoro-1-oxo-indan-4-yl]oxy-5-fluoro-benzonitrile (28 mg, 0.07 mmol), triethylamine (18.7 L, 0.13 mmol) and formic acid (7.6 μL, 0.2 mmol) in dichloromethane (0.5 mL) and then placed in a refrigerator at 4° C. overnight. The reaction mixture was directly purified on preparative TLC with EtOAc/hexane (40%) to give Compound 15 (23.4 mg, 0.06 mmol, 83% yield). The ee was determined to be greater than 95% by ¹⁹F NMR analysis of the corresponding Mosher ester. LCMS ESI (+) m/z 420 (M+H); ¹H NMR (400 MHz, CDCl₃): δ 7.94 (d, 1H), 7.33-6.98 (m, 4H), 6.44 (t, 1H), 5.51 (d, 1H), 3.61-3.45 (m, 2H).

### Example 2: Synthesis of (S)-3-((2,2-Difluoro-1-hydroxy-7-(methylsulfonyl)-2,3-dihydro-1H-inden-4-yl)oxy)-5-fluorobenzonitrile (Compound 163)

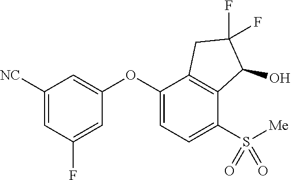 [SMILES: CS(=O)(=O)c1ccc(Oc2cc(F)cc(C#N)c2)c2c1[C@H](O)C(F)(F)C2]

Step A: Preparation of 4′-(3-bromo-5-fluoro-phenoxy)-7′-methylsulfonyl-spiro[1,3-dioxolane-2,1′-indane]: Cesium hydrogen carbonate (142 mg, 0.73 mmol) was added all at once to 4′-fluoro-7′-methylsulfonyl-spiro[1,3-dioxolane-2,1′-indane] (100 mg, 0.37 mmol) and 3-bromo-5-fluoro-phenol (105 mg, 0.55 mmol) in 1-methyl-2-pyrrolidone (1.5 mL) at room temperature in a microwave reaction vial equipped with a stir bar. The flask was flushed with nitrogen then sealed with a crimp cap. The reaction was heated to 150° C. for 7 hours, cooled to ambient temperature then purified directly on reverse phase silica gel (25+M, 14 CV, 20-100% MeCN/water) affording 4′-(3-bromo-5-fluoro-phenoxy)-7′-methylsulfonyl-spiro[1,3-dioxolane-2,1′-indane] (118 mg, 0.26 mmol, 72% yield).

Step B: Preparation of 3-fluoro-5-(7′-methylsulfonylspiro[1,3-dioxolane-2,1′-indane]-4′-yl)oxy-benzonitrile: Dichloro[1;1′-bis(diphenylphosphino)ferrocene]palladium (II) dichloromethane adduct (784 mg, 0.97 mmol) was quickly added to a degassed mixture of 4′-(3-bromo-5-fluoro-phenoxy)-7′-methylsulfonyl-spiro[1,3-dioxolane-2,1′-indane] (4.3 g, 9.7 mmol), zinc cyanide (1.14 g, 9.7 mmol) and zinc powder (761 mg, 11.6 mmol) in DMF (60 mL) under nitrogen. The reaction mixture was then warmed to 110° C. for 2 hours. After cooling, the mixture was filtered through a pad of celite. The filtrate was diluted with water (100 mL), extracted with MTBE (5×100 mL), washed with brine (100 mL), dried over MgSO₄, filtered and concentrated in vacuo. The crude product was purified on silica gel (100 g SNAP, 14 CV, 15-100% EtOAc/hexane) then purified again on silica gel (25 g Ultra SNAP, 14 CV, 0-20% dichloromethane/EtOAc) affording 3-fluoro-5-(7′-methylsulfonylspiro[1,3-dioxolane-2,1′-indane]-4′-yl)oxy-benzonitrile (3.77 g, 9.7 mmol, 100% yield).

Step C: Preparation of 3-fluoro-5-(7-methylsulfonyl-1-oxo-indan-4-yl)oxy-benzonitrile: Pyridinium para-toluenesulfonate (354 mg, 1.4 mmol) was added all at once to a solution of 3-fluoro-5-(7′-methylsulfonylspiro[1,3-dioxolane-2,1′-indane]-4′-yl)oxy-benzonitrile (550 mg, 1.4 mmol) in acetone (6 mL)/water (2 mL) at room temperature and then warmed to reflux until completion. The mixture was concentrated in vacuo then purified on silica gel (10 g SNAP, 14 CV, 20-100% EtOAc/hexane) affording 3-fluoro-5-(7-methylsulfonyl-1-oxo-indan-4-yl)oxy-benzonitrile (450 mg, 1.3 mmol, 92% yield).

Step D: Preparation of 3-[(E, Z)-1-butylimino-7-methylsulfonyl-indan-4-yl]oxy-5-fluoro-benzonitrile: Butan-1-amine (5.15 mL, 52 mmol) was added to 3-fluoro-5-(7-methylsulfonyl-1-oxo-indan-4-yl)oxy-benzonitrile (450 mg, 1.3 mmol) and trifluoroacetic acid (19.96 μL, 0.26 mmol) in benzene (10 mL) at room temperature then warmed to reflux with the azeotropic removal of water by a Dean-Stark apparatus. Progress of the reaction was monitored by H-NMR. When complete, the reaction was cooled to room temperature then concentrated in vacuo. The residue was diluted with water (10 mL), extracted with MTBE (3×10 mL), washed with brine and dried over Na₂SO₄, filtered and concentrated. Crude 3-[(E, Z)-1-butylimino-7-methylsulfonyl-indan-4-yl]oxy-5-fluoro-benzonitrile was used immediately without purification in the next step.

Step E: Preparation of 3-(2,2-difluoro-7-methylsulfonyl-1-oxo-indan-4-yl)oxy-5-fluoro-benzonitrile: Selectfluor® (1.15 g, 3.25 mmol) was added to crude 3-[(E, Z)-1-butylimino-7-methylsulfonyl-indan-4-yl]oxy-5-fluoro-benzonitrile (520 mg, 1.3 mmol) and sodium sulfate (369 mg, 2.6 mmol) in acetonitrile (10 mL) then warmed to reflux for 6 hours. The reaction was cooled to room temperature, concentrated HCl (1.0 mL, 12 mmol) was added and stirred for 15 minutes. The mixture was diluted with water (10 mL), extracted with EtOAc (3×10 mL), washed with brine (10 mL), dried over MgSO₄, filtered and concentrated in vacuo. The residue was purified on silica gel (25 g SNAP, 14 CV, 20-100% EtOAc/hexane) afforded 3-(2,2-difluoro-7-methylsulfonyl-1-oxo-indan-4-yl)oxy-5-fluoro-benzonitrile (437 mg, 1.2 mmol, 88% yield).

Step F: Preparation of (S)-3-((2,2-difluoro-1-hydroxy-7-(methylsulfonyl)-2,3-dihydro-1H-inden-4-yl)oxy)-5-fluorobenzonitrile (Compound 163): An ice cold solution of RuCl(p-cymene)[(R,R)-Ts-DPEN] (40.7 mg, 0.06 mmol) in CH₂Cl₂ (30 mL) was added by syringe under nitrogen to an ice cold solution of 3-(2,2-difluoro-7-methylsulfonyl-1-oxo-indan-4-yl)oxy-5-fluoro-benzonitrile (2.44 g, 6.4 mmol), triethylamine (1.78 mL, 12.8 mmol) and formic acid (724 μL, 19.2 mmol) in CH₂Cl₂ (30 mL). The reaction was placed in a refrigerator at 4° C. for 16 hours. The mixture was concentrated to 10 mL then purified directly on silica gel (25 g SNAP ULTRA, 14 CV, 10-50% EtOAc/hexane) affording Compound 163 (2.15 g, 5.6 mmol, 87% yield). Enantiomeric excess (98%) was determined by chiral HPLC. Retention time for (S)-enantiomer:1.93 minutes; retention time for (R)-enantiomer: 2.32 minutes. LCMS ESI (−) 428 (M+HCO₂). ¹HNMR (400 MHz, CDCl₃): δ 7.93 (d, 1H), 7.27-7.24 (m, 1H), 7.15-7.14 (m, 1H), 7.07-7.03 (m, 1H), 7.00 (d, 1H), 5.63-5.58 (m, 1H), 3.56-3.35 (m, 3H), 3.24 (s, 3H).

### Example 3: Synthesis of 3-[(1S,2S,3R)-2,3-difluoro-1-hydroxy-7-methylsulfonyl-indan-4-yl]oxy-5-fluoro-benzonitrile (Compound 289)

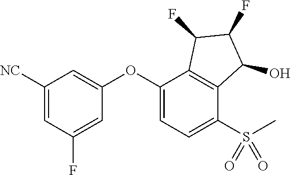 [SMILES: CS(=O)(=O)c1ccc(Oc2cc(F)cc(C#N)c2)c2c1[C@H](O)[C@H](F)[C@@H]2F]

Step A: [(1S,2R)-4-(3-cyano-5-fluoro-phenoxy)-2-fluoro-7-methylsulfonyl-indan-1-yl] acetate: To a stirred solution of 3-fluoro-5-[(1S,2R)-2-fluoro-1-hydroxy-7-methylsulfonyl-indan-4-yl]oxy-benzonitrile (2.00 g, 5.47 mmol) in DCM (27 mL) was added 4-(dimethylamino)pyridine (0.2 g, 1.64 mmol) and triethylamine (1.53 mL, 10.9 mmol). Acetic anhydride (1.00 mL, 10.9 mmol) was added dropwise at 0° C. under nitrogen. The reaction mixture was stirred at ambient temperature overnight. The reaction mixture was diluted with DCM, washed with saturated aqueous NaHCO₃ and brine, dried and concentrated. The residue was purified by flash chromatography on silica gel (20-40% EtOAc/hexane) to give [(1S,2R)-4-(3-cyano-5-fluoro-phenoxy)-2-fluoro-7-methylsulfonyl-indan-1-yl] acetate (1.95 g, 87%). LCMS ESI (+) m/z 408 (M+H).

Step B: [(1S,2S,3S)-3-bromo-4-(3-cyano-5-fluoro-phenoxy)-2-fluoro-7-methylsulfonyl-indan-1-yl] acetate and [(1S,2S,3R)-3-bromo-4-(3-cyano-5-fluoro-phenoxy)-2-fluoro-7-methylsulfonyl-indan-1-yl] acetate: To a stirred solution of [(1S,2R)-4-(3-cyano-5-fluoro-phenoxy)-2-fluoro-7-methylsulfonyl-indan-1-yl] acetate (1.95 g, 4.79 mmol) in 1,2-dichloroethane (24 mL) was added N-bromosuccinimide (0.94 g, 5.27 mmol) and 2,2′-azobisisobutyronitrile (8 mg, 0.05 mmol). The reaction mixture was heated at 80° C. for 3 hours. After cooling, the reaction mixture was diluted with DCM, washed with saturated aqueous NaHCO₃ and brine, dried and concentrated. The residue was purified by column chromatography on silica gel (20-30% EtOAc/hexane) to give [(1S,2S,3S)-3-bromo-4-(3-cyano-5-fluoro-phenoxy)-2-fluoro-7-methylsulfonyl-indan-1-yl] acetate (1.52 g, 65%). LCMS ESI (+) m/z 486, 488 (M+H). Further elution with 30-50% EtOAc/hexane gave the more polar product [(1S,2S,3R)-3-bromo-4-(3-cyano-5-fluoro-phenoxy)-2-fluoro-7-methylsulfonyl-indan-1-yl] acetate (0.583 g, 25%). LCMS ESI (+) m/z 486,488 (M+H).

Step C: [(1S,2R,3S)-4-(3-cyano-5-fluoro-phenoxy)-2-fluoro-3-hydroxy-7-methylsulfonyl-indan-1-yl] acetate: To a combined mixture of [(1S,2S,3S)-3-bromo-4-(3-cyano-5-fluoro-phenoxy)-2-fluoro-7-methylsulfonyl-indan-1-yl] acetate and [(1S,2S,3R)-3-bromo-4-(3-cyano-5-fluoro-phenoxy)-2-fluoro-7-methylsulfonyl-indan-1-yl] acetate prepared in Step B (2.05 g, 4.22 mmol) were added 1,2-dimethoxyethane (28 mL) and water (0.050 mL) followed by silver perchlorate hydrate (1.42 g, 6.32 mmol). The reaction mixture was heated at 70° C. for 2 hours. After cooling, the reaction mixture was diluted with EtOAc and filtered through Celite. The filtrate was washed with water and brine, dried and concentrated. The residue was purified by flash chromatography on silica gel (20-50%) to give [(1S,2R,3S)-4-(3-cyano-5-fluoro-phenoxy)-2-fluoro-3-hydroxy-7-methylsulfonyl-indan-1-yl] acetate (0.416 g, 23%) as the less polar product. LCMS ESI (+) m/z 441 (M+NH₄⁺). Further elution with 60% EtOAc/hexane gave [(1S,2R,3R)-4-(3-cyano-5-fluoro-phenoxy)-2-fluoro-3-hydroxy-7-methylsulfonyl-indan-1-yl] acetate (0.58 g, 32%). LCMS ESI (+) m/z 441 (M+NH₄⁺).

Step D: [(1S,2S,3R)-4-(3-cyano-5-fluoro-phenoxy)-2,3-difluoro-7-methylsulfonyl-indan-1-yl] acetate: To a stirred solution of [(1S,2R,3S)-4-(3-cyano-5-fluoro-phenoxy)-2-fluoro-3-hydroxy-7-methylsulfonyl-indan-1-yl] acetate (416 mg, 0.98 mmol) in DCM (10 mL) was added (diethylamino)sulfur trifluoride (DAST) (0.26 mL, 2.0 mmol) at −78° C. under nitrogen. The reaction mixture was allowed to warm to 0° C. and stirred for 15 minutes. The reaction was quenched by saturated aqueous NaHCO₃. The mixture was partitioned between EtOAc and water. The aqueous layer was extracted with EtOAc. The combined organic layers were washed with brine, dried and concentrated. The residue was purified by flash chromatography on silica gel (20-40% EtOAc/hexane) to give [(1S,2S,3R)-4-(3-cyano-5-fluoro-phenoxy)-2,3-difluoro-7-methylsulfonyl-indan-1-yl] acetate (310 mg, 74%). LCMS ESI (+) m/z 426 (M+H).

Step E: 3-[(1S,2S,3R)-2,3-difluoro-1-hydroxy-7-methylsulfonyl-indan-4-yl]oxy-5-fluoro-benzonitrile (Compound 289): To a stirred solution of [(1S,2S,3R)-4-(3-cyano-5-fluoro-phenoxy)-2,3-difluoro-7-methylsulfonyl-indan-1-yl] acetate (0.23 mmol) in tetrahydrofuran (1.5 mL) was added 0.5 N LiOH solution (0.68 mL, 0.34 mmol) at 0° C. under nitrogen. The reaction mixture was stirred at 0° C. for 1 hour. The reaction was then partitioned between EtOAc and water. The aqueous layer was extracted with EtOAc. The combined organic layers were washed with water and brine, dried and concentrated. The residue was purified by flash chromatography on silica gel (30-70% EtOAc/hexane) to give Compound 289. LCMS ESI (+) m/z 384 (M+H); ¹H NMR (400 MHz, CDCl₃): δ 8.13 (d, 1H), 7.31-7.25 (m, 1H), 7.23-7.19 (m, 1H), 7.14-7.09 (m, 1H), 7.04 (d, 1H), 6.09-5.91 (m, 1H), 5.87-5.80 (m, 1H), 5.25-5.05 (m, 1H), 3.32 (s, 3H), 2.95 (d, 1H).

### Example 4: Synthesis of (6R,7S)-4-(3,3-difluorocyclobutoxy)-6-fluoro-1-(trifluoromethyl)-6,7-dihydro-5H-cyclopenta[c]pyridin-7-ol (Compound 465) and (R)-4-(3,3-difluorocyclobutoxy)-1-(trifluoromethyl)-6,7-dihydro-5H-cyclopenta[c]pyridin-7-ol (Compound 466)

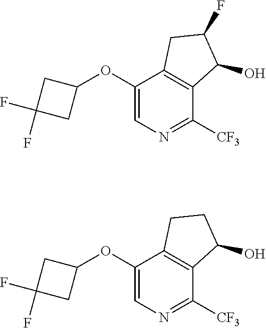 [SMILES: O[C@H]1c2c(C(F)(F)F)ncc(OC3CC(F)(F)C3)c2C[C@H]1F ; O[C@@H]1CCc2c(OC3CC(F)(F)C3)cnc(C(F)(F)F)c21]

Step A: Preparation of 4-bromo-1-(trifluoromethyl)-5,6-dihydro-7H-cyclopenta[c]pyridin-7-one: A suspension of 4-bromo-5,6-dihydrocyclopenta[c]pyridin-7-one (1.0 g, 4.72 mmol) and bis(((trifluoromethyl)sulfinyl)oxy)zinc (4.69 g, 14.15 mmol) in a mixture of dichloromethane (30 mL) and water (15 mL) at 0° C. was treated with tert-butyl hydroperoxide (70% in water, 2.58 mL, 18.86 mmol, added via pipette using a plastic tip) and stirred overnight. Additional portions of bis(((trifluoromethyl)sulfinyl)oxy)zinc (2.35 g, 7.07 mmol) and tert-butyl hydroperoxide (2.58 mL, 18.86 mmol) were added sequentially to drive the reaction to completion. After stirring for an additional day, the reaction vessel was placed into a water bath and carefully quenched by the addition of saturated NaHCO₃. Once effervescence ceased, the reaction mixture filtered through a pad of celite to remove. The pad of celite was rinsed with additional dichloromethane. The filtrate was separated and the aqueous portion extracted further with 2×20 mL CH₂Cl₂. The combined organics were rinsed with 10 mL of brine, dried with MgSO₄, filtered, and concentrated to dryness. Purification was achieved by chromatography on silica using 30-90% CH₂Cl₂/hexane to afford 4-bromo-1-(trifluoromethyl)-5,6-dihydro-7H-cyclopenta[c]pyridin-7-one as an off-white solid (390 mg, 30%). The desired regioisomer elutes first. LCMS ESI (+) (M+H) m/z 280/282.

Step B: Preparation of 4-bromo-1-(trifluoromethyl)-5,6-dihydrospiro[cyclopenta[c]pyridine-7,2′-[1,3]dioxolane] and 4-bromo-1-(trifluoromethyl)-7-(2-((trimethylsilyl)oxy)ethoxy)-5H-cyclopenta[c]pyridine: Trimethylsilyl trifluoromethanesulfonate (75.9 μL, 0.42 mmol) was added to a solution of 4-bromo-1-(trifluoromethyl)-5,6-dihydrocyclopenta[c]pyridin-7-one (389 mg, 1.39 mmol) and trimethyl(2-trimethylsilyloxyethoxy)silane (1.37 mL, 5.56 mmol) in dichloromethane (13.6 mL) cooled in an ice bath. The mixture was allowed to slowly warm to ambient temperature. After 5 h, an additional 1.3 mL of trimethyl(2-trimethylsilyloxyethoxy)silane and 76 μL of trimethylsilyl trifluoromethanesulfonate were added. After another 16 h, the reaction mixture was treated with triethylamine (770 μL, 5.56 mmol), stirred for 10 min, and then concentrated. The residue was treated with 20 mL EtOAc and 20 mL of water and the layers separated. The aqueous portion was extracted further with 2×20 mL of EtOAc. The combined organic extracts were washed with brine, dried over MgSO₄, filtered, and evaporated. Purification was achieved by chromatography on silica using 5-20% EtOAc/hexane to afford 4-bromo-1-(trifluoromethyl)-5,6-dihydrospiro[cyclopenta[c]pyridine-7,2′-[1,3]dioxolane] as a shite solid (262 mg, 58%) and 4-bromo-1-(trifluoromethyl)-7-(2-((trimethylsilyl)oxy)ethoxy)-5H-cyclopenta[c]pyridine as a white solid (170 mg, 31%). Data for 4-bromo-1-(trifluoromethyl)-5,6-dihydrospiro[cyclopenta[c]pyridine-7,2′-[1,3]dioxolane]: LCMS ESI (+) (M+H) m/z 324/326. Data for 4-bromo-1-(trifluoromethyl)-7-(2-((trimethylsilyl)oxy)ethoxy)-5H-cyclopenta[c]pyridine: ¹H NMR (400 MHz, CDCl₃): δ 8.56 (s, 1H), 5.59 (t, 1H), 4.10 (t, 2H), 3.96 (t, 2H), 3.36 (d, 2H), 0.15 (s, 9H).

Step C: Preparation of 4-bromo-6-fluoro-1-(trifluoromethyl)-5,6-dihydrospiro[cyclopenta[c]pyridine-7,2′-[1,3]dioxolane]: A solution of 2-[[4-bromo-1-(trifluoromethyl)-5H-cyclopenta[c]pyridin-7-yl]oxy]ethoxy-trimethyl-silane (146.6 mg, 0.37 mmol) and sodium sulfate (262.7 mg, 1.85 mmol) in acetonitrile (3.7 mL) was stirred for 10 min and then treated with Selectfluor® (145.2 mg, 0.41 mmol) and stirred at 25° C. for 1 h. Volatiles were removed by concentration under reduced pressure. The reaction mixture was poured into 30 mL of water and extracted with 3×15 mL EtOAc. The combined organics were rinsed with 10 mL of brine, dried with MgSO₄, filtered, and concentrated to dryness. Purification was achieved by chromatography on silica using 5-20% EtOAc/hexane to afford 4-bromo-6-fluoro-1-(trifluoromethyl)-5,6-dihydrospiro[cyclopenta[c]pyridine-7,2′-[1,3]dioxolane] as a white solid (96.2 mg, 76%). LCMS ESI (+) (M+H) m/z 342/344.

Step D: Preparation of 6-fluoro-1-(trifluoromethyl)-5,6-dihydrospiro[cyclopenta[c]pyridine-7,2′-[1,3]dioxolan]-4-ol and 1-(trifluoromethyl)-5,6-dihydrospiro[cyclopenta[c]pyridine-7,2′-[1,3]dioxolan]-4-ol: A solution of 4′-bromo-6′-fluoro-1′-(trifluoromethyl)spiro[1,3-dioxolane-2,7′-5,6-dihydrocyclopenta[c]pyridine](96.2 mg, 0.2800 mmol) and 2-(di-t-butylphosphino)-3,6-dimethoxy-2′,4′,6′-tri-i-propyl-1,1′-biphenyl (3.4 mg, 0.007 mmol) in 1,4-dioxane (5.0 mL) was sparged with nitrogen for 3 mins. The reaction mixture was then treated sequentially with potassium hydroxide (47.3 mg, 0.84 mmol), water (101 μL, 5.62 mmol) and [2-(2-aminophenyl)phenyl]-methylsulfonyloxy-palladium; di-t-butyl-[3,6-dimethoxy-2-(2,4,6-triisopropylphenyl)phenyl]phosphane (6.0 mg, 0.007 mmol) under continuous nitrogen stream. The vessel was sealed and heated to 80 C for 1 h and 30 min. The reaction mixture was quenched by the addition of acetic acid (64.3 μL, 1.13 mmol). The reaction mixture was poured into 75 mL of water and extracted with 4×20 mL EtOAc. The combined organics were dried with MgSO₄, filtered, and concentrated to dryness. The product was used without further purification (87 mg). During the reaction, some of the hydrodefluorinated product formed as an impurity. Data for 6-fluoro-1-(trifluoromethyl)-5,6-dihydrospiro[cyclopenta[c]pyridine-7,2′-[1,3]dioxolan]-4-ol: LCMS ESI (+) (M+H) m/z 280. Data for 1-(trifluoromethyl)-5,6-dihydrospiro[cyclopenta[c]pyridine-7,2′-[1,3]dioxolan]-4-ol: LCMS ESI (+) (M+H) m/z 262.

Step E: Preparation of 4-(3,3-difluorocyclobutoxy)-6-fluoro-1-(trifluoromethyl)-5,6-dihydrospiro[cyclopenta[c]pyridine-7,2′-[1,3]dioxolane] and 4-(3,3-difluorocyclobutoxy)-1-(trifluoromethyl)-5,6-dihydrospiro[cyclopenta[c]pyridine-7,2′-[1,3]dioxolane]: A solution of impure 6′-fluoro-1′-(trifluoromethyl)spiro[1,3-dioxolane-2,7′-5,6-dihydrocyclopenta[c]pyridine]-4′-ol (44.0 mg, 0.16 mmol), polymer supported triphenylphosphine (˜2.06 mmol/g, 306.2 mg, 0.63 mmol), and 3,3-difluoro-cyclobutanol (68.1 mg, 0.63 mmol) in tetrahydrofuran (3.2 mL) was treated with diisopropyl azodicarboxylate (120 μL, 0.61 mmol) and stirred at 60° C. for 2 h. The reaction mixture was filtered and the filter cake rinsed with 20 mL EtOAc. The filtrate was concentrated and purified by chromatography on silica using 10-30% EtOAc/hexane to afford a clear solid (39.0 mg, 67%) that was a 2:1 mixture of the fluorinated and hydrodefluorinated products. LCMS ESI (+) (M+H) m/z 370. Data for 4-(3,3-difluorocyclobutoxy)-6-fluoro-1-(trifluoromethyl)-5,6-dihydrospiro[cyclopenta[c]pyridine-7,2′-[1,3]dioxolane]: LCMS ESI (+) (M+H) m/z 370. Data for 4-(3,3-difluorocyclobutoxy)-1-(trifluoromethyl)-5,6-dihydrospiro[cyclopenta[c]pyridine-7,2′-[1,3]dioxolane]: LCMS ESI (+) (M+H) m/z 352.

Step F: Preparation of 4-(3,3-difluorocyclobutoxy)-6-fluoro-1-(trifluoromethyl)-5,6-dihydro-7H-cyclopenta[c]pyridin-7-one and 4-(3,3-difluorocyclobutoxy)-1-(trifluoromethyl)-5,6-dihydro-7H-cyclopenta[c]pyridin-7-one: A solution of impure 4′-(3,3-difluorocyclobutoxy)-6′-fluoro-1′-(trifluoromethyl)spiro[1,3-dioxolane-2,7′-5,6-dihydrocyclopenta[c]pyridine] (39.0 mg, 0.106 mmol) in dichloromethane (2.0 mL) at 0 C was treated with perchloric acid (70% in water, 200 μL) and stirred at 0 C for 3 h. The reaction mixture was quenched by the addition of 5 mL of saturated aqueous NaHCO₃. The resulting mixture was extracted with 3×15 mL CH₂Cl₂. The combined organics were rinsed with 10 mL of brine, dried with MgSO₄, filtered, and concentrated to dryness. The product was used without further purification as a 2:1 mixture of fluorinated and hydrodefluorinated ketones. LCMS ESI (+) (M+H) m/z 326. Data for 4-(3,3-difluorocyclobutoxy)-6-fluoro-1-(trifluoromethyl)-5,6-dihydro-7H-cyclopenta[c]pyridin-7-one: LCMS ESI (+) (M+H) m/z 326. Data for 4-(3,3-difluorocyclobutoxy)-1-(trifluoromethyl)-5,6-dihydro-7H-cyclopenta[c]pyridin-7-one: LCMS ESI (+) (M+H) m/z 308.

Step G: Preparation of (6R,7S)-4-(3,3-difluorocyclobutoxy)-6-fluoro-1-(trifluoromethyl)-6,7-dihydro-5H-cyclopenta[c]pyridin-7-ol (Compound 465) and (R)-4-(3,3-difluorocyclobutoxy)-1-(trifluoromethyl)-6,7-dihydro-5H-cyclopenta[c]pyridin-7-ol (Compound 466): A solution of impure 4-(3,3-difluorocyclobutoxy)-6-fluoro-1-(trifluoromethyl)-5,6-dihydrocyclopenta[c]pyridin-7-one (33.8 mg, 0.10 mmol) in dichloromethane (4.0 mL) was cooled to 0° C. and sparged with nitrogen for 5 min. During this time formic acid (11.8 μL, 0.31 mmol) and triethylamine (28.8 μL, 0.21 mmol) were sequentially added. Once sparging was complete, RuCl(p-cymene)[(R,R)-Ts-DPEN] (1.3 mg, 0.002 mmol) was added under a continuous stream of nitrogen. The reaction vessel was sealed and placed into the refrigerator to react overnight. Volatiles were removed by concentration under reduced pressure. The residue was purified by chromatography on silica using 4-18% EtOAc/CH₂Cl₂ to afford (6R,7S)-4-(3,3-difluorocyclobutoxy)-6-fluoro-1-(trifluoromethyl)-6,7-dihydro-5H-cyclopenta[c]pyridin-7-ol (Compound 465) as a clear solid (5.4 mg, 16%) and (R)-4-(3,3-difluorocyclobutoxy)-1-(trifluoromethyl)-6,7-dihydro-5H-cyclopenta[c]pyridin-7-ol (Compound 466) as a clear solid (7.4 mg, 23%). Data for (6R,7S)-4-(3,3-difluorocyclobutoxy)-6-fluoro-1-(trifluoromethyl)-6,7-dihydro-5H-cyclopenta[c]pyridin-7-ol (Compound 465): Retention time HPLC (14 min)=3.59 min; LCMS ESI (+) (M+H) m/z 328; ¹H NMR (400 MHz, CDCl₃): δ 8.04 (s, 1H), 5.46-5.26 (m, 2H), 4.89-4.79 (m, 1H), 3.36-3.08 (m, 4H), 2.91-2.74 (m, 2H), 2.60 (dd, 1H). Data for (R)-4-(3,3-difluorocyclobutoxy)-1-(trifluoromethyl)-6,7-dihydro-5H-cyclopenta[c]pyridin-7-ol (Compound 466): Retention time HPLC (14 min)=3.95 min; LCMS ESI (+) (M+H) m/z 310; ¹H NMR (400 MHz, CDCl₃): δ 7.98 (s, 1H), 5.59-5.54 (m, 1H), 4.88-4.79 (m, 1H), 3.24-3.07 (m, 3H), 2.89 (dd, 1H), 2.89-2.74 (m, 2H), 2.44-2.34 (m, 1H), 2.28-2.21 (m, 1H), 2.12-2.09 (m, 1H).

### Example 5: Synthesis of 3-fluoro-5-(((6R,7S)-6-fluoro-7-hydroxy-1-(trifluoromethyl)-6,7-dihydro-5H-cyclopenta[c]pyridin-4-yl)oxy)benzonitrile (Compound 467)

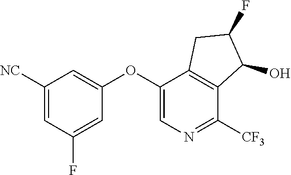 [SMILES: N#Cc1cc(F)cc(Oc2cnc(C(F)(F)F)c3c2C[C@@H](F)[C@H]3O)c1]

Step A: Preparation of 1-(trifluoromethyl)-5,6-dihydrospiro[cyclopenta[c]pyridine-7,2′-[1,3]dioxolan]-4-ol: A solution of 4′-bromo-1′-(trifluoromethyl)spiro[1,3-dioxolane-2,7′-5,6-dihydrocyclopenta[c]pyridine] (226.4 mg, 0.70 mmol) and 2-(di-t-butylphosphino)-3,6-dimethoxy-2′,4′,6′-tri-i-propyl-1,1′-biphenyl (8.5 mg, 0.017 mmol) in 1,4-dioxane (7.0 mL) was sparged with nitrogen for 3 mins. The reaction mixture was then treated sequentially with potassium hydroxide (117.6 mg, 2.10 mmol), water (252 μL, 13.97 mmol) and [2-(2-aminophenyl)phenyl]-methylsulfonyloxy-palladium; ditert-butyl-[3,6-dimethoxy-2-(2,4,6-triisopropylphenyl)phenyl]phosphane (14.9 mg, 0.017 mmol) under continuous nitrogen stream. The vessel was sealed and heated to 80 C for 1 h and 30 min. The reaction mixture was quenched by the addition of acetic acid (160 μL, 2.79 mmol). The reaction mixture was poured into 75 mL of water and extracted with 4×20 mL EtOAc. The combined organics were dried with MgSO4, filtered, and concentrated to dryness. The brown solid was used without further purification. LCMS ESI (−) (M−H) m/z 260.

Step B: Preparation of 3-fluoro-5-((1-(trifluoromethyl)-5,6-dihydrospiro[cyclopenta[c]pyridine-7,2′-[1,3]dioxolan]-4-yl)oxy)benzonitrile: A suspension of potassium tert-butoxide (28.4 mg, 0.25 mmol) in tetrahydrofuran (1.5 mL) at 0 C was treated with 1′-(trifluoromethyl)spiro[1,3-dioxolane-2,7′-5,6-dihydrocyclopenta[c]pyridine]-4′-ol (60 mg, 0.23 mmol) and stirred at 0 C for 15 min. The resulting mixture was treated with (3-cyano-5-fluoro-phenyl)-(4-methoxyphenyl)iodonium; 4-methylbenzenesulfonate (144.8 mg, 0.28 mmol) and heated to 40 C. The reaction mixture was filtered through a plastic filter cup using EtOAc to rinse. Volatiles were removed by concentration under reduced pressure. Purification was achieved by chromatography on silica using 10-40% EtOAc/hexane to afford a solid (42 mg, 48%). LCMS ESI (+) (M+H) m/z 381.

Step C: Preparation of 3-fluoro-5-((7-oxo-1-(trifluoromethyl)-6,7-dihydro-5H-cyclopenta[c]pyridin-4-yl)oxy)benzonitrile: A solution of 3-fluoro-5-[1′-(trifluoromethyl)spiro[1,3-dioxolane-2,7′-5,6-dihydrocyclopenta[c]pyridine]-4′-yl]oxy-benzonitrile (42.0 mg, 0.11 mmol) in dichloromethane (2.0 mL) at 0 C was treated with perchloric acid (70% in water, 240 μL) and stirred at 0 C for 30 min. The reaction mixture was carefully quenched by the addition of 15 mL of saturated NaHCO₃ and extracted with 3×15 mL CH₂Cl₂. The combined organics were rinsed with 10 mL of brine, dried with MgSO₄, filtered, and concentrated to dryness. The solid residue was used immediately in the next step without further purification. LCMS ESI (+) (M+H) m/z 337.

Step D: Preparation of 3-((7-((tert-butyldimethylsilyl)oxy)-1-(trifluoromethyl)-5H-cyclopenta[c]pyridin-4-yl)oxy)-5-fluorobenzonitrile: A solution of triethylamine (122 μL, 0.88 mmol) and 3-fluoro-5-[[7-oxo-1-(trifluoromethyl)-5,6-dihydrocyclopenta[c]pyridin-4-yl]oxy]benzonitrile (37.0 mg, 0.11 mmol) in dichloromethane (2.2 mL) at 0° C. was treated with tert-butyldimethylsilyl triflate (152 ul, 0.66 mmol). The ice bath was removed and the reaction mixture left to stir for 2 h. The reaction mixture was poured into 30 mL of saturated NaHCO₃ and extracted with 3×20 mL CH₂Cl₂. The combined organics were rinsed with 10 mL of brine, dried with MgSO₄, filtered, and concentrated to dryness. The product was used without further purification. LCMS ESI (+) (M+H) m/z 451.

Step E: Preparation of 3-fluoro-5-((6-fluoro-7-oxo-1-(trifluoromethyl)-6,7-dihydro-5H-cyclopenta[c]pyridin-4-yl)oxy)benzonitrile: A solution of 3-[[7-[tert-butyl(dimethyl)silyl]oxy-1-(trifluoromethyl)-5H-cyclopenta[c]pyridin-4-yl]oxy]-5-fluoro-benzonitrile (49.56 mg, 0.1100 mmol) in acetonitrile (2.2 mL) at 25° C. was treated with Selectfluor® (42.9 mg, 0.12 mmol) and stirred at 25° C. for 1 h. Volatiles were removed by concentration under reduced pressure The reaction mixture was poured into 30 mL of water and extracted with 3×10 mL EtOAc. The combined organics were rinsed with 10 mL of brine, dried with MgSO₄, filtered, and concentrated to dryness. Purification was achieved by chromatography on silica using 10-25% EtOAc/hexane to afford a thin film (37.8 mg, 97%). LCMS ESI (+) (M+H) m/z 355.

Step F: Preparation of 3-fluoro-5-(((6R,7S)-6-fluoro-7-hydroxy-1-(trifluoromethyl)-6,7-dihydro-5H-cyclopenta[c]pyridin-4-yl)oxy)benzonitrile (Compound 467): A solution of 3-fluoro-5-[[6-fluoro-7-oxo-1-(trifluoromethyl)-5,6-dihydrocyclopenta[c]pyridin-4-yl]oxy]benzonitrile (15.3 mg, 0.043 mmol) in dichloromethane (1.5 mL) was cooled to 0° C. and sparged with nitrogen for 5 min. During this time formic acid (4.9 μL, 0.13 mmol) and triethylamine (12.0 μL, 0.086 mmol) were sequentially added. Once the sparging was complete, RuCl(p-cymene)[(R,R)-Ts-DPEN] (0.5 mg, 0.00086 mmol) was added under a continuous stream of nitrogen. The reaction vessel was sealed and placed into the refrigerator to react overnight. Volatiles were removed by concentration under reduced pressure. The residue was purified by chromatography on silica using 10-30% EtOAc/hexane to afford 3-fluoro-5-(((6R,7S)-6-fluoro-7-hydroxy-1-(trifluoromethyl)-6,7-dihydro-5H-cyclopenta[c]pyridin-4-yl)oxy)benzonitrile (Compound 467) as a clear solid (11.8 mg, 77%). Retention time HPLC (14 min)=4.19 min; LCMS ESI (+) (M+H) m/z 357; H NMR (400 MHz, CDCl₃): δ 8.33 (s, 1H), 7.22 (ddd, 1H), 7.10-7.08 (m, 1H), 6.99 (dt, 1H), 5.54-5.46 (m, 1H), 5.46-5.28 (m, 1H), 3.26 (ddd, 1H), 3.11 (ddd, 1H), 2.67 (dd, 1H).

### Example 6: (S)-3-((2,2-difluoro-1-hydroxy-7-((trifluoromethyl)sulfonyl)-2,3-dihydro-1H-inden-4-yl)amino)-5-fluorobenzonitrile (Compound 489)

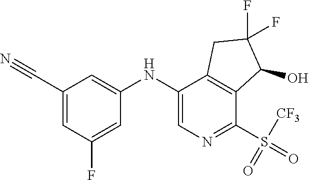 [SMILES: N#Cc1cc(F)cc(Nc2cnc(S(=O)(=O)C(F)(F)F)c3c2CC(F)(F)[C@H]3O)c1]

Step A: Preparation of 4-bromophenyl 3-chloropropanoate: A solution of 4-bromophenol (45.0 g, 260 mmol) in dichloromethane (1.0 L) was cooled to 0° C., treated with triethylamine (44.7 g, 442 mmol). A solution of 3-chloropropionyl chloride (36.3 g, 286 mmol) dissolved in dichloromethane (100 mL) was added dropwise to the reaction vessel. The reaction mixture was allowed to warm to ambient temperature and stirred overnight. Saturated NaCl was added to the reaction mixture, (300 mL). After stirring for 1 hour, the organic layer was separated. The aqueous layer was extracted with dichloromethane. The combined organics were washed with saturated NaCl, dried over Na₂SO₄, and concentrated in vacuo. The crude product was used without further purification.

Step B: Preparation of 4-bromo-7-hydroxy-2,3-dihydro-1H-inden-1-one: A flask containing crude (4-bromophenyl) 3-chloropropanoate (68.0 g, 258 mmol) was cooled to 0° C., then treated in several portions with aluminum trichloride (275 g, 2060 mmol). The reaction mixture was then heated at 155° C. under N₂ for 3 hours. Stirring became difficult as the reaction proceeded. HCl (g) which was generated from the reaction was trapped by a beaker containing 1 N NaOH. After cooling to ambient temperature, the reaction mixture was further cooled in an ice bath. Water was added very carefully (dropwise initially and then added in small volumes) to the reaction to quench excess AlCl₃. The mixture was then extracted with twice with ethyl acetate. The combined organic layers were washed with water and brine, dried and concentrated. The crude product was used without additional purification.

Step C: Preparation of O-(7-bromo-3-oxo-2,3-dihydro-1H-inden-4-yl) dimethylcarbamothioate: A mixture of 4-bromo-7-hydroxy-2,3-dihydro-1H-inden-1-one (900 mg, 4.0 mmol) dissolved in DMF (15 mL) was treated with DABCO 33LV (1.3 mL, 12 mmol) and N,N-dimethylcarbamothioyl chloride (1.5 g, 12 mmol) was stirred overnight at ambient temperature. The reaction was treated with water and ethyl acetate and separated. The aqueous layer was extracted with ethyl acetate then the combined organic layers were washed with water and saturated NaCl. After drying, the organic layer was concentrated in vacuo and purified by chromatography on SiO₂ eluting with a gradient of ethyl acetate/hexane, (670 mg, 54%). ¹H NMR (400 MHz, CDCl₃): δ 7.78-7.76 (d, 1H), 6.97-6.95 (d, 1H), 3.44 (s, 3H), 3.41 (s, 3H), 3.08 (m, 2H), 2.76-2.69 (m, 2H).

Step D: Preparation of S-(7-bromo-3-oxo-2,3-dihydro-1H-inden-4-yl) dimethylcarbamothioate: A mixture of O-(7-bromo-3-oxo-2,3-dihydro-1H-inden-4-yl) dimethylcarbamothioate (670 mg, 2.1 mmol) and diphenyl ether (15 mL) was heated at 220° C. under N₂ for 30 minutes. After cooling to ambient temperature, the mixture was diluted with hexane and the mixture was applied to a pad of SiO₂ and eluted with hexane. After removal of the diphenyl ether, the desired product was eluted with ethyl acetate. After concentration in vacuo, the crude product was used without further purification.

Step E: Preparation of 4-bromo-7-mercapto-2,3-dihydro-1H-inden-1-one: A solution of S-(7-bromo-3-oxo-2,3-dihydro-1H-inden-4-yl) dimethylcarbamothioate (670 mg, 2.1 mmol) dissolved in ethanol (25 mL) was treated with 3N sodium hydroxide) 10.7 mL, 32.1 mmol). The mixture was heated to reflux for 1 hour then cooled to 0° C. Aqueous HCl (3M) was added dropwise to neutralize the reaction. Ethanol was removed by concentration in vacuo followed by addition of aqueous HCl (1M) to adjust to pH 3-4. The aqueous was extracted twice with ethyl acetate and the combined organic layers were washed with saturated NaCl, dried and concentrated in vacuo. The crude product was used without further purification.

Step F: Preparation of 4-bromo-7-((trifluoromethyl)thio)-2,3-dihydro-1H-inden-1-one: Methyl viologen dichloride hydrate (0.11 g, 0.41 mmol), 4-bromo-7-mercapto-2,3-dihydro-1H-inden-1-one (2.0 g, 8.2 mmol) and triethylamine (1.25 g, 12.3 mmol) were dissolved in DMF (50 mL) and cooled to −50° C. The flask was placed under gentle vacuum then trifluoromethyl iodide (3.2 g, 16 mmol) gas was introduced using a balloon. This reaction was warmed to ambient temperature and stirred at overnight. The reaction mixture was diluted with ethyl acetate and water, filtered through a celite pad, and the layers were partitioned. The organic layer was washed with water, brine, dried over Na₂SO₄, filtered, and evaporated. The crude oil was then purified by flash column chromatography on SiO₂ eluting with petroleum ether/ethyl acetate, (0.96 g, 51.7%). ¹H NMR (400 MHz, CDCl₃): δ 7.72 (d, 1H), 7.41 (d, 1H), 3.10-3.07 (m, 2H), 2.79-2.77 (m, 2H).

Step G: Preparation of 4-bromo-7-((trifluoromethyl)sulfonyl)-2,3-dihydro-1H-inden-1-one: Ruthenium(III) chloride (19 mg, 0.09 mmol) was added to a mixture of 4-bromo-7-((trifluoromethyl)thio)-2,3-dihydro-1H-inden-1-one (0.96 g, 3.1 mmol) and sodium periodate (1.98 g, 9.26 mmol) in a mixture of carbon tetrachloride (20 mL), acetonitrile (20 mL), and water (40 mL). The mixture was stirred at ambient temperature for 3 hours. The reaction mixture was partitioned between dichloromethane and water. The organic layer was washed with brine, dried over MgSO₄, filtered, and concentrated in vacuo. The crude product was purified by flash column chromatography on SiO₂ eluting with petroleum ether/ethyl acetate, (1.7 g, 79%). ¹H NMR (400 MHz, CDCl₃): δ 8.05-8.02 (m, 2H), 3.21-3.18 (m, 2H), 2.89-2.86 (m, 2H).

Step H: Preparation of 4-bromo-7-((trifluoromethyl)sulfonyl)-2,3-dihydrospiro[indene-1,2′-[1,3]dioxolane]: Trimethylsilyl trifluoromethanesulfonate (177 mg, 0.80 mmol) was added dropwise to a pre-cooled (−78° C.) solution of 4-bromo-7-((trifluoromethyl)sulfonyl)-2,3-dihydro-1H-inden-1-one and trimethyl(2-trimethylsilyloxyethoxy)silane (410 mg, 2.0 mmol) dissolved in dichloromethane (50 mL). The reaction mixture was warmed to ambient temperature and stirred for 2 hours. The reaction was quenched by addition of triethylamine then concentrated in vacuo. The residue was redissolved in ethyl acetate and washed twice with water, and saturated NaCl. The organic layer was separated, dried over Na₂SO₄ and concentrated in vacuo. The crude product was purified by chromatography on SiO₂ eluting with ethyl acetate/isohexane, (600 mg, 77%).

Step I: Preparation of 4-bromo-7-((trifluoromethyl)sulfonyl)-2,3-dihydro-1H-inden-1-one: 4-Bromo-7-((trifluoromethyl)sulfonyl)-2,3-dihydrospiro[indene-1,2′-[1,3]dioxolane] (3.5 g, 9.1 mmol) was dissolved in THE (72 mL) and treated with 10% aqueous HCl (27 mL, 27 mmol). The mixture was stirred for several minutes then warmed to 60° C. for 2 hours. The mixture was cooled, diluted with diethyl ether and separated. The aqueous was washed with diethyl ether and the combined organics were washed with water, saturated NaHCO₃, saturated NaCl, dried over Na₂SO₄ and concentrated in vacuo to a yellowish solid, (3.09 g, quant.). H NMR (400 MHz, CDCl₃): δ 8.05-8.02 (m, 2H), 3.21-3.18 (m, 2H), 2.89-2.86 (m, 2H).

Step J: Preparation of (E,Z)-3-((4-bromo-7-((trifluoromethyl)sulfonyl)-2,3-dihydro-1H-inden-1-ylidene)amino)propan-1-ol: 4-Bromo-7-(trifluoromethylsulfonyl)indan-1-one (3.09 g, 9.02 mmol] was slurried in toluene (35 mL) and cyclohexane (35 mL) then treated with 3-methoxypropylamine (2.15 mL, 27.1 mmol) and pivalic acid (46 mg, 0.45 mmol). The mixture was refluxed through a Dean-Stark trap (sidearm pre-filled with cyclohexane) for 8 hours. The reaction mixture was cooled and concentrated in vacuo. The crude material was taken directly into the fluorination.

Step K: Preparation of 4-bromo-2,2-difluoro-7-((trifluoromethyl)sulfonyl)-2,3-dihydro-1H-inden-1-one: Crude (E,Z)-3-((4-bromo-7-((trifluoromethyl)sulfonyl)-2,3-dihydro-1H-inden-1-ylidene)amino)propan-1-ol (3.75 g, 9.1 mmol) was dissolved in dry acetonitrile (23 mL) and added dropwise to a warm (60° C.), suspension of Selectfluor (9.6 g, 27.2 mmol) and sodium sulfate (12.9 g, 90.5 mmol) slurried in acetonitrile (10 mL). After the addition, the mixture was heated to 60° C. for 10 minutes then cooled to ambient temperature and treated with 10% HCl (15 mL) and stirred for 20 minutes. The mixture was adjusted to pH 8 with solid NaHCO₃ then diluted with ethyl acetate and separated. The aqueous was washed with ethyl acetate and the combined organics were washed with saturated NaHCO₃, saturated NaCl, dried over Na₂SO₄ filtered, and concentrated in vacuo to dark oil. The crude material was chromatographed on SiO₂ eluting with a gradient of ethyl acetate/hexane. The desired product was concentrated to a light yellow solid, (2.27 g, 66%). H NMR (400 MHz, CDCl₃): δ 8.22-8.14 (m, 2H), 3.60-3.55 (t, 2H).

Step L: Preparation of (S)-4-bromo-2,2-difluoro-7-((trifluoromethyl)sulfonyl)-2,3-dihydro-1H-inden-1-ol: 4-Bromo-2,2-difluoro-7-((trifluoromethyl)sulfonyl)-2,3-dihydro-1H-inden-1-one (1.65 g, 4.35 mmol) was dissolved in isopropanol (21 mL) and treated with triethylamine (1.2 mL, 8.7 mmol), formic acid (0.49 mL, 13.1 mmol) and RuCl(p-cymene)[(R,R)-Ts-DPEN] (27.7 mg, 0.040 mmol). The reaction mixture was stirred at ambient temperature for 4 hours. The solvent was removed in vacuo then the crude material was chromatographed on SiO₂ eluting with a gradient of ethyl acetate/hexane. The product was isolated as a more pure fraction (1.83 g) and a slightly less pure fraction. Both of these fractions were successfully utilized in the coupling reaction. ¹H NMR (400 MHz, CDCl₃): δ 7.88-7.80 (m, 2H), 5.50-5.45 (m, 1H), 3.66-3.58 (m, 1H), 3.20 (m, 1H).

Step M: Preparation of (S)-3-((2,2-difluoro-1-hydroxy-7-((trifluoromethyl)sulfonyl)-2,3-dihydro-1H-inden-4-yl)amino)-5-fluorobenzonitrile: (S)-4-Bromo-2,2-difluoro-7-((trifluoromethyl)sulfonyl)-2,3-dihydro-1H-inden-1-ol (98 mg, 0.26 mmol) was dissolved in 1,4-dioxane (0.80 mL) and treated with benzonitrile, 3-amino-5-fluoro- (42 mg, 0.31 mmol), palladium (II) acetate (2.9 mg, 0.010 mmol), and Xantphos (14.9 mg, 0.030 mmol). The mixture was heated to 120° C. for 1.5 hours in the microwave reactor. The reaction mixture was cooled, diluted with ethyl acetate and water then separated. The aqueous was washed with ethyl acetate and the combined organics were washed with saturated NaHCO₃, saturated NaCl, dried over Na₂SO₄ and concentrated in vacuo. The crude dark oil was chromatographed on SiO₂ eluting with a gradient of ethyl acetate/hexane. The desired material was recovered in a slightly impure form. This material was re-chromatographed on reversed-phase SiO₂ eluting with a gradient of MeCN/water. A single fraction was collected and to light tan solid, (35 mg, 31%). LCMS ESI (−) m/z (M−H) 435; H NMR (400 MHz, CDCl₃): δ 7.87 (d, 1H), 7.31-7.29 (m, 2H), 7.21-7.19 (m, 2H), 6.18 (m, 1H), 5.42-5.38 (m, 1H), 3.52-3.41 (m, 1H), 3.32-3.24 (m, 1H).

### Example 7: Synthesis of (S,3R)-4-((3-chloro-5-fluorophenyl)thio)-2,2,3-trifluoro-7-(methylsulfonyl)-2,3-dihydro-1H-inden-1-ol (Compound 491)

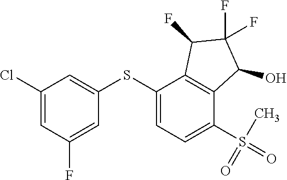 [SMILES: CS(=O)(=O)c1ccc(Sc2cc(F)cc(Cl)c2)c2c1[C@H](O)C(F)(F)[C@@H]2F]

Step A: Preparation of 4,7-difluoro-1H-indene-1,3(2H)-dione (0.52 g, 2.8 mmol) was slurried acetic anhydride (2.5 mL, 27 mmol) and treated with tert-butyl 3-oxobutanoate (0.52 mL, 3.1 mmol) and triethylamine (1.4 mL, 10 mmol). The mixture was stirred at ambient temperature for 60 hours. The reaction was cooled to 0° C. and treated with 10% aqueous hydrochloric acid (8.6 mL, 25 mmol) by dropwise addition. After the addition, the mixture was warmed to ambient temperature then heated to 75° C. for 10 minutes. After cooling, the mixture was diluted with water (20 mL) and extracted three times with methylene chloride (20 mL portions). The combined organics were dried over Na₂SO₄ and concentrated in vacuo to crude orange solid. This material was carried forward without purification.

Step B: Preparation of 2,2,4,7-tetrafluoro-H-indene-1,3(2H)-dione: 4,7-difluoro-1H-indene-1,3(2H)-dione (0.51 g, 2.8 mmol) was dissolved in acetonitrile (27 mL), placed in an ambient temperature water bath then treated with solid sodium carbonate (950 mg, 9.0 mmol) followed by Selectfluor® (2.18 g, 6.2 mmol). The mixture was stirred at ambient temperature for 1 hour. The mixture was filtered to removed undissolved solids, the solids were washed with ethyl acetate and the filtrate was concentrated in vacuo. The residue was redissolved in water (ca. 20 mL) and extracted four times with ethyl acetate (20 mL each). The combined organics were washed with saturated NaCl, dried over Na₂SO₄ and concentrated in vacuo to orange solid. The crude solid was chromatographed on SiO₂ eluting with an aggressive gradient of ethyl acetate/hexane. The desired material concentrated to orange solid, (493 mg, 81%). ¹H NMR (400 MHz, CDCl₃): δ 7.70-7.65 (2H).

Step C: Preparation of (S)-2,2,4,7-tetrafluoro-3-hydroxy-2,3-dihydro-1H-inden-1-one: 2,2,4,7-tetrafluoro-1H-indene-1,3(2H)-dione (5.81 g, 26.6 mmol) was suspended in methylene chloride (260 mL), cooled to 0° C., and treated with formic acid (1.01 mL, 26.6 mmol), triethylamine (2.60 mL, 18.6 mmol), then the reaction mixture was sparged with argon for 5 minutes. RuCl(p-cymene)[(S,S)-Ts-DPEN] (339 mg, 0.530 mmol) was added and the reaction was transferred to the refrigerator and allowed to stand at 4° C. for 20 hours. The cold reaction mixture was poured into cold 1N aqueous HCl (70 mL) and separated. The aqueous was washed twice with ethyl acetate then the combined organic layers were dried over Na₂SO₄ and concentrated in vacuo to a brown semi-solid. The crude material was chromatographed on SiO₂ eluting with a gradient of ethyl acetate/hexane. The product was recovered as yellow solid, (3.48 g, 59%). ¹H NMR (400 MHz, CDCl₃): δ 7.86-7.80 (m, 1H), 7.60-7.54 (m, 1H), 5.79-5.74 (m, 1H), 3.23-3.18 (m, 1H).

Step D: Preparation of (S)-2,2,4-trifluoro-3-hydroxy-7-(methylthio)-2,3-dihydro-1H-inden-1-one: (S)-2,2,4,7-tetrafluoro-3-hydroxy-2,3-dihydro-1H-inden-1-one (0.40 g, 1.8 mmol) was dissolved in dry acetonitrile (18 mL), cooled to 0° C., and sparged with argon for 5 minutes. The solution was treated in a single portion with sodium thiomethoxide (144 mg, 2.06 mmol) and after 5 minutes, the ice bath was removed and the reaction was stirred at ambient temperature for 3 hours. The reaction mixture was concentrated in vacuo and the residue was redissolved in water and ethyl acetate. After separation, the aqueous was washed twice with ethyl acetate and the combined organics were washed with saturated NaCl, dried over Na₂SO₄ and concentrated in vacuo. The orange residue was chromatographed on SiO₂ eluting with a gradient of ethyl acetate/hexane. The desired material was recovered as bright yellow solid, (314 mg, 70%). LCMS ESI (+) m/z (M+H) 249.

Step E: Preparation of (S)-2,2,4-trifluoro-3-hydroxy-7-(methylsulfonyl)-2,3-dihydro-1H-inden-1-one: (S)-2,2,4-trifluoro-3-hydroxy-7-(methylthio)-2,3-dihydro-1H-inden-1-one (0.40 g, 1.6 mmol) was dissolved in MeOH (10 mL) and the reaction was treated dropwise with a solution of Oxone® (2.2 g, 3.6 mmol) dissolved in water (10 mL). The mixture was stirred at ambient temperature for 14 hours. The reaction mixture was filtered, the solids were washed with ethyl acetate and the filtrate was concentrated in vacuo to remove volatile solvents. The aqueous filtrate was extracted three times with ethyl acetate then the combined organics were washed with saturated NaCl, dried over Na₂SO₄ and concentrated in vacuo to yellow solid, (467 mg, quant.). LCMS ESI (+) m/z (M+H) 281.

Step F: Preparation of (R)-2,2,3,4-tetrafluoro-7-(methylsulfonyl)-2,3-dihydro-1H-inden-1-one: (S)-2,2,4-trifluoro-3-hydroxy-7-(methylsulfonyl)-2,3-dihydro-1H-inden-1-one (0.45 g, 1.6 mmol) was dissolved in dichloromethane (16 mL), cooled to 0° C., and treated dropwise with diethylaminosulfur trifluoride (DAST) (0.32 mL, 2.4 mmol) and stirred at 0° C. for 14 hours. The reaction was treated with additional diethylaminosulfur trifluoride (0.32 mL, 2.4 mmol) and stirring continued for 6 hours at 0° C. The cold reaction was treated with saturated NaHCO₃ (10 mL) and stirred vigorously for 20 minutes. The mixture was diluted with additional methylene chloride and the layers were separated. The aqueous was re-extracted with methylene chloride and the combined organic layers were dried over Na₂SO₄ and concentrated in vacuo to a yellow solid. The crude material was chromatographed on SiO₂ eluting with a gradient of ethyl acetate/hexane. The desired material was recovered as pale yellow solid, (258 mg, 53%). LCMS ESI (+) m/z (M+H) 283.

Step G: Preparation of (1S,3R)-2,2,3,4-tetrafluoro-7-(methylsulfonyl)-2,3-dihydro-1H-inden-1-ol: (R)-2,2,3,4-tetrafluoro-7-(methylsulfonyl)-2,3-dihydro-1H-inden-1-one: (0.098 g, 0.35 mmol) was suspended in methylene chloride (3.3 mL), cooled to 0° C., and treated with triethylamine (97 μL, 0.69 mmol), formic acid (39 μL, 1.0 mmol) and RuCl(p-cymene)[(R,R)-Ts-DPEN] (2.2 mg, 0.003 mmol). The solution was allowed to stand at 4° C. in the refrigerator for 60 hours. The reaction mixture was concentrated in a stream of nitrogen gas then chromatographed on SiO₂ eluting with a gradient of ethyl acetate/hexane. The desired fractions were concentrated to colorless film, (53 mg, 53%). LCMS ESI (+) m/z (M+H) 285.

Step H: Preparation of (1S,3R)-4-((3-chloro-5-fluorophenyl)thio)-2,2,3-trifluoro-7-(methylsulfonyl)-2,3-dihydro-1H-inden-1-ol: (1S,3R)-2,2,3,4-tetrafluoro-7-(methylsulfonyl)-2,3-dihydro-1H-inden-1-ol (0.005 g, 0.02 mmol) was treated with cesium bicarbonate (17 mg, 0.090 mmol) and suspended in DMF (0.1 mL) then stirred at ambient temperature for 1 hour. 3-Chloro-5-fluorothiophenol (14 mg, 0.090 mmol) was added and the mixture was stirred at ambient temperature for 18 hours. The reaction was concentrated in a stream of nitrogen gas to remove DMF. The residue was chromatographed on SiO₂ eluting with a stepped gradient of ethyl acetate/hexane. Compound 491 was concentrated to light pink oil, (7 mg, 93%). LCMS ESI (+) m/z (M+Na) 449; ¹H NMR (400 MHz, CDCl₃): δ 7.99-7.95 (m, 1H), 7.33-7.32 (m, 1H), 7.24-7.19 (m, 2H), 7.16-7.13 (m, 1H), 5.75 (dd, 1H), 5.68-5.65 (m, 1H), 3.37-3.36 (m, 1H), 3.23 (s, 3H).

### Example 8: Synthesis of 4-(2-hydroxyethyl)-7-((trifluoromethyl)sulfonyl)-2,3-dihydro-1H-inden-1-ol (Compound 495)

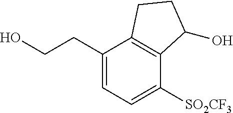

Step A: Preparation of diethyl 2-[7′-(trifluoromethylsulfonyl)spiro[1,3-dioxolane-2,1′-indane]-4′-yl]propanedioate: Tetrahydrofuran (12.0 mL) was added all at once to sodium hydride (735.6 mg, 18.39 mmol) at 0° C. under nitrogen followed by the slow addition of diethyl malonate (1.86 mL, 12.26 mmol). Stirred for 15 min then a solution of 4′-fluoro-7′-(trifluoromethylsulfonyl)spiro[1,3-dioxolane-2,1′-indane] (1.0 g, 3.07 mmol) in tetrahydrofuran (3.0 mL) was added by syringe over 2 minutes. The reaction mixture was then removed from the cooling bath and stirred at room temperature overnight. Additional sodium hydride (200 mg) was added as well as diethyl malonate (0.5 mL) and stirred an additional 6 h. Cooled to 0° C., quenched with water, extracted with ethyl acetate, washed with brine, dried over sodium sulfate, filtered and concentrated in vacuo. Purified on silica gel (25 g SNAP Ultra, 10-100% ethyl acetate/hexane) affording diethyl 2-[7′-(trifluoromethylsulfonyl)spiro[1,3-dioxolane-2,1′-indane]-4′-yl]propanedioate (940 mg, 2.0 mmol, 66% yield).

Step B: Preparation of 2-[1-oxo-7-(trifluoromethylsulfonyl)indan-4-yl]acetic acid: HCl (4.84 mL, 29.03 mmol) was added to diethyl 2-[7′-(trifluoromethylsulfonyl)spiro[1,3-dioxolane-2,1′-indane]-4′-yl]propanedioate (300.0 mg, 0.64 mmol) then warmed to 100° C. for 6 h. Cooled to room temperature, extracted with MTBE, washed with water, brine, dried over Na₂SO₄, filtered and concentrated in vacuo affording 2-[1-oxo-7-(trifluoromethylsulfonyl)indan-4-yl]acetic acid (200.0 mg, 0.62 mmol, 96% yield). Used without further purification.

Step C: Preparation of 4-(2-hydroxyethyl)-7-(trifluoromethylsulfonyl)indan-1-ol: Borane dimethylsulfide complex (434.4 μL, 0.87 mmol) was added slowly to 2-[1-oxo-7-(trifluoromethylsulfonyl)indan-4-yl]acetic acid (70.0 mg, 0.22 mmol) in tetrahydrofuran (1.5 mL) at room temperature and stirred for 2 h. Cooled to 0° C. and quenched with 1 N HCl, extracted with ethyl acetate, washed with brine, dried over MgSO₄, filtered and concentrated in vacuo. Purified on silica gel (10 g SNAP Ultra, 14 CV, 60-100% ethyl acetate/hexane) affording 4-(2-hydroxyethyl)-7-(trifluoromethylsulfonyl)indan-1-ol (36.0 mg, 0.12 mmol, 53% yield). Hexane was added to the clear oil and then cooled to −78° C. with scratching until a white gum was observed, warmed to room temperature and continued scratching until a white powder formed. Hexane was then removed under a stream of nitrogen to afford Compound 495. LC-MS (−) ESI m/z 309 (M−H). H-NMR (400 MHz, CDCl₃): δ 7.83 (d, 1H), 7.47 (d, 1H), 5.61 (d, 1H), 3.95-3.92 (m, 1H), 3.27-3.18 (m, 1H), 3.10 (s, 1H), 2.99-2.96 (m, 3H), 2.41-2.26 (m, 2H).

### Example 9: HIF-2α Scintillation Proximity Assay (SPA)

The total assay volume was about 100 μL in the following configuration: 2 μL compound in 100% DMSO, 88 μL buffer with protein and probe and 10 μL of SPA beads. The compound was diluted in a master plate consisting of a 10-point dose response with a 3-fold compound dilution from 100 μM to 5 nM. Assays were run on a 96-well plate in which one column, designated as the high signal control, contained DMSO with no compound and another column, designated as the low signal control, contained no protein. Prior to plating out of compound, a buffer solution, consisting of 25 mM TRIS pH 7.5 (Sigma), 150 mM NaCl (Sigma), 15% Glycerol (Sigma), 0.15% BSA (Sigma), 0.001% Tween-20 (Sigma), 150 nM N-(3-Chlorophenyl-4,6-t₂)-4-nitrobenzo[c][1,2,5]oxadiazol-5-amine (Compound 183) and 100 nM HIF-2α HIS TAG-PASB Domain, was made and allowed to equilibrate for 30 minutes. Compounds that were to be tested were then plated in to a 96-well white clear bottom Isoplate-96 SPA plate (Perkin Elmer). To the compounds was added 88 μL of the buffer solution, then the plate covered with a plastic cover and aluminum foil, placed onto a shaker and equilibrated for 1 hour. After equilibration, 10 μL of a 2 mg/mL solution of YSi Cu His tagged SPA beads (Perkin Elmer) were then added to each well of the plate, covered and equilibrated for another 2 hours. The plates were then removed from the shaker, placed into a 1450 LSC and luminescence counter MicroBeta Trilux (Perkin Elmer) to measure the extent of probe displacement. The percent inhibition was determined and IC₅₀ values were calculated using the Dotmatics system based on the following equation: % inhibition=[(high control−sample)/(high control−low control)]×100.

### Example 10: VEGF ELISA Assay

About 7500 786-O cells in 180 μL of growth medium were seeded into each well of a 96-well, white, clear bottom plate (07-200-566, Fisher Scientific) on day one. Four hours later, serial dilutions of 10× compound stocks were made in growth medium from 500×DMSO stocks, and 20 μL of those 10× stocks were added to each well to make final concentrations as follows (μM): 20, 6.67, 2.22, 0.74, 0.25, 0.082, 0.027, 0.009, 0.003, 0.001, and 0. Each concentration was plated in duplicate. About 20 hours later, medium was removed by suction and each well was supplied with 180 μL of growth medium. About 20 μl freshly-made 10× compound stocks were added to each well. About 24 hours later, cell culture medium was removed and the VEGF concentration determined using an ELISA kit purchased from R&D systems, following the manufacturer's suggested method. The EC₅₀ was calculated by GraphPad Prism using the dose-response-inhibition (four parameter) equation. The cell-seeded plate was then subjected to CellTiter-Glo luminescence cell viability assay (Promega) by adding 50 μL of Celltiter Glo reagent into each well and shaking the plate for 8 minutes at 550 rpm (Thermomixer R, Eppendorf) then the luminescence signal immediately read in a plate reader (3 second delay, 0.5 second/well integration time, Synergy 2 multi Detection Microplate reader).

### Example 11: Luciferase Assay

786-O-Hif-Luc single clone cells were obtained by infecting 786-0 cells (ATCC® CRL-1932′) with commercial lentivirus that delivers a luciferase gene driven by multiple HIF responsive elements (Cignal Lenti HIF Reporter (luc): CLS-007L, Qiagen) at Multiplicity of Infection (MOI) of 25 for 24 hours. The cells were replenished with fresh medium (Dulbecco's Modified Eagle's Medium (DMEM, D5796, Sigma) supplemented with 10% FBS (F6178, Sigma), 100 units penicillin and 100 g streptomycin/mL (P4333, Sigma)) for another 24 hours. A pool of infected cells were then selected against 2 g/mL of puromycin (P8833, Sigma) for 10 days followed by limited dilution to select single clones. The clones were tested for their response to HIF-2 inhibitors and the ones that showed the biggest dynamic range (786-O-Hif-Luc) were expanded and used for the luciferase assay. For the luciferase assay, about 7500 786-O-Hif-Luc cells in 90 μL growth medium were seeded into each well of a 96-well white opaque plate (08-771-26, Fisher scientific) a day before treatment.

On treatment day, serial dilutions of 10× compound stocks were made in growth medium from 500×DMSO stocks, and 10 μL of the 1× stocks were added to each well to make final concentrations as follows (M): 20, 6.67, 2.22, 0.74, 0.25, 0.08, 0.027, 0.009, 0.003, 0.001, and 0. Each concentration was tested in triplicate. After about 24 hours, luciferase activity was determined using ONE-Glo Luciferase Assay Reagent (E6110, Promega) following the manufacturer's recommended procedure. EC₅₀ were calculated using Dotmatics software.

Table 3 shows biological activities of selected compounds in Luciferase, VEGF ELISA and Scintillation Proximity assays. Compound numbers correspond to the numbers and structures provided in Table 1 and Examples 1-8.

TABLE 3

| | Less than | 50 nM to | 250 nM to | Greater than |
|---|---|---|---|---|
| | 50 nM (++++) | 249 nM (+++) | 1000 nM (++) | 1000 nM (+) |
| Scintillation | 1, 2, 6, 7a, 8, 9, | 17, 18, 21, 33, 38, | 3, 5, 16, 22, 24, | 4, 7b, 12, 13, 14, |
| Proximity | 11, 15, 25, 26, 29, | 39, 41, 50, 54, 74, | 27, 31, 40, 42, 43, | 19, 20, 23, 28, 35, |
| Assay | 30, 32, 34, 52, 55, | 75, 90, 93, 94, 99, | 46, 48, 53, 70, 71, | 36, 37, 44, 45, 47, |
| IC₅₀ (nM) | 56, 57, 58, 59, 60, | 100, 102, 107, | 72, 76, 78, 81, 91, | 49, 51, 66, 68, 69, |
| | 61, 62, 63, 64, 65, | 116, 117, 118, | 103, 104, 106, | 73, 77, 79, 82, 83, |
| | 67, 80, 92, 98, | 119, 128, 136, | 115, 120, 131, | 84, 85, 86, 87, 88, |
| | 101, 111, 112, | 141, 145, 146, | 132, 133, 134, | 89, 95, 96, 97, |
| | 123, 124, 140, | 147, 148, 153, | 135, 137, 152, | 105, 108, 109, |
| | 143, 144, 155, | 156, 162, 165, | 157, 172, 178, | 110, 113, 114, |
| | 158, 159, 160, | 187, 192, 195, | 182, 184, 190, | 121, 122, 125, |
| | 161, 163, 166, | 203, 204, 214, | 193, 197, 202, | 126, 127, 129, |
| | 167, 168, 179, | 224, 237, 241, | 207, 209, 212, | 130, 138, 139, |
| | 185, 186, 188, | 242, 252, 254, | 213, 218, 220, | 142, 149, 150, |
| | 191, 194, 196, | 260, 265, 267, | 243, 244, 249, | 151, 154, 164, |
| | 198, 200, 201, | 270, 274, 275, | 258, 261, 264, | 169, 170, 171, |
| | 206, 215, 221, | 276, 277, 285, | 269, 271, 281, | 173, 174, 175, |
| | 223, 225, 227, | 295, 302, 308, | 287, 293, 307, | 176, 177, 180, |
| | 228, 229, 230, | 312, 324, 325, | 339, 346, 369, | 181, 189, 199, |
| | 231, 232, 233, | 333, 334, 336, | 374, 411, 422, | 205, 208, 210, |
| | 234, 235, 236, | 353, 358, 361, | 423, 432, 433, | 211, 216, 217, |
| | 240, 245, 247, | 370, 378, 381, | 434, 471, 476, | 219, 222, 226, |
| | 251, 256, 263, | 383, 387, 388, | 481, 482, 487, | 238, 239, 246, |
| | 266, 273, 286, | 399, 405, 409, | 496, 505, 506, | 248, 250, 253, |
| | 289, 290, 292, | 412, 414, 421, | 511, 513, 522, | 255, 257, 259, |
| | 303, 304, 305, | 424, 426, 427, | 523, 531, 542, | 262, 268, 272, |
| | 306, 309, 310, | 437, 442, 444, | 575, 580, 591, | 278, 279, 280, |
| | 314, 315, 316, | 462, 468, 477, | 595, 601, 603, | 282, 283, 284, |
| | 317, 319, 321, | 480, 488, 517, | 612, 622, 637, | 288, 291, 294, |
| | 327, 328, 329, | 545, 547, 551, | 644, 648, 678, | 296, 297, 298, |
| | 331, 338, 340, | 554, 563, 569, | 703, 707, 711, | 299, 300, 301, |
| | 341, 342, 344, | 584, 586, 598, | 735, 748, 749, | 311, 313, 318, |
| | 347, 348, 349, | 604, 609, 614, | 750, 754, 762, | 320, 322, 323, |
| | 355, 356, 357, | 616, 620, 660, | 765, 770, 775, | 326, 330, 332, |
| | 359, 360, 364, | 663, 669, 672, | 781, 812, 816, | 335, 337, 343, |
| | 365, 368, 371, | 676, 681, 682, | 827 | 345, 350, 351, |
| | 372, 375, 376, | 686, 696, 697, | | 352, 354, 362, |
| | 379, 386, 389, | 700, 701, 706, | | 363, 366, 367, |
| | 392, 397, 398, | 714, 717, 718, | | 373, 377, 380, |
| | 401, 403, 430, | 730, 734, 737, | | 382, 384, 385, |
| | 431, 435, 436, | 747, 751, 755, | | 390, 391, 393, |
| | 440, 443, 446, | 759, 768, 769, | | 394, 395, 396, |
| | 451, 458, 465, | 771, 782, 784, | | 400, 402, 404, |
| | 466, 467, 472, | 788, 795, 808, | | 406, 407, 408, |
| | 473, 478, 483, | 815, 818 | | 410, 413, 415, |
| | 485, 486, 489, | | | 416, 417, 418, |
| | 491, 507, 509, | | | 419, 420, 425, |
| | 532, 549, 550, | | | 428, 429, 438, |
| | 557, 571, 572, | | | 439, 441, 445, |
| | 573, 574, 576, | | | 447, 448, 449, |
| | 577, 578, 579, | | | 450, 452, 453, |
| | 581, 582, 585, | | | 454, 455, 456, |
| | 587, 593, 594, | | | 457, 459, 460, |
| | 602, 605, 607, | | | 461, 463, 464, |
| | 608, 610, 617, | | | 469, 470, 474, |
| | 623, 626, 654, | | | 475, 484, 490, |
| | 655, 656, 657, | | | 492, 493, 494, |
| | 658, 659, 661, | | | 495, 497, 498, |
| | 675, 677, 699, | | | 499, 500, 501, |
| | 704, 709, 710, | | | 502, 503, 504, |
| | 712, 713, 715, | | | 508, 510, 512, |
| | 716, 727, 728, | | | 514, 515, 516, |
| | 731, 736, 739, | | | 518, 519, 520, |
| | 740, 741, 742, | | | 521, 524, 525, |
| | 743, 744, 746, | | | 526, 527, 528, |
| | 753, 756, 760, | | | 529, 530, 533, |
| | 761, 766, 774, | | | 534, 535, 536, |
| | 778, 783, 786, | | | 537, 538, 539, |
| | 787, 789, 790, | | | 540, 541, 543, |
| | 794, 796, 811, | | | 544, 546, 548, |
| | 813, 814, 825, | | | 552, 553, 555, |
| | 828, 831 | | | 556, 558, 559, |
| | | | | 560, 561, 562, |
| | | | | 564, 565, 566, |
| | | | | 567, 568, 570, |
| | | | | 583, 588, 589, |
| | | | | 590, 592, 596, |
| | | | | 597, 599, 600, |
| | | | | 606, 611, 613, |
| | | | | 615, 618, 619, |
| | | | | 621, 624, 625, |
| | | | | 627, 628, 629, |
| | | | | 630, 631, 632, |
| | | | | 633, 634, 635, |
| | | | | 636, 638, 639, |
| | | | | 640, 641, 642, |
| | | | | 643, 645, 646, |
| | | | | 647, 649, 650, |
| | | | | 651, 652, 653, |
| | | | | 662, 664, 665, |
| | | | | 666, 667, 668, |
| | | | | 670, 671, 673, |
| | | | | 674, 679, 680, |
| | | | | 683, 684, 685, |
| | | | | 687, 688, 689, |
| | | | | 690, 691, 692, |
| | | | | 693, 694, 695, |
| | | | | 698, 702, 705, |
| | | | | 708, 719, 720, |
| | | | | 721, 722, 723, |
| | | | | 724, 725, 726, |
| | | | | 729, 732, 733, |
| | | | | 738, 745, 752, |
| | | | | 757, 758, 763, |
| | | | | 764, 767, 772, |
| | | | | 773, 776, 777, |
| | | | | 779, 780, 785, |
| | | | | 791, 792, 793, |
| | | | | 797, 807, 809, |
| | | | | 810, 817, 819, |
| | | | | 820, 821, 822, |
| | | | | 823, 824, 826, |
| | | | | 829, 830, 833 |
| Mean | 8, 9, 11, 15, 25, | 2, 17, 67, 155, | 1, 34, 41, 74, 78, | 98, 133, 179, |
| VEGF | 55, 60, 63, 64, | 166, 188, 191, | 80, 99, 102, 124, | 274 |
| ELISA | 158, 159, 161, | 225, 231, 234, | 132, 165, 203, | |
| EC₅₀ (nM) | 163, 167, 185, | 240, 245, 252, | 267, 353, 387, | |
| | 186, 196, 228, | 254, 256, 303, | 424, 473, 495, | |
| | 230, 233, 235, | 304, 310, 325, | 577, 734 | |
| | 236, 251, 273, | 342, 347, 349, | | |
| | 289, 292, 305, | 355, 357, 360, | | |
| | 306, 309, 316, | 365, 372, 398, | | |
| | 317, 364, 368, | 399, 421, 431, | | |
| | 375, 389, 403, | 467, 507, 574, | | |
| | 446, 451, 458, | 576, 578, 605, | | |
| | 465, 478, 489, | 610, 620, 659, | | |
| | 571, 572, 579, | 706, 710, 713, | | |
| | 617, 654, 655, | 736, 740, 743, | | |
| | 656, 657, 658, | 760, 761, 783, | | |
| | 709, 712, 742, | 784, 825 | | |
| | 744, 813, 828 | | | |
| Mean | 8, 9, 11, 15, 25, | 1, 2, 6, 17, 26, 27, | 3, 5, 7a, 34, 38, | 16, 18, 20, 21, 22, |
| Luciferase | 55, 63, 64, 65, | 56, 57, 58, 59, 60, | 39, 41, 42, 43, 52, | 31, 32, 33, 35, 40, |
| EC₅₀ (nM) | 158, 159, 160, | 61, 62, 67, 155, | 54, 74, 80, 81, 90, | 46, 50, 53, 75, 85, |
| | 161, 163, 166, | 162, 165, 187, | 92, 93, 94, 99, | 91, 98, 100, 103, |
| | 167, 185, 186, | 188, 191, 200, | 101, 107, 112, | 104, 110, 111, |
| | 196, 215, 221, | 206, 223, 224, | 115, 124, 144, | 114, 116, 117, |
| | 225, 227, 228, | 237, 241, 245, | 145, 146, 156, | 118, 119, 120, |
| | 229, 230, 231, | 251, 252, 254, | 168, 192, 194, | 128, 131, 132, |
| | 232, 233, 234, | 260, 267, 270, | 198, 203, 207, | 134, 135, 136, |
| | 235, 236, 240, | 274, 276, 277, | 213, 242, 243, | 140, 143, 147, |
| | 247, 256, 266, | 285, 286, 290, | 263, 265, 271, | 148, 151, 152, |
| | 273, 289, 292, | 302, 310, 314, | 275, 287, 293, | 157, 172, 181, |
| | 303, 304, 305, | 315, 334, 336, | 294, 295, 308, | 182, 190, 195, |
| | 306, 309, 316, | 342, 348, 355, | 312, 324, 325, | 201, 202, 209, |
| | 317, 338, 347, | 357, 360, 365, | 339, 353, 359, | 212, 214, 218, |
| | 349, 364, 368, | 369, 372, 381, | 361, 370, 377, | 220, 222, 226, |
| | 371, 375, 380, | 383, 387, 388, | 378, 390, 411, | 244, 249, 250, |
| | 386, 389, 399, | 392, 398, 401, | 414, 416, 422, | 253, 255, 258, |
| | 403, 430, 431, | 405, 409, 421, | 426, 432, 434, | 259, 261, 264, |
| | 435, 446, 451, | 423, 424, 436, | 437, 440, 443, | 268, 269, 272, |
| | 458, 465, 467, | 473, 477, 480, | 444, 462, 466, | 279, 291, 296, |
| | 478, 489, 571, | 486, 488, 507, | 468, 471, 472, | 300, 301, 307, |
| | 572, 576, 579, | 523, 547, 549, | 482, 483, 505, | 313, 352, 356, |
| | 582, 584, 602, | 573, 574, 578, | 517, 531, 532, | 358, 363, 374, |
| | 605, 617, 654, | 593, 594, 598, | 550, 557, 563, | 376, 379, 385, |
| | 655, 656, 657, | 604, 610, 620, | 569, 577, 585, | 394, 397, 400, |
| | 658, 661, 675, | 623, 626, 659, | 591, 595, 608, | 412, 427, 428, |
| | 704, 709, 710, | 676, 677, 681, | 616, 622, 644, | 433, 439, 441, |
| | 712, 716, 727, | 686, 696, 697, | 660, 663, 669, | 455, 456, 464, |
| | 728, 742, 743, | 699, 706, 713, | 678, 682, 707, | 470, 474, 476, |
| | 744, 746, 783, | 715, 717, 730, | 711, 714, 718, | 481, 485, 487, |
| | 786, 787, 789, | 731, 734, 736, | 747, 748, 750, | 491, 492, 495, |
| | 790, 794, 813, | 740, 760, 761, | 753, 756, 759, | 496, 506, 509, |
| | 828, 831 | 781, 782, 784, | 762, 766, 768, | 511, 513, 516, |
| | | 795, 814, 818, | 769, 771, 774, | 534, 538, 542, |
| | | 825 | 775, 788, 796 | 545, 551, 554, |
| | | | | 558, 565, 570, |
| | | | | 575, 580, 581, |
| | | | | 586, 601, 603, |
| | | | | 607, 609, 612, |
| | | | | 614, 618, 619, |
| | | | | 637, 639, 665, |
| | | | | 670, 672, 683, |
| | | | | 692, 700, 701, |
| | | | | 703, 721, 722, |
| | | | | 723, 724, 725, |
| | | | | 726, 735, 737, |
| | | | | 739, 741, 749, |
| | | | | 751, 752, 754, |
| | | | | 755, 764, 765, |
| | | | | 767, 770, 778, |
| | | | | 779, 780, 808, |
| | | | | 811, 812, 833 |

### Example 12: Improvement in Disease Activity Index and Colon Length in Murine Colitis Model Treated with a HIF-2α Inhibitor

6-8 week old female C57BI6 mice were tagged and weighed, then treated with 2% dextran sulfate sodium (DSS) for 6 days. A 2 week recovery phase of regular water was completed over days 7-19. On day 20, a second cycle of 2.5% DSS was started, along with treatment of vehicle (MCT; BID), Compound 231 (60 mg/kg, BID) or filgotinib (30 mg/kg, QD). Mice were monitored daily and scored for body weight loss, stool consistency/diarrhea, and blood in the feces/rectum. The disease activity index was calculated according to Table 4. The second DSS cycle and treatment regimen continued for 7 days, and then on day 27 the animals were sacrificed. Colons were harvested and the texture, appearance and length of the colons assessed. Colon tissues were prepared for H&E histology, immune gene expression analysis, and immune phenotyping of immune cell infiltrate by flow cytometry.

TABLE 4

| | Score |
|---|---|
| Weight Loss | |
| None | 0 |
| 1-5% | 1 |
| 6-10% | 2 |
| 11-20% | 3 |
| >20% | 4 |
| Stool Consistency | |
| Normal | 0 |
| | 1 |
| Loose Stool | 2 |
| | 3 |
| Diarrhea | 4 |
| Blood in Feces | |
| None | 0 |
| | 1 |
| Occult Bleeding | 2 |
| | 3 |
| Gross Bleeding | 4 |

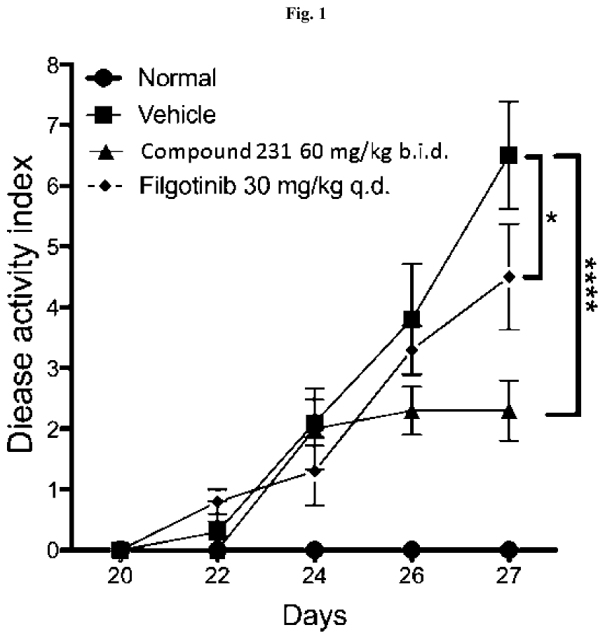

As shown in FIG. 1, the disease activity index score of the vehicle treated group at the end of the second DSS cycle was 6-7. Filgotinib-treated animals showed a slight decrease in the disease activity index, but only in the later stage of the disease progression. Animals treated with Compound 231 displayed an early and significant control of the disease progression, with a disease activity index score of approximately 2 observed at the endpoint of the experiment.

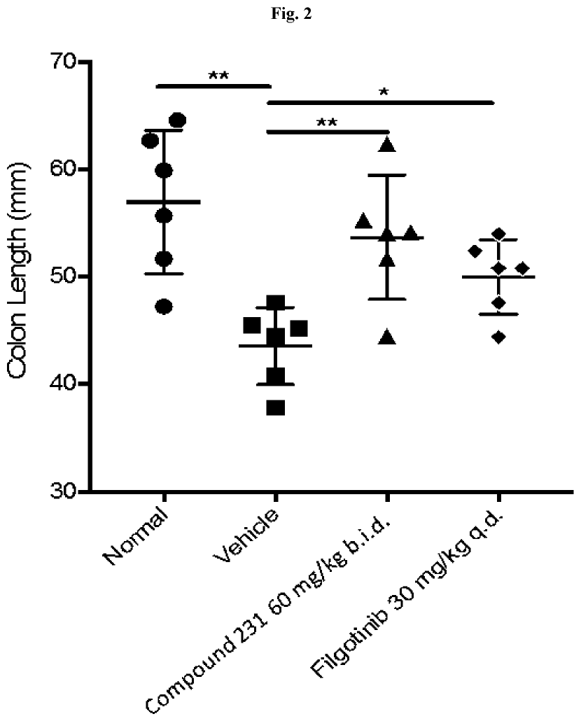

As shown in FIG. 2, DSS treatment results in a significantly shortened colon. This effect was reversed with both filgotinib and Compound 231, with Compound 231-treated mice showing the greatest improvement in colon length. The “normal” group shown in FIG. 1 and FIG. 2 represents animals that were not given DSS and thus did not develop DSS-induced colitis.

### Example 13: Effects of a HIF-2α Inhibitor Administered in a 10-Day DSS-Induced Acute Ulcerative Colitis Murine Model

A study was conducted to evaluate the effects of Compound 231 administered in female C57Bl/6 mice with dextran sulfate sodium (DSS)-induced chronic ulcerative colitis (UC) according to the following protocols: On study day 0, mice were given 2.5% DSS in drinking water for 7 days to induce colitis. Twice per day (BID), mice were orally administered (PO) vehicle (0.5% methyl cellulose and 0.5% Tween 80 in sterile water) or Compound 231 (100 mg/kg, prepared by adding Compound 231 to a necessary volume of vehicle and sonicating until a homogenous suspension was reached for PO dosing at 10 mL/kg) or were administered positive control cyclosporin A at 75 mg/kg once per day (QD, prepared in Kolliphor EL (Sigma, Cat #C5135, Lot #BCBQ5632V) and 1% CMC (BBP, Batch 2018, Lot #1)) from study days 0-10. Body weights were recorded daily. Test article efficacy was evaluated based on animal body weight measurements, colon weights and lengths, disease activity index (DAI) scores, and colon histopathology. As shown in Table 5, vehicle treatment (Group 2) results in shortened colon length. Treatment with Compound 231 (Group 4) inhibited colon length decrease as compared to that of vehicle control in a statistically significant manner. Group 1 animals were not given DSS and thus did not develop DSS-induced colitis.

TABLE 5

| | | | | Disease | | | |
|---|---|---|---|---|---|---|---|
| | | Body | | Activity | | | Full |
| | | Weight | | Index | Full | Full | Colon |
| | | Change | Colon | (DAI) | Colon | Colon | Mucosal |
| | | Day 1- | Length | Summed | Edema | Summed | Thickness |
| Group | Treatment | 10 (g) | (cm) | Score | (μM) | Scores | (μM) |
| 1 | Naïve | †0.89 | †7.35 | †0.35 | †0.0 | †0.0 | †195.0 |
| | | (0.27) | (0.10) | (0.35) | (0.00) | (0.00) | (5.00) |
| 2 | Vehicle PO, BID | −3.29 | 4.60 | 25.05 | 65.0 | 8.7 | 340.0 |
| | | (0.47) | (0.08) | (1.83) | (6.12) | (0.52) | (15.00) |
| 3 | Cyclosporin A | *−0.78 | *5.50 | 22.70 | 65.0 | *6.6 | 295.0 |
| | (75 mg/kg) PO, QD | (0.36) | (0.13) | (1.33) | (10.00) | (0.71) | (9.35) |
| 4 | Compound 231 | 2.31 | *5.30 | 24.00 | 80.0 | 7.9 | 325.0 |
| | (30 mg/kg) PO, BID | (0.47) | (0.14) | (1.78) | (9.35) | (1.30) | (33.54) |

(SE) = Standard errors are displayed in parenthesis
†p < 0.05 Student's t-test or Mann-Whitney U Test vs. Vehicle control
*p < 0.05 ANOVA or K-W (w/Dunnett's or Dunn's) vs. Vehicle control

1. A method of reducing inflammation of the digestive system in a subject in need thereof, comprising administering to the subject an effective amount of a HIF-2α inhibitor, wherein the HIF-2α inhibitor is a compound of Formula I: 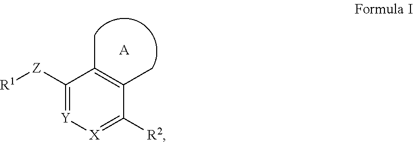
or a pharmaceutically acceptable salt or prodrug thereof, wherein:
X is selected from CR³ and N;
Y is selected from CR⁴ and N;
Z is selected from —O—, —S—, —S(O)—, —S(O)₂—, —C(O)—, —C(HR⁵)—, —N(R⁶)—, C₁-C₃ alkylene, C₁-C₃ heteroalkylene, C₁-C₃ alkenylene or absent;
A is selected from C₃₋₁₂ carbocycle and 3- to 12-membered heterocycle, each of which is optionally substituted with one or more R²⁰;
R¹ is selected from C₁₋₆ alkyl, C₃₋₁₂ carbocycle and 3- to 12-membered heterocycle, each of which is optionally substituted with one or more R²⁰;
R², R³, R⁴ and R⁵ are each independently selected from hydrogen and R²⁰;
R⁶ is selected from R²¹;
R²⁰ is independently selected at each occurrence from:
halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, and ═N(R²¹);
C₁₋₁₀ alkyl, C₂₋₁₀ alkenyl, and C₂₋₁₀ alkynyl, each of which is independently optionally substituted at each occurrence with one or more substituents selected from R²⁴; and
C₃₋₁₂ carbocycle and 3- to 12-membered heterocycle, each of which is independently optionally substituted at each occurrence with one or more substituents selected from R²⁵;
R²¹ is independently selected at each occurrence from hydrogen and C₁₋₂₀ alkyl, C₂₋₂₀ alkenyl, C₂₋₂₀ alkynyl, 1- to 6-membered heteroalkyl, C₃₋₁₂ carbocycle, and 3- to 12-membered heterocycle, each of which is optionally substituted by halogen, —CN, —NO₂, —NH₂, —NHCH₃, —NHCH₂CH₃, ═O, —OH, —OCH₃, —OCH₂CH₃, C₃₋₁₂ carbocycle, or 3- to 6-membered heterocycle;
R²² and R²³ are taken together with the nitrogen atom to which they are attached to form a heterocycle, optionally substituted with one or more R²⁰;
R²⁴ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₃₋₁₂ carbocycle, and 3- to 12-membered heterocycle, wherein each C₃₋₁₂ carbocycle and 3- to 12-membered heterocycle is independently optionally substituted with one or more substituents selected from R²⁵; and
R²⁵ is independently selected at each occurrence from halogen, —NO₂, —CN, —OR²¹, —SR²¹, —N(R²¹)₂, —NR²²R²³, —S(═O)R²¹, —S(═O)₂R²¹, —S(═O)(═NR²¹)R²¹, —S(═O)₂N(R²¹)₂, —S(═O)₂NR²²R²³, —NR²¹S(═O)₂R²¹, —NR²¹S(═O)₂N(R²¹)₂, —NR²¹S(═O)₂NR²²R²³, —C(O)R²¹, —C(O)OR²¹, —OC(O)R²¹, —OC(O)OR²¹, —OC(O)N(R²¹)₂, —OC(O)NR²²R²³, —NR²¹C(O)R²¹, —NR²¹C(O)OR²¹, —NR²¹C(O)N(R²¹)₂, —NR²¹C(O)NR²²R²³, —C(O)N(R²¹)₂, —C(O)NR²²R²³, —P(O)(OR²¹)₂, —P(O)(R²¹)₂, ═O, ═S, ═N(R²¹), C₁₋₆ alkyl, C₁₋₆ haloalkyl, C₂₋₆ alkenyl, and C₂₋₆ alkynyl.

2. The method of claim 1, wherein the subject suffers from inflammatory bowel disease.

3. The method of claim 1, wherein the subject suffers from Crohn's disease or colitis.

4. The method of claim 1, wherein the subject suffers from ulcerative colitis.

5-11. (canceled)

12. The method of claim 1, wherein A is selected from C₅ carbocycle and 5-membered heterocycle.

13. The method of claim 1, wherein A is substituted with at least one substituent selected from halogen, —OH, —OR²¹, —N(R²¹)₂, —NR²²R²³, C₁₋₁₀ alkyl, C₂₋₁₀ alkenyl, and C₂₋₁₀ alkynyl.

14. The method of claim 13, wherein A is substituted with at least one substituent selected from —F and —OH.

15. The method of claim 1, wherein the HIF-2α inhibitor is a compound of Formula I-C or a pharmaceutically acceptable salt thereof: 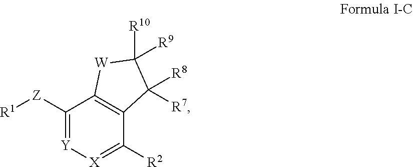
wherein:
W is selected from O, S, CR¹¹R¹² and NR⁶; and
R⁷, R⁸, R⁹, R¹⁰, R¹¹ and R¹² are each independently selected from hydrogen and R²⁰, or R⁷ and R¹ in combination form oxo or oxime.

16. The method of claim 15, wherein:
R⁷ is selected from hydrogen, halogen, —OR²¹, —N(R²¹)₂ and —NR²²R²³;
R⁸ is selected from hydrogen, C₁₋₁₀ alkyl, C₂₋₁₀ alkenyl, and C₂₋₁₀ alkynyl; and
R⁹, R¹⁰, R¹¹ and R¹² are each independently selected from hydrogen, halogen, —OR²¹, C₁₋₁₀ alkyl and 2- to 10-membered heteroalkyl.

16-20. (canceled)

21. The method of claim 15, wherein the HIF-2α inhibitor is a compound of Formula I-H, I-I, I-J or I-K:

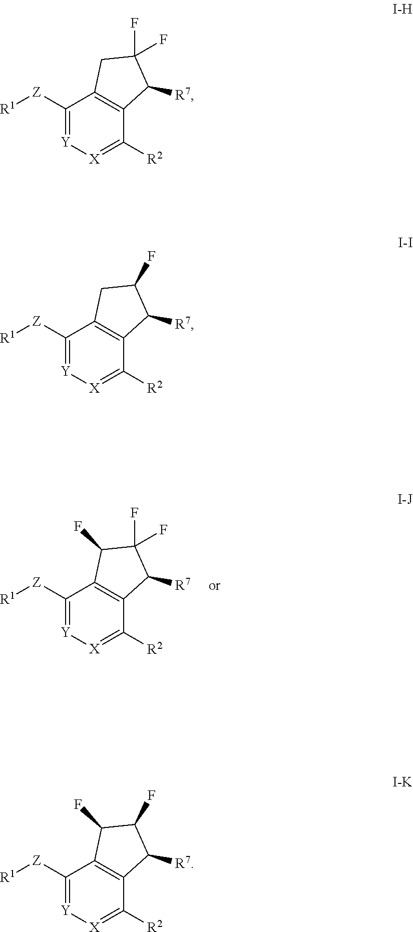

22-23. (canceled)

24. The method of claim 21, wherein R⁷ is —OH.

25. (canceled)

26. The method of claim 21, wherein R¹ is selected from phenyl and pyridyl.

27. (canceled)

28. The method of claim 21, wherein R¹ is substituted with at least one substituent selected from halogen, —CN, C₁₋₄ alkyl and C₁₋₄ alkoxy.

29-30. (canceled)

31. The method of claim 21, wherein R² is selected from —S(═O)₂CH₃, —S(═O)₂CHF₂, —S(═O)(═N—CN)CH₃ and CF₃.

32-33. (canceled)

34. The method of claim 21, wherein Z is —O—.

35. The method of claim 15, wherein:
R² is selected from —S(═O)₂R²¹, —S(═O)(═NR²¹)R²¹ and C₁₋₃ fluoroalkyl;
Z is —O—;
R⁷ is —OH; and
R⁸ is hydrogen.

36-38. (canceled)

39. The method of claim 21, wherein X is CR³ and Y is N.

40. The method of claim 21, wherein X is CR³ and Y is CR⁴.

41. (canceled)

42. The method of claim 1, wherein the HIF-2α inhibitor is:

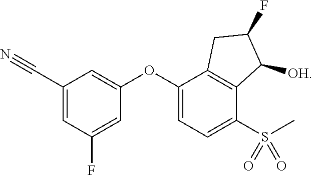 [SMILES: CS(=O)(=O)c1ccc(Oc2cc(F)cc(C#N)c2)c2c1[C@H](O)[C@H](F)C2]

43. The method of claim 1, wherein the HIF-2α inhibitor is selected from the group consisting of:

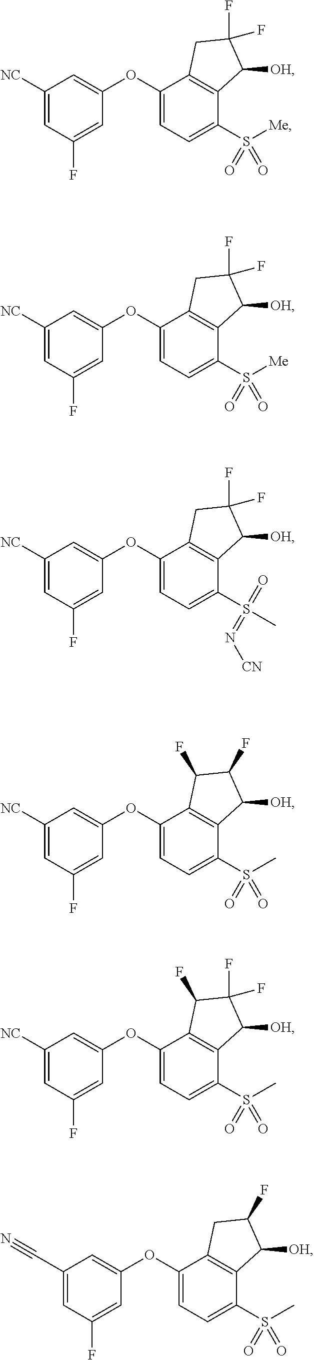 [SMILES: CS(=O)(=O)c1ccc(Oc2cc(F)cc(C#N)c2)c2c1[C@H](O)C(F)(F)C2 ; CS(=O)(=O)c1ccc(Oc2cc(F)cc(C#N)c2)c2c1[C@H](O)C(F)(F)C2 ; CS(=O)(=NC#N)c1ccc(Oc2cc(F)cc(C#N)c2)c2c1[C@H](O)C(F)(F)C2 ; CS(=O)(=O)c1ccc(Oc2cc(F)cc(C#N)c2)c2c1[C@H](O)[C@H](F)[C@@H]2F ; CS(=O)(=O)c1ccc(Oc2cc(F)cc(C#N)c2)c2c1[C@H](O)C(F)(F)[C@@H]2F ; CS(=O)(=O)c1ccc(Oc2cc(F)cc(C#N)c2)c2c1[C@H](O)[C@H](F)C2]

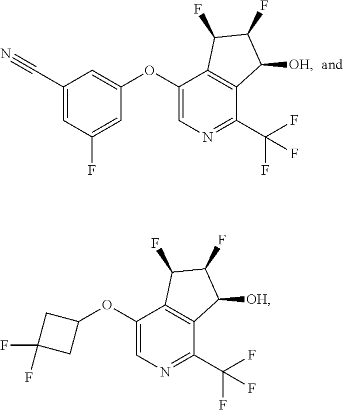 [SMILES: N#Cc1cc(F)cc(Oc2cnc(C(F)(F)F)c3c2[C@@H](F)[C@@H](F)[C@H]3O)c1 ; O[C@H]1c2c(C(F)(F)F)ncc(OC3CC(F)(F)C3)c2[C@@H](F)[C@H]1F]

44-50. (canceled)
